(19) United States
(12) Patent Application Publication
(10) Pub. No.: US 2026/0132140 A1
(43) Pub. Date: May 14, 2026

(54) IMIDAZOLYL-AMINO-PYRAZINE-CARBONITRILS AS CHK-1 INHIBITORS

(71) Applicant: BenevolentAI Bio Limited, London (GB)

(72) Inventors: Michael J. RAWLING, London (GB); Christine WATSON, London (GB); Andrew G. MALCOLM, London (GB); Michael K. CARTER, London (GB); Márton VASS, London (GB)

(21) Appl. No.: 19/113,409

(22) PCT Filed: Sep. 21, 2023
(86) PCT No.: PCT/GB2023/052446

§ 371 Date: Mar. 19, 2025

(30) Foreign Application Priority Data
Sep. 21, 2022 (GB) 2213792.1

(51) Int. Cl. C07D 487/10; A61K 31/497; A61K 31/4985; A61K 31/4995; A61K 31/506; A61K 31/5377; A61K 31/5386; A61K 31/551; A61P 35/00; C07B 59/00; C07D 401/14; C07D 403/12; C07D 403/14; C07D 405/14; C07D 413/14; C07D 471/10; C07D 487/04; C07D 487/08; C07D 498/08

(52) U.S. Cl. CPC C07D 487/10; A61K 31/497; A61K 31/4985; A61K 31/4995; A61K 31/506; A61K 31/5377; A61K 31/5386; A61K 31/551; A61P 35/00; C07B 59/002; C07D 401/14; C07D 403/12; C07D 403/14; C07D 405/14; C07D 413/14; C07D 471/10; C07D 487/04; C07D 487/08; C07D 498/08

(57) ABSTRACT

The present invention relates to compounds of Formula (I) that may act as inhibitors of checkpoint kinase 1 (Chk1). The invention also relates to pharmaceutical compositions comprising those compounds, and to their use in the treatment of disease and conditions susceptible to Chk1 inhibition, such as cancer.

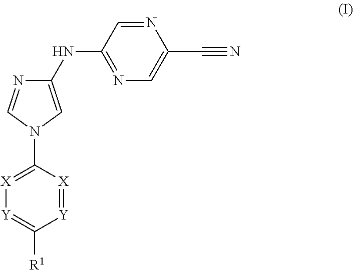

### FIELD OF THE INVENTION

The present invention relates to compounds of Formula (I) that may act as inhibitors of checkpoint kinase 1 (Chk1). The invention also relates to pharmaceutical compositions comprising those compounds, and to their use in the treatment of disease and conditions susceptible to Chk1 inhibition, such as cancer.

### BACKGROUND

Chk1 is a serine/threonine kinase involved in cell cycle regulation and maintenance of DNA integrity. DNA damage can be intrinsic or induced (e.g. by genotoxic agents or ionising radiation) and results in single or double strand breaks in the DNA or stalled replication forks. After DNA damage, cell proliferation is halted at the checkpoints, allowing time for DNA repair or, if the damage is too severe, induction of apoptosis. There are four major cell cycle checkpoints which are responsible for regulating cell cycle progression: G1/S, intra-S, G2/M and spindle assembly checkpoints. Chk1 activates the intra-S, S and G2/M checkpoints and as such, forms part of a network of signal pathways called the DNA damage response (DDR) pathway. Inhibition of Chk1 kinase may result in inhibition of DNA repair and therefore it may lead to increased cell death (Dai, Y. et al Clin Cancer Res. 2010, 16, 376).

Cancer is a disease of aberrant cell proliferation, and defects in DNA repair pathways are common in cancer cells. Such defects can make cancer cells more dependent than healthy cells on the remaining DNA repair pathways, including the cell cycle checkpoints. For example, many cancers have defects in the tumour suppressor p53 (Ozaki, T. Cancers 2011, 3, 994) which results in loss of control of the G1/S checkpoint and increased reliance on intra-S, S and G2/M checkpoints. These cancers can have increased sensitivity to Chk1 inhibitors. Other mutations or changes in expression within the DNA repair pathways may increase sensitivity to Chk1 inhibitors. Examples include ataxia telangiectasia mutated (ATM) kinase deficiency, deficiencies in the Fanconi anaemia homologous repair pathway, and mutations that result in disruption of RAD50 signalling. Another factor that can result in increased sensitivity to Chk1 inhibition is MYC overexpression. MYC overexpression combined with Chk1 inhibition can result in synthetic lethality (Chen, H. et al Sig. Transduct. Target Ther. 2018, 3, 5). The MYC oncogene is a central driver in many cancers including breast, liver and colorectal cancers (Wang, C. et al Sig. Transduct. Target Ther. 2021, 6, 117). Blosser reported that cyclin E dysregulation can result in increased sensitivity to Chk1 inhibition (Blosser, W. D. et al Oncotarget 2020, 11, 216) and a E2F/G2M/SAC gene expression signature was linked to sensitivity to a Chk1 inhibitor while the expression of immunity genes was linked to resistance.

Conventional chemotherapeutics, such as topoisomerase inhibitors and antimetabolites, and ionising radiation induce DNA damage which is mitigated by the DNA damage repair pathway, including Chk1. Activation of the repair pathway protects cancer cells from conventional chemo- or radio-therapy. Therefore, combined treatment of Chk1 inhibitors with chemo- or radio-therapy can overcome resistance (Neizer-Ashun, F. et al Cancer Lett. 2021, 497, 202).

A number of small molecule inhibitors of Chk1 have previously been described. Examples include prexasertib (Angius, G. et al Cancer Chemother. Pharmacol. 2020, 85, 9), MK-8776 (SCH900776) (Guzi, T. J. et al Mol. Cancer Ther. 2011, 10, 591), CCT245737 (SRA737) (Walton, M. I. et al Oncotarget 2016, 7, 2329), GDC-0425 (Infante, J. R. et al Clin. Cancer. Res. 2017, 23, 2423), rabusertib (King, C. et al Invest. New Drugs 2014 32, 213), CCT244747 (Lainchbury, M. et al J. Med. Chem. 2012, 55, 10229). Further Chk1 inhibitor examples have been reported in patents such as WO 201520390, WO 2021043208, WO 2021104461 and WO202119236.

One area of kinase drug discovery which remains challenging is the identification of central nervous system (CNS) penetrant kinase inhibitors (Heffron, T. P. Neuro. Oncol 2018, 20, 307). AChk1 inhibitor which crosses the blood brain barrier may have additional utility in the treatment of CNS cancers such as glioma and medulloblastoma or brain metastases. For adenosine triphosphate (ATP) competitive kinase inhibitors, hydrogen bond interaction with the kinase hinge region is usually indispensable for potent inhibition (Xing, L. et al Bioorg. Med. Chem. Lett. 2015, 23, 6520). However, for improved CNS penetration, a low hydrogen bond count is preferred. In particular, a low number of hydrogen bond donating groups favours CNS penetration (Wager, T. T. et al ACS Chem. Neurosci. 2016, 7, 767; Shi, Y. et al Bioorg. Med. Chem. Lett. 2018, 28, 1981). Reducing hydrogen bond count, especially hydrogen bond donor count (for improved CNS penetration), whilst maintaining good activity through hinge binding interactions remains a challenge in the identification of kinase inhibitors, which may have scope for use in CNS disorders. Similarly, a reduced topological polar surface area (tPSA) favours CNS penetration but hydrogen bonding groups in kinase inhibitors often form key binding interactions. Thus maintaining good biological activity in kinase inhibitors with low PSA remains a challenge (Wager, T. T. et al ACS Chem. Neurosci. 2016, 7, 767).

A number of Chk1 inhibitors which include a pyrazole-NH-pyrazine moiety have been reported such as prexasertib (Angius, G. et al Cancer Chemother. Pharmacol. 2020, 85, 9; WO20110144126) and examples in WO2022114189, WO2021119236, CN112457306, WO2017132928, WO2015120390. Based on docking studies, the pyrazole functional group in these examples provides two hydrogen bonding interactions (donor and acceptor) to Cys87 in the kinase hinge region (Xi, J. et al 2021 Based on Computational Study, 10.21203/rs.3.rs-509168/v1). Given the importance of hinge binding interactions, removal or weakening this key interaction would be expected to be detrimental to biological activity. In this invention, an imidazole-NH-pyrazine motif has been identified with good Chk1 activity. The reduced hydrogen bond donor capacity of this motif results in increased utility in the treatment of cancers, particularly CNS cancers.

Another challenge in the field of kinase drug discovery is kinase selectivity. Most small molecule kinase inhibitors interact with multiple members of the protein kinase family (Davis, M. et al Nat. Biotechnol. 2011, 29, 1046). Such ‘off target’ activity can impact biological efficacy and may drive undesired effects (Yang, X. et al J. Biomed. Inform. 2010, 43, 376), thus selective agents may provide a therapeutic benefit. Several Chk1 inhibitors have been reported to also inhibit Chk2 (e.g. examples in WO2010077758; Zabludoff, S. D. et al Mol. Cancer Ther. 2008, 7, 2955). However, selectivity over Chk2 is advantageous in overriding the S-phase checkpoint (Reader, J. C. et al J. Med. Chem. 2011, 54, 8328). Ablation of Chk2 in addition to Chk1 was shown to be inferior to Chk1 ablation alone, in RNAi studies (Guzi, T. J. et al Mol. Cancer Ther. 2011, 10, 591).

Another family of kinase proteins of relevance to the field of Chk1 inhibitors is the ribosomal S6 kinase family (RSKs). RSKs phosphorylate Chk1 at an inhibitory site, Ser280 (Ray-David, H. et al Oncogene 2013, 32, 4480). RSK inhibitors such as SL0101 increase Chk1 kinase activity through increased phosphorylation of Chk1 Ser345. Therefore, Chk1 inhibitors with reduced RSK activity may have a biological advantage. Several Chk1 inhibitors have also been reported to inhibit RSK1 and/or RSK2 (Ditano, J. P. ACS Pharmacol. Transl. Sci. 2021, 4, 730; Walton, M. I. et al Oncotarget 2016, 7, 2329).

Therefore, there is still a need for compounds that can act as Chk1 inhibitors with improved properties, which may include improved brain/CNS penetration and/or improved kinase selectivity, particularly regarding Chk2 and RSKs.

In this invention, compounds containing an imidazole-NH-pyrazine motif, according to Formula (I), have been identified with suitable selectivity over Chk2 and RSK proteins. These compounds may have utility in the treatment of cancer.

### SUMMARY OF INVENTION

It has been found that compounds of Formula (I) may act as Chk1 inhibitors and therefore may treat diseases and conditions susceptible to Chk1 inhibition, such as cancer. Further, they have certain beneficial properties leading to increased potential for use as a drug compared to known compounds. This may be due to their efficacy, solubility, selectivity profiles, safety profile and/or other notable pharmacokinetic properties. In particular, an advantage may be found in the selectivity of the compounds, particularly in relation to kinase selectivity. For example, the compounds may be selective for Chk1 inhibition over ribosomal S6 kinase (RSK) inhibition, which may lead to an improved ability to treat cancer and/or fewer side effects. Additionally, another particular advantage of the compounds may be their improved brain/CNS penetration, which may be demonstrated by their lower brain efflux compared to known compounds. This may lead to an improved ability to treat cancers relating to the brain and CNS.

Consequently, the invention relates to a compound of Formula (I),

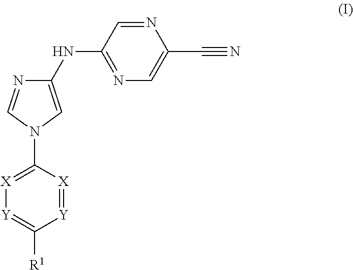

or a pharmaceutically acceptable salt, solvate, hydrate, tautomer, enantiomer, diastereoisomer, isotopic form, N-oxide, and/or prodrug thereof, wherein
   each X is independently N or CR²;
   each Y is independently N or CR³;
   0, 1, or 2 of X and Y are N;
   R¹, R² and R³ are independently selected from
   (a) H, halo, (C₁-C₆)alkyl, (C₁-C₆)haloalkyl, (C₃-C₉)cycloalkyl, —OR⁴, —NR⁵R⁶, —C(O)R⁷, wherein the (C₁-C₆)alkyl, (C₁-C₆)haloalkyl, and (C₃-C₉)cycloalkyl are optionally substituted with one or more R⁸;
   (b) 4- to 7-membered non-aromatic heterocycle optionally substituted with one or more R⁹; and
   (c) 7- to 10-membered spiro, bridged or fused heterocyclic system, each of which is optionally substituted with one or more of (C₁-C₆)alkyl, (C₁-C₆)haloalkyl, and —NR₂;
   R⁴ is selected from (C₁-C₆)alkyl, (C₁-C₆)haloalkyl, (C₁-C₆)alkylene-NR₂, 4- to 6-membered non-aromatic heterocycle, and 6- to 9-membered heterocyclic spiro system, wherein the 4- to 6-membered non-aromatic heterocycle and 6- to 9-membered heterocyclic spiro system are optionally substituted with one or more of halo and (C₁-C₆)alky;
   R⁵ is selected from H, (C₁-C₆)alkyl, and (C₁-C₆)haloalkyl;
   R⁶ is selected from H, (C₁-C₆)alkyl, (C₁-C₆)haloalkyl, and 4- to 7-membered non-aromatic heterocycle, wherein the heterocycle is optionally substituted with one or more of halo and (C₁-C₆)alkyl;
   R⁷ is selected from H, (C₁-C₆)alkyl, (C₁-C₆)haloalkyl, —NR₂, and 4- to 7-membered non-aromatic heterocycle, wherein the heterocycle is optionally substituted with one or more of halo and (C₁-C₆)alkyl;
   R⁸ is selected from —NR₂, and —(O)ₙ-(4- to 7-membered non-aromatic heterocycle), wherein the 4- to 7-membered non-aromatic heterocycle is optionally substituted with one or more of halo and (C₁-C₆)alkyl;
   R⁹ is selected from halo, (C₁-C₆)alkyl, (C₁-C₆)haloalkyl, (C₃-C₆)cycloalkyl, (C₁-C₆)alkoxy, —NR₂, —N(R)(C₁-C₆)hydroxyalkyl, (C₁-C₆)alkylene-NR₂, 4- to 6-membered non-aromatic heterocycle, and 5- to 8-membered heterocyclic spiro system, wherein the (C₃-C₆)cycloalkyl, 4- to 6-membered non-aromatic heterocycle, and 5- to 8-membered heterocyclic spiro system are optionally substituted with one or more of halo and (C₁-C₆)alkyl; and
   each R is independently selected from H, (C₁-C₆)alkyl and (C₁-C₆)haloalkyl.

These compounds are compounds of the invention.

In the compounds of the invention, each X is independently N or CR², and each Y is independently N or CR³, as long as at most two out of the four ring atoms that are X and Y are N. This means that in the compounds of the invention, 0, 1, or 2 of X and Y are N. When none of X and Y are N, then each X is CR², each Y is CR³. This means that the ring containing X and Y in Formula (I) is a C₆-aryl, i.e. an optionally substituted phenyl. In this case, the compounds of the invention are of Formula (II).

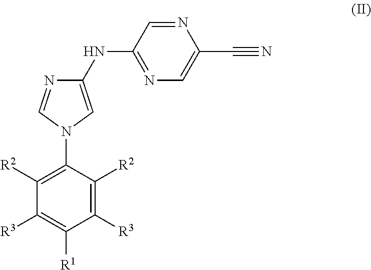

The ring containing the X and Y atom may be an optionally substituted pyridyl. This is the case when one of the four X and Y groups is N. For instance, if one X is N and the other X is CR², with each Y being CR³, then the compounds of the invention are of Formula (III). Similarly, if one Y is N and the other Y is CR³, with each X being CR², then the compounds of the invention are of Formula (IV).

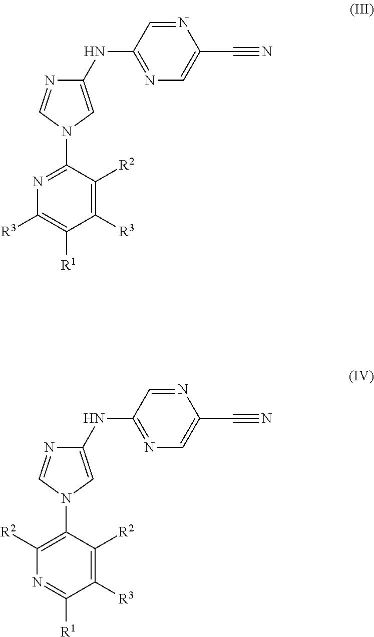

The ring containing X and Y may be a pyrazinyl, pyridazyl, or pyrimidyl. This is the case when two of the four X and Y groups are N, with the remaining groups being CR² or CR³, as appropriate. The compounds may therefore be represented by formulae (V) to (VIII).

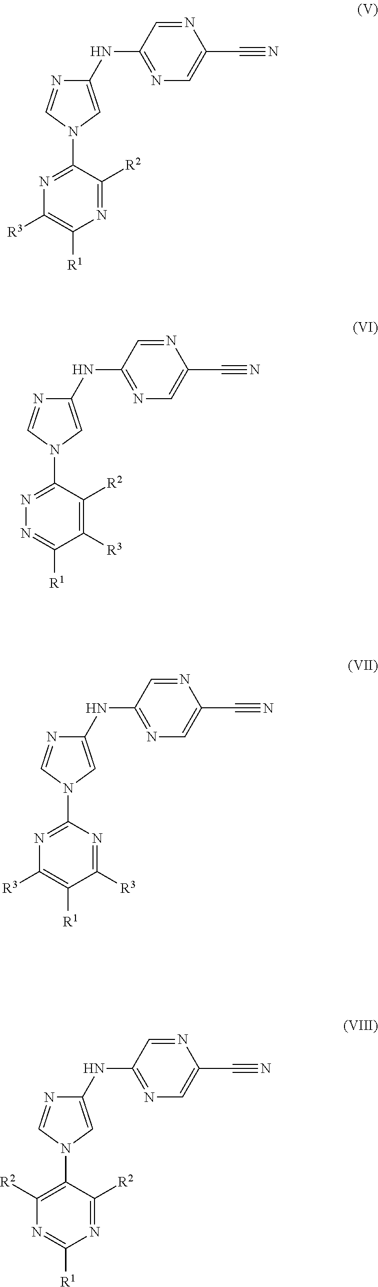

It is preferrable that in the compounds of invention that 0 or 1 of the X and Y groups are N. This means that the preferred compounds of the invention are of Formula (II), Formula (III) or Formula (IV) (most preferably Formula (II) or Formula (III)), or a pharmaceutically acceptable salt, solvate, hydrate, tautomer, enantiomer, diastereoisomer, isotopic form, N-oxide, and/or prodrug thereof, with the remainder of the groups as herein defined. This means that the ring containing X and Y is preferably an optionally substituted phenyl or pyridinyl. This means that each Y is preferably CR³.

Each R¹, R² and R³ is independently selected from H, halo, (C₁-C₆)alkyl, (C₁-C₆)haloalkyl, (C₃-C₉)cycloalkyl, —OR⁴, —NR⁵R⁶, —C(O)R⁷, 4- to 7-membered non-aromatic heterocycle, and 7- to 10-membered spiro, bridged or fused heterocyclic system. The (C₁-C₆)alkyl, (C₁-C₆)haloalkyl, and (C₃-C₉)cycloalkyl are optionally substituted with one or more R⁸. The 4- to 7-membered non-aromatic heterocycle is optionally substituted with one or more R⁹. The 7- to 10-membered spiro, bridged or fused heterocyclic system is optionally substituted with one or more of (C₁-C₆)alkyl, (C₁-C₆)haloalkyl, and —NR₂.

The terms “optional” or “optionally” denotes that the subsequently described event or circumstance may, but need not, occur, and that the description includes instances where the event or circumstance occurs and instances in which it does not.

The term “substituted” denotes that the group to which it refers has one or more hydrogen atoms substituted for a different group. For instance, “substituted alkyl” refers to a monovalent radical of an alkane with one or more hydrogens attached to the alkyl being replaced with another group.

In view of the above, the term “optionally substituted” means that the group to which it refers may or may not be substituted, e.g. for instance, with one or more halo.

The term “independently selected from” denotes that each feature is individually chosen from a list without regard to the selection of the other features.

As used herein, the term “halo” denotes a halogen atom, and is preferably, F, Cl, Br or I, more preferably F or Cl.

The term “(C₁-C₆)alkyl” denotes a linear or branched alkyl group having 1 to 6 carbon atoms, i.e. 1, 2, 3, 4, 5, or 6 carbon atoms. For parts of the range “(C₁-C₆)alkyl” all subgroups thereof are contemplated, such as (C₁-C₅)alkyl, (C₁-C₄)alkyl, (C₁-C₃)alkyl, (C₁-C₂)alkyl, (C₁)alkyl, (C₂-C₅)alkyl, (C₂-C₅)alkyl, (C₂-C₄)alkyl, (C₂-C₃)alkyl, (C₂)alkyl, (C₃-C₆)alkyl, (C₃-C₅)alkyl, (C₃-C₄)alkyl, (C₃)alkyl, (C₄-C₆)alkyl, (C₄-C₅)alkyl, (C₄)alkyl, (C₅-C₅)alkyl, (C₅)alkyl, and (C₆)alkyl. Examples of “(C₁-C₆)alkyl” include methyl, ethyl, n-propyl, isopropyl, n-butyl, isobutyl, sec-butyl, t-butyl, and linear or branched pentyl and hexyl.

When a term denotes a range, for instance “C₁-C₆” or “1 to 6 carbon atoms” as present in the definition of “(C₁-C₆)alkyl”, each integer is considered to be disclosed, i.e. 1, 2, 3, 4, 5 and 6.

The term “(C₁-C₆)haloalkyl” denotes a (C₁-C₆)alkyl group in which one or more of the hydrogen atoms are independently replaced with a halo atom, e.g. F, Cl, Br, or I, preferably F or Cl, more preferably F. Each halo-substituted carbon atom in (C₁-C₆)haloalkyl may be mono-, di- or, where possible, trisubstituted with an independently selected halo atom. For parts of the range “(C₁-C₆)haloalkyl” all subgroups thereof are contemplated, such as (C₁-C₅)haloalkyl, (C₁-C₄)haloalkyl, (C₁-C₃)haloalkyl, (C₁-C₂)haloalkyl, (C₁)haloalkyl, (C₂-C₆)haloalkyl, (C₂-C₅)haloalkyl, (C₂-C₄)haloalkyl, (C₂-C₃)haloalkyl, (C₂)haloalkyl, (C₃-C₆)haloalkyl, (C₃-C₅)haloalkyl, (C₃-C₄)haloalkyl, (C₃)haloalkyl, (C₄-C₆)haloalkyl, (C₄-C₅)haloalkyl, (C₄)haloalkyl, (C₅-C₆)haloalkyl, (C₅)haloalkyl, and (C₆)haloalkyl. Examples of “(C₁-C₆)haloalkyl” include mono-, di-, and tri-halomethyl wherein the halo atoms are independently F, Cl, Br, or I, such as —CH₂F, —CF₂H, —CF₃′—CH₂Cl, —CCl₂H, —CCl₃, —CHFCl, —CF₂Cl, —CCl₂F, mono-, di- and tri-bromomethyl, mono-, di- and tri-iodomethyl. Also included is ethyl substituted with 1, 2, 3, 4, or 5 independently selected halo atoms; n-propyl and isopropyl substituted with 1, 2, 3, 4, 5, 6 or 7 independently selected halo atoms; n-butyl, isobutyl, sec-butyl, and t-butyl substituted with 1, 2, 3, 4, 5, 6, 7, 8 or 9 independently selected halo atoms; linear or branched pentyl substituted with 1, 2, 3, 4, 5, 6, 7, 8, 9 10, or 11 independently selected halo atoms; and linear or branched hexyl substituted with 1, 2, 3, 4, 5, 6, 7, 8, 9 10, 11, 12 or 13 independently selected halo atoms.

The term “(C₁-C₃)haloalkoxy” denotes —O—(C₁-C₃)haloalkyl, in which “(C₁-C₃)haloalkyl” is as defined above. For parts of the range “(C₁-C₃)haloalkoxy” all subgroups thereof are contemplated, such as (C₁-C₂)haloalkoxy, (C₁)haloalkoxy, (C₂-C₃)haloalkoxy, (C₂)haloalkoxy, and (C₃)haloalkoxy.

The term “(C₃-C₉)cycloalkyl” denotes a monocyclic alkyl group having 3 to 9 carbon atoms. For parts of the range “(C₃-C₉)cycloalkyl” all subgroups thereof are contemplated, such as (C₃-C₉)cycloalkyl, (C₃-C₈)cycloalkyl, (C₃-C₇)cycloalkyl, (C₃-C₆)cycloalkyl, (C₃-C₅)cycloalkyl, (C₃-C₄)cycloalkyl, (C₃)cycloalkyl, (C₄-C₉)cycloalkyl, (C₄-C₈)cycloalkyl, (C₄-C₇)cycloalkyl, (C₄-C₆)cycloalkyl, (C₄-C₅)cycloalkyl, (C₄)cycloalkyl, (C₅-C₉)cycloalkyl, (C₅-C₆)cycloalkyl, (C₅-C₇)cycloalkyl, (C₅-C₆)cycloalkyl, (C₅)cycloalkyl, (C₆-C₉)cycloalkyl, (C₅-C₈)cycloalkyl, (C₆-C₇)cycloalkyl, (C₆)cycloalkyl, (C₇-C₉)cycloalkyl, (C₇-C₈)cycloalkyl, (C₇)cycloalkyl, (C₈-C₉)cycloalkyl, (C₈)cycloalkyl and (C₉)cycloalkyl. Examples of these cycloalkyl groups include cyclopropyl, cyclobutyl, cyclopentyl, cyclohexyl, cycloheptyl, cyclooctyl and cyclononyl.

There is a feature of the invention wherein any of R¹, R², and R³ are selected to be a (C₃-C₉)cycloalkyl optionally substituted with one or more R⁸. In this case, any of R¹, R², and R³ preferably may be a (C₃-C₆)cycloalkyl optionally substituted with one or more R⁸, more preferably cyclopropyl or cyclobutyl, each of which is optionally substituted with —NR₂.

The term “heteroatom” denotes O, N, or S.

The term “4- to 7-membered non-aromatic heterocycle” denotes a monocyclic non-aromatic ring containing 4, 5, 6 or 7 atoms in the ring, wherein at least one of those atoms (e.g. 1, 2, 3, or 4, preferably 1 or 2) is a heteroatom, e.g. O, N, or S, preferably N or O. Non-aromatic heterocycles comprising other numerical ranges of ring atoms, such as 4- to 6-members, 5- to 7-members, 4- or 5-members, and 5- or 6-members, comprise the appropriate number of atoms. Examples of 4- to 7-membered non-aromatic heterocycles include, but are not limited to,

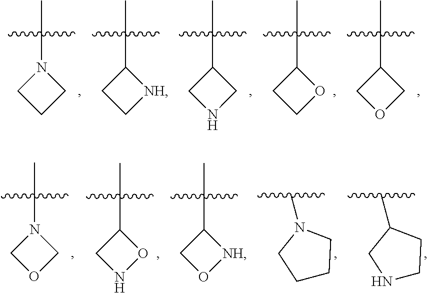

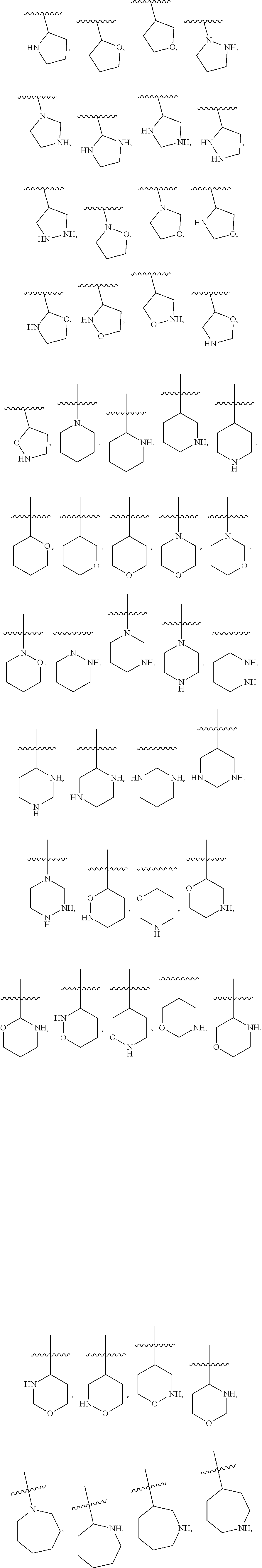

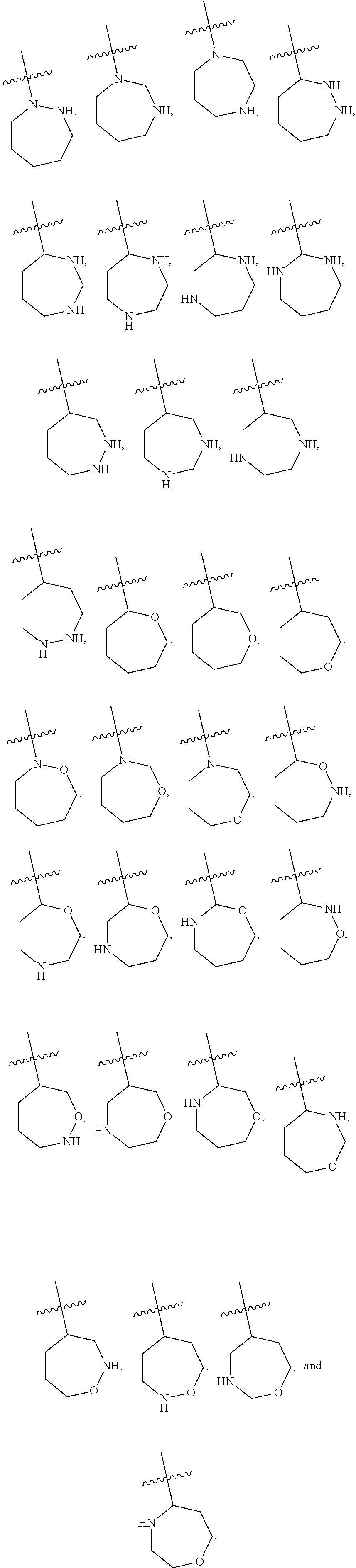

each of which may be substituted as defined herein. Further examples contain 3 or 4 heteroatoms.

Preferable examples of 4- to 7-membered non-aromatic heterocycles include the following

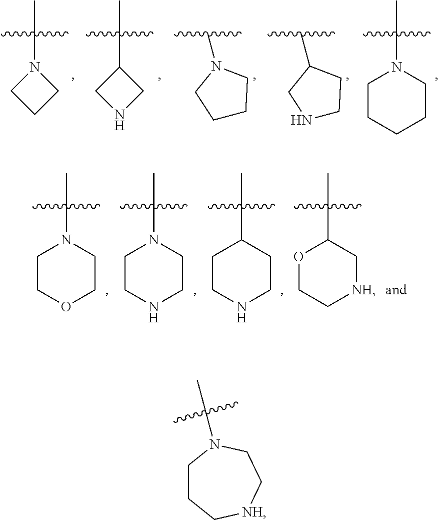

each of which may be substituted as described herein.

The term “—(O)ₙ-(4- to 7-membered non-aromatic heterocycle)” denotes a “4- to 7-membered non-aromatic heterocycle” as defined herein, optionally linked to the rest of the compound via an oxygen group. n can be 0 or 1. This means that when ‘n’ is 1, then the oxygen linker is present and “—(O)ₙ-(4- to 7-membered non-aromatic heterocycle)” is —O-(4- to 7-membered non-aromatic heterocycle). When ‘n’ is 0 the oxygen linker is not present and it refers to a “4- to 7-membered non-aromatic heterocycle”. Non-limiting examples of “—(O)ₙ-(4- to 7-membered non-aromatic heterocycle)” when n is 0 include all of those listed above for “4- to 7-membered non-aromatic heterocycle”, each of which is optionally substituted with one or more of halo or (C₁-C₆)alkyl. Non-limiting examples of “—(O)ₙ-(4- to 7-membered non-aromatic heterocycle)” when n is 1 include any of the 4- to 7-membered non-aromatic heterocycles listed above, wherein the heterocycle is linked to the rest of the compound via an oxygen linker. Thus, non-limiting examples of “—(O)ₙ-(4- to 7-membered non-aromatic heterocycle)” when n is 1 include the following

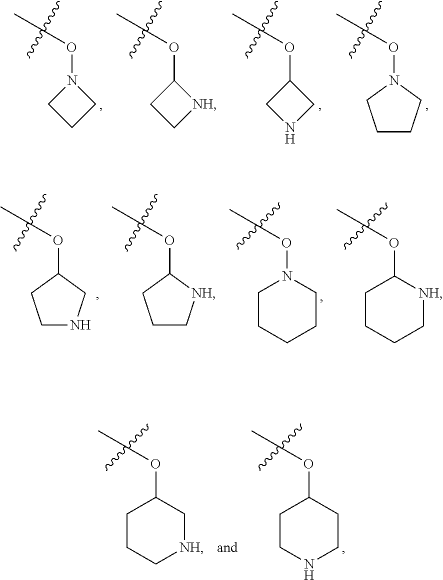

each of which is optionally substituted with one or more of halo or (C₁-C₆)alkyl. Preferably, examples of “—(O)ₙ-(4- to 7-membered non-aromatic heterocycle)” wherein n is 1 include

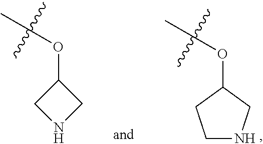 [SMILES: *OC1CNC1 ; *OC1CCNC1]

each of which is optionally substituted with one or more of halo or (C₁-C₈)alkyl.

The term “7- to 10-membered spiro, bridged or fused heterocyclic system” denotes a non-aromatic ring system containing 7, 8, 9 or 10 atoms in the system (i.e. the one or more rings in the system), wherein at least one of those atoms (e.g. 1, 2, 3, or 4 atoms) is a heteroatom, e.g. O, N, or S, preferably N or O. those “atoms in the system” do not include any optional substituents. An “8- to 10-membered spiro or fused heterocyclic system” is the same, but wherein the non-aromatic ring system contains 8, 9 or 10 atoms in the system, and the system is a spiro group or a fused bicycle.

The heterocyclic ring system may be a spiro group, i.e. a group comprising two rings joined by a common tetrahedral carbon atom. In this case, it is preferably a 7- to 10-membered spiro heterocyclic system, such as one selected from the following non-limiting examples

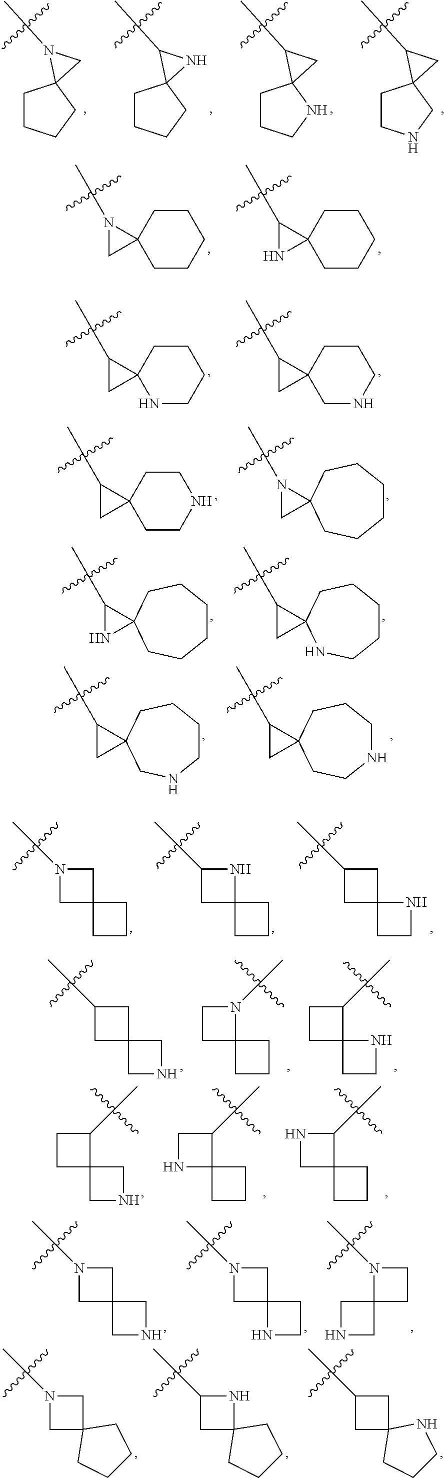

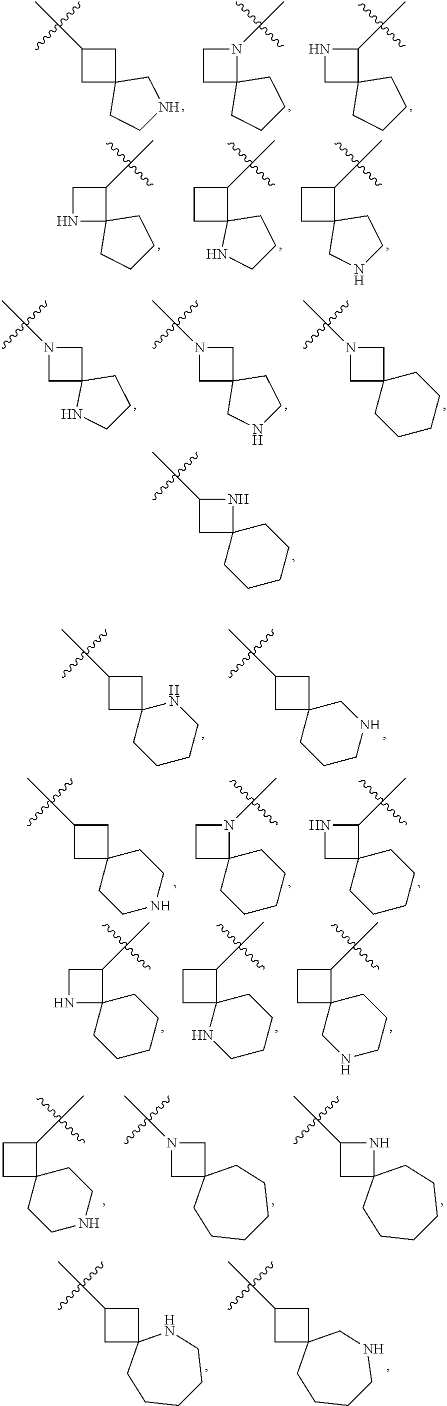

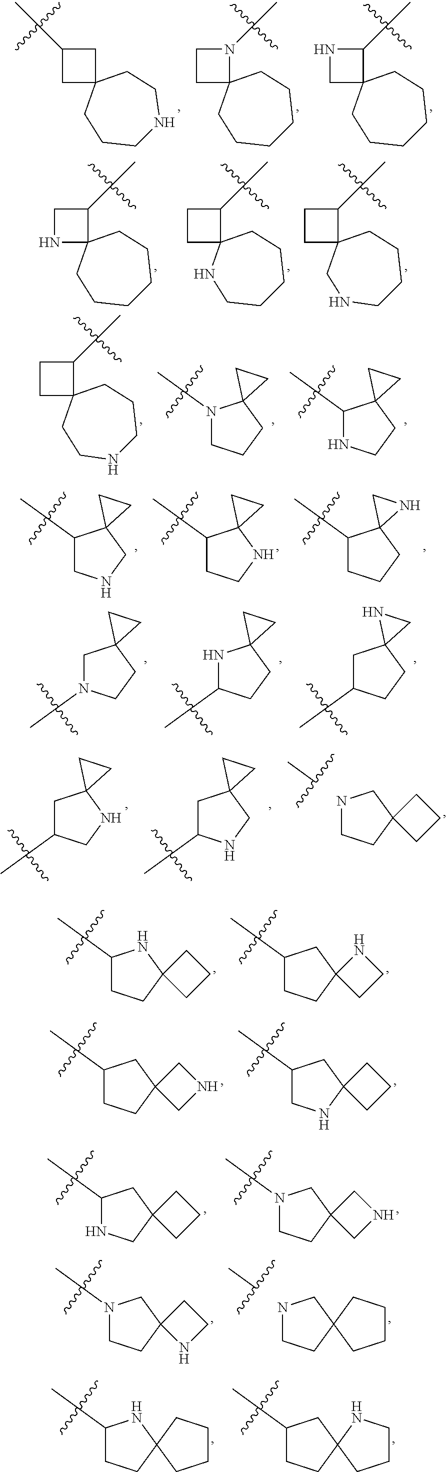

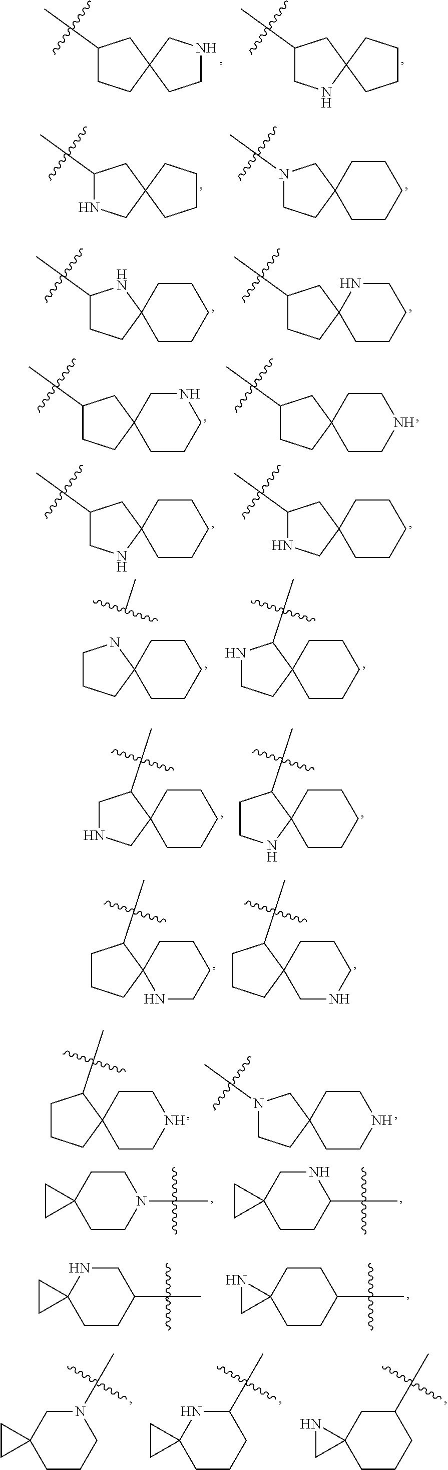

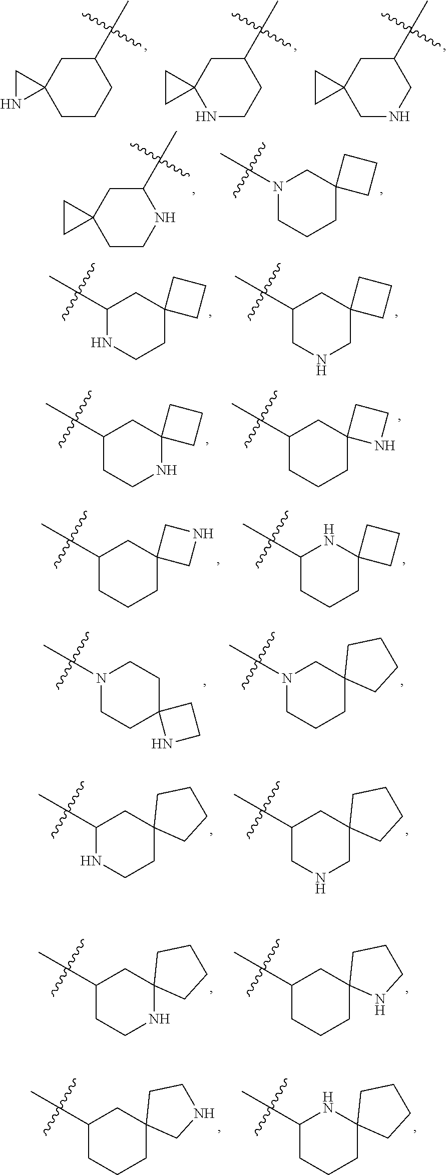

with further examples comprising more than one heteroatom in the spiro ring system, and/or where the point of attachment to the rest of the compound may be from any of the atoms (including the heteroatoms) in the ring. The above non-limited examples may be optionally substituted as defined herein. In particular, each of these examples may be optionally substituted with one or more of (C₁-C₆)alkyl, (C₁-C₆)haloalkyl, and —NR₂. Preferable examples of a 7- to 10-membered spiro heterocyclic system include

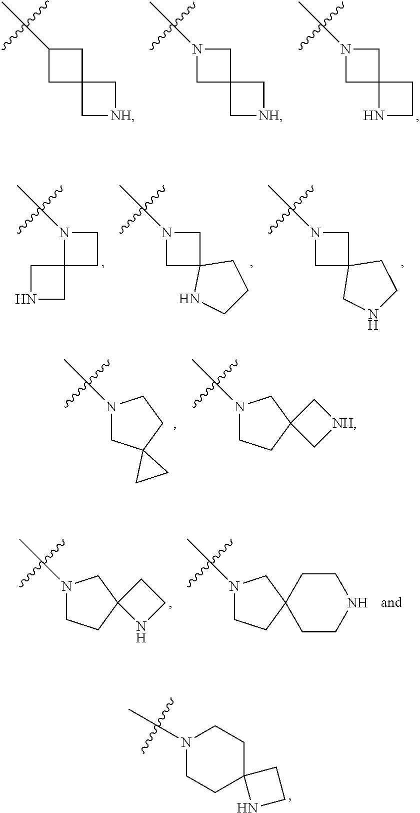

each of which is optionally substituted with one or more of (C₁-C₆)alkyl, (C₁-C₆)haloalkyl, and —NR₂.

A6- to 9-membered heterocyclic spiro system is the same as the spiro heterocycle described above, but containing 6, 7, 8 or 9 ring atoms. A 7- to 9-membered heterocyclic spiro system is the same as the spiro heterocycle described above, but containing 7, 8 or 9 ring atoms. A 5- to 8-membered heterocyclic spiro system is the same as the spiro heterocycle described above, but containing 5, 6, 7 or 8 ring atoms. A6- to 8-membered heterocyclic spiro system is the same as the spiro heterocycle described above, but containing 5, 6, 7 or 8 ring atoms.

The heterocyclic ring system may be a bridged group. This is a 7- to 10-membered bridged heterocycle that is formed from a 4- to 9-membered monocycle in which two non-adjacent atoms are linked by a 1- to 3-membered bridge (and in which the total amount of atoms in the system (excluding any substituents) is from 7 to 10). At least one of the atoms in the 7- to 10-membered bridged heterocycle is a heteroatom. Heteroatoms may be in the monocyclic ring, the bridge potion, or both the monocyclic ring and the bridge. As will be appreciated, any ring within a bridge group may be considered the “monocycle”, leaving the remaining part to be the “bridge”. In this regard, a 6-membered monocycle with a 1-membered bridge between the 1- and 4-position of the monocycle, may also be considered a 5-membered monocycle with a 2-membered bridge between the 1- and 3-position of the monocycle. Non-limiting examples of a 7- to 10-membered bridged heterocyclic system include

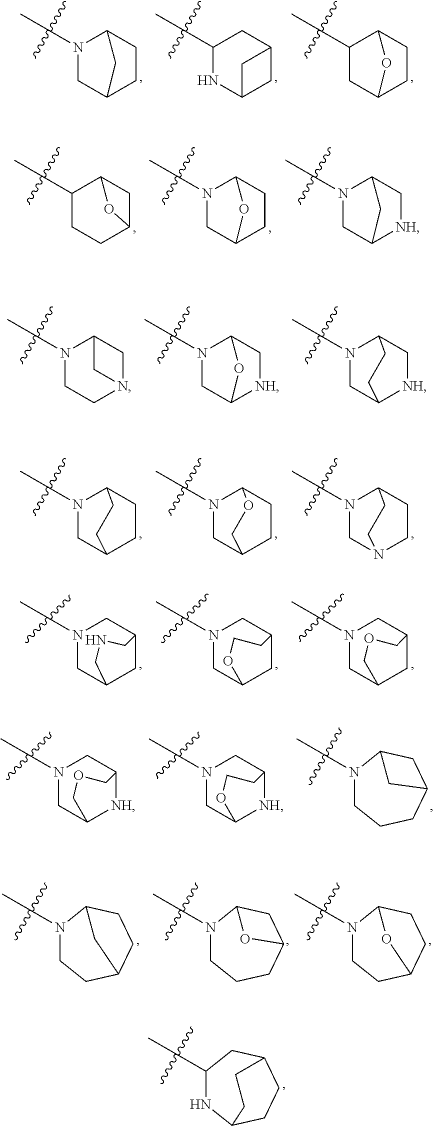

each of which is optionally substituted with one or more of (C₁-C₆)alkyl, (C₁-C₆)haloalkyl, and —NR₂. Preferable examples of a 7- to 10-membered bridged heterocyclic system include

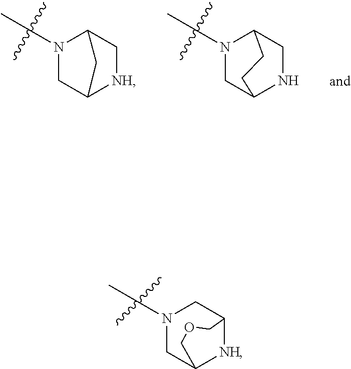 [SMILES: *N1CC2CC1CN2 ; *N1CC2CCC1CN2 ; *N1CC2COCC(C1)N2]

each of which is optionally substituted with one or more of (C₁-C₆)alkyl, (C₁-C₆)haloalkyl, and —NR₂, preferably (C₁-C₃)alkyl and more preferably methyl.

The heterocyclic ring system may be a fused bicycle. As used herein, a “fused” ring system is usually two rings, or a bicycle, that share two ring atoms, as in the fused rings exemplified below. In this case, it is preferably a 7- to 10-membered fused bicyclic heterocycle. Heteroatoms may be on either of the two rings, including the atoms shared between the two rings. Non-limiting examples of a 7- to 10-membered fused heterocyclic system include

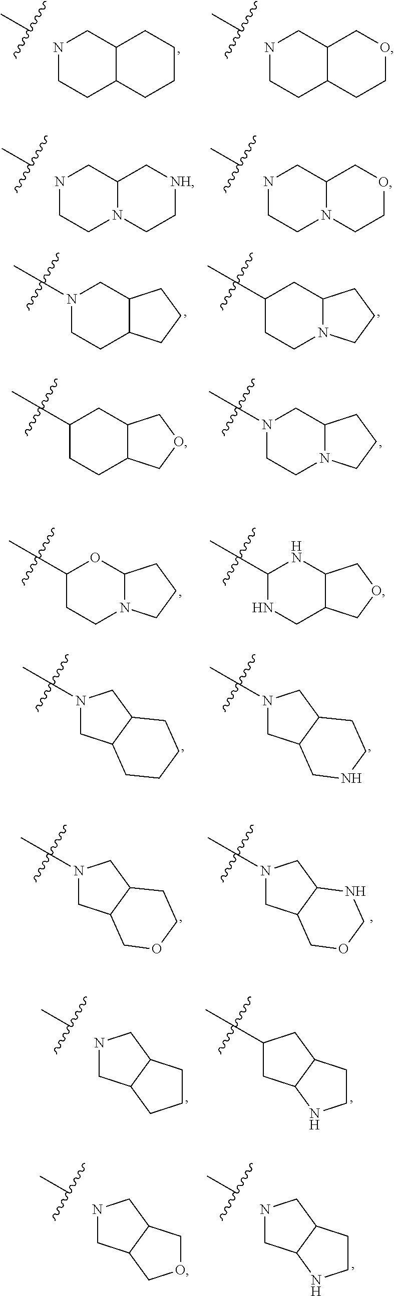

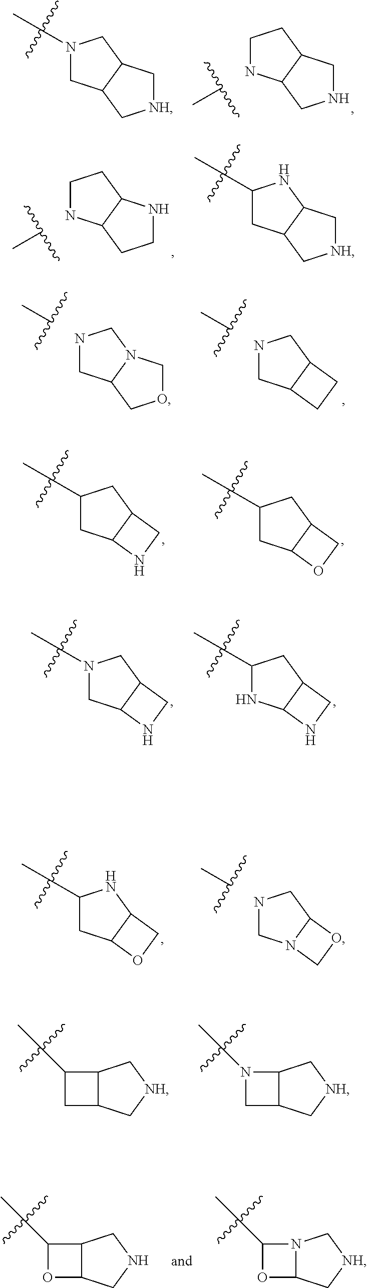

each of which is optionally substituted with one or more of (C₁-C₆)alkyl, (C₁-C₆)haloalkyl, and —NR₂. Preferable examples of a 7- to 10-membered fused heterocyclic system include

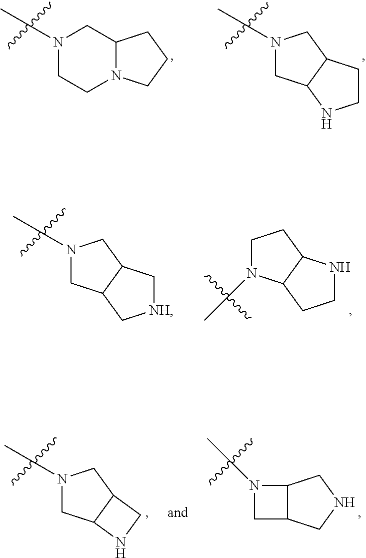 [SMILES: *N1CCN2CCCC2C1 ; *N1CC2CCNC2C1 ; *N1CC2CNCC2C1 ; *N1CCC2NCCC21 ; *N1CC2CNC2C1 ; *N1CC2CNCC21]

each of which is optionally substituted with one or more of (C₁-C₆)alkyl, (C₁-C₆)haloalkyl, and —NR₂.

The term “(C₁-C₆)alkylene” denotes a linear or branched chain diradical of (C₁-C₆)alkyl. For parts of the range “(C₁-C₆)alkylene” all subgroups thereof are contemplated, such as (C₁-C₆)alkylene, (C₁-C₄)alkylene, (C₁-C₃)alkylene, (C₁-C₂)alkylene, (C₁)alkylene, (C₂-C₆)alkylene, (C₂-C₆)alkylene, (C₂-C₄)alkylene, (C₂-C₃)alkylene, (C₂)alkylene, (C₃-C₆)alkylene, (C₃-C₆)alkylene, (C₃-C₄)alkylene, (C₃)alkylene, (C₄-C₆)alkylene, (C₄-C₆)alkylene, (C₄)alkylene, (C₅-C₈)alkylene, (C₅)alkylene, and (C₆)alkylene. Non-limiting examples of “(C₁-C₆)alkylene” include methylene, ethylene, n-propylene, isopropylene, n-butylene, isobutylene, sec-butylene, t-butylene, and linear or branched pentylene and hexylene.

Each R in “—NR₂” is independently selected from H, (C₁-C₆)alkyl, and (C₁-C₆)haloalkyl. In view of this, the term “—NR₂” may be a primary, secondary or tertiary amine. As such, examples of “—NR₂” include but are not limited to —NH₂,

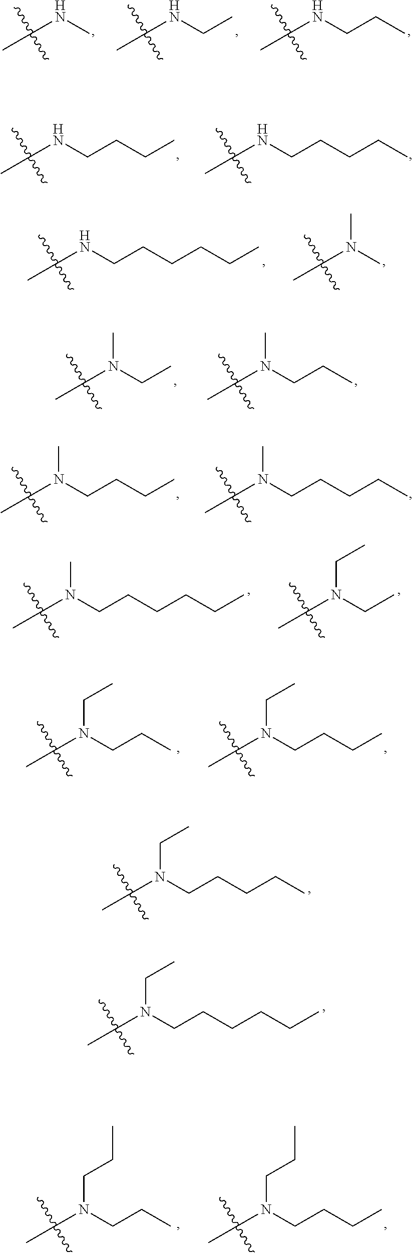

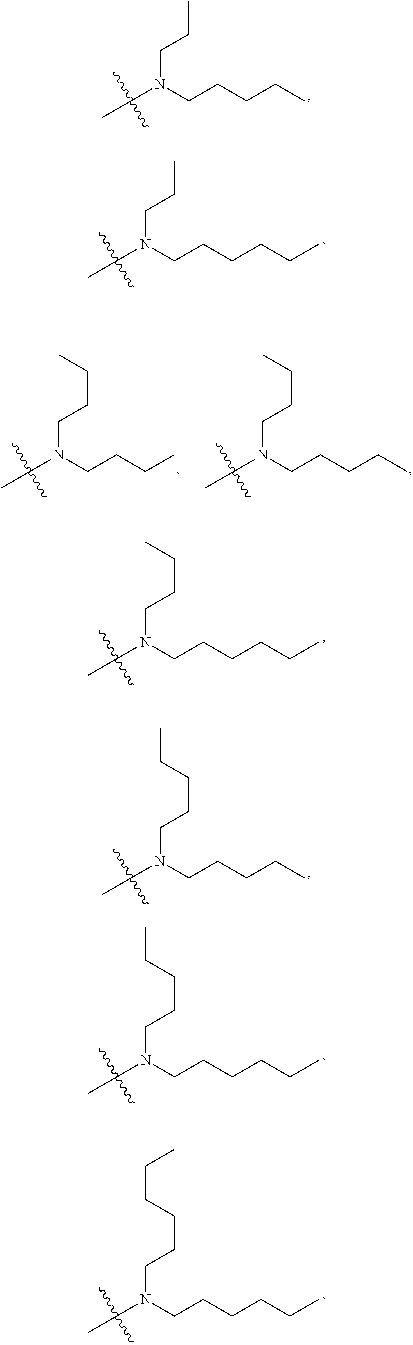

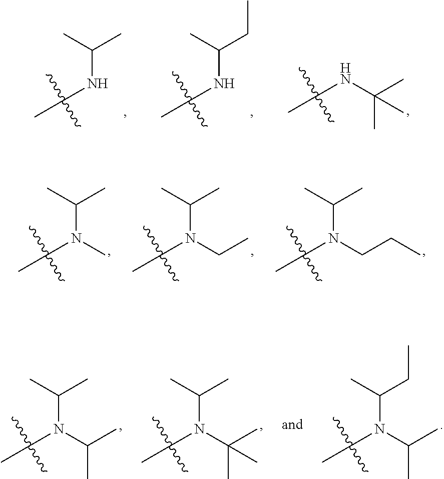

Further examples include groups in which each R may independently be any linear or branched (C₁-C₆)alkyl. Any of the hydrogen atoms on the above alkyl chains may be independently substituted for a halo atom. This forms a (C₁-C₆)haloalkyl. When R is a (C₁-C₆)haloalkyl there can be independently 0, 1, 2 or 3 halo atoms on each carbon atom (where valency permits), provided that there is at least one halo atom present. Examples of —NR₂ wherein at least one R is a (C₁-C₆)haloalkyl include, but are not limited to,

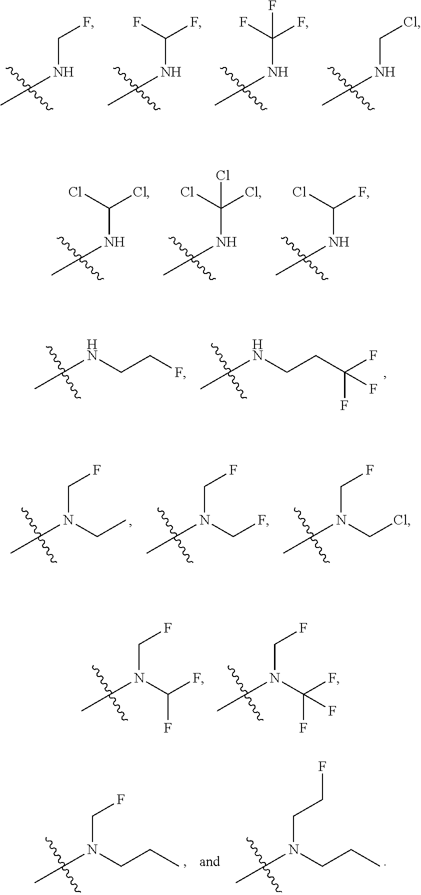

In view of the above, the term “(C₁-C₆)alkylene-NR₂” denotes an alkylamine, in which “(C₁-C₆)alkylene” and “—NR₂” are as defined herein. Preferable examples of “(C₁-C₆)alkylene-NR₂” include

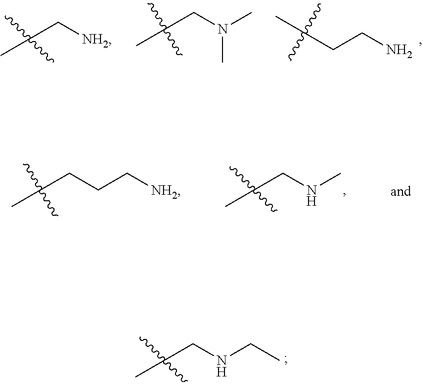

more preferably

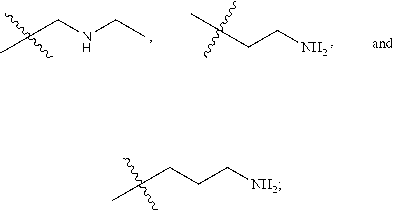

and most preferably

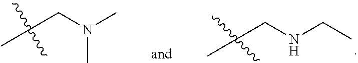

The term “—O—(C₁-C₆)alkylene-NR₂” denotes an oxy-linked “(C₁-C₆)alkylene-NR₂” group, in which the “(C₁-C₆)alkylene-NR₂” is as defined above. Preferable non-limiting examples of “—O—(C₁-C₆)alkylene-NR₂” include

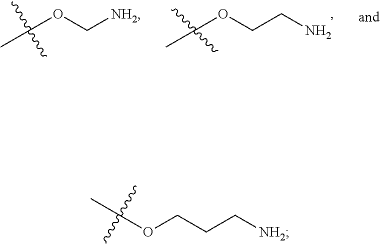

and most preferably

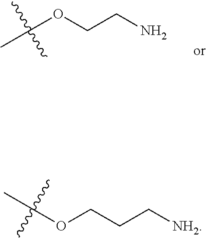 [SMILES: *OCCN ; *OCCCN]

The term “(C₁-C₆)alkoxy” denotes —O—(C₁-C₆alkyl) in which the (C₁-C₆)alkyl group is as defined above. For parts of the range “(C₁-C₆)alkoxy” all subgroups thereof are contemplated, such as (C₁-C₅)alkoxy, (C₁-C₄)alkoxy, (C₁-C₃)alkoxy, (C₁-C₂)alkoxy, (C₁)alkoxy, (C₂-C₆)alkoxy, (C₂-C₅)alkoxy, (C₂-C₄)alkoxy, (C₂-C₃)alkoxy, (C₂)alkoxy, (C₃-C₆)alkoxy, (C₃-C₅)alkoxy, (C₃-C₄)alkoxy, (C₃)alkoxy, (C₄-C₆)alkoxy, (C₄-C₅)alkoxy, (C₄)alkoxy, (C₅-C₆)alkoxy, (C₅)alkoxy, and (C₆)alkoxy. Non-limiting examples of “(C₁-C₆)alkoxy” include methoxy, ethoxy, n-propoxy, isopropoxy, n-butoxy, isobutoxy, sec-butoxy, t-butoxy and linear and branched-chain pentoxy and hexoxy.

The term “(C₁-C₆)hydroxyalkyl” denotes a (C₁-C₆)alkyl group in which one or more of the hydrogen atoms are independently replaced with a hydroxy group (—OH). Each hydroxy-substituted carbon atom in (C₁-C₆)hydroxyalkyl may be mono-, di- or, where possible, trisubstituted with a hydroxy group. For parts of the range “(C₁-C₆)hydroxyalkyl” all subgroups thereof are contemplated, such as (C₁-C₅)hydroxyalkyl, (C₁-C₄)hydroxyalkyl, (C₁-C₃)hydroxyalkyl, (C₁-C₂)hydroxyalkyl, (C₁)hydroxyalkyl, (C₂-C₆)hydroxyalkyl, (C₂-C₅)hydroxyalkyl, (C₂-C₄)hydroxyalkyl, (C₂-C₃)hydroxyalkyl, (C₂)hydroxyalkyl, (C₃-C₆)hydroxyalkyl, (C₃-C₅)hydroxyalkyl, (C₃-C₄)hydroxyalkyl, (C₃)hydroxyalkyl, (C₄-C₆)hydroxyalkyl, (C₄-C₅)hydroxyalkyl, (C₄)hydroxyalkyl, (C₅-C₆)hydroxyalkyl, (C₅)hydroxyalkyl, and (C₆)hydroxyalkyl. Examples of “(C₁-C₆)hydroxyalkyl” include mono-, di-, and tri-hydroxymethyl. Also included is ethyl substituted with 1, 2, 3, 4, or 5 hydroxy groups; n-propyl and isopropyl substituted with 1, 2, 3, 4, 5, 6 or 7 hydroxy groups; n-butyl, isobutyl, sec-butyl, and t-butyl substituted with 1, 2, 3, 4, 5, 6, 7, 8 or hydroxy groups; linear or branched pentyl substituted with 1, 2, 3, 4, 5, 6, 7, 8, 9 10, or hydroxy groups; and linear or branched hexyl substituted with 1, 2, 3, 4, 5, 6, 7, 8, 9 10, 11, 12 or 13 hydroxy groups.

The term “—N(R)(C₁-C₆)hydroxyalkyl” denotes a nitrogen atom linked to the rest of the compound, wherein the nitrogen atom is substituted with an R group, and a “(C₁-C₆)hydroxyalkyl” as defined above. Preferably, non-limiting examples of “—N(R)(C₁-C₆)hydroxyalkyl” include

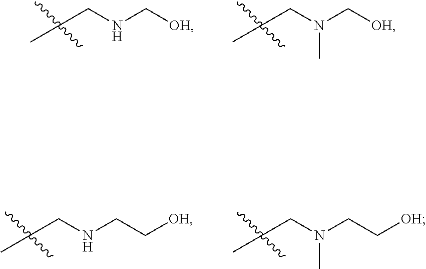

and most preferably

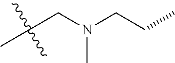

The term “—N(H)(C₁-C₃)alkyl” denotes a “—NR₂” group, wherein one of the R groups is H and the other is a “(C₁-C₃)alkyl” as defined above.

The term “—N((C₁-C₃)alkyl)₂” denotes a “—NR₂” group, wherein each R group is independently a “(C₁-C₃)alkyl” as defined above.

The term “—N(H)(C₁-C₃)haloalkyl” denotes a “—NR₂” group, wherein one of the R groups is H and the other is a “(C₁-C₃)haloalkyl” as defined above. Preferably, non-limiting examples of “—N(H)(C₁-C₃)haloalkyl” include

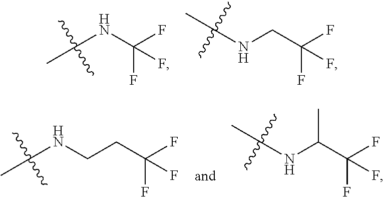

and most preferably

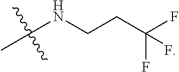 [SMILES: *NCCC(F)(F)F]

The term “(C₁-C₃)alkylene-N((C₁-C₃)alkyl)₂” denotes an alkylamine, in which “(C₁-C₃)alkylene” and “—N((C₁-C₃)alkyl)₂” are as defined herein. Preferably, non-limiting examples of “(C₁-C₃)alkylene-N((C₁-C₃)alkyl)₂” include

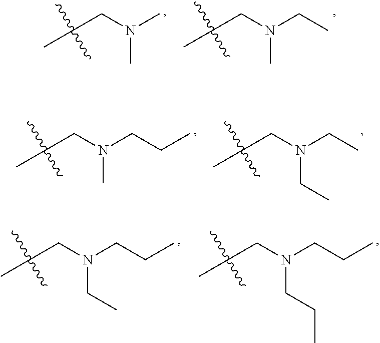

and further examples wherein the (C₁-C₃)alkylene is ethylene or propylene. Most preferably “(C₁-C₃)alkylene-N((C₁-C₃)alkyl)₂” is

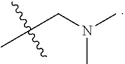 [SMILES: *CN(C)C]

In a preferred feature of the invention, R¹ is selected from 4- to 7-membered non-aromatic heterocycle optionally substituted with one or more R⁹; and 7- to 10-membered spiro, bridged or fused heterocyclic system optionally substituted with one or more of (C₁-C₆)alkyl, (C₁-C₆)haloalkyl, and —NR₂. In this case, R⁹ may be selected from halo, (C₁-C₃)alkyl, (C₁-C₃)haloalkyl, (C₃-C₆)cycloalkyl, (C₁-C₃)alkoxy, —NR₂, (C₁-C₃)alkylene-NR₂, 4- to 6-membered non-aromatic heterocycle, and 5- to 8-membered heterocyclic spiro system. The (C₃-C₆)cycloalkyl, 4- to 6-membered non-aromatic heterocycle, and 5- to 8-membered heterocyclic spiro system are optionally substituted with one or more of halo and (C₁-C₆)alkyl. Additionally, each R is independently selected from H, (C₁-C₆)alkyl and (C₁-C₆)haloalkyl. Preferably R¹ is linked to the rest of the compound by a heteroatom (preferably N) in the heterocycle or heterocyclic ring system of R¹.

In a more preferred feature of the invention, R⁹ is selected from —NR₂, (C₁-C₃)alkylene-NR₂, 4- to 6-membered non-aromatic heterocycle, and 5- to 8-membered heterocyclic spiro system, wherein the 4- to 6-membered non-aromatic heterocycle and 5- to 8-membered heterocyclic spiro system are optionally substituted with one or more of halo and (C₁-C₆)alkyl. Most preferably, R⁹ is selected from F, methyl, methoxy, —NH₂, —N(H)Me, —N(H)Et, —N(H)ⁱPr, —NMe₂, —N(Me)Et

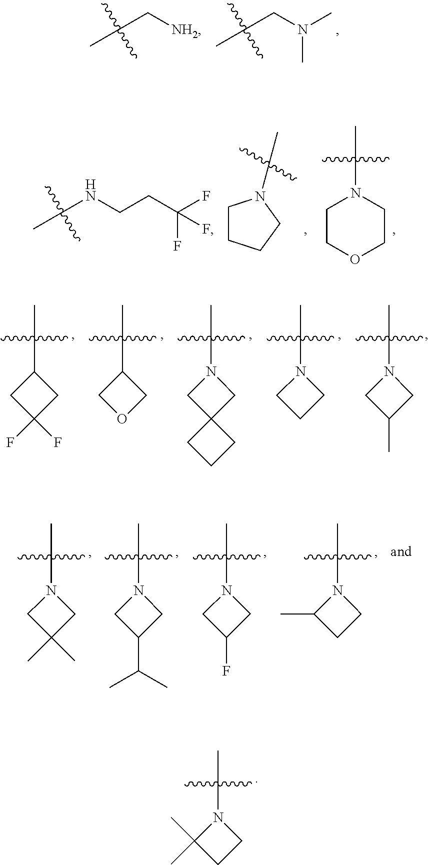

More preferably, R¹ is

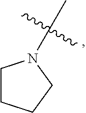 [SMILES: *N1CCCC1]

and is optionally substituted with one or more of halo, (C₁-C₃)alkyl, (C₁-C₃)alkoxy, —NH₂, —N(H)(C₁-C₃)alkyl, —N((C₁-C₃)alkyl)₂, —N(H)(C₁-C₃)haloalkyl, —(C₁-C₃)alkylene-N((C₁-C₃)alkyl)₂

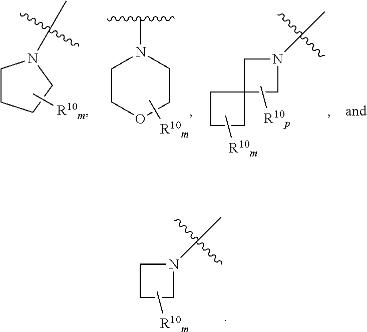

Each R¹⁰ is independently selected from halo and (C₁-C₃)alkyl. m can be 0, 1, 2, or 3. This means that 0, 1, 2 or 3 carbon atoms on the ring may be substituted with R¹⁰. p can be 0, 1 or 2. This means that 0, 1 or 2 carbon atoms on the ring may be substituted with R¹⁰. In spiro systems, the tetrahedral carbon atom common to the two rings cannot be substituted with R¹⁰.

Most preferably R¹ is

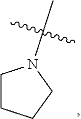 [SMILES: *N1CCCC1]

and is optionally substituted with one or more of F, methyl, methoxy, —NH₂, N(H)Me, —N(H)Et, —N(H)ⁱPr, —NMe₂, —N(Me)Et,

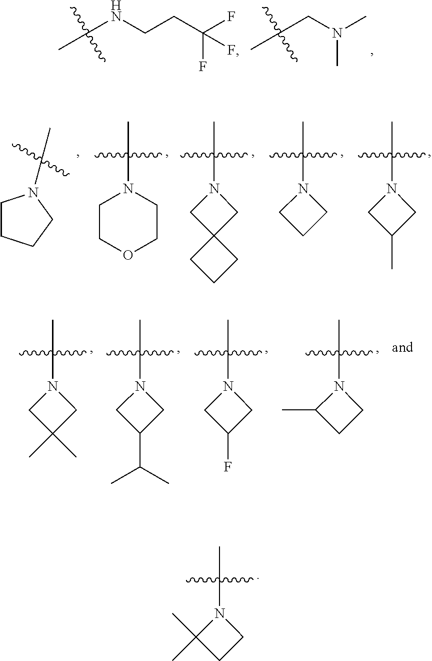

In an alternative feature of the invention, R¹ is a (C₁-C₃)alkyl, preferably methyl, optionally substituted with one or more R⁸. In this case, R⁸ may be selected from —NR₂, and —(O)ₙ-(4- to 7-membered non-aromatic heterocycle), wherein the 4- to 7-membered non-aromatic heterocycle is optionally substituted with one or more of halo and (C₁-C₃)alkyl. Additionally, n is 0 or 1, and each R is independently selected from H, (C₁-C₆)alkyl and (C₁-C₆)haloalkyl. In this feature, R¹ is most preferably

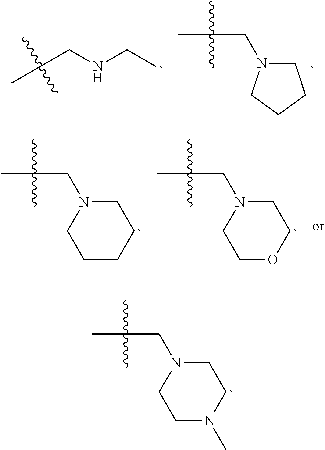

wherein each of

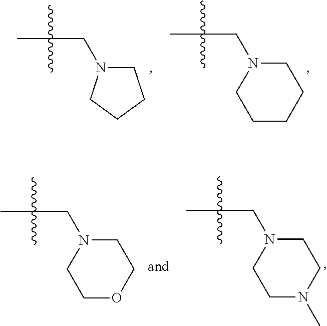

are optionally substituted with F.

In an alternative feature of the invention, R¹ is —NR⁵R⁶. In this feature, R⁵ is selected from H, (C₁-C₆)alkyl, and (C₁-C₆)haloalkyl, preferably H and (C₁-C₆)alkyl, more preferably H and Me. Additionally, R⁶ is selected from H, (C₁-C₆)alkyl, (C₁-C₆)haloalkyl, and 4- to 7-membered non-aromatic heterocycle, wherein the 4- to 7-membered non-aromatic heterocycle is optionally substituted with one or more of halo and (C₁-C₆)alkyl. Preferably, R⁶ is a 5- or 6-membered non-aromatic heterocycle optionally substituted with one or more (C₁-C₃)alkyl, more preferably R⁶ is

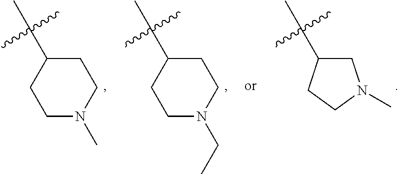

Most preferably R⁵ is H or methyl, and R⁶ is

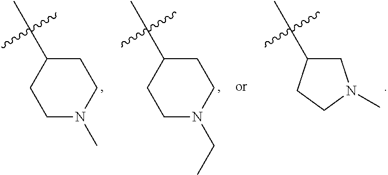

Alternatively, R¹ is —OR⁴, with R⁴ being selected from (C₁-C₆)alkyl, (C₁-C₆)haloalkyl, (C₁-C₆)alkylene-NR₂, 4- to 6-membered non-aromatic heterocycle, and 6- to 9-membered heterocyclic spiro system. The 4- to 6-membered non-aromatic heterocycle and 6- to 9-membered heterocyclic spiro system are optionally substituted with one or more of halo and (C₁-C₆)alkyl.

In this feature of the invention, each R is independently selected from H, (C₁-C₆)alkyl and (C₁-C₆)haloalkyl, and is preferably H.

More preferably, R⁴ is selected from (C₁-C₃)alkylene-NR₂, 4- or 5-membered non-aromatic heterocycle, and 7- to 9-membered heterocyclic spiro system, wherein the 4- or 5-membered non-aromatic heterocycle and 7- to 9-membered heterocyclic spiro system are optionally substituted with one or more of halo and (C₁-C₆)alkyl. In this case, each R is independently selected from H, (C₁-C₆)alkyl and (C₁-C₆)haloalkyl, preferably each R is H. Most preferably R⁴ is

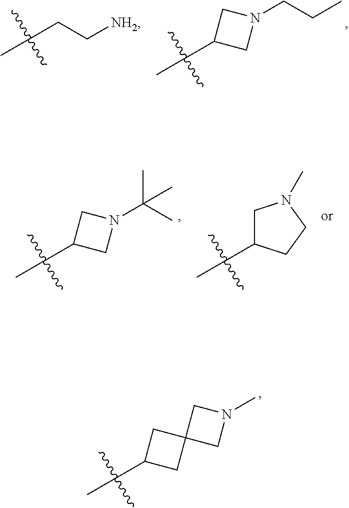

each of which is optionally substituted with halo, preferably F.

In alternative feature of the invention, R¹ is —C(O)R⁷ and R⁷ is selected from H, (C₁-C₆)alkyl, (C₁-C₆)haloalkyl, —NR₂, and 4- to 7-membered non-aromatic heterocycle, wherein the 4- to 7-membered non-aromatic heterocycle is optionally substituted with one or more of halo and (C₁-C₆)alkyl, and each R is independently selected from H, (C₁-C₆)alkyl and (C₁-C₆)haloalkyl. Preferably, R⁷ is a 4- to 7-membered non-aromatic heterocycle, optionally substituted with one or more (C₁-C₃)alkyl. Most preferably R⁷ is

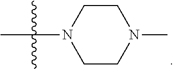 [SMILES: *N1CCN(C)CC1]

R² is present in the compound when at least one of the X groups is CR². That is, at least one of the X groups is not N. If two R² groups are present (i.e. both X groups are CR²), then each R² is chosen independently.

In a particularly preferred embodiment, R² is selected from H, halo, (C₁-C₃)alkyl, (C₁-C₃)haloalkyl, (C₁-C₃)alkoxy, (C₁-C₃)haloalkoxy (preferably —OCF₃), —NR₂ and —O(C₁-C₆)alkylene-NR₂ (preferably —O-propylene-NH₂), and R is independently selected from H, (C₁-C₆)alkyl and (C₁-C₆)haloalkyl. More preferably, R² is selected from H, halo, (C₁-C₃)alkyl (preferably methyl), (C₁-C₃)haloalkyl (preferably —CF₃), and —NR₂ (preferably —NMe₂). Even more preferably R² is H or halo, most preferably H or F. For the reasons se out below, it may be preferable that R² is not (C₁-C₃)alkoxy, and in particular no methoxy.

There is also a preference for one X to be N or CH. This means that one X can be N or CH, and the other X can be N or CR². When one X is N or CH, compounds of the invention can be

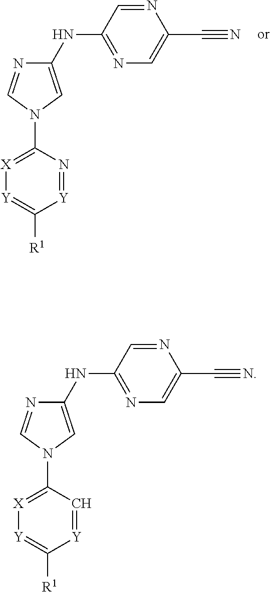

In this case, examples of compounds of the invention may include

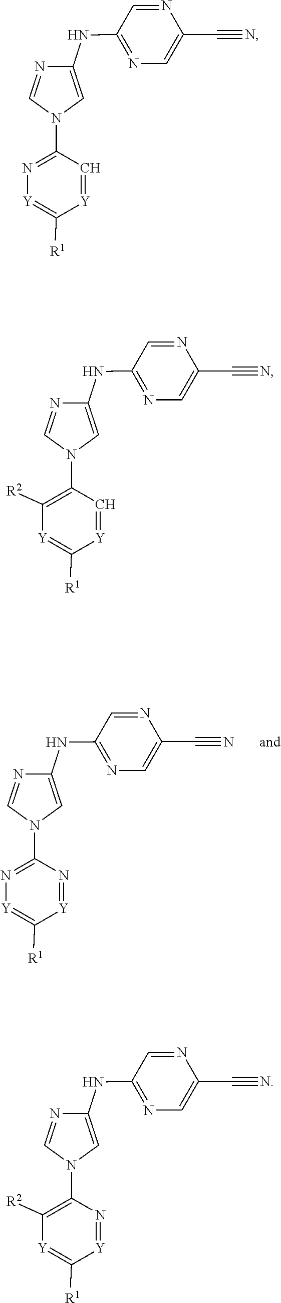

Without wishing to be bound by theory, it has surprisingly been found that when one X is N or CH, the activity of the compounds is improved. This may be due to the reduced steric hindrance offered by the smaller groups in the X or R² positions, which may allow the compounds to bind more effectively to their target, possibly leading to improved Chk1 inhibition.

The compounds of the invention are tolerant to a (C₁-C₃)alkoxy being present in the R² position or positions. However, the presence of those (C₁-C₃)alkoxy groups, and in particular the presence of a methoxy group in the R² position, is not essential for the activity of the compounds. If fact, it may impact the inhibition of Chk1, and thus the potential to treat diseases related to Chk1, such as cancer, when compared to other compounds of the invention. That said, compounds of the invention in which one or two R² are (C₁-C₃)alkoxy (i.e. methoxy) may still be advantageous over previously known compounds.

R³ is present in the compounds of the inventio when at least one of the Y groups is CR³. That is, at least one of the Y groups is not N. If two R³ groups are present (that is, both Y groups are CR³), then each R³ is chosen independently.

In a particularly preferred feature of the invention, R³ is selected from H, halo (preferably F or Cl), (C₁-C₆)alkyl (preferably methyl), (C₁-C₆)haloalkyl (preferably —CF₃), —OR⁴; a 5- to 7-membered non-aromatic heterocycle optionally substituted with one or more R⁹; and a 8- to 10-membered spiro or fused heterocyclic system optionally substituted with one or more of halo and (C₁-C₃)alkyl.

The 5- to 7-membered non-aromatic heterocycle is preferably selected from

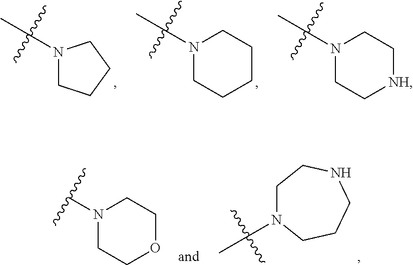 [SMILES: *N1CCCC1 ; *N1CCCCC1 ; *N1CCNCC1 ; *N1CCOCC1 ; *N1CCCNCC1]

each of which is optionally substituted with one or more R⁹.

The 8- to 10-membered spiro or fused heterocyclic system is preferably selected from

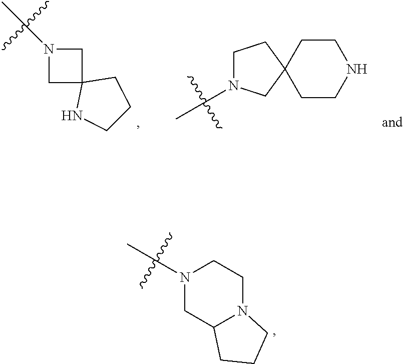 [SMILES: *N1CC2(CCCN2)C1 ; *N1CCC2(CCNCC2)C1 ; *N1CCN2CCCC2C1]

each of which is optionally substituted with one or more of halo and (C₁-C₃)alkyl, preferably F or methyl.

In this case, R⁴ is preferably selected from (C₁-C₆)alkylene-NR₂, a 4- to 6-membered non-aromatic heterocycle, and a 6- to 8-membered heterocyclic spiro system, wherein each of the 4- to 6-membered non-aromatic heterocycle or 6- to 8-membered heterocyclic spiro system is optionally substituted with one or more of (C₁-C₃)alkyl or halo. Preferably R⁴ is (C₁-C₃)alkylene-NH₂ (more preferably propylene-NH₂),

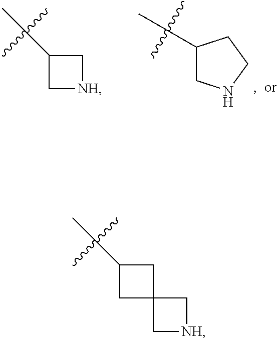 [SMILES: *C1CNC1 ; *C1CCNC1 ; *C1CC2(CNC2)C1]

wherein each of

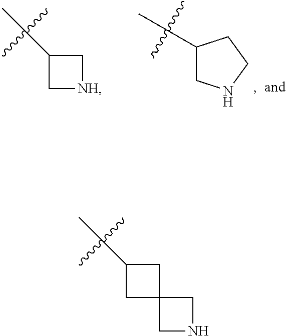 [SMILES: *C1CNC1 ; *C1CCNC1 ; *C1CC2(CNC2)C1]

is optionally substituted with one or more of (C₁-C₃)alkyl or halo.

Additionally, in this feature of the invention, R⁹ is selected from halo, (C₁-C₃)alkyl (preferably methyl), —NR₂, —NR((C₁-C₃)hydroxyalkyl), and (C₁-C₃)alkylene-NR₂ (preferably methylene-NR₂); and each R is independently selected from H, (C₁-C₆)alkyl and (C₁-C₆)haloalkyl, preferably H or methyl.

In a feature of the invention, R¹ is H or methyl, at least one Y is CR³. In this case, R³ is preferably selected from —OR⁴, a 5- to 7-membered non-aromatic heterocycle optionally substituted with one or more R⁹; and an 8- to 10-membered spiro or fused heterocyclic system optionally substituted with one or more of halo and (C₁-C₃)alkyl. In this case, R⁴ may be selected from (C₁-C₆)alkylene-NR₂, a 4- to 6-membered non-aromatic heterocycle, and a 6- to 8-membered heterocyclic spiro system, wherein each of the 4- to 6-membered non-aromatic heterocycle or 6- to 8-membered heterocyclic spiro system is optionally substituted with one or more of (C₁-C₃)alkyl or halo. Additionally, R⁹ may be selected from halo, (C₁-C₃)alkyl, —NR₂, —NR((C₁-C₃)hydroxyalkyl), and (C₁-C₃)alkylene-NR₂; and each R is independently selected from H, (C₁-C₆)alkyl and (C₁-C₆)haloalkyl. Even more preferably, R³ is

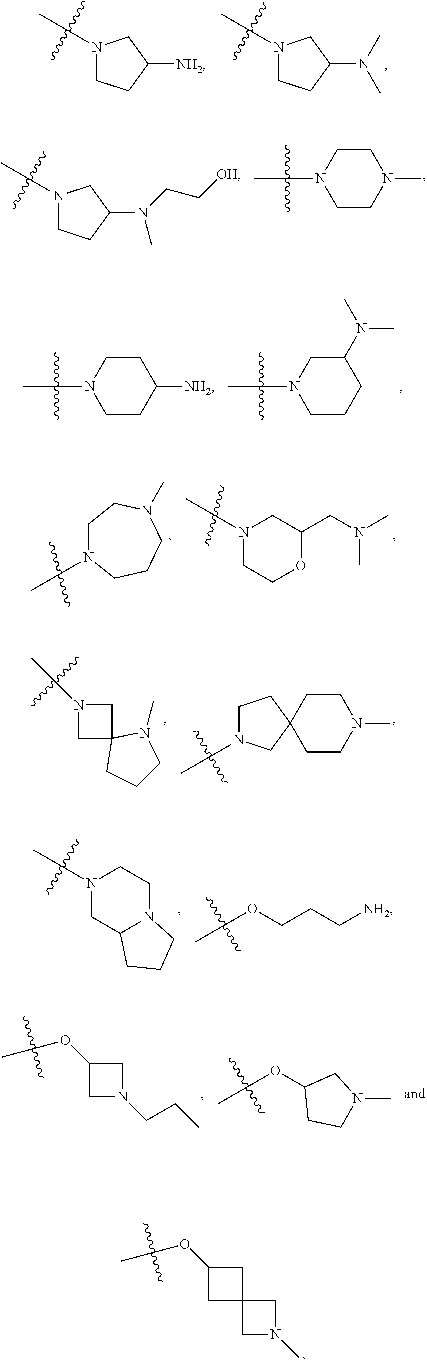

each of which is optionally substituted with one or more halo, preferably F.

Particularly advantageous compounds of the invention are
5-((1-(4-(6-Methyl-3,6-diazabicyclo[3.2.0]heptan-3-yl)phenyl)-1H-imidazol-4-yl)amino)pyrazine-2-carbonitrile
5-((1-(3-Chloro-4-((3-fluoropyrrolidin-1-yl)methyl)phenyl)-1H-imidazol-4-yl)amino)pyrazine-2-carbonitrile
5-((1-(2-Methoxy-4-(4-methylpiperazin-1-yl)phenyl)-1H-imidazol-4-yl)amino)pyrazine-2-carbonitrile
5-((1-(4-(4-Fluoro-1-methylpiperidin-4-yl)phenyl)-1H-imidazol-4-yl)amino)pyrazine-2-carbonitrile
5-((1-(2-Fluoro-4-((3-fluoropyrrolidin-1-yl)methyl)-6-methoxyphenyl)-1H-imidazol-4-yl)amino)pyrazine-2-carbonitrile
5-((1-(2-Fluoro-4-((3-fluoropyrrolidin-1-yl)methyl)phenyl)-1H-imidazol-4-yl)amino)pyrazine-2-carbonitrile
5-((1-(4-((4-Methylpiperazin-1-yl)methyl)phenyl)-1H-imidazol-4-yl)amino)pyrazine-2-carbonitrile
5-((1-(3-(4-Methylpiperazin-1-yl)phenyl)-1H-imidazol-4-yl)amino)pyrazine-2-carbonitrile
5-((1-(4-(5-Methyl-2,5-diazabicyclo[2.2.1]heptan-2-yl)phenyl)-1H-imidazol-4-yl)amino)pyrazine-2-carbonitrile
5-((1-(4-(6-Methyl-2,6-diazaspiro[3.3]heptan-2-yl)phenyl)-1H-imidazol-4-yl)amino)pyrazine-2-carbonitrile
5-((1-(4-(3-(Dimethylamino)pyrrolidin-1-yl)phenyl)-1H-imidazol-4-yl)amino)pyrazine-2-carbonitrile
5-((1-(2-Methoxy-4-((1-methylpiperidin-4-yl)amino)phenyl)-1H-imidazol-4-yl)amino)pyrazine-2-carbonitrile
5-((1-(2-Methoxy-4-(methyl(1-methylpiperidin-4-yl)amino)phenyl)-1H-imidazol-4-yl)amino)pyrazine-2-carbonitrile
5-((1-(4-(3-(Dimethylamino)pyrrolidin-1-yl)phenyl)-1H-imidazol-4-yl)amino)pyrazine-2-carbonitrile
5-((1-(4-(2,4-Dimethylpiperazin-1-yl)phenyl)-1H-imidazol-4-yl)amino)pyrazine-2-carbonitrile
5-((1-(5-(4-Methylpiperazin-1-yl)pyridin-2-yl)-1H-imidazol-4-yl)amino)pyrazine-2-carbonitrile
5-((1-(6-(4-Methylpiperazin-1-yl)pyridin-3-yl)-1H-imidazol-4-yl)amino)pyrazine-2-carbonitrile
5-((1-(6-(4-methyl-1,4-Diazepan-1-yl)pyridin-3-yl)-1H-imidazol-4-yl)amino)pyrazine-2-carbonitrile
5-((1-(3-Fluoro-4-(4-methylpiperazin-1-yl)phenyl)-1H-imidazol-4-yl)amino)pyrazine-2-carbonitrile
5-((1-(4-(Methyl(1-methylpiperidin-4-yl)amino)phenyl)-1H-imidazol-4-yl)amino)pyrazine-2-carbonitrile
5-((1-(2-Fluoro-4-(4-methylpiperazin-1-yl)phenyl)-1H-imidazol-4-yl)amino)pyrazine-2-carbonitrile
5-((1-(4-(((4-Fluoro-1-methylpyrrolidin-3-yl)oxy)methyl)phenyl)-1H-imidazol-4-yl)amino)pyrazine-2-carbonitrile
5-((1-(2-Chloro-4-(4-methylpiperazin-1-yl)phenyl)-1H-imidazol-4-yl)amino)pyrazine-2-carbonitrile
5-((1-(4-((1-Methylpiperidin-4-yl)amino)phenyl)-1H-imidazol-4-yl)amino)pyrazine-2-carbonitrile
5-((1-(4-(4-Ethylpiperazin-1-yl)-2-methoxyphenyl)-1H-imidazol-4-yl)amino)pyrazine-2-carbonitrile
5-((1-(3-Methyl-4-(4-methylpiperazin-1-yl)phenyl)-1H-imidazol-4-yl)amino)pyrazine-2-carbonitrile
5-((1-(4-(4-Methylpiperazin-1-yl)-2-(trifluoromethoxy)phenyl)-1H-imidazol-4-yl)amino)pyrazine-2-carbonitrile
5-((1-(4-(Hexahydropyrrolo[1,2-a]pyrazin-2(1H)-yl)-2-methoxyphenyl)-1H-imidazol-4-yl)amino)pyrazine-2-carbonitrile
5-((1-(4-(2,4-Dimethylpiperazin-1-yl)-2-methoxyphenyl)-1H-imidazol-4-yl)amino)pyrazine-2-carbonitrile
5-((1-(4-(3-(Dimethylamino)azetidin-1-yl)phenyl)-1H-imidazol-4-yl)amino)pyrazine-2-carbonitrile
5-((1-(3-(4-Methyl-1,4-diazepan-1-yl)phenyl)-1H-imidazol-4-yl)amino)pyrazine-2-carbonitrile
5-((1-(3-(5-Methyl-2,5-diazaspiro[3.4]octan-2-yl)phenyl)-1H-imidazol-4-yl)amino)pyrazine-2-carbonitrile
5-((1-(3-(Hexahydropyrrolo[1,2-a]pyrazin-2(1H)-yl)phenyl)-1H-imidazol-4-yl)amino)pyrazine-2-carbonitrile
5-((1-(2-Methoxy-4-(methyl((1-methylpiperidin-4-yl)methyl)amino)phenyl)-1H-imidazol-4-yl)amino)pyrazine-2-carbonitrile
5-((1-(4-(4-(Dimethylamino)piperidin-1-yl)phenyl)-1H-imidazol-4-yl)amino)pyrazine-2-carbonitrile
5-((1-(3-(3-(Dimethylamino)pyrrolidin-1-yl)phenyl)-1H-imidazol-4-yl)amino)pyrazine-2-carbonitrile
5-((1-(3-(8-Methyl-2,8-diazaspiro[4.5]decan-2-yl)phenyl)-1H-imidazol-4-yl)amino)pyrazine-2-carbonitrile
5-((1-(4-(4-Ethyl-2-methylpiperazin-1-yl)phenyl)-1H-imidazol-4-yl)amino)pyrazine-2-carbonitrile
5-((1-(4-((1-Ethylpiperidin-4-yl)amino)-2-methoxyphenyl)-1H-imidazol-4-yl)amino)pyrazine-2-carbonitrile
5-((1-(4-(5-Methyl-2,5-diazabicyclo[2.2.2]octan-2-yl)phenyl)-1H-imidazol-4-yl)amino)pyrazine-2-carbonitrile
5-((1-(3-(Methyl(1-methylpiperidin-4-yl)amino)phenyl)-1H-imidazol-4-yl)amino)pyrazine-2-carbonitrile
5-((1-(2-Methoxy-4-(-5-methyl-2,5-diazabicyclo[2.2.1]heptan-2-yl)phenyl)-1H-imidazol-4-yl)amino)pyrazine-2-carbonitrile
5-((1-(4-((1-(tert-Butyl)azetidin-3-yl)oxy)phenyl)-1H-imidazol-4-yl)amino)pyrazine-2-carbonitrile
5-((1-(2-Methoxy-4-(6-methyl-2,6-diazaspiro[3.3]heptan-2-yl)phenyl)-1H-imidazol-4-yl)amino)pyrazine-2-carbonitrile
5-((1-(4-(3-(Dimethylamino)pyrrolidin-1-yl)-2-methoxyphenyl)-1H-imidazol-4-yl)amino)pyrazine-2-carbonitrile
5-((1-(3-((1-Propylazetidin-3-yl)oxy)phenyl)-1H-imidazol-4-yl)amino)pyrazine-2-carbonitrile
5-((1-(4-((1-Propylazetidin-3-yl)oxy)phenyl)-1H-imidazol-4-yl)amino)pyrazine-2-carbonitrile
5-((1-(5-(3-(Dimethylamino)pyrrolidin-1-yl)pyridin-2-yl)-1H-imidazol-4-yl)amino)pyrazine-2-carbonitrile
5-((1-(4-([1,3′-Bipyrrolidin]-1′-yl)-2-methoxyphenyl)-1H-imidazol-4-yl)amino)pyrazine-2-carbonitrile
5-((1-(5-(3-(Dimethylamino)pyrrolidin-1-yl)pyrazin-2-yl)-1H-imidazol-4-yl)amino)pyrazine-2-carbonitrile
5-((1-(4-((1-(tert-Butyl)azetidin-3-yl)oxy)-2-methoxyphenyl)-1H-imidazol-4-yl)amino)pyrazine-2-carbonitrile
5-((1-(4-((3-Fluoropyrrolidin-1-yl)methyl)-2-methyl-6-(trifluoromethyl)phenyl)-1H-imidazol-4-yl)amino)pyrazine-2-carbonitrile
5-((1-(2-Methoxy-4-(1-methylhexahydropyrrolo[3,4-b]pyrrol-5(1H)-yl)phenyl)-1H-imidazol-4-yl)amino)pyrazine-2-carbonitrile
5-((1-(3-((-4-fluoro-1-methylpyrrolidin-3-yl)oxy)phenyl)-1H-imidazol-4-yl)amino)pyrazine-2-carbonitrile
5-((1-(4-((3-Fluoropyrrolidin-1-yl)methyl)-2,6-dimethylphenyl)-1H-imidazol-4-yl)amino)pyrazine-2-carbonitrile
5-((1-(3-(3-(Dimethylamino)pyrrolidin-1-yl)-4-methylphenyl)-1H-imidazol-4-yl)amino)pyrazine-2-carbonitrile
5-((1-(5-(3-(Dimethylamino)pyrrolidin-1-yl)-6-methylpyridin-2-yl)-1H-imidazol-4-yl)amino)pyrazine-2-carbonitrile
5-((1-(4-(8-Methyl-2,8-diazaspiro[4.5]decan-2-yl)phenyl)-1H-imidazol-4-yl)amino)pyrazine-2-carbonitrile
5-((1-(4-(1-Methyl-1,6-diazaspiro[3.3]heptan-6-yl)phenyl)-1H-imidazol-4-yl)amino)pyrazine-2-carbonitrile
5-((1-(4-(-3-(Dimethylamino)-4-fluoropyrrolidin-1-yl)phenyl)-1H-imidazol-4-yl)amino)pyrazine-2-carbonitrile
5-((1-(6-(3-(Dimethylamino)pyrrolidin-1-yl)pyridin-3-yl)-1H-imidazol-4-yl)amino)pyrazine-2-carbonitrile
5-((1-(4-(3-(Dimethylamino)pyrrolidin-1-yl)-2-fluorophenyl)-1H-imidazol-4-yl)amino)pyrazine-2-carbonitrile
5-((1-(4-(2-((Dimethylamino)methyl)pyrrolidin-1-yl)phenyl)-1H-imidazol-4-yl)amino)pyrazine-2-carbonitrile
5-((1-(4-(-3-Methyl-3,6-diazabicyclo[3.2.0]heptan-6-yl)phenyl)-1H-imidazol-4-yl)amino)pyrazine-2-carbonitrile
5-((1-(4-(2-Methyl-2,6-diazaspiro[3.4]octan-6-yl)phenyl)-1H-imidazol-4-yl)amino)pyrazine-2-carbonitrile
5-((1-(4-(3-(Dimethylamino)pyrrolidin-1-yl)-3-(trifluoromethyl)phenyl)-1H-imidazol-4-yl)amino)pyrazine-2-carbonitrile
5-((1-(4-(3-(Dimethylamino)pyrrolidin-1-yl)-2-(trifluoromethyl)phenyl)-1H-imidazol-4-yl)amino)pyrazine-2-carbonitrile
5-((1-(4-(3-Morpholinopyrrolidin-1-yl)phenyl)-1H-imidazol-4-yl)amino)pyrazine-2-carbonitrile
5-((1-(4-(-3-(Dimethylamino)-4-methylpyrrolidin-1-yl)phenyl)-1H-imidazol-4-yl)amino)pyrazine-2-carbonitrile
5-((1-(4-(-4-(Dimethylamino)-2-methylpyrrolidin-1-yl)phenyl)-1H-imidazol-4-yl)amino)pyrazine-2-carbonitrile
5-((1-(4-(3-(Dimethylamino)-4-methylpyrrolidin-1-yl)phenyl)-1H-imidazol-4-yl)amino)pyrazine-2-carbonitrile
5-((1-(5-(3-(Dimethylamino)pyrrolidin-1-yl)-6-(trifluoromethyl)pyridin-2-yl)-1H-imidazol-4-yl)amino)pyrazine-2-carbonitrile
5-((1-(4-(3-(Dimethylamino)-3-methylpyrrolidin-1-yl)phenyl)-1H-imidazol-4-yl)amino)pyrazine-2-carbonitrile
5-((1-(4-(3-(Dimethylamino)pyrrolidin-1-yl)-3-fluorophenyl)-1H-imidazol-4-yl)amino)pyrazine-2-carbonitrile
5-((1-(2-Fluoro-4-(3-methyl-3,6-diazabicyclo[3.2.0]heptan-6-yl)phenyl)-1H-imidazol-4-yl)amino)pyrazine-2-carbonitrile
5-((1-(5-(3-(Dimethylamino)pyrrolidin-1-yl)pyrimidin-2-yl)-1H-imidazol-4-yl)amino)pyrazine-2-carbonitrile
5-((1-(2-(Dimethylamino)-4-(3-(dimethylamino)pyrrolidin-1-yl)phenyl)-1H-imidazol-4-yl)amino)pyrazine-2-carbonitrile
5-((1-(4-(3-(Bis(methyl-d3)amino)pyrrolidin-1-yl)phenyl)-1H-imidazol-4-yl)amino)pyrazine-2-carbonitrile
5-((1-(5-(-4-(Dimethylamino)-2-methylpyrrolidin-1-yl)pyridin-2-yl)-1H-imidazol-4-yl)amino)pyrazine-2-carbonitrile
5-((1-(4-(-3-(Dimethylamino)-2-methylpyrrolidin-1-yl)phenyl)-1H-imidazol-4-yl)amino)pyrazine-2-carbonitrile
5-((1-(4-(1-Methylhexahydropyrrolo[3,4-b]pyrrol-5(1H)-yl)phenyl)-1H-imidazol-4-yl)amino)pyrazine-2-carbonitrile
5-((1-(4-(3-(Ethylamino)-4-fluoropyrrolidin-1-yl)phenyl)-1H-imidazol-4-yl)amino)pyrazine-2-carbonitrile
5-((1-(4-(3-(Ethyl(methyl)amino)pyrrolidin-1-yl)phenyl)-1H-imidazol-4-yl)amino)pyrazine-2-carbonitrile
5-((1-(5-(3-(Ethyl(methyl)amino)pyrrolidin-1-yl)pyridin-2-yl)-1H-imidazol-4-yl)amino)pyrazine-2-carbonitrile
5-((1-(4-(4-Methylhexahydropyrrolo[3,2-b]pyrrol-1(2H)-yl)phenyl)-1H-imidazol-4-yl)amino)pyrazine-2-carbonitrile
5-((1-(4-(3-Fluoro-4-(methylamino)pyrrolidin-1-yl)phenyl)-1H-imidazol-4-yl)amino)pyrazine-2-carbonitrile
5-((1-(5-(3-((3,3,3-Trifluoropropyl)amino)pyrrolidin-1-yl)pyridin-2-yl)-1H-imidazol-4-yl)amino)pyrazine-2-carbonitrile
5-((1-(4-(Methyl(1-methylpyrrolidin-3-yl)amino)phenyl)-1H-imidazol-4-yl)amino)pyrazine-2-carbonitrile
5-((1-(4-(3-(Isopropylamino)pyrrolidin-1-yl)phenyl)-1H-imidazol-4-yl)amino)pyrazine-2-carbonitrile
5-((1-(4-(3,3,4-Trimethylpiperazin-1-yl)phenyl)-1H-imidazol-4-yl)amino)pyrazine-2-carbonitrile
5-((1-(4-(4-(Azetidin-1-yl)-2-methylpyrrolidin-1-yl)phenyl)-1H-imidazol-4-yl)amino)pyrazine-2-carbonitrile
5-((1-(4-(6-Methyl-1,6-diazaspiro[3.3]heptan-1-yl)phenyl)-1H-imidazol-4-yl)amino)pyrazine-2-carbonitrile
5-((1-(4-(1-Methyl-1,7-diazaspiro[3.5]nonan-7-yl)phenyl)-1H-imidazol-4-yl)amino)pyrazine-2-carbonitrile
5-((1-(4-(5-Methylhexahydropyrrolo[3,4-c]pyrrol-2(1H)-yl)phenyl)-1H-imidazol-4-yl)amino)pyrazine-2-carbonitrile
5-((1-(5-(3-(Azetidin-1-yl)pyrrolidin-1-yl)pyridin-2-yl)-1H-imidazol-4-yl)amino)pyrazine-2-carbonitrile
5-((1-(4-(3-(Azetidin-1-yl)pyrrolidin-1-yl)-2-methoxyphenyl)-1H-imidazol-4-yl)amino)pyrazine-2-carbonitrile
5-((1-(4-(3-(Azetidin-1-yl)pyrrolidin-1-yl)-3-methylphenyl)-1H-imidazol-4-yl)amino)pyrazine-2-carbonitrile
5-((1-(4-(4-Methylmorpholin-2-yl)phenyl)-1H-imidazol-4-yl)amino)pyrazine-2-carbonitrile
5-((1-(3-(2-((Dimethylamino)methyl)morpholino)phenyl)-1H-imidazol-4-yl)amino)pyrazine-2-carbonitrile
5-((1-(3-(3-((2-Hydroxyethyl)(methyl)amino)pyrrolidin-1-yl)phenyl)-1H-imidazol-4-yl)amino)pyrazine-2-carbonitrile
5-((1-(3-(3-(Dimethylamino)piperidin-1-yl)phenyl)-1H-imidazol-4-yl)amino)pyrazine-2-carbonitrile
5-((1-(4-(3-(Azetidin-1-yl)pyrrolidin-1-yl)phenyl)-1H-imidazol-4-yl)amino)pyrazine-2-carbonitrile
5-((1-(4-(3-((Dimethylamino)methyl)pyrrolidin-1-yl)phenyl)-1H-imidazol-4-yl)amino)pyrazine-2-carbonitrile
5-((1-(4-(Hexahydropyrrolo[1,2-a]pyrazin-2(1H)-yl)phenyl)-1H-imidazol-4-yl)amino)pyrazine-2-carbonitrile
5-((1-(4-(6-Methyl-1,6-diazaspiro[3.4]octan-1-yl)phenyl)-1H-imidazol-4-yl)amino)pyrazine-2-carbonitrile
5-((1-(4-(3-(Dimethylamino)piperidin-1-yl)phenyl)-1H-imidazol-4-yl)amino)pyrazine-2-carbonitrile
5-((1-(4-(3-(3,3-Dimethylazetidin-1-yl)pyrrolidin-1-yl)phenyl)-1H-imidazol-4-yl)amino)pyrazine-2-carbonitrile
5-((1-(4-(3-(3-Methylazetidin-1-yl)pyrrolidin-1-yl)phenyl)-1H-imidazol-4-yl)amino)pyrazine-2-carbonitrile
5-((1-(4-(1-Methyl-1,6-diazaspiro[3.4]octan-6-yl)phenyl)-1H-imidazol-4-yl)amino)pyrazine-2-carbonitrile
5-((1-(4-(3-(3-Fluoroazetidin-1-yl)pyrrolidin-1-yl)phenyl)-1H-imidazol-4-yl)amino)pyrazine-2-carbonitrile
5-((1-(4-(3-(2-Azaspiro[3.3]heptan-2-yl)pyrrolidin-1-yl)phenyl)-1H-imidazol-4-yl)amino)pyrazine-2-carbonitrile
5-((1-(4-(3-(3-Isopropylazetidin-1-yl)pyrrolidin-1-yl)phenyl)-1H-imidazol-4-yl)amino)pyrazine-2-carbonitrile
5-((1-(4-(3-(2-Methylazetidin-1-yl)pyrrolidin-1-yl)phenyl)-1H-imidazol-4-yl)amino)pyrazine-2-carbonitrile
5-((1-(4-(3-(2,2-Dimethylazetidin-1-yl)pyrrolidin-1-yl)phenyl)-1H-imidazol-4-yl)amino)pyrazine-2-carbonitrile
1-(4-(4-((5-Cyanopyrazin-2-yl)amino)-1H-imidazol-1-yl)phenyl)-N,N-dimethylpyrrolidin-3-amine oxide
5-((1-(4-(Morpholinomethyl)phenyl)-1H-imidazol-4-yl)amino)pyrazine-2-carbonitrile
5-((1-(4-(4-Methylpiperazin-1-yl)phenyl)-1H-imidazol-4-yl)amino)pyrazine-2-carbonitrile
5-((1-(4-(4-Methylpiperazine-1-carbonyl)phenyl)-1H-imidazol-4-yl)amino)pyrazine-2-carbonitrile
5-((1-(2-methyl-4-(4-Methylpiperazin-1-yl)phenyl)-1H-imidazol-4-yl)amino)pyrazine-2-carbonitrile
5-((1-(4-([1,3′-Bipyrrolidin]-1′-yl)phenyl)-1H-imidazol-4-yl)amino)pyrazine-2-carbonitrile
5-((1-(3-(3-Aminopropoxy)phenyl)-1H-imidazol-4-yl)amino)pyrazine-2-carbonitrile
5-((1-(2-(3-Aminopropoxy)-6-methoxyphenyl)-1H-imidazol-4-yl)amino)pyrazine-2-carbonitrile
5-((1-(4-(2-Aminoethoxy)phenyl)-1H-imidazol-4-yl)amino)pyrazine-2-carbonitrile
5-((1-(3-((3-Amino-4-fluoropyrrolidin-1-yl)phenyl)-1H-imidazol-4-yl)amino)pyrazine-2-carbonitrile
5-((1-(4-(3-Amino-4-fluoropyrrolidin-1-yl)phenyl)-1H-imidazol-4-yl)amino)pyrazine-2-carbonitrile
5-((1-(4-(3-Aminopyrrolidin-1-yl)phenyl)-1H-imidazol-4-yl)amino)pyrazine-2-carbonitrile
5-((1-(4-(7-Amino-5-azaspiro[2.4]heptan-5-yl)phenyl)-1H-imidazol-4-yl)amino)pyrazine-2-carbonitrile
5-((1-(4-(3-(Methylamino)pyrrolidin-1-yl)phenyl)-1H-imidazol-4-yl)amino)pyrazine-2-carbonitrile
5-((1-(4-(3-Amino-3-methylpyrrolidin-1-yl)phenyl)-1H-imidazol-4-yl)amino)pyrazine-2-carbonitrile
5-((1-(4-(3-Amino-4-fluoropyrrolidin-1-yl)phenyl)-1H-imidazol-4-yl)amino)pyrazine-2-carbonitrile
5-((1-(4-(3-(Aminomethyl)azetidin-1-yl)phenyl)-1H-imidazol-4-yl)amino)pyrazine-2-carbonitrile
5-((1-(4-(3-(Aminomethyl)-3-fluoroazetidin-1-yl)phenyl)-1H-imidazol-4-yl)amino)pyrazine-2-carbonitrile
5-((1-(4-(1,6-Diazaspiro[3.3]heptan-6-yl)phenyl)-1H-imidazol-4-yl)amino)pyrazine-2-carbonitrile
5-((1-(4-(3,6-Diazabicyclo[3.2.0]heptan-3-yl)phenyl)-1H-imidazol-4-yl)amino)pyrazine-2-carbonitrile
5-((1-(4-(3-(Ethylamino)pyrrolidin-1-yl)phenyl)-1H-imidazol-4-yl)amino)pyrazine-2-carbonitrile
5-((1-(4-(3-Methoxy-4-(methylamino)pyrrolidin-1-yl)phenyl)-1H-imidazol-4-yl)amino)pyrazine-2-carbonitrile
5-((1-(4-(3-Fluoro-4-(methylamino)pyrrolidin-1-yl)-2-methoxyphenyl)-1H-imidazol-4-yl)amino)pyrazine-2-carbonitrile
5-((1-(4-(4-Amino-2-methylpyrrolidin-1-yl)phenyl)-1H-imidazol-4-yl)amino)pyrazine-2-carbonitrile
5-((1-(3-(4-Amino-3,3-difluoropiperidin-1-yl)phenyl)-1H-imidazol-4-yl)amino)pyrazine-2-carbonitrile
5-((1-(2-(3-Aminopropoxy)phenyl)-1H-imidazol-4-yl)amino)pyrazine-2-carbonitrile
5-((1-(4-((3-Fluoropyrrolidin-1-yl)methyl)-2-methoxyphenyl)-1H-imidazol-4-yl)amino)pyrazine-2-carbonitrile)
5-((1-(4-((Ethylamino)methyl)-2-methoxyphenyl)-1H-imidazol-4-yl)amino)pyrazine-2-carbonitrile
5-((1-(2-Methoxy-4-(pyrrolidin-1-ylmethyl)phenyl)-1H-imidazol-4-yl)amino)pyrazine-2-carbonitrile
5-((1-(4-((4,4-Difluoropiperidin-1-yl)methyl)-2-methoxyphenyl)-1H-imidazol-4-yl)amino)pyrazine-2-carbonitrile
5-((1-(2-Methoxy-4-(morpholinomethyl)phenyl)-1H-imidazol-4-yl)amino)pyrazine-2-carbonitrile
5-((1-(4-((4-Fluoropiperidin-1-yl)methyl)phenyl)-1H-imidazol-4-yl)amino)pyrazine-2-carbonitrile
5-((1-(3-((2-Methyl-2-azaspiro[3.3]heptan-6-yl)oxy)phenyl)-1H-imidazol-4-yl)amino)pyrazine-2-carbonitrile
5-((1-(4-(1-Methylazetidin-3-yl)phenyl)-1H-imidazol-4-yl)amino)pyrazine-2-carbonitrile
5-((1-(4-(9-Methyl-3-oxa-7,9-diazabicyclo[3.3.1]nonan-7-yl)phenyl)-1H-imidazol-4-yl)amino)pyrazine-2-carbonitrile
5-((1-(4-((2-Methyl-2-azaspiro[3.3]heptan-6-yl)oxy)phenyl)-1H-imidazol-4-yl)amino)pyrazine-2-carbonitrile
5-((1-(4-(1-(Diethylamino)cyclopropyl)phenyl)-1H-imidazol-4-yl)amino)pyrazine-2-carbonitrile
5-((1-(2-Methoxy-4-((2-methyl-2-azaspiro[3.3]heptan-6-yl)oxy)phenyl)-1H-imidazol-4-yl)amino)pyrazine-2-carbonitrile
5-((1-(4-(4-(3,3-Difluorocyclobutyl)piperazin-1-yl)phenyl)-1H-imidazol-4-yl)amino)pyrazine-2-carbonitrile
5-((1-(4-(4-(Oxetan-3-yl)piperazin-1-yl)phenyl)-1H-imidazol-4-yl)amino)pyrazine-2-carbonitrile
5-((1-(4-(4-(2-Fluoroethyl)piperazin-1-yl)phenyl)-1H-imidazol-4-yl)amino)pyrazine-2-carbonitrile
5-((1-(4-(4-(3,3,3-Trifluoropropyl)piperazin-1-yl)phenyl)-1H-imidazol-4-yl)amino)pyrazine-2-carbonitrile
5-((1-(4-(4-Ethylpiperazin-1-yl)phenyl)-1H-imidazol-4-yl)amino)pyrazine-2-carbonitrile
5-((1-(4-(4-Isopropylpiperazin-1-yl)phenyl)-1H-imidazol-4-yl)amino)pyrazine-2-carbonitrile
   or a pharmaceutically acceptable salt, solvate, hydrate, tautomer, enantiomer, diastereoisomer, isotopic form, N-oxide, and/or prodrug thereof.

More preferred compounds of the invention include
5-((1-(4-((1R,5R)-6-Methyl-3,6-diazabicyclo[3.2.0]heptan-3-yl)phenyl)-1H-imidazol-4-yl)amino)pyrazine-2-carbonitrile
(R)-5-((1-(3-Chloro-4-((3-fluoropyrrolidin-1-yl)methyl)phenyl)-1H-imidazol-4-yl)amino)pyrazine-2-carbonitrile
5-((1-(2-Methoxy-4-(4-methylpiperazin-1-yl)phenyl)-1H-imidazol-4-yl)amino)pyrazine-2-carbonitrile
5-((1-(4-(4-Fluoro-1-methylpiperidin-4-yl)phenyl)-1H-imidazol-4-yl)amino)pyrazine-2-carbonitrile
(R)-5-((1-(2-Fluoro-4-((3-fluoropyrrolidin-1-yl)methyl)-6-methoxyphenyl)-1H-imidazol-4-yl)amino)pyrazine-2-carbonitrile
(R)-5-((1-(2-Fluoro-4-((3-fluoropyrrolidin-1-yl)methyl)phenyl)-1H-imidazol-4-yl)amino)pyrazine-2-carbonitrile
5-((1-(4-((4-Methylpiperazin-1-yl)methyl)phenyl)-1H-imidazol-4-yl)amino)pyrazine-2-carbonitrile
5-((1-(3-(4-Methylpiperazin-1-yl)phenyl)-1H-imidazol-4-yl)amino)pyrazine-2-carbonitrile
5-((1-(4-((1R,4R)-5-Methyl-2,5-diazabicyclo[2.2.1]heptan-2-yl)phenyl)-1H-imidazol-4-yl)amino)pyrazine-2-carbonitrile
5-((1-(4-((1S,4S)-5-Methyl-2,5-diazabicyclo[2.2.1]heptan-2-yl)phenyl)-1H-imidazol-4-yl)amino)pyrazine-2-carbonitrile
5-((1-(4-(6-Methyl-2,6-diazaspiro[3.3]heptan-2-yl)phenyl)-1H-imidazol-4-yl)amino)pyrazine-2-carbonitrile
(R)-5-((1-(4-(3-(Dimethylamino)pyrrolidin-1-yl)phenyl)-1H-imidazol-4-yl)amino)pyrazine-2-carbonitrile
5-((1-(2-Methoxy-4-((1-methylpiperidin-4-yl)amino)phenyl)-1H-imidazol-4-yl)amino)pyrazine-2-carbonitrile
5-((1-(2-Methoxy-4-(methyl(1-methylpiperidin-4-yl)amino)phenyl)-1H-imidazol-4-yl)amino)pyrazine-2-carbonitrile
(S)-5-((1-(4-(3-(Dimethylamino)pyrrolidin-1-yl)phenyl)-1H-imidazol-4-yl)amino)pyrazine-2-carbonitrile
(R)-5-((1-(4-(2,4-Dimethylpiperazin-1-yl)phenyl)-1H-imidazol-4-yl)amino)pyrazine-2-carbonitrile
(S)-5-((1-(4-(2,4-Dimethylpiperazin-1-yl)phenyl)-1H-imidazol-4-yl)amino)pyrazine-2-carbonitrile
5-((1-(5-(4-Methylpiperazin-1-yl)pyridin-2-yl)-1H-imidazol-4-yl)amino)pyrazine-2-carbonitrile
5-((1-(6-(4-Methylpiperazin-1-yl)pyridin-3-yl)-1H-imidazol-4-yl)amino)pyrazine-2-carbonitrile
5-((1-(6-(4-methyl-1,4-Diazepan-1-yl)pyridin-3-yl)-1H-imidazol-4-yl)amino)pyrazine-2-carbonitrile
5-((1-(3-Fluoro-4-(4-methylpiperazin-1-yl)phenyl)-1H-imidazol-4-yl)amino)pyrazine-2-carbonitrile
5-((1-(4-(Methyl(1-methylpiperidin-4-yl)amino)phenyl)-1H-imidazol-4-yl)amino)pyrazine-2-carbonitrile
5-((1-(2-Fluoro-4-(4-methylpiperazin-1-yl)phenyl)-1H-imidazol-4-yl)amino)pyrazine-2-carbonitrile
5-((1-(4-((((3S,4S)-4-Fluoro-1-methylpyrrolidin-3-yl)oxy)methyl)phenyl)-1H-imidazol-4-yl)amino)pyrazine-2-carbonitrile
5-((1-(2-Chloro-4-(4-methylpiperazin-1-yl)phenyl)-1H-imidazol-4-yl)amino)pyrazine-2-carbonitrile
5-((1-(4-((1-Methylpiperidin-4-yl)amino)phenyl)-1H-imidazol-4-yl)amino)pyrazine-2-carbonitrile
5-((1-(4-(4-Ethylpiperazin-1-yl)-2-methoxyphenyl)-1H-imidazol-4-yl)amino)pyrazine-2-carbonitrile
5-((1-(3-Methyl-4-(4-methylpiperazin-1-yl)phenyl)-1H-imidazol-4-yl)amino)pyrazine-2-carbonitrile
5-((1-(4-(4-Methylpiperazin-1-yl)-2-(trifluoromethoxy)phenyl)-1H-imidazol-4-yl)amino)pyrazine-2-carbonitrile
(S)-5-((1-(4-(Hexahydropyrrolo[1,2-a]pyrazin-2(1H)-yl)-2-methoxyphenyl)-1H-imidazol-4-yl)amino)pyrazine-2-carbonitrile
(S)-5-((1-(4-(2,4-Dimethylpiperazin-1-yl)-2-methoxyphenyl)-1H-imidazol-4-yl)amino)pyrazine-2-carbonitrile
5-((1-(4-(3-(Dimethylamino)azetidin-1-yl)phenyl)-1H-imidazol-4-yl)amino)pyrazine-2-carbonitrile
5-((1-(3-(4-Methyl-1,4-diazepan-1-yl)phenyl)-1H-imidazol-4-yl)amino)pyrazine-2-carbonitrile
5-((1-(3-(5-Methyl-2,5-diazaspiro[3.4]octan-2-yl)phenyl)-1H-imidazol-4-yl)amino)pyrazine-2-carbonitrile
(S)-5-((1-(3-(Hexahydropyrrolo[1,2-a]pyrazin-2(1H)-yl)phenyl)-1H-imidazol-4-yl)amino)pyrazine-2-carbonitrile
5-((1-(2-Methoxy-4-(methyl((1-methylpiperidin-4-yl)methyl)amino)phenyl)-1H-imidazol-4-yl)amino)pyrazine-2-carbonitrile
5-((1-(4-(4-(Dimethylamino)piperidin-1-yl)phenyl)-1H-imidazol-4-yl)amino)pyrazine-2-carbonitrile
(R)-5-((1-(3-(3-(Dimethylamino)pyrrolidin-1-yl)phenyl)-1H-imidazol-4-yl)amino)pyrazine-2-carbonitrile
5-((1-(3-(8-Methyl-2,8-diazaspiro[4.5]decan-2-yl)phenyl)-1H-imidazol-4-yl)amino)pyrazine-2-carbonitrile
(R)-5-((1-(4-(4-Ethyl-2-methylpiperazin-1-yl)phenyl)-1H-imidazol-4-yl)amino)pyrazine-2-carbonitrile
5-((1-(4-((1-Ethylpiperidin-4-yl)amino)-2-methoxyphenyl)-1H-imidazol-4-yl)amino)pyrazine-2-carbonitrile
5-((1-(4-((1S,4S)-5-Methyl-2,5-diazabicyclo[2.2.2]octan-2-yl)phenyl)-1H-imidazol-4-yl)amino)pyrazine-2-carbonitrile
5-((1-(3-(Methyl(1-methylpiperidin-4-yl)amino)phenyl)-1H-imidazol-4-yl)amino)pyrazine-2-carbonitrile
5-((1-(2-Methoxy-4-((1R,4R)-5-methyl-2,5-diazabicyclo[2.2.1]heptan-2-yl)phenyl)-1H-imidazol-4-yl)amino)pyrazine-2-carbonitrile
5-((1-(4-((1-(tert-Butyl)azetidin-3-yl)oxy)phenyl)-1H-imidazol-4-yl)amino)pyrazine-2-carbonitrile
5-((1-(2-Methoxy-4-(6-methyl-2,6-diazaspiro[3.3]heptan-2-yl)phenyl)-1H-imidazol-4-yl)amino)pyrazine-2-carbonitrile
(R)-5-((1-(4-(3-(Dimethylamino)pyrrolidin-1-yl)-2-methoxyphenyl)-1H-imidazol-4-yl)amino)pyrazine-2-carbonitrile
5-((1-(3-((1-Propylazetidin-3-yl)oxy)phenyl)-1H-imidazol-4-yl)amino)pyrazine-2-carbonitrile
5-((1-(4-((1-Propylazetidin-3-yl)oxy)phenyl)-1H-imidazol-4-yl)amino)pyrazine-2-carbonitrile
(R)-5-((1-(5-(3-(Dimethylamino)pyrrolidin-1-yl)pyridin-2-yl)-1H-imidazol-4-yl)amino)pyrazine-2-carbonitrile
(R)-5-((1-(4-([1,3′-Bipyrrolidin]-1′-yl)-2-methoxyphenyl)-1H-imidazol-4-yl)amino)pyrazine-2-carbonitrile
(R)-5-((1-(5-(3-(Dimethylamino)pyrrolidin-1-yl)pyrazin-2-yl)-1H-imidazol-4-yl)amino)pyrazine-2-carbonitrile
5-((1-(4-((1-(tert-Butyl)azetidin-3-yl)oxy)-2-methoxyphenyl)-1H-imidazol-4-yl)amino)pyrazine-2-carbonitrile
(R)-5-((1-(4-((3-Fluoropyrrolidin-1-yl)methyl)-2-methyl-6-(trifluoromethyl)phenyl)-1H-imidazol-4-yl)amino)pyrazine-2-carbonitrile
5-((1-(2-Methoxy-4-((3aR,6aR)-1-methylhexahydropyrrolo[3,4-b]pyrrol-5(1H)-yl)phenyl)-1H-imidazol-4-yl)amino)pyrazine-2-carbonitrile
5-((1-(3-(((3R,4S)-4-fluoro-1-methylpyrrolidin-3-yl)oxy)phenyl)-1H-imidazol-4-yl)amino)pyrazine-2-carbonitrile
(R)-5-((1-(4-((3-Fluoropyrrolidin-1-yl)methyl)-2,6-dimethylphenyl)-1H-imidazol-4-yl)amino)pyrazine-2-carbonitrile
(R)-5-((1-(3-(3-(Dimethylamino)pyrrolidin-1-yl)-4-methylphenyl)-1H-imidazol-4-yl)amino)pyrazine-2-carbonitrile
(R)-5-((1-(5-(3-(Dimethylamino)pyrrolidin-1-yl)-6-methylpyridin-2-yl)-1H-imidazol-4-yl)amino)pyrazine-2-carbonitrile
5-((1-(4-(8-Methyl-2,8-diazaspiro[4.5]decan-2-yl)phenyl)-1H-imidazol-4-yl)amino)pyrazine-2-carbonitrile
5-((1-(4-(1-Methyl-1,6-diazaspiro[3.3]heptan-6-yl)phenyl)-1H-imidazol-4-yl)amino)pyrazine-2-carbonitrile
5-((1-(4-((3S,4R)-3-(Dimethylamino)-4-fluoropyrrolidin-1-yl)phenyl)-1H-imidazol-4-yl)amino)pyrazine-2-carbonitrile
5-((1-(4-((3S,4S)-3-(Dimethylamino)-4-fluoropyrrolidin-1-yl)phenyl)-1H-imidazol-4-yl)amino)pyrazine-2-carbonitrile
(R)-5-((1-(6-(3-(Dimethylamino)pyrrolidin-1-yl)pyridin-3-yl)-1H-imidazol-4-yl)amino)pyrazine-2-carbonitrile
(R)-5-((1-(4-(3-(Dimethylamino)pyrrolidin-1-yl)-2-fluorophenyl)-1H-imidazol-4-yl)amino)pyrazine-2-carbonitrile
(S)-5-((1-(4-(2-((Dimethylamino)methyl)pyrrolidin-1-yl)phenyl)-1H-imidazol-4-yl)amino)pyrazine-2-carbonitrile
5-((1-(4-((1R,5S)-3-Methyl-3,6-diazabicyclo[3.2.0]heptan-6-yl)phenyl)-1H-imidazol-4-yl)amino)pyrazine-2-carbonitrile
5-((1-(4-((1S,5R)-3-Methyl-3,6-diazabicyclo[3.2.0]heptan-6-yl)phenyl)-1H-imidazol-4-yl)amino)pyrazine-2-carbonitrile
5-((1-(4-(2-Methyl-2,6-diazaspiro[3.4]octan-6-yl)phenyl)-1H-imidazol-4-yl)amino)pyrazine-2-carbonitrile
(R)-5-((1-(4-(3-(Dimethylamino)pyrrolidin-1-yl)-3-(trifluoromethyl)phenyl)-1H-imidazol-4-yl)amino)pyrazine-2-carbonitrile
(R)-5-((1-(4-(3-(Dimethylamino)pyrrolidin-1-yl)-2-(trifluoromethyl)phenyl)-1H-imidazol-4-yl)amino)pyrazine-2-carbonitrile
(R)-5-((1-(4-(3-Morpholinopyrrolidin-1-yl)phenyl)-1H-imidazol-4-yl)amino)pyrazine-2-carbonitrile
5-((1-(4-((3R,4S)-3-(Dimethylamino)-4-methylpyrrolidin-1-yl)phenyl)-1H-imidazol-4-yl)amino)pyrazine-2-carbonitrile
5-((1-(4-((2R,4R)-4-(Dimethylamino)-2-methylpyrrolidin-1-yl)phenyl)-1H-imidazol-4-yl)amino)pyrazine-2-carbonitrile
5-((1-(4-((2S,4R)-4-(Dimethylamino)-2-methylpyrrolidin-1-yl)phenyl)-1H-imidazol-4-yl)amino)pyrazine-2-carbonitrile
5-((1-(4-((3R,4R)-3-(Dimethylamino)-4-methylpyrrolidin-1-yl)phenyl)-1H-imidazol-4-yl)amino)pyrazine-2-carbonitrile
(R)-5-((1-(5-(3-(Dimethylamino)pyrrolidin-1-yl)-6-(trifluoromethyl)pyridin-2-yl)-1H-imidazol-4-yl)amino)pyrazine-2-carbonitrile
(R)-5-((1-(4-(3-(Dimethylamino)-3-methylpyrrolidin-1-yl)phenyl)-1H-imidazol-4-yl)amino)pyrazine-2-carbonitrile
(R)-5-((1-(4-(3-(Dimethylamino)pyrrolidin-1-yl)-3-fluorophenyl)-1H-imidazol-4-yl)amino)pyrazine-2-carbonitrile
5-((1-(2-Fluoro-4-((1R,5S)-3-methyl-3,6-diazabicyclo[3.2.0]heptan-6-yl)phenyl)-1H-imidazol-4-yl)amino)pyrazine-2-carbonitrile
5-((1-(2-Fluoro-4-((1S,5R)-3-methyl-3,6-diazabicyclo[3.2.0]heptan-6-yl)phenyl)-1H-imidazol-4-yl)amino)pyrazine-2-carbonitrile
(R)-5-((1-(5-(3-(Dimethylamino)pyrrolidin-1-yl)pyrimidin-2-yl)-1H-imidazol-4-yl)amino)pyrazine-2-carbonitrile
(R)-5-((1-(2-(Dimethylamino)-4-(3-(dimethylamino)pyrrolidin-1-yl)phenyl)-1H-imidazol-4-yl)amino)pyrazine-2-carbonitrile
(R)-5-((1-(4-(3-(Bis(methyl-d3)amino)pyrrolidin-1-yl)phenyl)-1H-imidazol-4-yl)amino)pyrazine-2-carbonitrile
5-((1-(5-((2R,4R)-4-(Dimethylamino)-2-methylpyrrolidin-1-yl)pyridin-2-yl)-1H-imidazol-4-yl)amino)pyrazine-2-carbonitrile
5-((1-(4-((2R,3R)-3-(Dimethylamino)-2-methylpyrrolidin-1-yl)phenyl)-1H-imidazol-4-yl)amino)pyrazine-2-carbonitrile
5-((1-(4-((3aR,6aR)-1-Methylhexahydropyrrolo[3,4-b]pyrrol-5(1H)-yl)phenyl)-1H-imidazol-4-yl)amino)pyrazine-2-carbonitrile
5-((1-(4-((3S,4R)-3-(Ethylamino)-4-fluoropyrrolidin-1-yl)phenyl)-1H-imidazol-4-yl)amino)pyrazine-2-carbonitrile
(R)-5-((1-(4-(3-(Ethyl(methyl)amino)pyrrolidin-1-yl)phenyl)-1H-imidazol-4-yl)amino)pyrazine-2-carbonitrile
(R)-5-((1-(5-(3-(Ethyl(methyl)amino)pyrrolidin-1-yl)pyridin-2-yl)-1H-imidazol-4-yl)amino)pyrazine-2-carbonitrile
5-((1-(4-((3aR,6aR)-4-Methylhexahydropyrrolo[3,2-b]pyrrol-1(2H)-yl)phenyl)-1H-imidazol-4-yl)amino)pyrazine-2-carbonitrile
5-((1-(4-((3R,4S)-3-Fluoro-4-(methylamino)pyrrolidin-1-yl)phenyl)-1H-imidazol-4-yl)amino)pyrazine-2-carbonitrile
(R)-5-((1-(5-(3-((3,3,3-Trifluoropropyl)amino)pyrrolidin-1-yl)pyridin-2-yl)-1H-imidazol-4-yl)amino)pyrazine-2-carbonitrile
(R)-5-((1-(4-(Methyl(1-methylpyrrolidin-3-yl)amino)phenyl)-1H-imidazol-4-yl)amino)pyrazine-2-carbonitrile
(S)-5-((1-(4-(Methyl(1-methylpyrrolidin-3-yl)amino)phenyl)-1H-imidazol-4-yl)amino)pyrazine-2-carbonitrile
(R)-5-((1-(4-(3-(Isopropylamino)pyrrolidin-1-yl)phenyl)-1H-imidazol-4-yl)amino)pyrazine-2-carbonitrile
5-((1-(4-(3,3,4-Trimethylpiperazin-1-yl)phenyl)-1H-imidazol-4-yl)amino)pyrazine-2-carbonitrile
5-((1-(4-((2R,4R)-4-(Azetidin-1-yl)-2-methylpyrrolidin-1-yl)phenyl)-1H-imidazol-4-yl)amino)pyrazine-2-carbonitrile
5-((1-(4-(6-Methyl-1,6-diazaspiro[3.3]heptan-1-yl)phenyl)-1H-imidazol-4-yl)amino)pyrazine-2-carbonitrile
5-((1-(4-(1-Methyl-1,7-diazaspiro[3.5]nonan-7-yl)phenyl)-1H-imidazol-4-yl)amino)pyrazine-2-carbonitrile
5-((1-(4-((3aR,6aS)-5-Methylhexahydropyrrolo[3,4-c]pyrrol-2(1H)-yl)phenyl)-1H-imidazol-4-yl)amino)pyrazine-2-carbonitrile
(R)-5-((1-(5-(3-(Azetidin-1-yl)pyrrolidin-1-yl)pyridin-2-yl)-1H-imidazol-4-yl)amino)pyrazine-2-carbonitrile
(R)-5-((1-(4-(3-(Azetidin-1-yl)pyrrolidin-1-yl)-2-methoxyphenyl)-1H-imidazol-4-yl)amino)pyrazine-2-carbonitrile
(R)-5-((1-(4-(3-(Azetidin-1-yl)pyrrolidin-1-yl)-3-methylphenyl)-1H-imidazol-4-yl)amino)pyrazine-2-carbonitrile
5-((1-(4-(4-Methylmorpholin-2-yl)phenyl)-1H-imidazol-4-yl)amino)pyrazine-2-carbonitrile (Enantiomer 1)
5-((1-(4-(4-Methylmorpholin-2-yl)phenyl)-1H-imidazol-4-yl)amino)pyrazine-2-carbonitrile (Enantiomer 2)
5-((1-(3-(2-((Dimethylamino)methyl)morpholino)phenyl)-1H-imidazol-4-yl)amino)pyrazine-2-carbonitrile (Enantiomer 1)
5-((1-(3-(2-((Dimethylamino)methyl)morpholino)phenyl)-1H-imidazol-4-yl)amino)pyrazine-2-carbonitrile (Enantiomer 2)
5-((1-(3-(3-((2-Hydroxyethyl)(methyl)amino)pyrrolidin-1-yl)phenyl)-1H-imidazol-4-yl)amino)pyrazine-2-carbonitrile (Enantiomer 1)
5-((1-(3-(3-((2-Hydroxyethyl)(methyl)amino)pyrrolidin-1-yl)phenyl)-1H-imidazol-4-yl)amino)pyrazine-2-carbonitrile (Enantiomer 2)
5-((1-(3-(3-(Dimethylamino)piperidin-1-yl)phenyl)-1H-imidazol-4-yl)amino)pyrazine-2-carbonitrile (Enantiomer 1)
5-((1-(3-(3-(Dimethylamino)piperidin-1-yl)phenyl)-1H-imidazol-4-yl)amino)pyrazine-2-carbonitrile (Enantiomer 2)
5-((1-(4-(3-(Azetidin-1-yl)pyrrolidin-1-yl)phenyl)-1H-imidazol-4-yl)amino)pyrazine-2-carbonitrile (Enantiomer 1)
5-((1-(4-(3-(Azetidin-1-yl)pyrrolidin-1-yl)phenyl)-1H-imidazol-4-yl)amino)pyrazine-2-carbonitrile (Enantiomer 2)
5-((1-(4-(3-((Dimethylamino)methyl)pyrrolidin-1-yl)phenyl)-1H-imidazol-4-yl)amino)pyrazine-2-carbonitrile (Enantiomer 1)
5-((1-(4-(3-((Dimethylamino)methyl)pyrrolidin-1-yl)phenyl)-1H-imidazol-4-yl)amino)pyrazine-2-carbonitrile (Enantiomer 2)
5-((1-(4-(Hexahydropyrrolo[1,2-a]pyrazin-2(1H)-yl)phenyl)-1H-imidazol-4-yl)amino)pyrazine-2-carbonitrile (Enantiomer 1)
5-((1-(4-(Hexahydropyrrolo[1,2-a]pyrazin-2(1H)-yl)phenyl)-1H-imidazol-4-yl)amino)pyrazine-2-carbonitrile (Enantiomer 2)
5-((1-(4-(6-Methyl-1,6-diazaspiro[3.4]octan-1-yl)phenyl)-1H-imidazol-4-yl)amino)pyrazine-2-carbonitrile (Enantiomer 1)
5-((1-(4-(6-Methyl-1,6-diazaspiro[3.4]octan-1-yl)phenyl)-1H-imidazol-4-yl)amino)pyrazine-2-carbonitrile (Enantiomer 2)
5-((1-(4-(3-(Dimethylamino)piperidin-1-yl)phenyl)-1H-imidazol-4-yl)amino)pyrazine-2-carbonitrile (Enantiomer 1)
5-((1-(4-(3-(Dimethylamino)piperidin-1-yl)phenyl)-1H-imidazol-4-yl)amino)pyrazine-2-carbonitrile (Enantiomer 2)
5-((1-(4-(3-(3,3-Dimethylazetidin-1-yl)pyrrolidin-1-yl)phenyl)-1H-imidazol-4-yl)amino)pyrazine-2-carbonitrile (Enantiomer 1)
5-((1-(4-(3-(3,3-Dimethylazetidin-1-yl)pyrrolidin-1-yl)phenyl)-1H-imidazol-4-yl)amino)pyrazine-2-carbonitrile (Enantiomer 2)
5-((1-(4-(3-(3-Methylazetidin-1-yl)pyrrolidin-1-yl)phenyl)-1H-imidazol-4-yl)amino)pyrazine-2-carbonitrile (Enantiomer 1)
5-((1-(4-(3-(3-Methylazetidin-1-yl)pyrrolidin-1-yl)phenyl)-1H-imidazol-4-yl)amino)pyrazine-2-carbonitrile (Enantiomer 2)
5-((1-(4-(1-Methyl-1,6-diazaspiro[3.4]octan-6-yl)phenyl)-1H-imidazol-4-yl)amino)pyrazine-2-carbonitrile (Enantiomer 1)
5-((1-(4-(1-Methyl-1,6-diazaspiro[3.4]octan-6-yl)phenyl)-1H-imidazol-4-yl)amino)pyrazine-2-carbonitrile (Enantiomer 2)
5-((1-(4-(3-(3-Fluoroazetidin-1-yl)pyrrolidin-1-yl)phenyl)-1H-imidazol-4-yl)amino)pyrazine-2-carbonitrile (Enantiomer 1)
5-((1-(4-(3-(3-Fluoroazetidin-1-yl)pyrrolidin-1-yl)phenyl)-1H-imidazol-4-yl)amino)pyrazine-2-carbonitrile (Enantiomer 2)
5-((1-(4-(3-(2-Azaspiro[3.3]heptan-2-yl)pyrrolidin-1-yl)phenyl)-1H-imidazol-4-yl)amino)pyrazine-2-carbonitrile (Enantiomer 1)
5-((1-(4-(3-(2-Azaspiro[3.3]heptan-2-yl)pyrrolidin-1-yl)phenyl)-1H-imidazol-4-yl)amino)pyrazine-2-carbonitrile (Enantiomer 2)
5-((1-(4-(3-(3-Isopropylazetidin-1-yl)pyrrolidin-1-yl)phenyl)-1H-imidazol-4-yl)amino)pyrazine-2-carbonitrile (Enantiomer 1)
5-((1-(4-(3-(3-Isopropylazetidin-1-yl)pyrrolidin-1-yl)phenyl)-1H-imidazol-4-yl)amino)pyrazine-2-carbonitrile (Enantiomer 2)
5-((1-(4-(3-((R)-2-Methylazetidin-1-yl)pyrrolidin-1-yl)phenyl)-1H-imidazol-4-yl)amino)pyrazine-2-carbonitrile (Diastereomer 1)
5-((1-(4-(3-((R)-2-Methylazetidin-1-yl)pyrrolidin-1-yl)phenyl)-1H-imidazol-4-yl)amino)pyrazine-2-carbonitrile (Diastereomer 2)
5-((1-(4-(3-((S)-2-Methylazetidin-1-yl)pyrrolidin-1-yl)phenyl)-1H-imidazol-4-yl)amino)pyrazine-2-carbonitrile (Diastereomer 1)
5-((1-(4-(3-((S)-2-Methylazetidin-1-yl)pyrrolidin-1-yl)phenyl)-1H-imidazol-4-yl)amino)pyrazine-2-carbonitrile (Diastereomer 2)
5-((1-(4-(3-(2,2-Dimethylazetidin-1-yl)pyrrolidin-1-yl)phenyl)-1H-imidazol-4-yl)amino)pyrazine-2-carbonitrile (Enantiomer 1)
5-((1-(4-(3-(2,2-Dimethylazetidin-1-yl)pyrrolidin-1-yl)phenyl)-1H-imidazol-4-yl)amino)pyrazine-2-carbonitrile (Enantiomer 2)
(R)-1-(4-(4-((5-Cyanopyrazin-2-yl)amino)-1H-imidazol-1-yl)phenyl)-N,N-dimethylpyrrolidin-3-amine oxide
5-((1-(4-(Morpholinomethyl)phenyl)-1H-imidazol-4-yl)amino)pyrazine-2-carbonitrile
5-((1-(4-(4-Methylpiperazin-1-yl)phenyl)-1H-imidazol-4-yl)amino)pyrazine-2-carbonitrile
5-((1-(4-(4-Methylpiperazine-1-carbonyl)phenyl)-1H-imidazol-4-yl)amino)pyrazine-2-carbonitrile
5-((1-(2-methyl-4-(4-Methylpiperazin-1-yl)phenyl)-1H-imidazol-4-yl)amino)pyrazine-2-carbonitrile
5-((1-(4-((((3S,4R)-4-fluoro-1-Methylpyrrolidin-3-yl)oxy)methyl)phenyl)-1H-imidazol-4-yl)amino)pyrazine-2-carbonitrile
(R)-5-((1-(4-([1,3′-Bipyrrolidin]-1′-yl)phenyl)-1H-imidazol-4-yl)amino)pyrazine-2-carbonitrile
5-((1-(3-(3-Aminopropoxy)phenyl)-1H-imidazol-4-yl)amino)pyrazine-2-carbonitrile
5-((1-(2-(3-Aminopropoxy)-6-methoxyphenyl)-1H-imidazol-4-yl)amino)pyrazine-2-carbonitrile
5-((1-(4-(2-Aminoethoxy)phenyl)-1H-imidazol-4-yl)amino)pyrazine-2-carbonitrile
5-((1-(3-((3R,4S)-3-Amino-4-fluoropyrrolidin-1-yl)phenyl)-1H-imidazol-4-yl)amino)pyrazine-2-carbonitrile
5-((1-(4-((3S,4S)-3-Amino-4-fluoropyrrolidin-1-yl)phenyl)-1H-imidazol-4-yl)amino)pyrazine-2-carbonitrile
(R)-5-((1-(4-(3-Aminopyrrolidin-1-yl)phenyl)-1H-imidazol-4-yl)amino)pyrazine-2-carbonitrile
(R)-5-((1-(4-(7-Amino-5-azaspiro[2.4]heptan-5-yl)phenyl)-1H-imidazol-4-yl)amino)pyrazine-2-carbonitrile
(R)-5-((1-(4-(3-(Methylamino)pyrrolidin-1-yl)phenyl)-1H-imidazol-4-yl)amino)pyrazine-2-carbonitrile
(R)-5-((1-(4-(3-Amino-3-methylpyrrolidin-1-yl)phenyl)-1H-imidazol-4-yl)amino)pyrazine-2-carbonitrile
5-((1-(4-((3S,4R)-3-Amino-4-fluoropyrrolidin-1-yl)phenyl)-1H-imidazol-4-yl)amino)pyrazine-2-carbonitrile
5-((1-(4-(3-(Aminomethyl)azetidin-1-yl)phenyl)-1H-imidazol-4-yl)amino)pyrazine-2-carbonitrile
5-((1-(4-(3-(Aminomethyl)-3-fluoroazetidin-1-yl)phenyl)-1H-imidazol-4-yl)amino)pyrazine-2-carbonitrile
5-((1-(4-(1,6-Diazaspiro[3.3]heptan-6-yl)phenyl)-1H-imidazol-4-yl)amino)pyrazine-2-carbonitrile
5-((1-(4-((1R,5R)-3,6-Diazabicyclo[3.2.0]heptan-3-yl)phenyl)-1H-imidazol-4-yl)amino)pyrazine-2-carbonitrile
(R)-5-((1-(4-(3-(Ethylamino)pyrrolidin-1-yl)phenyl)-1H-imidazol-4-yl)amino)pyrazine-2-carbonitrile
5-((1-(4-((3R,4S)-3-Methoxy-4-(methylamino)pyrrolidin-1-yl)phenyl)-1H-imidazol-4-yl)amino)pyrazine-2-carbonitrile
5-((1-(4-((3R,4S)-3-Fluoro-4-(methylamino)pyrrolidin-1-yl)-2-methoxyphenyl)-1H-imidazol-4-yl)amino)pyrazine-2-carbonitrile
5-((1-(4-((2R,4R)-4-Amino-2-methylpyrrolidin-1-yl)phenyl)-1H-imidazol-4-yl)amino)pyrazine-2-carbonitrile
5-((1-(3-(4-Amino-3,3-difluoropiperidin-1-yl)phenyl)-1H-imidazol-4-yl)amino)pyrazine-2-carbonitrile (Enantiomer 1)
5-((1-(3-(4-Amino-3,3-difluoropiperidin-1-yl)phenyl)-1H-imidazol-4-yl)amino)pyrazine-2-carbonitrile (Enantiomer 2)
5-((1-(2-(3-Aminopropoxy)phenyl)-1H-imidazol-4-yl)amino)pyrazine-2-carbonitrile
(R)-5-((1-(4-((3-Fluoropyrrolidin-1-yl)methyl)-2-methoxyphenyl)-1H-imidazol-4-yl)amino)pyrazine-2-carbonitrile)
5-((1-(4-((Ethylamino)methyl)-2-methoxyphenyl)-1H-imidazol-4-yl)amino)pyrazine-2-carbonitrile
5-((1-(2-Methoxy-4-(pyrrolidin-1-ylmethyl)phenyl)-1H-imidazol-4-yl)amino)pyrazine-2-carbonitrile
5-((1-(4-((4,4-Difluoropiperidin-1-yl)methyl)-2-methoxyphenyl)-1H-imidazol-4-yl)amino)pyrazine-2-carbonitrile
5-((1-(2-Methoxy-4-(morpholinomethyl)phenyl)-1H-imidazol-4-yl)amino)pyrazine-2-carbonitrile
5-((1-(4-((4-Fluoropiperidin-1-yl)methyl)phenyl)-1H-imidazol-4-yl)amino)pyrazine-2-carbonitrile
5-((1-(3-((2-Methyl-2-azaspiro[3.3]heptan-6-yl)oxy)phenyl)-1H-imidazol-4-yl)amino)pyrazine-2-carbonitrile
5-((1-(4-(1-Methylazetidin-3-yl)phenyl)-1H-imidazol-4-yl)amino)pyrazine-2-carbonitrile
5-((1-(4-((1R,5S)-9-Methyl-3-oxa-7,9-diazabicyclo[3.3.1]nonan-7-yl)phenyl)-1H-imidazol-4-yl)amino)pyrazine-2-carbonitrile
5-((1-(4-((2-Methyl-2-azaspiro[3.3]heptan-6-yl)oxy)phenyl)-1H-imidazol-4-yl)amino)pyrazine-2-carbonitrile
5-((1-(4-(1-(Diethylamino)cyclopropyl)phenyl)-1H-imidazol-4-yl)amino)pyrazine-2-carbonitrile
5-((1-(2-Methoxy-4-((2-methyl-2-azaspiro[3.3]heptan-6-yl)oxy)phenyl)-1H-imidazol-4-yl)amino)pyrazine-2-carbonitrile
5-((1-(4-(4-(3,3-Difluorocyclobutyl)piperazin-1-yl)phenyl)-1H-imidazol-4-yl)amino)pyrazine-2-carbonitrile
5-((1-(4-(4-(Oxetan-3-yl)piperazin-1-yl)phenyl)-1H-imidazol-4-yl)amino)pyrazine-2-carbonitrile
5-((1-(4-(4-(2-Fluoroethyl)piperazin-1-yl)phenyl)-1H-imidazol-4-yl)amino)pyrazine-2-carbonitrile
5-((1-(4-(4-(3,3,3-Trifluoropropyl)piperazin-1-yl)phenyl)-1H-imidazol-4-yl)amino)pyrazine-2-carbonitrile
5-((1-(4-(4-Ethylpiperazin-1-yl)phenyl)-1H-imidazol-4-yl)amino)pyrazine-2-carbonitrile
5-((1-(4-(4-Isopropylpiperazin-1-yl)phenyl)-1H-imidazol-4-yl)amino)pyrazine-2-carbonitrile
   or a pharmaceutically acceptable salt, solvate, hydrate, tautomer, enantiomer, diastereoisomer, isotopic form, N-oxide, and/or prodrug thereof. In some cases, particular stereoisomers of compounds of the invention have particularly favourable properties. For some of the compounds in the list above, enantiomers and diastereomers have been separated out into ‘Enantiomer 1’ and ‘Enantiomer 2’ or ‘Diastereomer 1’ and ‘Diastereomer 2’. It will be appreciated that one of ‘Enantiomer 1’ and ‘Enantiomer 2’ is the R-enantiomer, and the other will be the S-enantiomer. Similarly, the absolute configuration of one or more of the chiral centres in the compounds listed as ‘Diastereomer 1’ and ‘Diastereomer 2’ is known, whereas one chiral centre may be R- or S- in one diastereoisomer, and the opposite configuration in the other diastereoisomer. Details of how to prepare those enantiomers and diastereomers are set out in the examples below. In any case, the skilled person would know how to prepare and separate particular enantiomers and diastereomers of the compounds above and identify their stereochemistry using known techniques and methods of the art.

The compounds of the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, tautomers, enantiomers, diastereoisomers, isotopic forms, N-oxides, and/or prodrug forms thereof. It is to be understood that the compounds of the invention include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereoisomers, are intended unless otherwise indicated. The terms “diastereoisomer” and “diastereomer” are used herein interchangeably. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereoisomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereoisomeric compound then being obtained as the end product.

The term “isotopic form” refers to the fact that compounds of the invention may include isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. As used herein, deuterium labelled compounds may include ‘-d’ to show that deuterium is included, i.e. “methyl-d3” indicates the group —CD₃.

The term “N-oxide” denotes a compound containing the N⁺—O⁻ functional group, such as in the following example.

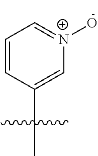 [SMILES: *c1ccc[n+]([O-])c1]

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow-release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

An object of the present invention relates to the compounds of the invention for use as a medicament. The term ‘medicament’ denotes a substance used for medical treatment or as a medicine.

The compounds of the invention may be useful as inhibitors of Chk1. As such, they are useful in the treatment or prevention of medical conditions (conditions or diseases) that are affected by Chk1. Therefore, there is provided a method of treating a disease or condition responsive to Chk1 inhibition, such as cancer, in a subject in need thereof, comprising administering to the subject a therapeutically effective amount of a compound of the invention.

The compounds of the invention may be for use in medicine. This use may be in the treatment of cancer.

The compounds of the invention may also be useful in the manufacture of a medicament. That medicament may be of use in the treatment of cancer.

The compounds of the invention may be useful in the treatment of a range of cancers, particularly those that are susceptible to Chk1 inhibition. Non-limiting cancers include CNS cancer (including glioblastoma multiforme, glioma, neuroblastoma, medulloblastoma, DIPG and secondary brain tumours such as brain metastasis), sarcoma (including osterosarcoma, rhabdomyosarcoma, Kaposi's sarcoma, leiomyosarcoma), ovarian cancer (including high grade serous ovarian cancer), lung cancer (including small cell lung cancer, non-small cell lung cancer), breast cancer, endometrial cancer, prostate cancer, pancreatic cancer, renal cell carcinoma, colorectal cancer (including colon cancer, rectal cancer, anal cancer), gastrointestinal cancer (including gastric cancer), thyroid cancer, bladder cancer, kidney cancer, melanoma, squamous cancer (including squamous cell carcinoma head and neck), leukemia or lymphoma.

Preferably, the compounds of the invention are useful in the treatment of CNS cancer (including glioblastoma multiforme, glioma, neuroblastoma, medulloblastoma, DIPG and secondary brain tumours such as brain metastasis, sarcoma (including osteosarcoma, rhabdomyosarcoma, Kaposi's sarcoma, leiomyosarcoma), bladder cancer, endometrial cancer, lung cancer (including small cell lung cancer, non-small cell lung cancer), breast cancer, squamous cancer (including squamous cell carcinoma head and neck), colorectal cancer (including colon cancer, rectal cancer, anal cancer), gastric cancer, or ovarian cancer (including high grade serous ovarian cancer).

The term “treatment” and “treating” as used herein may include prophylaxis of the named disorder or condition, or amelioration or elimination of the disorder or condition once it has been established. The term “prevention” refers to prophylaxis of the named disorder or condition.

As used herein, the terms “administration” or “administering” mean a route of administration for a compound disclosed herein. Exemplary routes of administration include, but are not limited to, oral, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

“A therapeutically effective amount” refers to an amount of a compound of the invention that confers a therapeutic effect on the treated subject. The therapeutic effect may be objective (i.e. measurable by some test or marker) or subjective (i.e. subject gives an indication of or feels an effect).

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The compounds of the invention may be used as a mono therapy. They may also be used as part of a combination therapy. When used as a combination therapy they may be administered in combination with one or more of anticancer treatments including ionising radiation; a DNA topoisomerase I or II inhibitor; an antimetabolite or thymidylate synthase inhibitor; an agent related to the DNA damage and repair mechanism; a microtubule targeted agent; platinum based antineoplastic agents; alkylating agents and immune checkpoint inhibitors.

Anticancer agents related to the DNA damage and repair mechanism include, but are not limited to, Wee1 inhibitors such as Adavosertib; PARP inhibitors such as Olaparib, Veliparib, Niraparib, Rucaparib, Talazoparib and senaparib; ATR inhibitors such as ceralasetib, gartisertib; HDAC inhibitors such as entinostat, vorinostat; PARG inhibitors such as PDD00017273 and JA2131; and ATM inhibitors such as AZD0156, AZD1390.

Topoisomerase inhibitors are exemplified by doxorubicin, irinotecan, camptothesin and etoposide. Antimetabolites are exemplified by gemcitabine, pemetrexed and cytarabine. Platinum based antineoplastic agents are exemplified by cisplatin and carboplatin. Alkylating agents are exemplified by temozolomide. Immune checkpoint inhibitors are exemplified by ipilimumab, nivolumab, pembrolizumab, atezolizumab, durvalumab and cemiplimab. Other chemotherapy agents which may be used in combination therapy include paclitaxel, MEK inhibitors such as binimetinib, cobimetinib and trametinib, farnesyltransferase inhibitors such as tipifarnib and rapamycin.

As used herein, being “administered in combination” with one or more anticancer treatments means that the compounds are administered concomitantly or sequentially and in any suitable order, as will be appreciated by the skilled person.

The invention provides a method of monitoring treatment progress. The method includes the step of determining a level of diagnostic marker (Marker) (e.g. any target or cell type delineated herein modulated by a compound herein) or diagnostic measurement (e.g. screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred features of the invention, a second level of Marker in the subject is determined at a time point later than the determination of the first level, and the two levels are compared to monitor the course of disease or the efficacy of the therapy. In certain preferred features of the invention, a pre-treatment level of Marker in the subject is determined prior to beginning treatment according to this invention; this pre-treatment level of Marker can then be compared to the level of Marker in the subject after the treatment commences, to determine the efficacy of the treatment.

A level of Marker or Marker activity in a subject may be determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, is ELISA, radiolabelling/assay techniques, blotting/chemiluminescence methods, real-time PCR, and the like.

Compounds of the invention may be disclosed by the name or chemical structure. If a discrepancy exists between the name of a compound and its associated chemical structure, then the chemical structure prevails.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### Preparation of Compounds of the Invention

The compounds of the invention may be prepared by, or in analogy with, conventional methods. The preparation of intermediates and compounds according to the examples of the present invention may in particular be illuminated by the following Schemes. Definitions of variables in the structures in schemes herein are commensurate with those of corresponding positions in the formulae delineated herein.

The following abbreviations have been used:
AcOH acetic acid
AdBrettPhos 2-(di-1-adamantylphosphino)-3,6-dimethoxy-2′,4′,6′-tri-i-propyl-1,1′-biphenyl
AdBrettPhos Pd G3 methanesulfonato[2-(di-1-adamantylphosphino)-3,6-dimethoxy-2′,4′,6′-tri-i-propyl-1,1′-biphenyl](2′-amino-1,1′-biphenyl-2-yl)palladium(II)
aq aqueous
BCRP breast cancer resistance protein
BINAP 2,2′-bis(diphenylphosphino)-1,1′-binaphthalene
Boc tert-butyloxycarbonyl
n-BuLi n-butyl lithium
Cbz benzyloxycarbonyl
CNS Central nervous system
Cu(OAc)₂ copper(II) acetate
Cu(OTf)₂ copper(II) trifluoromethanesulfonate
DAD diode-array detector
DAST (diethylamino)sulfur trifluoride
DCM dichloromethane
DEA N,N-diethylaniline
DIAD diisopropyl azodicarboxylate
DIEA N,N-diisopropylethylamine
DMF N,N-dimethylformamide
DMSO dimethyl sulfoxide
DXN 1,4-dioxane
EDTA ethylenediaminetetraacetic acid
Ephos dicyclohexyl(3-isopropoxy-2′,4′,6′-triisopropyl-[1,1′-biphenyl]-2-yl)phosphane
Ephos Pd G4 methanesulfonato{dicyclohexyl[3-(1-methylethoxy)-2′,4′,6′-tris(1-methylethyl)[1,1′-biphenyl]-2-yl]phosphine}(2′-methylamino-1,1′-biphenyl-2-yl)palladium(II)
ES⁺ electrospray ionisation
EtI iodoethane
Et₃N triethylamine
EtOAc ethyl acetate
EtOH ethanol
Ex. example
FA formic acid
h hour(s)
HBD H-bond donor
HEPES N-2-hydroxyethylpiperazine-N′-2-ethanesulfonic acid
Hex hexane
HPLC high-performance liquid chromatography
Int. intermediate
IPA isopropyl alcohol
IV intravenous
KOAc potassium acetate
LCMS liquid chromatography-mass spectrometry
mCPBA 3-chloroperbenzoic acid
MDCK Madin-Darby canine kidney
MDR1 multidrug resistance protein 1
MeCN acetonitrile
Mel iodomethane
MeOH methanol
Me₄tBuXPhos 2-di-tert-butylphosphino-3,4,5,6-tetramethyl-2′,4′,6′-triisopropyl-1,1′-biphenyl
Min minute(s)
MTBE methyl tert-butyl ether
NaOAc sodium acetate
NaOtBu sodium tert-butoxide
NP normal phase
n/r not recorded
OTf trifluoromethanesulfonate Pₐₚₚ apparent permeability coefficient
PDA photodiode-array detector
Pd₂dba₃ tris(dibenzylideneacetone)dipalladium(0)
Pd(dppf)Cl₂ [1,1′-bis(diphenylphosphino)ferrocene]dichloropalladium(II)
Pd(dppf)Cl₂-DCM [1,1′-bis(diphenylphosphino)ferrocene]dichloropalladium(II), complex with dichloromethane
PE petroleum ether
P-gp P-glycoprotein
PhMe toluene
PPh₃ triphenylphosphine
Prep-HPLC preparative high-performance liquid chromatography
Prep-SFC preparative supercritical fluid chromatography
Prep-TLC preparative thin layer chromatography
RP reverse phase
RT room temperature
Rt retention time
sat saturated
SFC supercritical fluid chromatography
STAB sodium triacetoxyborohydride
tBuXPhos Pd G3 [(2-di-tert-butylphosphino-2′,4′,6′-triisopropyl-1,1′-biphenyl)-2-(2′-amino-1,1′-biphenyl)]palladium(II) methanesulfonate
TFA trifluoroacetic acid
THE tetrahydrofuran
tPSA topological polar surface area
UPLC ultra-performance liquid chromatography
XPhos 2-dicyclohexylphosphino-2′,4′,6′-triisopropylbiphenyl
XPhos Pd G3 (2-dicyclohexylphosphino-2′,4′,6′-triisopropyl-1,1′-biphenyl)[2-(2′-amino-1,1′-biphenyl)]palladium(II) methanesulfonate

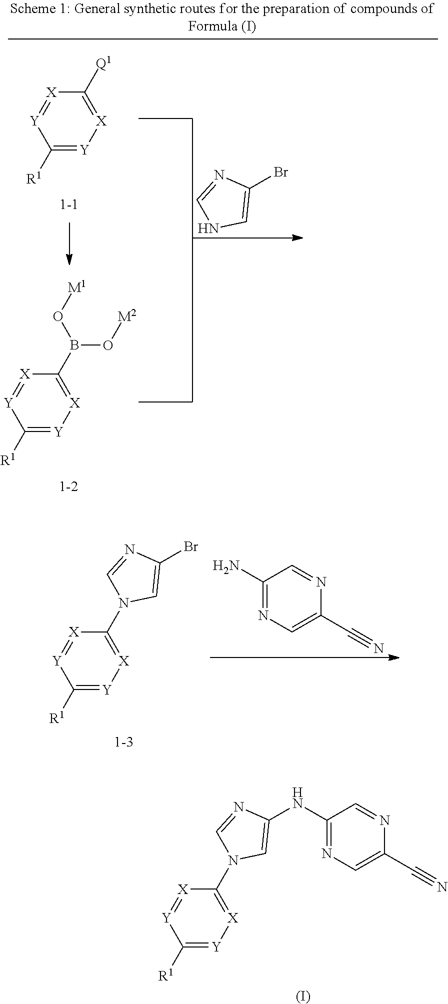

wherein R¹, X and Y are as defined herein, Q¹ is halo, and M¹ and M² are H, alkyl or M¹ and M² together may form a ring, such as in pinacol esters.

Compounds of Formula (I) can be prepared by Buchwald-Hartwig cross-coupling reactions of compounds of general formula 1-3 with 5-aminopyrazine-2-carbonitrile. Compounds of formula 1-3 can be prepared from compounds of general formula 1-1 by SNAr reaction with 4-bromo-1H-imidazole. Alternatively, compounds of general formula 1-3 can be prepared from compounds of general formula 1-2 by Chan-Lam reaction with 4-bromo-1H-imidazole. Compounds of general formula 1-2 can be prepared from commercially available boronic acid/ester reagents of the same general formula but with suitable functional handles at R¹, X or Y, using standard chemistry reactions including Mitsunobu, SNAr, and reductive amination reactions. Alternatively, compounds of general formula 1-2 can be prepared from compounds of general formula 1-1 (where Q¹ is Br) by transformation of suitable functional handles at R¹, X or Y by standard chemistry reactions including lithium-halogen exchange followed by reaction with a suitable electrophile and alkylation reactions, then conversion of Br (Q¹) to the corresponding boronic acid/ester.

Compounds of general formulae 1-3 or (I) can be converted into another compound of the same formula using standard chemistry reactions in one or more synthetic steps. For example, if R¹, X or Y contains a suitable halogen, then SNAr or Buchwald-Hartwig coupling reactions with the appropriate commercially available amine, or amines of general formula 2-2 (Scheme 2), can be used to give other compounds of general formulae 1-3 or (I). Alternatively, if R¹, X or Y contains a suitable aldehyde or ketone then reductive amination or carbonyl reduction reactions can be performed to give other compounds of general formulae 1-3 or (I). Alternatively, if R¹, X or Y contains a suitable OH group then Mitsunobu, Appel, alkylation or oxidation reactions can be performed to give other compounds of general formulae 1-3 or (I). Alternatively, if R¹, X or Y contains a suitable amine then N-oxidation, reductive amination, or alkylation reactions can be employed to give other compounds of general formulae 1-3 or (I). Where appropriate, amines can be installed via the corresponding NO₂ group using standard chemistry transformations, or via SN2 reaction with a suitable reactant. Standard protecting group strategies can be applied as needed at any stage to facilitate the chemical transformations mentioned above. Where compounds contain one or more chiral centres, compounds can be made as racemates, as single enantiomers, or as diastereomeric mixtures of known or unknown absolute stereochemistry. Mixtures of enantiomers or diastereomers may be optionally separated by standard techniques at any stage in the synthetic sequence.

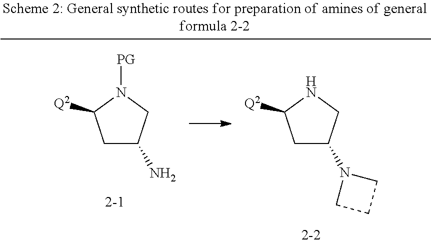

wherein PG is a protecting group, and Q² is H or methyl.

Compounds of general formula 2-2 can be prepared from compounds of general formula 2-1 by reductive amination or alkylation followed by protecting group (PG) removal using standard conditions. Amines of general formula 2-2 can be reacted with intermediates of general formula 1-3 (Scheme 1, where R¹ is a halogen) by standard Buchwald-Hartwig coupling reactions to give other compounds of general formula 1-3.

### EXAMPLES AND INTERMEDIATE COMPOUNDS

### Experimental Methods

All reactants and reagents were sourced commercially unless otherwise stated, in which case synthetic routes have been provided. All reagents were commercial-grade and were used as received without further purification unless otherwise specified. Reagent-grade solvents were used unless otherwise specified. Anhydrous solvents were used where appropriate. Solvent ratios are by volume. Reactions were conducted at RT unless otherwise specified. n-BuLi was used as a 2.5M solution in hexanes unless otherwise stated. Formaldehyde was used as a 37% by weight solution in water unless otherwise stated. NaH was used as a 60% dispersion in mineral oil unless otherwise stated. The reactions facilitated by microwave heating were performed on a Biotage Initiator+ system using process vials fitted with aluminium caps and septa. Amounts of reagents used and product yields were typically adjusted for purity where known. The Intermediates and Examples were named using IUPAC nomenclature.

Column chromatography was performed using Teledyne ISCO CombiFlash RF200, CombiFlash RF+ or Combiflash RF+ Lumen systems equipped with RediSep® Rf, RediSep® Rf C18 Reverse Phase, or RediSep® Silver silica columns. Preparative RP HPLC was performed on either a Teledyne Isco ACCQPrep HP125 system with 200-400 nm UV variable wavelength detector and a CombiFlash® Purlon™ S mass spectrometer, or an ACCQPrep HP150 system with 200-400 nm UV variable wavelength detector, equipped with Avantor® ACE® 5 AQ, 100×21.2 mm, 5 μm columns, Avantor® ACE® SuperC18™, 100×21.2 mm, 5 μm columns, or Phenomenex® Kinetex® EVO C18, 100×21.2 mm, 5 μm columns. For RP column chromatography and RP HPLC modifiers used included TFA, FA and NH₃. Catch and elute purification was performed using Biotage ISOLUTE® Flash SCX-2 cartridges unless otherwise stated. Atypical catch-and-elute procedure was to dissolve the crude material in DCM, load the solution onto the cartridge, wash with MeOH, elute with approximately 1 N NH₃ in MeCOH then concentrate in vacuo.

Chiral prep-HPLC was performed using a Gilson GX-281 chiral prep-HPLC with a 159 UV-VIS detector and 322 pump using the following methods:
Chiral Prep-HPLC Method 1: Column: CHIRALPAK IG, 2×25 cm, 5 μm; Mobile Phase A: Hex:MTBE=1:1 (0.5% 2M NH₃-MeOH), Mobile Phase B: MeOH
Chiral Prep-HPLC Method 2: Column: CHIRALCEL AY-H, 2×25 cm, 5 μm; Mobile Phase A: Hex (10 mM NH₃-MeOH), Mobile Phase B: EtOH
Chiral Prep-HPLC Method 3: Column: CHIRAL ART Cellulose-SZ, 3×25 cm, 5 μm; Mobile Phase A: Hex (10 mM NH₃-MeOH), Mobile Phase B: EtOH
Chiral Prep-HPLC Method 4: Column: CHIRALPAK 1A-3, 4.6×5 mm, 3 μm; Mobile Phase A: MTBE (0.1% DEA):EtOH=70:30
Chiral Prep-HPLC Method 5: Column: CHIRALPAK ID-3, 4.6×50 mm 3 μm; Mobile Phase A: MTBE (0.1% DEA):MeOH=70:30
Chiral Prep-HPLC Method 6: Column: CHIRALPAK IA, 2.12×15 cm, 5 μm; Mobile Phase A: MTBE (10 mM NH₃-MeOH), Mobile Phase B: MeOH
Chiral Prep-HPLC Method 7: Column: CHIRAL ART Amylose-SA, 3×25 cm, 5 μm; Mobile Phase A: MTBE:DCM=1 1 (10 mM NH₃), Mobile Phase B: MeOH
Chiral Prep-HPLC Method 8: Column: CHIRAL ART Amylose-SA, 3×25 cm, 5 μm; Mobile Phase A: MTBE (10 mM NH₃-MeOH), Mobile Phase B: MeOH
Chiral Prep-HPLC Method 9: Column: CHIRALPAK ID, 3×25 cm, 5 μm; Mobile Phase A: MTBE (2 mM NH₃-MeOH), Mobile Phase B: MeOH
Chiral Prep-HPLC Method 10: Column: CHIRALPAK IA-3, 4.6×50 mm, 3 μm; Mobile Phase A: MTBE (0.1% DEA):MeCOH=50:50

Prep-SFC was performed on a Waters SFC-150 with a Waters 2489 UV detector, a Waters preparative fraction selector, a Waters 2545 co-solvent pump and a Waters P200X CO₂ pump using the following methods:
Prep-SFC Method 1: Column: CHIRALPAK IH, 3×25 cm, 5 μm; Mobile Phase A: CO₂, Mobile Phase B: MeOH
Prep-SFC Method 2: Column: CHIRALPAK IH, 3×25 cm, 5 μm; Mobile Phase A: CO₂, Mobile Phase B: MeOH (0.1% 2M NH₃-MeOH)
Prep-SFC Method 3: Column: (R,R)-WHELK-01-Kromasil, 5×25 cm, 5 μm; Mobile Phase A: MTBE:DCM=1:1, Mobile Phase B: MeOH
Prep-SFC Method 4: Column: Lux 5 μm Cellulose-3, 2.12×25 cm, 5 μm; Mobile Phase A: Hex (10 mM NH₃-MeOH), Mobile Phase B: EtOH
Prep-SFC Method 5: Column: CHIRALPAK IA, 2×25 cm, 5 μm; Mobile Phase A: MeOH:DCM=2:1 (0.1% 2M NH₃-MeOH), Mobile Phase B: MeOH
Prep-SFC Method 6: Column: CHIRALPAK ID, 2×25 cm, 5 μm; Mobile Phase A: MTBE (10 mM NH₃-MeOH), Mobile Phase B: MeOH

Compound analysis was performed by UPLC, HPLC and LCMS. UPLC data was collected using an Agilent 1290 Infinity or Infinity II system with DAD using a Phenomenex Kinetex XB-C18 column. HPLC and LCMS data was collected using a Waters ACQUITY H-class HPLC with Phenomenex Kinetex XB-C18 column and ACQUITY QDa mass detector connector, or a Shimadzu LCMS-2020 system with PDA: SPD-M20A or SPD-MP40.

### Intermediates

### Intermediate 1

### tert-Butyl (3-(2-(4,4,5,5-tetramethyl-1,3,2-dioxaborolan-2-yl)phenoxy)propyl)carbamate

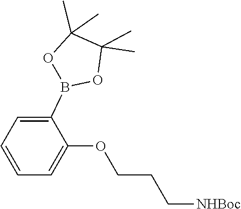 [SMILES: CC(C)(C)OC(=O)NCCCOc1ccccc1B1OC(C)(C)C(C)(C)O1]

To 2-(4,4,5,5-tetramethyl-1,3,2-dioxaborolan-2-yl)phenol (1.00 g, 4.50 mmol), tert-butyl N-(3-hydroxypropyl)carbamate (1.19 g, 6.8 mmol) and PPh₃ (1.79 g, 6.82 mmol) in THF (20 mL) at 0° C. was added DIAD (1.38 g, 6.82 mmol) dropwise. The mixture was allowed to warm to RT and stir for 4 h. The mixture was quenched with water then concentrated in vacuo and purified via RP column chromatography to afford the title compound (570 mg, 33.3%) as a light-yellow solid. LCMS (ES⁺): 378.3 [MH]⁺.

### Intermediate 2

### (4-(Morpholinomethyl)phenyl)boronic acid

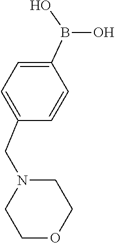 [SMILES: OB(O)c1ccc(CN2CCOCC2)cc1]

4-Formylphenylboronic acid (1.00 g, 6.67 mmol) and morpholine (0.58 g, 6.67 mmol) in MeCOH (15 mL) was allowed to stir at RT for 1 h. To the mixture was added NaBH₃CN (0.42 g, 6.67 mmol) at 0° C. The mixture was then allowed to warm to RT and stir for a further 12 h. The reaction was quenched with water, extracting with EtOAc (3×100 mL). The combined organic layers were washed with brine (100 mL), dried (Na₂SO₄), and concentrated in vacuo. Purification via RP column chromatography afforded the title compound (451 mg, 30.6%) as a white solid. LCMS (ES⁺): 225.3 [MH]⁺.

### Intermediate 3

### (4-(4-Fluoro-1-methylpiperidin-4-yl)phenyl)boronic acid

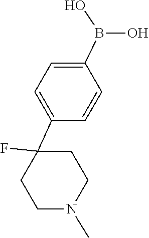 [SMILES: CN1CCC(F)(c2ccc(B(O)O)cc2)CC1]

Step 1: To dibromobenzene (2.00 g, 8.48 mmol) in THF (20 mL) was added n-BuLi (3.40 mL, 8.50 mmol) dropwise at −78° C. The mixture was then stirred for 30 min before addition of 1-methylpiperidin-4-one (0.96 g, 8.48 mmol) dropwise. The mixture was allowed to warm to RT and stir for a further 1 h. Reaction quenched with water, extracting with EtOAc (2×20 mL). The combined organic layers were washed with brine (2×10 mL), dried (Na₂SO₄), and concentrated in vacuo. Purification via RP column chromatography afforded 4-(4-bromophenyl)-1-methylpiperidin-4-ol (1.66 g, 72.5%) as a yellow oil. LCMS (ES⁺): 270.3 [MH]⁺.

Step 2: To a mixture of 4-(4-bromophenyl)-1-methylpiperidin-4-ol (1.00 g, 3.70 mmol) in THF (10 mL) was added DAST (1.19 g, 7.40 mmol) dropwise at −78° C. under N₂. The mixture was allowed to warm to RT and stirred for 5 h. Reaction quenched with ice water, extracting with EtOAc (2×10 mL). The combined organic layers were washed with water (2×10 mL), dried (Na₂SO₄), and concentrated in vacuo. Purification via NP column chromatography afforded 4-(4-bromophenyl)-4-fluoro-1-methylpiperidine (760 mg, 75.4%) as a brown solid. LCMS (ES⁺): 272.3 [MH]⁺.

Step 3: A mixture of 4-(4-bromophenyl)-4-fluoro-1-methylpiperidine (760 mg, 2.79 mmol), bis(pinacolato)diboron (1.42 g, 5.58 mmol), Pd(dppf)Cl₂·DCM (1.14 g, 1.40 mmol), KOAc (548 mg, 5.58 mmol) in DXN (10 mL) was stirred for 16 h at 100° C. under N₂. The mixture was filtered, washing with DXN, the filtrate was then concentrated in vacuo. Purification via RP column chromatography afforded 4-fluoro-1-methyl-4-(4-(4,4,5,5-tetramethyl-1,3,2-dioxaborolan-2-yl)phenyl)piperidine (400 mg, 44.9%) as a yellow oil. LCMS (ES⁺): 320.2 [MH]⁺.

Step 4: A mixture of 4-fluoro-1-methyl-4-(4-(4,4,5,5-tetramethyl-1,3,2-dioxaborolan-2-yl)phenyl)piperidine (400 mg, 1.26 mmol) and NaIO₄ (810 mg, 3.76 mmol) in THF (4.0 mL) and water (1.0 mL) was stirred for 4 h. The mixture was filtered, washing with MeCOH, the filtrate was then concentrated in vacuo. Purification via RP column chromatography afforded the title compound (240 mg, 80.8%) as a yellow oil. LCMS (ES⁺): 238.1 [MH]⁺.

### Intermediate 4

### (4-(4-Methylmorpholin-2-yl)phenyl)boronic acid

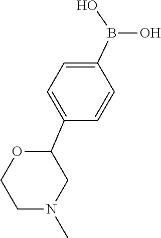 [SMILES: CN1CCOC(c2ccc(B(O)O)cc2)C1]

Step 1: To a mixture of methylethanolamine (1.62 g, 21.6 mmol) was added FA (1.0 mL) dropwise at 0° C. under N₂. Then 2-bromo-1-(4-bromophenyl) ethanone (3.00 g, 10.8 mmol) was added and the mixture was heated to 100° C. and allowed to stir for 16 h. The reaction was quenched with water, extracting with EtOAc (50 mL). Excess K₂CO₃ was added to the aqueous which was further extracted with EtOAc (3×100 mL). The combined organic layers were washed with brine (50 mL), dried (Na₂SO₄), and concentrated in vacuo. Purification via RP column chromatography afforded 2-(4-bromophenyl)-4-methylmorpholine (1.42 g, 51.4%) as a yellow oil. LCMS (ES⁺): 256.1 [MH]⁺.

Step 2: 2-(4-Bromophenyl)-4-methylmorpholine (1.42 g, 5.54 mmol), bis(pinacolato)diboron (2.11 g, 8.32 mmol,), Pd(dppf)Cl₂·DCM (230 mg, 0.282 mmol) and NaOAc (1.53 mg, 18.7 mmol) in DXN (20 mL) was stirred for 16 h at 80° C. under N₂. The mixture was concentrated in vacuo and purified via RP column chromatography to afford 4-methyl-2-(4-(4,4,5,5-tetramethyl-1,3,2-dioxaborolan-2-yl)phenyl)morpholine (704 mg, 41.9%) as a brown oil. LCMS (ES⁺): 304.3 [MH]⁺.

Step 3: 4-Methyl-2-(4-(4,4,5,5-tetramethyl-1,3,2-dioxaborolan-2-yl)phenyl)morpholine (704 mg, 2.32 mmol) and NaIO₄ (2.44 g, 11.4 mmol) in THF (20 mL) and water (4.0 mL) was stirred for 36 h. The mixture was concentrated in vacuo and purified via RP column chromatography to afford the title compound (432 mg, 84.1%) as a yellow solid. LCMS (ES⁺): 222.2 [MH]⁺.

### Intermediate 5

### (4-(1-(tert-Butoxycarbonyl)azetidin-3-yl)phenyl)boronic acid

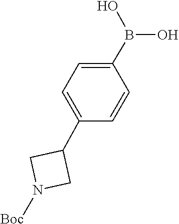 [SMILES: CC(C)(C)OC(=O)N1CC(c2ccc(B(O)O)cc2)C1]

To tert-butyl 3-(4-bromophenyl)azetidine-1-carboxylate (500 mg, 1.60 mmol) in THF (10 mL) was added n-BuLi (0.75 mL, 1.92 mmol) dropwise at −78° C. under N₂. The mixture was allowed to stir for 30 min before addition of triisopropyl borate (451 mg, 2.40 mmol) dropwise. The mixture was then allowed to warm to RT and stir for a further 2 h. The mixture was concentrated in vacuo and purified via NP column chromatography to afford the title compound (227 mg, 51.2%) as a yellow oil. LCMS (ES⁺): 278.1 [MH]⁺.

### Intermediate 6

### (6-(4-Methyl-1,4-diazepan-1-yl)pyridin-3-yl)boronic acid

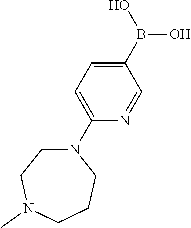 [SMILES: CN1CCCN(c2ccc(B(O)O)cn2)CC1]

6-Fluoropyridin-3-ylboronic acid (3.00 g, 21.3 mmol), N-methylhomopiperazine (4.86 mL, 42.6 mmol) and K₂CO₃ (4.41 g, 31.9 mmol) in DMF (50 mL) was heated to 110° C. and allowed to stir for 16 h. The mixture was concentrated in vacuo to afford the title compound (2.80 g, 56.0%) as a yellow solid. LCMS (ES⁺): 236.1 [MH]⁺.

### Intermediate 7

### (3-Fluoro-4-(4-methylpiperazin-1-yl)phenyl)boronic acid

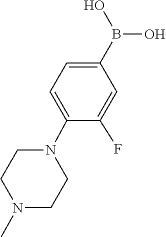 [SMILES: CN1CCN(c2ccc(B(O)O)cc2F)CC1]

Step 1: 1-(4-Bromo-2-fluorophenyl)-4-methylpiperazine (1.00 g, 3.66 mmol), bis(pinacolato)diboron (1.39 g, 5.48 mmol), Pd(dppf)Cl₂ (26.8 mg, 0.37 mmol) and NaOAc (601 mg, 7.32 mmol) in DXN (25 mL) was heated to 100° C. and allowed to stir for 12 h under N₂. The mixture was concentrated in vacuo to afford crude 1-(2-fluoro-4-(4,4,5,5-tetramethyl-1,3,2-dioxaborolan-2-yl)phenyl)-4-methylpiperazine which was used in the next step without further purification. LCMS (ES⁺): 321.2 [MH]⁺.

Step 2: Crude 1-(2-fluoro-4-(4,4,5,5-tetramethyl-1,3,2-dioxaborolan-2-yl)phenyl)-4-methylpiperazine, and NaIO₄ (3.22 g, 15.1 mmol) in THF (20 mL) was heated to 40° C. and allowed to stir for 5 h. The mixture was filtered, washing with EtOAc, the filtrate was then concentrated in vacuo. Purification via RP column chromatography afforded the title compound (130 mg, 19% yield over 2 steps) as a light-yellow oil. LCMS (ES⁺): 239.2 [MH]⁺.

### Intermediate 8

### (2-(3-((tert-Butoxycarbonyl)amino)propoxy)-6-methoxyphenyl)boronic acid

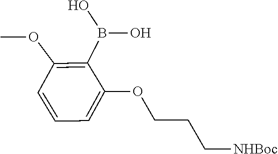 [SMILES: COc1cccc(OCCCNC(=O)OC(C)(C)C)c1B(O)O]

Step 1: 2-Bromo-3-methoxyphenol (3.00 g, 14.8 mmol), tert-butyl N-(3-bromopropyl)carbamate (3.52 g, 14.8 mmol), NaI (2.21 g, 14.8 mmol) and K₂CO₃ (4.08 g, 29.6 mmol) in DMF (20 mL) was stirred for 16 h at 60° C. Reaction quenched with water extracting with EtOAc (2×200 mL). The combined organics were washed with brine, dried (Na₂SO₄), and concentrated in vacuo. Purification via NP column chromatography afforded tert-butyl (3-(2-bromo-3-methoxyphenoxy)propyl)carbamate (3.20 g, 60.1%) as a colourless oil. LCMS (ES⁺): 360.1 [MH]⁺.

Step 2: To tert-butyl (3-(2-bromo-3-methoxyphenoxy)propyl)carbamate (1.00 g, 2.78 mmol) in THF (10 mL) was added n-BuLi (2.50 mL, 6.25 mmol) dropwise at −78° C. under N₂. To the mixture was added trimethyl borate (288 mg, 2.78 mmol) dropwise. The mixture was then allowed to warm to RT and stir for a further 1 h. Reaction quenched with water, extracting with EtOAc (2×100 mL). The combined organic layers were washed with brine, dried (Na₂SO₄), and concentrated in vacuo. Purification via NP column chromatography afforded the title compound (460 mg, 51.0%) as a colourless oil. LCMS (ES⁺): 326.2 [MH]⁺.

### Intermediate 9

### (3R,5R)-N,N,5-Trimethylpyrrolidin-3-amine

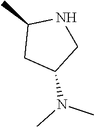

Step 1: tert-Butyl (2R,4R)-4-amino-2-methylpyrrolidine-1-carboxylate (1.00 g, 4.99 mmol) and formaldehyde (1.12 mL, 15.0 mmol) in MeCOH (20 mL) was stirred at RT for 30 min. The mixture was cooled to 0° C. and STAB (4.23 g, 20.0 mmol) was added in portions. The mixture was then allowed to warm to RT and stir for a further 2 h. The mixture was concentrated in vacuo and purified via NP column chromatography. Drying in a vacuum oven at 60° C. for 1 h afforded tert-butyl (2R,4R)-4-(dimethylamino)-2-methylpyrrolidine-1-carboxylate (778 mg, 68.2%) as a colourless oil. LCMS (ES⁺): 229.2 [MH]⁺. UPLC: Rt 3.23 min, 100% purity.

Step 2: To tert-butyl (2R,4R)-4-(dimethylamino)-2-methylpyrrolidine-1-carboxylate (778 mg, 3.40 mmol) in DCM (11 mL) was added TFA (2.5 mL). The mixture was allowed to stir for 2 h. The mixture was concentrated in vacuo giving crude title compound (763 mg) as a brown oil which was used directly in the next step without further purification. LCMS (ES⁺): 129.1 [MH]⁺.

### Intermediate 10

### (2R,4R)-4-(Azetidin-1-yl)-2-methylpyrrolidine

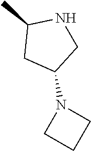

Step 1: tert-Butyl (2R,4R)-4-amino-2-methylpyrrolidine-1-carboxylate (3.00 g, 15.0 mmol), 1,3-dibromopropane (2.50 mL, 24.6 mmol), and K₂CO₃ (10.4 g, 75.2 mmol) in MeCN (200 mL) was heated to 90° C. and stirred for 21 h. The mixture was filtered, washing with MeCN, the filtrate was concentrated in vacuo and purified via NP column chromatography to afford crude tert-butyl (2R,4R)-4-(azetidin-1-yl)-2-methylpyrrolidine-1-carboxylate which was used directly in the next step without further purification.

Step 2: To crude tert-butyl (2R,4R)-4-(azetidin-1-yl)-2-methylpyrrolidine-1-carboxylate in DCM (12 mL) was added TFA (4.0 mL). The mixture was concentrated in vacuo and the crude material was purified via a catch and release cartridge to afford the title compound (617 mg, 29.4% over 2 steps) as a brown oil. LCMS (ES⁺): 141.0 [MH]⁺.

### Intermediate 11

### (R)-3-(Azetidin-1-yl)pyrrolidine

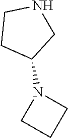

Step 1: To benzyl (R)-3-aminopyrrolidine-1-carboxylate (15.0 g, 68.1 mmol) and DIEA (35.6 mL, 205 mmol) in MeCN (500 mL) was added 1,3-dibromopropane (11.0 mL, 108 mmol) dropwise. The mixture was then heated to 80° C. and allowed to stir for 12 h. The mixture was concentrated in vacuo and purified via RP column chromatography to afford benzyl (R)-3-(azetidin-1-yl)pyrrolidine-1-carboxylate (6.70 g, 37.8%) as a yellow oil. LCMS (ES⁺): 261.2 [MH]⁺.

Step 2: To benzyl (R)-3-(azetidin-1-yl)pyrrolidine-1-carboxylate (6.30 g, 24.2 mmol) in EtOAc (200 mL) was added Pd/C (10% by weight, 6.31 g, 5.93 mmol). The mixture was allowed to stir for 3 h under an atmosphere of H₂. The mixture was filtered washing with EtOAc, the filtrate was concentrated in vacuo to afford crude title compound (2.60 g) as a yellow oil which was used in the next step without further purification. LCMS (ES⁺): 127.3 [MH]⁺.

### Intermediate 12

### 1-(4-(4-Bromo-1H-imidazol-1-yl)phenyl)-4-methylpiperazine

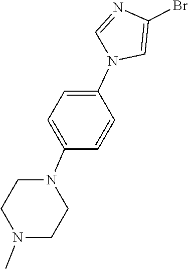 [SMILES: CN1CCN(c2ccc(-n3cnc(Br)c3)cc2)CC1]

A mixture of 4-(4-methylpiperazin-1-yl)phenylboronic acid (749 mg, 3.40 mmol), 4-bromo-1H-imidazole (200 mg, 1.36 mmol), NaOH (82.0 mg, 2.04 mmol) and CuCl₂ (22.0 mg, 0.16 mmol) in MeCOH (10 mL) was stirred overnight at 60° C. under O₂. The mixture was cooled to RT, filtered, and the filter cake washed with MeCOH (2×10 mL). The filtrate was concentrated in vacuo and purified via prep-TLC (DCM/MeOH 10:1) to afford the title compound (220 mg, 50.3%) as a yellow solid. LCMS (ES⁺): 321.10 [MH]⁺.

### Intermediate 13

### tert-Butyl (3-(2-(4-bromo-1H-imidazol-1-yl)phenoxy)propyl)carbamate

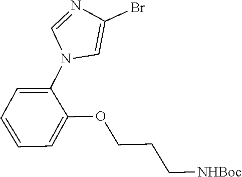 [SMILES: CC(C)(C)OC(=O)NCCCOc1ccccc1-n1cnc(Br)c1]

Intermediate 13 was prepared similarly to Intermediate 12 via Chan-Lam coupling of Intermediate 1 with 4-bromo-1H-imidazole using CuCl₂ and NaOH then purification via NP column chromatography to afford the title compound (40.1 mg, 38.2%) as a white solid. LCMS (ES⁺): 395.9 [MH]⁺.

### Intermediate 14

### (4-(4-Bromo-1H-imidazol-1-yl)phenyl)(4-methylpiperazin-1-yl)methanone

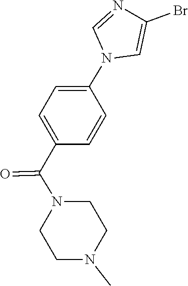 [SMILES: CN1CCN(C(=O)c2ccc(-n3cnc(Br)c3)cc2)CC1]

A mixture of 4-(4-methylpiperazine-1-carbonyl)phenylboronic acid (500 mg, 1.51 mmol), 4-bromo-1H-imidazole (300 mg, 2.04 mmol), boric acid (190 mg, 3.07 mmol), and Cu(OAc)₂ (85.0 mg, 0.47 mmol) in MeCN (2.5 mL) was heated in a sealed tube at 80° C. for 28 h. The reaction mixture was diluted with EtOAc (20 mL) and filtered through Celite®, washing with further EtOAc. The filtrate was then concentrated in vacuo and purified via NP column chromatography to afford the title compound (121 mg, 21.9%) as a colourless gum. LCMS (ES⁺): 349.1 [MH]⁺.

### Intermediate 15

### 4-(4-(4-Bromo-1H-imidazol-1-yl)benzyl)morpholine

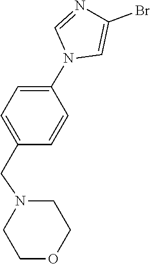 [SMILES: Brc1cn(-c2ccc(CN3CCOCC3)cc2)cn1]

To a stirred solution of Intermediate 2 (412 mg, 1.86 mmol) and 4-bromo-1H-imidazole (274 mg, 1.86 mmol) in DMF (15 mL) were added DIEA (723 mg, 5.59 mmol) and Cu(OAc)₂ (677 mg, 3.73 mmol) at RT. The resulting mixture was stirred for 12 h at 60° C. under O₂ then cooled to RT, filtered, and the filtrate concentrated in vacuo and purified via NP column chromatography to afford the title compound (155 mg, 25.8%) as a red solid. LCMS (ES⁺): 322.15 [MH]⁺.

### Intermediates 16 to 20

Intermediates 16 to 20 were prepared similarly to Intermediate 15 via Chan-Lam coupling of 4-bromo-1H-imidazole with the appropriate boronic acid or boronic ester using Cu(OAc)₂ and either DIEA or pyridine as the base; see Table 1 below.

TABLE 1
Chan-Lam Coupling of 4-bromo-1H-imidazole with the appropriate
boronic acid or boronic ester using Cu(OAc)₂

| | | | Reactant used; |
|---|---|---|---|
| Int. | Structure | Name | Form; Yield; LCMS |
| 16 | 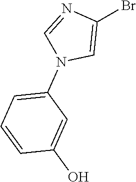 [SMILES: Oc1cccc(-n2cnc(Br)c2)c1] | 3-(4-Bromo-1H-imidazol-1- yl)phenol | CAS: 87199-18-6; Dark blue solid; Yield 3.50 g, 25.2%; LCMS (ES⁺): 239.1 [MH]⁺ |
| 17 | 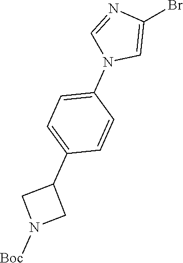 [SMILES: CC(C)(C)OC(=O)N1CC(c2ccc(-n3cnc(Br)c3)cc2)C1] | tert-Butyl 3-(4-(4-bromo-1H- imidazol-1- yl)phenyl)azetidine-1- carboxylate | Intermediate 5; Light yellow oil; Yield 60.0 mg, 19.7%; LCMS (ES⁺): 378.3 [MH]⁺ |
| 18 | 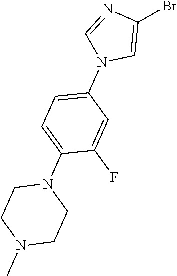 [SMILES: CN1CCN(c2ccc(-n3cnc(Br)c3)cc2F)CC1] | 1-(4-(4-Bromo-1H-imidazol-1- yl)-2-fluorophenyl)-4- methylpiperazine | Intermediate 7; Yellow solid; Yield 40.0 mg, 56.1%; LCMS (ES⁺): 339.3 [MH]⁺ |
| 19 | 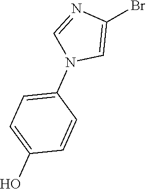 [SMILES: Oc1ccc(-n2cnc(Br)c2)cc1] | 4-(4-Bromo-1H-imidazol-1- yl)phenol | CAS: 71597-85-8; Dark blue solid; Yield 8.00 g, 30.8%; LCMS (ES⁺): 239.1 [MH]⁺ |
| 20 | 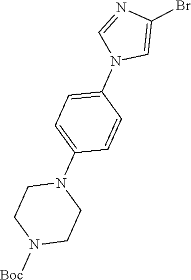 [SMILES: CC(C)(C)OC(=O)N1CCN(c2ccc(-n3cnc(Br)c3)cc2)CC1] | tert-Butyl 4-(4-(4-bromo-1H- imidazol-1- yl)phenyl)piperazine-1- carboxylate | CAS: 457613-78-4; Light brown solid; Yield 215 mg, 80.8%; LCMS (ES⁺): 347.1 [MH]⁺ |

### Intermediate 21

### 4-Bromo-1-(4-bromo-2-methoxyphenyl)-1H-imidazole

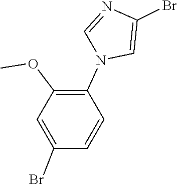

(4-Bromo-2-methoxy-phenyl)boronic acid (4.92 g, 21.3 mmol), 4-bromo-1H-imidazole (4.70 g, 32.0 mmol), pyridine (5.15 mL, 63.9 mmol) and Cu(OTf)₂ (15.4 g, 42.6 mmol) in DMF (30 mL) were heated to 40° C. and stirred for 17 h. The reaction mixture was concentrated in vacuo, diluted with EtOAc (30 mL) and water (30 mL), the phases separated, and the aqueous phase further extracted with EtOAc (30 mL). The combined organic phases were washed with brine (30 mL), concentrated in vacuo and purified via NP column chromatography to afford the title compound (1.36 g, 19.2%) as a white solid. LCMS (ES⁺): 330.7 [MH]⁺.

### Intermediates 22 to 38

Intermediates 22 to 38 were prepared similarly to Intermediate 21 via Chan-Lam coupling of 4-bromo-1H-imidazole with the appropriate boronic acid or boronic ester using Cu(OTf)₂ and either pyridine, DIEA or Et₃N as the base; see Table 2 below.

TABLE 2
Chan-Lam Coupling of 4-bromo-1H-imidazole with the appropriate
boronic acid or boronic ester using Cu(OTf)₂

| | | | Reactant used; |
|---|---|---|---|
| Int. | Structure | Name | Form; Yield; LCMS |
| 22 | 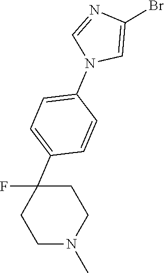 [SMILES: CN1CCC(F)(c2ccc(-n3cnc(Br)c3)cc2)CC1] | 4-(4-(4-Bromo-1H- imidazol-1-yl)phenyl)-4- fluoro-1-methylpiperidine | Intermediate 3; Brown solid; Yield 76.0 mg, 38.1%; LCMS (ES⁺): 338.1 [MH]⁺ |
| 23 | 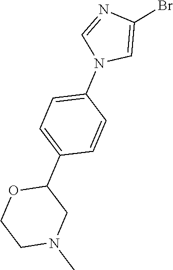 [SMILES: CN1CCOC(c2ccc(-n3cnc(Br)c3)cc2)C1] | 2-(4-(4-Bromo-1H- imidazol-1-yl)phenyl)-4- methylmorpholine | Intermediate 4; Yellow oil; Yield 131 mg, 69.1%; LCMS (ES⁺): 322.2 [MH]⁺ |
| 24 | 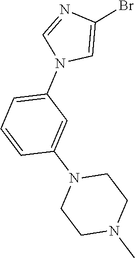 [SMILES: CN1CCN(c2cccc(-n3cnc(Br)c3)c2)CC1] | 1-(3-(4-Bromo-1H- imidazol-1-yl)phenyl)-4- methylpiperazine | CAS: 1139717-76-2; Yellow solid; Yield 100 mg, 13.7%; LCMS (ES⁺): 321.1 [MH]⁺ |
| 25 | 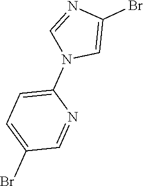 [SMILES: Brc1ccc(-n2cnc(Br)c2)nc1] | 2-Bromo-5-(4-bromo-1H- imidazol-1-yl)pyridine | CAS: 223463-14-7; Yellow solid; Yield 789 mg, 26.3%; LCMS (ES⁺): 304.15 [MH]⁺ |
| 26 | 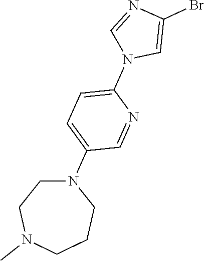 [SMILES: CN1CCCN(c2ccc(-n3cnc(Br)c3)nc2)CC1] | 1-(5-(4-Bromo-1H- imidazol-1-yl)pyridin-2- yl)-4-methyl-1,4- diazepane | Intermediate 6; Yellow liquid; Yield 600 mg, 11.9%; LCMS (ES⁺): 336.3 [MH]⁺ |
| 27 | 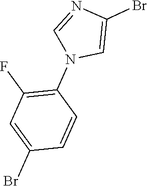 [SMILES: Fc1cc(Br)ccc1-n1cnc(Br)c1] | 4-Bromo-1-(4-bromo-2- fluorophenyl)-1H- imidazole | CAS: 216393-64-5; Yellow solid; Yield 1.70 g, 58.1%; LCMS (ES⁺): 321.9 [MH]⁺ |
| 28 | 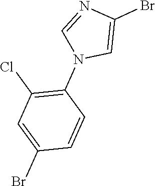 [SMILES: Clc1cc(Br)ccc1-n1cnc(Br)c1] | 4-Bromo-1-(4-bromo-2- chlorophenyl)-1H- imidazole | CAS: 1046861-20-4; Yellow solid; Yield 510 mg, 35.7%; LCMS (ES⁺): 316.2 [MH]⁺ |
| 29 | 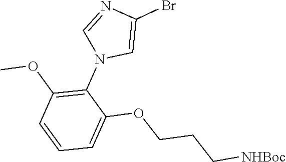 [SMILES: COc1cccc(OCCCNC(=O)OC(C)(C)C)c1-n1cnc(Br)c1] | tert-Butyl 3-(2-(4-bromo- 1H-imidazol-1-yl)-3- methoxyphenoxy)propyl) carbamate | Intermediate 8; Colourless oil; Yield 90.0 mg, 14.9%; LCMS (ES⁺): 426.1 [MH]⁺ |
| 30 | 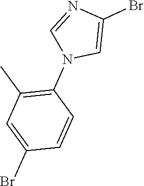 | 4-Bromo-1-(4-bromo-2- methylphenyl)-1H- imidazole | CAS: 221006-71-9; Yellow solid; Yield 448 mg, 30.5%; LCMS (ES⁺): 315.5 [MH]⁺ |
| 31 | 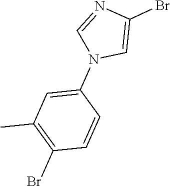 | 4-Bromo-1-(4-bromo-3- methylphenyl)-1H- imidazole | CAS: 221006-67-3; White solid; Yield 1.20 g, 27.2%; LCMS (ES⁺): 316.2 [MH]⁺ |
| 32 | 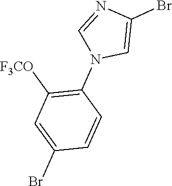 [SMILES: FC(F)(F)Oc1cc(Br)ccc1-n1cnc(Br)c1] | 4-Bromo-1-(4-bromo-2- (trifluoromethoxy)phenyl)- 1H-imidazole | CAS: 1048990-22-2; Brown oil; Yield 511 mg, 37.7%; LCMS (ES⁺): 386.0 [MH]⁺ |
| 33 | 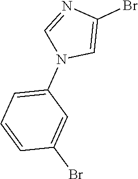 [SMILES: Brc1cccc(-n2cnc(Br)c2)c1] | 4-Bromo-1-(3- bromophenyl)-1H- imidazole | CAS: 89598-96-9; Off-white oil; Yield 4.00 g, 26.6%; LCMS (ES⁺): 301.6 [MH]⁺ |
| 34 | 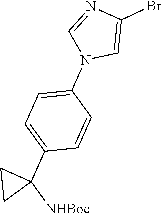 [SMILES: CC(C)(C)OC(=O)NC1(c2ccc(-n3cnc(Br)c3)cc2)CC1] | tert-Butyl (1-(4-(4-bromo- 1H-imidazol-1- yl)phenyl)cyclopropyl) carbamate | CAS: 1217500-58-7; Yellow solid; Yield 468 mg, 38.1%; LCMS (ES⁺): 378.2 [MH]⁺ |
| 35 | 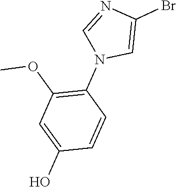 | 4-(4-Bromo-1H-imidazol- 1-yl)-3-methoxyphenol | CAS: 507462-88-6; Light brown solid; Yield 645 mg, 60.0%; LCMS (ES⁺): 269.1 [MH]⁺ |
| 36 | 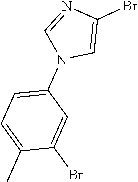 | 4-Bromo-1-(3-bromo-4- methylphenyl)-1H- imidazole | CAS: 1312765-69-7; White solid; Yield 600 mg, 45.3%; LCMS (ES⁺): 316.9 [MH]⁺ |
| 37 | 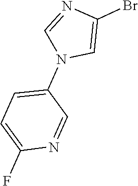 [SMILES: Fc1ccc(-n2cnc(Br)c2)cn1] | 5-(4-Bromo-1H-imidazol- 1-yl)-2-fluoropyridine | CAS: 351019-18-6; Off-white solid; Yield 2.44 g, 70.9%; LCMS (ES⁺): 243.1 [MH]⁺ |
| 38 | 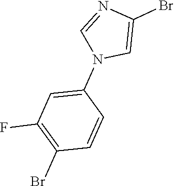 [SMILES: Fc1cc(-n2cnc(Br)c2)ccc1Br] | 4-Bromo-1-(4-bromo-3- fluorophenyl)-1H- imidazole | CAS: 374790-97-3; White solid; Yield 2.30 g, 78.7%; LCMS (ES⁺): 321.0 [MH]⁺ |

### Intermediate 39

### tert-Butyl 6-(4-(4-bromo-1H-imidazol-1-yl)-3-methoxyphenoxy)-2-azaspiro[3.3]heptane-2-carboxylate

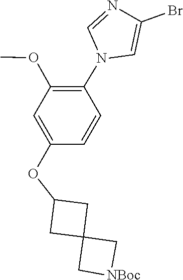 [SMILES: COc1cc(OC2CC3(C2)CN(C(=O)OC(C)(C)C)C3)ccc1-n1cnc(Br)c1]

To a stirred mixture of Intermediate 35 (650 mg, 2.42 mmol) in DMF (3.0 mL) was added NaH (116 mg, 4.83 mmol) at 0° C. The resulting mixture was stirred at 0° C. for 0.5 h then tert-butyl 6-(methanesulfonyloxy)-2-azaspiro[3.3]heptane-2-carboxylate (1.05 g, 3.62 mmol) was added dropwise at 0° C. The resulting mixture was stirred for additional 2 h at 80° C. then concentrated in vacuo and purified via prep-TLC (PE/EtOAc 1:1) to afford the title compound (756 mg, 67.4%) as a yellow oil. LCMS (ES⁺): 466.10 [MH]⁺.

### Intermediate 40

### 4-(4-Bromo-1H-imidazol-1-yl)-2-chlorobenzaldehyde

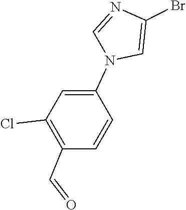 [SMILES: O=Cc1ccc(-n2cnc(Br)c2)cc1Cl]

A mixture of 4-bromo-1H-imidazole (200 mg, 1.36 mmol), 2-chloro-4-fluorobenzaldehyde (216 mg, 1.36 mmol) and K₂CO₃ (282 mg, 2.04 mmol) in DMSO (2.0 mL) was stirred at 60° C. for 16 h. The reaction mixture was allowed to cool to RT, and poured into rapidly stirring water (15 mL). The resulting solid was collected via filtration, washed with water (4×30 mL), then dried in the vacuum oven at 50° C. for 3 h to afford the title compound (350 mg, 77.3%) as a white solid. LCMS (ES⁺): 284.9 [MH]⁺.

### Intermediates 41 to 44

Intermediates 41 to 44 were prepared similarly to Intermediate 40 via nucleophilic aromatic substitution of the appropriate halogenated aromatic substrate with 4-bromo-1H-imidazole in DMSO using K₂CO₃ as the base; see Table 3 below.

TABLE 3
Nucleophilic aromatic substitution of the appropriate halogenated
aromatic substrate with 4-bromo-1H-imidazole

| | | | Reactant used; |
|---|---|---|---|
| Int. | Structure | Name | Form; Yield; LCMS |
| 41 | 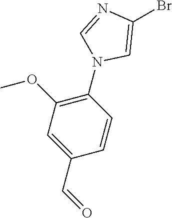 [SMILES: COc1cc(C=O)ccc1-n1cnc(Br)c1] | 4-(4-Bromo-1H-imidazole-1- yl)-3-methoxybenzaldehyde | CAS: 128495-46-5; White solid; Yield 850 mg, 22.2%; LCMS (ES⁺): 280.9 [MH]⁺ |
| 42 | 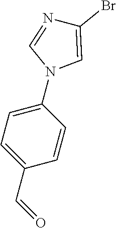 [SMILES: O=Cc1ccc(-n2cnc(Br)c2)cc1] | 4-(4-Bromo-1H-imidazol-1- yl)benzaldehyde | CAS: 459-57-4; White solid; Yield 940 mg, 76.2%; LCMS (ES⁺): 251.1 [MH]⁺ |
| 43 | 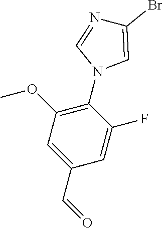 [SMILES: COc1cc(C=O)cc(F)c1-n1cnc(Br)c1] | 4-(4-Bromo-1H-imidazol-1- yl)-3-fluoro-5- methoxybenzaldehyde | CAS: 881190-46-1; White solid; Yield 61.0 mg, 14.4%; LCMS (ES⁺): 298.6 [MH]⁺ |
| 44 | 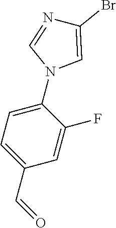 [SMILES: O=Cc1ccc(-n2cnc(Br)c2)c(F)c1] | 4-(4-Bromo-1H-imidazol-1- yl)-3-fluorobenzaldehyde | CAS: 34036-07-2; White solid; Yield 136 mg, 37.1%; LCMS (ES⁺): 268.6 [MH]⁺ |

### Intermediate 45

### 4-Bromo-1-(4-bromophenyl)-1H-imidazole

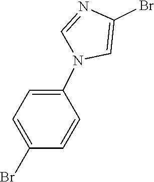 [SMILES: Brc1ccc(-n2cnc(Br)c2)cc1]

A mixture of 4-bromo-1H-imidazole (5.00 g, 34.0 mmol), 1-bromo-4-fluorobenzene (7.50 mL, 69.3 mmol) and K₃PO₄ (36.3 g, 171 mmol) in DMF (100 mL) was heated to 150° C. for 20 h. The reaction mixture was slowly poured into rapidly stirring water (400 mL), filtered, then the residue was washed with water (20 mL) and dried in a vacuum oven at 60° C. for 5 h to afford the title compound (7.93 g, 74.3%) as a white solid. LCMS (ES⁺): 300.8 [MH]⁺.

### Intermediates 46 to 48

Intermediates 46 to 48 were prepared similarly to Intermediate 45 via nucleophilic aromatic substitution of the appropriate halogenated aromatic substrate with 4-bromo-1H-imidazole in DMF using K₃PO₄ as the base; see Table 4 below.

TABLE 4
Nucleophilic aromatic substitution of the appropriate halogenated
aromatic substrate with 4-bromo-1H-imidazole

| | | | Reactant used; |
|---|---|---|---|
| Int. | Structure | Name | Form; Yield; LCMS |
| 46 | 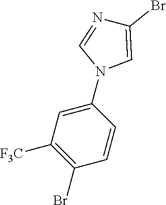 [SMILES: FC(F)(F)c1cc(-n2cnc(Br)c2)ccc1Br] | 4-Bromo-1-(4-bromo-3- (trifluoromethyl)phenyl)-1H-imidazole | CAS: 40161-55-5; Beige solid; Yield 800 mg, 62.4%; LCMS (ES⁺): 368.7 [MH]⁺ |
| 47 | 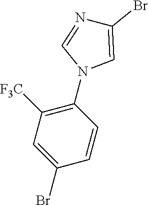 [SMILES: FC(F)(F)c1cc(Br)ccc1-n1cnc(Br)c1] | 4-Bromo-1-(4-bromo-2- (trifluoromethyl)phenyl)-1H-imidazole | CAS: 393-37-3; White solid; Yield 900 mg, 70.4%; LCMS (ES⁺): 368.9 [MH]⁺ |
| 48 | 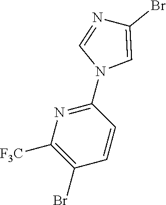 [SMILES: FC(F)(F)c1nc(-n2cnc(Br)c2)ccc1Br] | 3-Bromo-6-(4-bromo-1H-imidazol-1- yl)-2-(trifluoromethyl)pyridine | CAS: 1227563-63-4; White solid; Yield 610 mg, 72.5%; LCMS (ES⁺): 369.9 [MH]⁺ |

### Intermediate 49

### 5-Bromo-2-(4-bromo-1H-imidazol-1-yl)pyridine

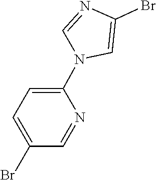 [SMILES: Brc1ccc(-n2cnc(Br)c2)nc1]

To a stirred mixture of 5-bromo-2-fluoropyridine (1.00 g, 5.68 mmol) and 4-bromo-1H-imidazole (1.25 g, 8.52 mmol) in DMF (20 mL) was added Cs₂CO₃ (3.70 g, 11.4 mmol) at RT. The resulting mixture was stirred for 1 h at 100° C. then quenched with water (100 mL), extracted with EtOAc (5×100 mL) and the combined organic layers were washed with brine (50 mL), dried (Na₂SO₄), concentrated in vacuo and purified via NP column chromatography to afford the title compound (1.10 g, 63.9%) as a yellow solid. LCMS (ES⁺): 304.1 [MH]⁺.

### Intermediate 50

### 2-(4-Bromo-1H-imidazol-1-yl)-5-chloropyrazine

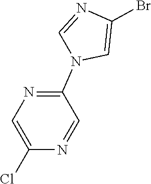 [SMILES: Clc1cnc(-n2cnc(Br)c2)cn1]

To a solution of 4-bromo-1H-imidazole (493 mg, 3.36 mmol) in DMF (10 mL) was added NaH (162 mg, 4.05 mmol) at 0° C. The mixture was stirred for 1 h then 2,5-dichloro-pyrazine (500 mg, 3.36 mmol) was added. The mixture was warmed to 100° C., stirred for 1 h then quenched via water and extracted with DCM (3×25 mL). The combined organic layers were washed with brine, dried (Na₂SO₄), concentrated in vacuo and purified via NP column chromatography to afford the title compound (400 mg, 45.9%) as a light-yellow solid. LCMS (ES⁺): 259.0 [MH]⁺.

### Intermediate 51

### 4-(4-Bromo-1H-imidazol-1-yl)-3-methyl-5-(trifluoromethyl)benzaldehyde

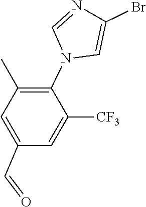 [SMILES: Cc1cc(C=O)cc(C(F)(F)F)c1-n1cnc(Br)c1]

To a stirred mixture of 4-fluoro-3-methyl-5-(trifluoromethyl)benzaldehyde (2.00 g, 9.70 mmol) and 4-bromo-1H-imidazole (2.14 g, 14.6 mmol) in MeCN (5.0 mL) was added DIEA (3.38 mL, 19.4 mmol) dropwise at RT. The mixture was stirred at 80° C. for 16 h then concentrated in vacuo and purified via NP column chromatography to afford the title compound (1.30 g, 40.2%) as a white solid. LCMS (ES⁺): 333.0 [MH]⁺.

### Intermediate 52

### 4-(4-Bromo-1H-imidazol-1-yl)-3,5-dimethylbenzaldehyde

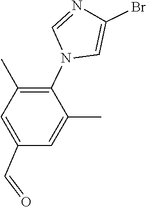 [SMILES: Cc1cc(C=O)cc(C)c1-n1cnc(Br)c1]

To a stirred mixture of 4-fluoro-3,5-dimethylbenzaldehyde (1.00 g, 6.57 mmol) and 4-bromo-1H-imidazole (1.93 g, 13.1 mmol) in DMF (50 mL) was added K₂CO₃ (1.82 g, 13.1 mmol) at RT under N₂. The resulting mixture was stirred for 2 h at 100° C., quenched with water, extracted with EtOAc (3×25 mL) then the combined organic layers were washed with brine, dried (Na₂SO₄), concentrated in vacuo and purified via NP column chromatography to afford the title compound (103 mg, 5.60%) as a light-yellow powder. LCMS (ES⁺): 279.10 [MH]⁺.

### Intermediate 53

### 3-Bromo-6-(4-bromo-1H-imidazol-1-yl)-2-methylpyridine

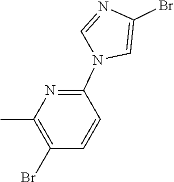

A solution of 3-bromo-6-fluoro-2-methylpyridine (2.00 g, 10.5 mmol), 4-bromo-1H-imidazole (2.19 g, 14.9 mmol) and DIEA (2.58 g, 20.0 mmol) in DMF (10 mL) was stirred overnight at 100° C. then quenched with water and extracted with EtOAc (3×10 mL). The combined organic layers were washed with brine (3×10 mL), dried (Na₂SO₄), concentrated in vacuo and purified via NP column chromatography to afford the title compound (356 mg, 10.7%) as a white solid. LCMS (ES⁺): 317.9 [MH]⁺.

### Intermediate 54

### 5-Bromo-2-(4-bromo-1H-imidazol-1-yl)pyrimidine

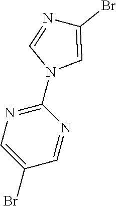 [SMILES: Brc1cnc(-n2cnc(Br)c2)nc1]

Intermediate 54 was prepared similarly to Intermediate 53 via nucleophilic aromatic substitution of 5-bromo-2-chloropyrimidine with 4-bromo-1H-imidazole in DMF using DIEA as the base then purification via NP column chromatography to afford the title compound (420 mg, 17.8%) as a white solid. LCMS (ES⁺): 304.9 [MH]⁺.

### Intermediate 55

### 4-Bromo-1-(4-bromo-2-nitrophenyl)-1H-imidazole

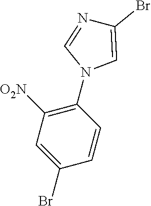 [SMILES: O=[N+]([O-])c1cc(Br)ccc1-n1cnc(Br)c1]

Intermediate 55 was prepared similarly to Intermediate 53 via nucleophilic aromatic substitution of 4-bromo-1-fluoro-2-nitrobenzene with 4-bromo-1H-imidazole in MeCN using DIEA as the base then purification via NP column chromatography to afford the title compound (6.30 g, 93.2%) as a yellow solid. LCMS (ES⁺): 347.6 [MH]⁺.

### Intermediate 56

### tert-Butyl (3S,4S)-3-((4-(4-bromo-1H-imidazol-1-yl)benzyl)oxy)-4-fluoropyrrolidine-1-carboxylate

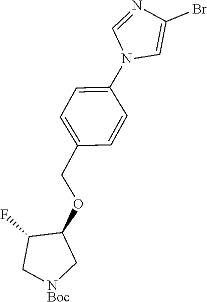 [SMILES: CC(C)(C)OC(=O)N1C[C@H](OCc2ccc(-n3cnc(Br)c3)cc2)[C@@H](F)C1]

Step 1: To a solution of Intermediate 42 (1.00 g, 3.98 mmol) in MeCOH (20 mL) was added NaBH₄ (301 mg, 7.96 mmol) at 0° C. The reaction mixture was stirred for 1 h at RT then quenched with water (50 mL) at 0° C. and the resulting mixture was extracted with EtOAc (3×100 mL). The combined organic layers were washed with brine (1×50 mL), dried (Na₂SO₄), and concentrated in vacuo to afford [4-(4-bromoimidazol-1-yl)phenyl]methanol (990 mg, 98.2%) as a white solid. LCMS (ES⁺): 253.0 [MH]⁺.

Step 2: To a stirred mixture of [4-(4-bromoimidazol-1-yl)phenyl]methanol (3.60 g, 14.2 mmol) and CBr₄ (9.43 g, 28.5 mmol) in DCM (30 mL) was added PPh₃ (7.46 g, 28.4 mmol) at 0° C. under N₂. The mixture was stirred for 4 h at 0° C. then concentrated in vacuo and purified via NP column chromatography to afford 4-bromo-1-[4-(bromomethyl)phenyl]imidazole (3.00 g, 66.8%) as a white solid. LCMS (ES⁺): 316.8 [MH]⁺.

Step 3: To a solution of tert-butyl (3S,4S)-3-fluoro-4-hydroxypyrrolidine-1-carboxylate (500 mg, 2.44 mmol) in DMF (10 mL) was added NaH (128 mg, 3.20 mmol) at 0° C. The mixture was stirred for 15 min then 4-bromo-1-[4-(bromomethyl)phenyl]imidazole (500 mg, 1.58 mmol) was added, the mixture warmed to RT, then stirred for 1 h. The reaction was quenched with water (10 mL), extracted with EtOAc (3×50 mL) and the combined organic layers were washed with brine (3×10 mL), dried (Na₂SO₄), concentrated in vacuo and purified via NP column chromatography to afford the title compound (530 mg, 76.1%) as a yellow oil. LCMS (ES⁺): 439.9 [MH]⁺.

### Intermediate 57

### tert-Butyl (3S,4R)-3-((4-(4-bromo-1H-imidazol-1-yl)benzyl)oxy)-4-fluoropyrrolidine-1-carboxylate

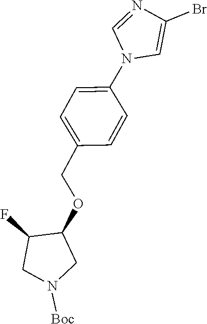 [SMILES: CC(C)(C)OC(=O)N1C[C@@H](F)[C@@H](OCc2ccc(-n3cnc(Br)c3)cc2)C1]

Intermediate 57 was prepared similarly to Intermediate 56, via aldehyde reduction of Intermediate 42 using NaBH₄ followed by Appel reaction using CBr₄ and PPh₃, then alkylation with tert-butyl (3R,4S)-3-fluoro-4-hydroxypyrrolidine-1-carboxylate to afford the title compound (660 mg, 94.7%) as a yellow solid. LCMS (ES⁺): 439.9 [MH]⁺.

### Intermediate 58

### 1-(4-(4-Bromo-1H-imidazol-11)benzyl)-4-methylpiperazine

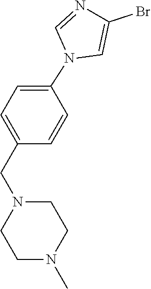 [SMILES: CN1CCN(Cc2ccc(-n3cnc(Br)c3)cc2)CC1]

To a stirred mixture of Intermediate 42 (600 mg, 2.39 mmol) and STAB (1.00 g, 4.78 mmol) in DCM (12 mL) was added 1-methyl-piperazine (359 mg, 3.58 mmol) at RT under N₂. The mixture was stirred for 2 h then concentrated in vacuo and purified via NP column chromatography to afford the title compound (329 mg, 41.1%) as a yellow solid. LCMS (ES⁺): 335.3 [MH]⁺.

### Intermediate 59

(R)-4-Bromo-1-(3-chloro-4-((3-fluoropyrrolidin-1-yl)methyl)phenyl)-1H-imidazole

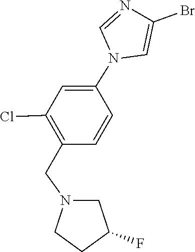 [SMILES: F[C@@H]1CCN(Cc2ccc(-n3cnc(Br)c3)cc2Cl)C1]

Intermediate 40 (50.0 mg, 0.15 mmol, 85.8% purity), (R)-(−)-3-fluoropyrrolidine-HCl (18.9 mg, 0.15 mmol) and MP-trimethylammonium cyanoborohydride resin (3.82 mmol/g, 101 mg, 0.38 mmol) in IPA (1.5 mL) and AcOH (150 μL) was heated using a microwave reactor (100° C.) for 25 min. The reaction mixture was diluted with DCM (5.0 mL), passed through a thin Celite® pad, concentrated in vacuo and purified via RP HPLC. The combined fractions were passed through a catch and elute cartridge to afford the title compound (26.0 mg, 48.3%) as a colourless gum. LCMS (ES⁺): 357.6 [MH]⁺.

### Intermediate 60

### (R)-4-Bromo-1-(2-fluoro-4-((3-fluoropyrrolidin-1-yl)methyl)-6-methoxyphenyl)-1H-imidazole

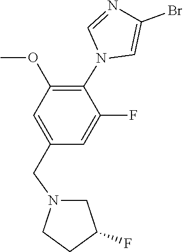 [SMILES: COc1cc(CN2CC[C@@H](F)C2)cc(F)c1-n1cnc(Br)c1]

Intermediate 60 was prepared similarly to Intermediate 59 via reductive amination of Intermediate 43 with (R)-(−)-3-fluoropyrrolidine·HCl using MP-trimethylammonium cyanoborohydride resin to afford the title compound (26.0 mg, 35.3%) as a colourless gum. LCMS (ES⁺): 371.7 [MH]⁺.

### Intermediate 61

### (R)-4-Bromo-1-(2-fluoro-4-((3-fluoropyrrolidin-1-yl)methyl)phenyl)-1H-imidazole

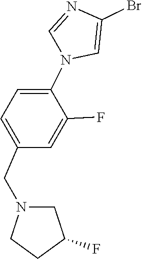 [SMILES: Fc1cc(CN2CC[C@@H](F)C2)ccc1-n1cnc(Br)c1]

Intermediate 61 was prepared similarly to Intermediate 59 via reductive amination of Intermediate 44 with (R)-(−)-3-fluoropyrrolidine-HCl using MP-trimethylammonium cyanoborohydride resin to afford the title compound (34.0 mg, 45.5%) as a colourless gum. LCMS (ES⁺): 341.6 [MH]⁺.

### Intermediate 62

### (R)-4-Bromo-1-(4-((3-fluoropyrrolidin-1-yl)methyl)-2-methyl-6-(trifluoromethyl)phenyl)-1H-imidazole

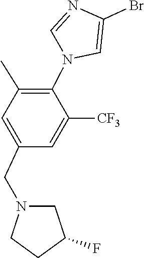 [SMILES: Cc1cc(CN2CC[C@@H](F)C2)cc(C(F)(F)F)c1-n1cnc(Br)c1]

To a stirred mixture of Intermediate 51 (1.30 g, 3.90 mmol) and (3R)-3-fluoropyrrolidine (0.70 g, 7.81 mmol) in MeCOH (10 mL) was added NaBH₃CN (0.49 g, 7.81 mmol) in portions at 0° C. The resulting mixture was stirred for 16 h at RT then quenched with water, concentrated in vacuo and purified via RP column chromatography to afford the title compound (579 mg, 36.5%) as a yellow oil. LCMS (ES⁺): 406.1 [MH]⁺.

### Intermediate 63

### (R)-4-Bromo-1-(4-((3-fluoropyrrolidin-1-yl)methyl)-2,6-dimethylphenyl)-1H-imidazole

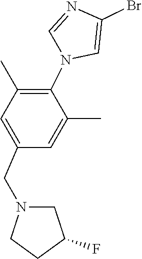 [SMILES: Cc1cc(CN2CC[C@@H](F)C2)cc(C)c1-n1cnc(Br)c1]

Intermediate 63 was prepared similarly to Intermediate 62 via reductive amination of Intermediate 52 with (R)-(−)-3-fluoropyrrolidine-HCl using NaBH₃CN to afford the title compound (100 mg, 77.7%) as a light-yellow solid. LCMS (ES⁺): 354.1 [MH]⁺.

### Intermediate 64

### (R)-1-(5-(4-Bromo-1H-imidazol-1-yl)pyrazin-2-yl)-N,N-dimethylpyrrolidin-3-amine

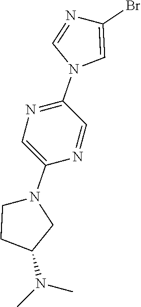 [SMILES: CN(C)[C@@H]1CCN(c2cnc(-n3cnc(Br)c3)cn2)C1]

A mixture of Intermediate 50 (400 mg, 1.54 mmol), DIEA (598 mg, 4.62 mmol) and (3R)-N,N-dimethylpyrrolidin-3-amine (176 mg, 1.54 mmol) in DMF (10 mL) was stirred for 2 h at 100° C. The resulting mixture was dried under vacuum and purified via NP column chromatography to afford the title compound (300 mg, 57.7%) as a light-yellow solid. LCMS (ES⁺): 337.2 [MH]⁺.

### Intermediate 65

### (R)-1-(5-(4-Bromo-1H-imidazol-1-yl)pyridin-2-yl)-N,N-dimethylpyrrolidin-3-amine

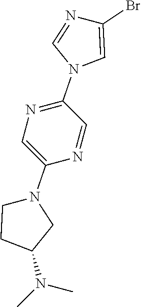 [SMILES: CN(C)[C@@H]1CCN(c2cnc(-n3cnc(Br)c3)cn2)C1]

DIEA (3.60 mL, 20.7 mmol) was added to a stirred mixture of Intermediate 37 (2.50 g, 10.3 mmol) and (3R)-N,N-dimethylpyrrolidin-3-amine (1.97 mL, 15.5 mmol) in DMF (25 mL) at RT, then the mixture stirred for 18 h. Water (100 mL) was added, and the resulting mixture extracted with EtOAc (4×100 mL). The combined organic layers were washed with brine (3×10 mL), dried (Na₂SO₄), concentrated in vacuo and purified via NP column chromatography to afford the title compound (976 mg, 28.1%) as a purple solid. LCMS (ES⁺): 336.2 [MH]⁺.

### Intermediate 66

### 4-Bromo-1-(4-((1-(tert-butyl)azetidin-3-yl)oxy)phenyl)-1H-imidazole

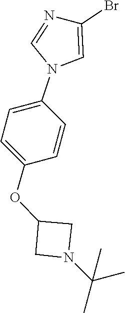 [SMILES: CC(C)(C)N1CC(Oc2ccc(-n3cnc(Br)c3)cc2)C1]

To a stirred solution of Intermediate 19 (300 mg, 1.26 mmol), 1-tert-butylazetidin-3-ol (243 mg, 1.88 mmol) and PPh₃ (494 mg, 1.88 mmol) in THF (5.0 mL) was added DIAD (381 mg, 1.88 mmol) dropwise at 0° C. under N₂. The resulting mixture was stirred for 2 h at 40° C. under N₂ then cooled to RT, quenched with water, concentrated in vacuo, and purified via NP column chromatography to afford the title compound (110 mg, 25.0%) as a brown solid. LCMS (ES⁺): 345.0 [MH]⁺.

### Intermediates 67 to 74

Intermediates 67 to 74 were prepared similarly to Intermediate 66 via Mitsunobu reaction of Intermediates 16, 19, and 35 with the appropriate alcohol using DIAD and PPh₃; see Table 5 below.

TABLE 5
Mitsunobu reactions of Intermediates 16, 19 and 35 using DIAD and PPh₃

| | | | Reactant(s) used; |
|---|---|---|---|
| Int. | Structure | Name | Form; Yield; LCMS |
| 67 | 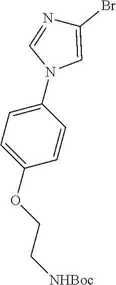 [SMILES: CC(C)(C)OC(=O)NCCOc1ccc(-n2cnc(Br)c2)cc1] | tert-Butyl (2-(4-(4-bromo-1H- imidazol-1- yl)phenoxy)ethyl)carbamate | Intermediate 19, CAS: 26690-80-2; Yellow solid; Yield 430 mg, 57.4%; LCMS (ES⁺): 382.0 [MH]⁺ |
| 68 | 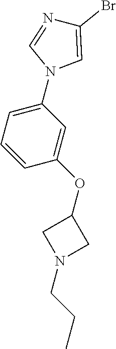 [SMILES: CCCN1CC(Oc2cccc(-n3cnc(Br)c3)c2)C1] | 4-Bromo-1-(3-((1-propylazetidin- 3-yl)oxy)phenyl)-1H-imidazole | Intermediate 16, CAS: 1340301-52-1; Light yellow solid; Yield 53.0 mg, 37.7%; LCMS (ES⁺): 336.1 [MH]⁺ |
| 69 | 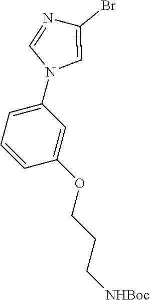 [SMILES: CC(C)(C)OC(=O)NCCCOc1cccc(-n2cnc(Br)c2)c1] | tert-Butyl (3-(3-(4-bromo-1H- imidazol-1- yl)phenoxy)propyl)carbamate | Intermediate 16, CAS: 58885-58-8; Brown oil; Yield 85.0 mg, 17.1%; LCMS (ES⁺): 396.5 [MH]⁺ |
| 70 | 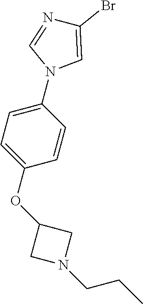 [SMILES: CCCN1CC(Oc2ccc(-n3cnc(Br)c3)cc2)C1] | 4-Bromo-1-(4-((1-propylazetidin- 3-yl)oxy)phenyl)-1H-imidazole | Intermediate 19, CAS: 1340301-52-1; Yellow solid; Yield 40.0 mg, 28.4%; LCMS (ES⁺): 336.1 [MH]⁺ |
| 71 | 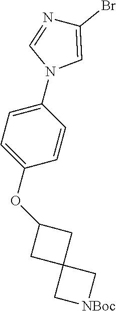 [SMILES: CC(C)(C)OC(=O)N1CC2(CC(Oc3ccc(-n4cnc(Br)c4)cc3)C2)C1] | tert-Butyl 6-(4-(4-bromo-1H- imidazol-1-yl)phenoxy)-2- azaspiro[3.3]heptane-2- carboxylate | Intermediate 19, CAS: 1147557-97-8; Black oil; Yield 260 mg, 47.7%; LCMS (ES⁺): 436.2 [MH]⁺ |
| 72 | 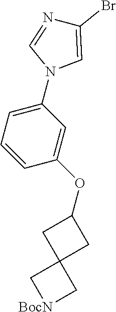 [SMILES: CC(C)(C)OC(=O)N1CC2(CC(Oc3cccc(-n4cnc(Br)c4)c3)C2)C1] | tert-Butyl 6-(3-(4-bromo-1H- imidazol-1-yl)phenoxy)-2- azaspiro[3.3]heptane-2- carboxylate | Intermediate 16, CAS: 1147557-97-8; Light yellow oil; Yield 105 mg, 19.3%; LCMS (ES⁺): 434.0 [MH]⁺ |
| 73 | 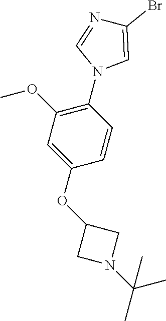 [SMILES: COc1cc(OC2CN(C(C)(C)C)C2)ccc1-n1cnc(Br)c1] | 4-Bromo-1-(4-((1-(tert- butyl)azetidin-3-yl)oxy)-2- methoxyphenyl)-1H-imidazole | Intermediate 35, CAS: 13156-04-2; Brown oil; Yield 179 mg, 63.3%; LCMS (ES⁺): 380.3 [MH]⁺ |
| 74 | 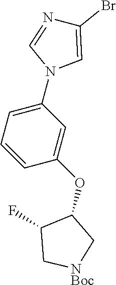 [SMILES: CC(C)(C)OC(=O)N1C[C@H](F)[C@H](Oc2cccc(-n3cnc(Br)c3)c2)C1] | tert-butyl (3R,4S)-3-(3-(4-bromo- 1H-imidazol-1-yl)phenoxy)-4- fluoropyrrolidine-1-carboxylate | Intermediate 16, CAS: 869481-93-6; White solid; Yield 410 mg, 76.7%; LCMS (ES⁺): 378.3 [MH]⁺ |

### Intermediate 75

### (1R,4R)-2-(4-(4-Bromo-1H-imidazol-1-yl)phenyl)-5-methyl-2,5-diazabicyclo[2.2.1]heptane

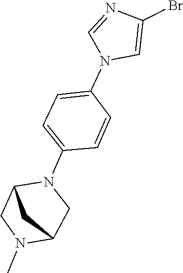 [SMILES: CN1C[C@H]2C[C@@H]1CN2c1ccc(-n2cnc(Br)c2)cc1]

Intermediate 45 (500 mg, 1.66 mmol), (1R,4R)-2-methyl-2,5-diazabicyclo[2.2.1]heptane (279 mg, 2.48 mmol), XPhos (79.0 mg, 0.17 mmol), XPhos Pd G3 (140 mg, 0.17 mmol) and Cs₂CO₃ (1.08 g, 3.32 mmol) in DXN (10 mL) were stirred overnight at 100° C. under N₂ then cooled to RT, concentrated in vacuo and purified via NP column chromatography to afford the title compound (110 mg, 19.9%) as a yellow solid. LCMS (ES⁺): 333.1 [MH]⁺.

### Intermediates 76 to 161

Intermediates 76 to 161 were prepared similarly to Intermediate 75 via XPhos Pd G3-catalysed Buchwald-Hartwig coupling of the specified (hetero)aryl bromide Intermediate with the appropriate amine; see Table 6 below.

TABLE 6
XPhos Pd G3-catalysed Buchwald-Hartwig coupling of the specified
(hetero)aryl bromide Intermediate with the appropriate amine

| | | | Reactant(s) used; |
|---|---|---|---|
| Int. | Structure | Name | Form; Yield; LCMS |
| 76 | 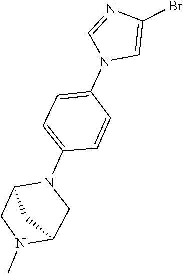 [SMILES: CN1C[C@@H]2C[C@H]1CN2c1ccc(-n2cnc(Br)c2)cc1] | (1S,4S)-2-(4-(4-Bromo-1H- imidazol-1-yl)phenyl)-5- methyl-2,5- diazabicyclo[2.2.1]heptane | Intermediate 45, CAS: 134679-22-4; Yellow solid; Yield 100 mg, 30.2%; LCMS (ES⁺): 333.1 [MH]⁺ |
| 77 | 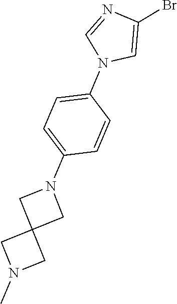 [SMILES: CN1CC2(C1)CN(c1ccc(-n3cnc(Br)c3)cc1)C2] | 2-(4-(4-Bromo-1H-imidazol- 1-yl)phenyl)-6-methyl-2,6- diazaspiro[3.3]heptane | Intermediate 45, CAS: 1203567-11-6; Yellow solid; Yield 188 mg, 34.1%; LCMS (ES⁺): 334.3 [MH]⁺ |
| 78 | 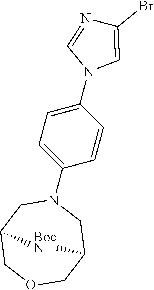 [SMILES: CC(C)(C)OC(=O)N1[C@@H]2COC[C@H]1CN(c1ccc(-n3cnc(Br)c3)cc1)C2] | tert-Butyl (1R,5S)-7-(4-(4- bromo-1H-imidazol-1- yl)phenyl)-3-oxa-7,9- diazabicyclo[3.3.1]nonane- 9-carboxylate | Intermediate 45, CAS: 1251010-45-3; Yellow solid; Yield 350 mg, 36.2%; LCMS (ES⁺): 449.20 [MH]⁺ |
| 79 | 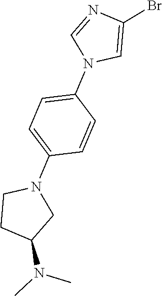 [SMILES: CN(C)[C@H]1CCN(c2ccc(-n3cnc(Br)c3)cc2)C1] | (S)-1-(4-(4-Bromo-1H- imidazol-1-yl)phenyl)-N,N- dimethylpyrrolidin-3-amine | Intermediate 45, CAS: 132883-44-4; Red solid; Yield 120 mg, 36.0%; LCMS (ES⁺): 335.1 [MH]⁺ |
| 80 | 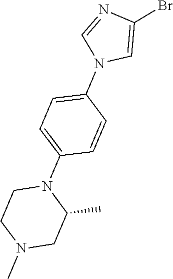 [SMILES: C[C@@H]1CN(C)CCN1c1ccc(-n2cnc(Br)c2)cc1] | (R)-1-(4-(4-Bromo-1H- imidazol-1-yl)phenyl)-2,4- dimethylpiperazine | Intermediate 45, CAS: 1033717-21-3; Yellow solid; Yield 200 mg, 36.0%; LCMS (ES⁺): 335.1 [MH]⁺ |
| 81 | 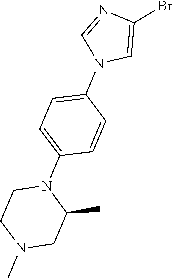 [SMILES: C[C@H]1CN(C)CCN1c1ccc(-n2cnc(Br)c2)cc1] | (S)-1-(4-(4-Bromo-1H- imidazol-1-yl)phenyl)-2,4- dimethylpiperazine | Intermediate 45, CAS: 1152367-80-0; Yellow solid; Yield 280 mg, 50.4%; LCMS (ES⁺): 335.1 [MH]⁺ |
| 82 | 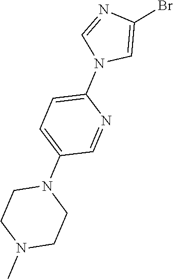 [SMILES: CN1CCN(c2ccc(-n3cnc(Br)c3)nc2)CC1] | 1-(6-(4-Bromo-1H-imidazol- 1-yl)pyridin-3-yl)-4- methylpiperazine | Intermediate 49, CAS: 109-01-3; Yellow solid; Yield 100 mg, 37.6%; LCMS (ES⁺): 322.3 [MH]⁺ |
| 83 | 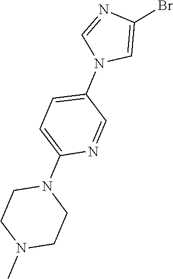 [SMILES: CN1CCN(c2ccc(-n3cnc(Br)c3)cn2)CC1] | 1-(5-(4-Bromo-1H-imidazol- 1-yl)pyridin-2-yl)-4- methylpiperazine | Intermediate 25, CAS: 109-01-3; Yellow solid; Yield 130 mg, 18.0%; LCMS (ES⁺): 322.3 [MH]⁺ |
| 84 | 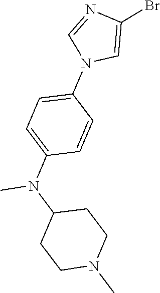 [SMILES: CN1CCC(N(C)c2ccc(-n3cnc(Br)c3)cc2)CC1] | N-(4-(4-Bromo-1H-imidazol- 1-yl)phenyl)-N,1- dimethylpiperidin-4-amine | Intermediate 45, CAS: 73579-08-5; Light brown solid; Yield 210 mg, 23.3%; LCMS (ES⁺): 349.3 [MH]⁺ |
| 85 | 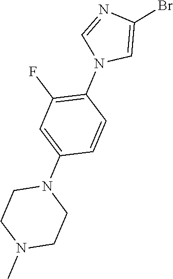 [SMILES: CN1CCN(c2ccc(-n3cnc(Br)c3)c(F)c2)CC1] | 1-(4-(4-Bromo-1H-imidazol- 1-yl)-3-fluorophenyl)-4- methylpiperazine | Intermediate 27, CAS: 109-01-3; Yellow solid; Yield 400 mg, 47.2%; LCMS (ES⁺): 339.2 [MH]⁺ |
| 86 | 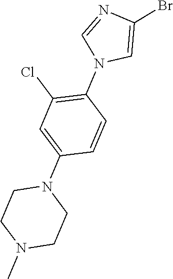 [SMILES: CN1CCN(c2ccc(-n3cnc(Br)c3)c(Cl)c2)CC1] | 1-(4-(4-Bromo-1H-imidazol- 1-yl)-3-chlorophenyl)-4- methylpiperazine | Intermediate 28, CAS: 109-01-3; Yellow solid; Yield 195 mg, 36.2%; LCMS (ES⁺): 355.6 [MH]⁺ |
| 87 | 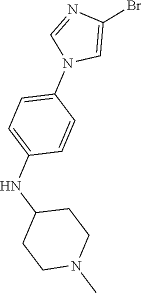 [SMILES: CN1CCC(Nc2ccc(-n3cnc(Br)c3)cc2)CC1] | N-(4-(4-Bromo-1H-imidazol- 1-yl)phenyl)-1- methylpiperidin-4-amine | Intermediate 45, CAS: 41838-46-4; Light brown solid; Yield 300 mg, 30.0%; LCMS (ES⁺): 335.2 [MH]⁺ |
| 88 | 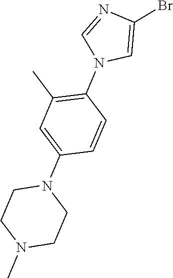 | 1-(4-(4-Bromo-1H-imidazol- 1-yl)-3-methylphenyl)-4- methylpiperazine | Intermediate 30, CAS: 109-01-3; Brown solid; Yield 505 mg, 56.0%; LCMS (ES⁺): 355.1 [MH]⁺ |
| 89 | 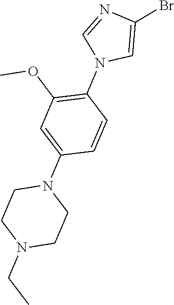 [SMILES: CCN1CCN(c2ccc(-n3cnc(Br)c3)c(OC)c2)CC1] | 1-(4-(4-Bromo-1H-imidazol- 1-yl)-3-methoxyphenyl)-4- ethylpiperazine | Intermediate 21, CAS: 5308-25-8; Yellow solid; Yield 170 mg, 38.6%; LCMS (ES⁺): 365.1 [MH]⁺ |
| 90 | 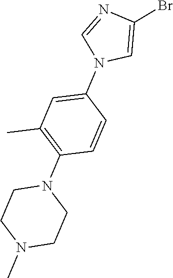 | 1-(4-(4-Bromo-1H-imidazol- 1-yl)-2-methylphenyl)-4- methylpiperazine | Intermediate 31, CAS: 109-01-3; Yellow solid; Yield 190 mg, 16.3%; LCMS (ES⁺): 355.6 [MH]⁺ |
| 91 | 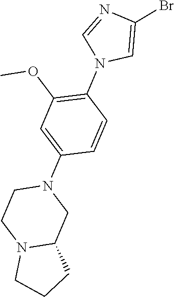 [SMILES: COc1cc(N2CCN3CCC[C@H]3C2)ccc1-n1cnc(Br)c1] | (S)-2-(4-(4-Bromo-1H- imidazol-1-yl)-3- methoxyphenyl) octahydropyrrolo [1,2-a]pyrazine | Intermediate 21, CAS: 93643-24-4; Yellow solid; Yield 75.0 mg, 22.2%; LCMS (ES⁺): 377.1 [MH]⁺ |
| 92 | 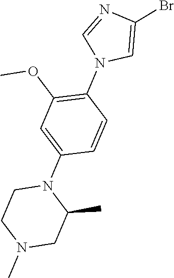 [SMILES: COc1cc(N2CCN(C)C[C@@H]2C)ccc1-n1cnc(Br)c1] | (S)-1-(4-(4-Bromo-1H- imidazol-1-yl)-3- methoxyphenyl)-2,4- dimethylpiperazine | Intermediate 21, CAS: 1152367-80-0; Yellow solid; Yield 180 mg, 54.5%; LCMS (ES⁺): 365.3 [MH]⁺ |
| 93 | 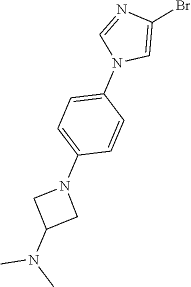 [SMILES: CN(C)C1CN(c2ccc(-n3cnc(Br)c3)cc2)C1] | 1-(4-(4-Bromo-1H-imidazol- 1-yl)phenyl)-N,N- dimethylazetidin-3-amine | Intermediate 45, CAS: 138022-85-2; Light yellow oil; Yield 56.0 mg, 35.1%; LCMS (ES⁺): 321.2 [MH]⁺ |
| 94 | 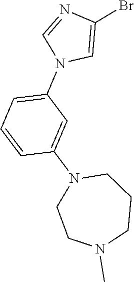 [SMILES: CN1CCCN(c2cccc(-n3cnc(Br)c3)c2)CC1] | 1-(3-(4-Bromo-1H-imidazol- 1-yl)phenyl)-4-methyl-1,4- diazepane | Intermediate 33, CAS: 4318-37-0; Yellow solid; Yield 127 mg, 38.0%; LCMS (ES⁺): 335.2 [MH]⁺ |
| 95 | 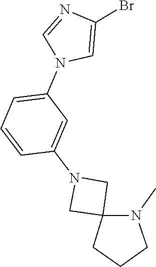 [SMILES: CN1CCCC12CN(c1cccc(-n3cnc(Br)c3)c1)C2] | 2-(3-(4-Bromo-1H-imidazol- 1-yl)phenyl)-5-methyl-2,5- diazaspiro[3.4]octane | Intermediate 33, CAS: 1421374-01-7; Yellow solid; Yield 110 mg, 23.9%; LCMS (ES⁺): 349.1 [MH]⁺ |
| 96 | 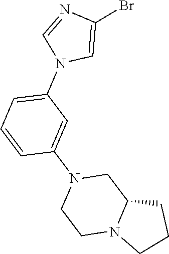 [SMILES: Brc1cn(-c2cccc(N3CCN4CCC[C@H]4C3)c2)cn1] | (S)-2-(3-(4-Bromo-1H- imidazol-1- yl)phenyl)octahydropyrrolo [1,2-a]pyrazine | Intermediate 33, CAS: 93643-24-4; Yellow solid; Yield 120 mg, 26.1%; LCMS (ES⁺): 347.3 [MH]⁺ |
| 97 | 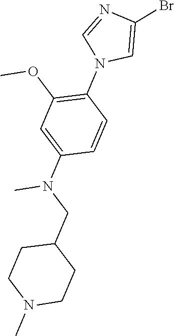 [SMILES: COc1cc(N(C)CC2CCN(C)CC2)ccc1-n1cnc(Br)c1] | 4-(4-Bromo-1H-imidazol-1- yl)-3-methoxy-N-methyl-N- ((1-methylpiperidin-4- yl)methyl)aniline | Intermediate 21, CAS: 405928-19-0; Yellow solid; Yield 103 mg, 29.0%; LCMS (ES⁺): 395.1 [MH]⁺ |
| 98 | 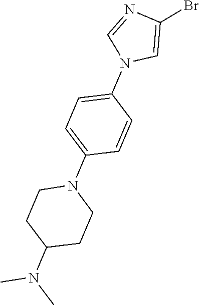 [SMILES: CN(C)C1CCN(c2ccc(-n3cnc(Br)c3)cc2)CC1] | 1-(4-(4-Bromo-1H-imidazol- 1-yl)phenyl)-N,N- dimethylpiperidin-4-amine | Intermediate 45, CAS: 172281-90-2; Yellow solid; Yield 150 mg, 32.3%; LCMS (ES⁺): 349.2 [MH]⁺ |
| 99 | 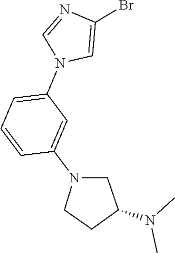 [SMILES: CN(C)[C@@H]1CCN(c2cccc(-n3cnc(Br)c3)c2)C1] | (R)-1-(3-(4-Bromo-1H- imidazol-1-yl)phenyl)-N,N- dimethylpyrrolidin-3-amine | Intermediate 33, CAS: 132958-72-6; Yellow solid; Yield 130 mg, 29.3%; LCMS (ES⁺): 335.1 [MH]⁺ |
| 100 | 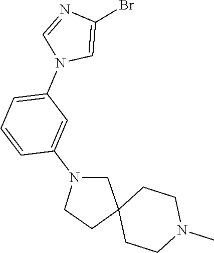 [SMILES: CN1CCC2(CC1)CCN(c1cccc(-n3cnc(Br)c3)c1)C2] | 2-(3-(4-Bromo-1H-imidazol- 1-yl)phenyl)-8-methyl-2,8- diazaspiro[4.5]decane | Intermediate 33, CAS: 1158750-98-1; Yellow solid; Yield 150 mg, 40.2%; LCMS (ES⁺): 375.1 [MH]⁺ |
| 101 | 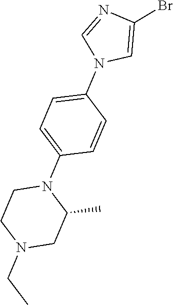 [SMILES: CCN1CCN(c2ccc(-n3cnc(Br)c3)cc2)[C@H](C)C1] | (R)-1-(4-(4-Bromo-1H- imidazol-1-yl)phenyl)-4- ethyl-2-methylpiperazine | Intermediate 45, CAS: 321860-21-3; Yellow solid; Yield 148 mg, 25.6%; LCMS (ES⁺): 349.1 [MH]⁺ |
| 102 | 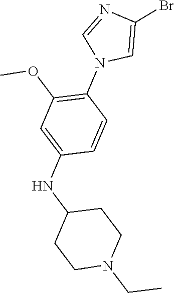 [SMILES: CCN1CCC(Nc2ccc(-n3cnc(Br)c3)c(OC)c2)CC1] | N-(4-(4-Bromo-1H-imidazol- 1-yl)-3-methoxyphenyl)-1- ethylpiperidin-4-amine | Intermediate 21, CAS: 50534-45-7; Yellow solid; Yield 165 mg, 48.1%; LCMS (ES⁺): 381.1 [MH]⁺ |
| 103 | 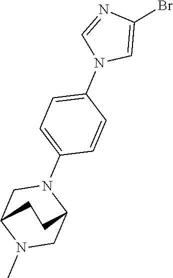 [SMILES: CN1C[C@@H]2CC[C@H]1CN2c1ccc(-n2cnc(Br)c2)cc1] | (1S,4S)-2-(4-(4-Bromo-1H- imidazol-1-yl)phenyl)-5- methyl-2,5- diazabicyclo[2.2.2]octane | Intermediate 45, CAS: 1187928-76-2; Brown yellow solid; Yield 142 mg, 24.7%; LCMS (ES⁺): 347.1 [MH]⁺ |
| 104 | 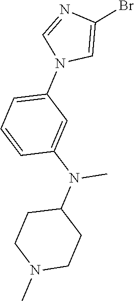 [SMILES: CN1CCC(N(C)c2cccc(-n3cnc(Br)c3)c2)CC1] | N-(3-(4-Bromo-1H-imidazol- 1-yl)phenyl)-N,1- dimethylpiperidin-4-amine | Intermediate 33, CAS: 73579-08-5; Yellow solid; Yield 100 mg, 43.2%; LCMS (ES⁺): 349.1 [MH]⁺ |
| 105 | 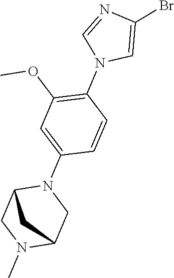 [SMILES: COc1cc(N2C[C@H]3C[C@@H]2CN3C)ccc1-n1cnc(Br)c1] | (1R,4R)-2-(4-(4-Bromo-1H- imidazol-1-yl)-3- methoxyphenyl)-5-methyl-2,5- diazabicyclo[2.2.1]heptane | Intermediate 21, CAS: 52321-26-3; Yellow oil; Yield 208 mg, 63.4%; LCMS (ES⁺): 363.0 [MH]⁺ |
| 106 | 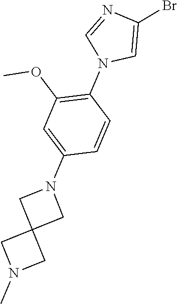 [SMILES: COc1cc(N2CC3(CN(C)C3)C2)ccc1-n1cnc(Br)c1] | 2-(4-(4-Bromo-1H-imidazol- 1-yl)-3-methoxyphenyl)-6- methyl-2,6- diazaspiro[3.3]heptane | Intermediate 21, CAS: 1203567-11-6; Yellow solid; Yield 115 mg, 31.9%; LCMS (ES⁺): 363.1 [MH]⁺ |
| 107 | 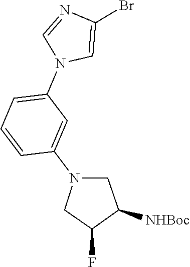 [SMILES: CC(C)(C)OC(=O)N[C@@H]1CN(c2cccc(-n3cnc(Br)c3)c2)C[C@@H]1F] | tert-Butyl ((3R,4S)-1-(3-(4- bromo-1H-imidazol-1- yl)phenyl)-4-fluoropyrrolidin- 3-yl)carbamate | Intermediate 33, CAS: 1033718-91-0; Yellow solid; Yield 169 mg, 40.0%; LCMS (ES⁺): 425.1 [MH]⁺ |
| 108 | 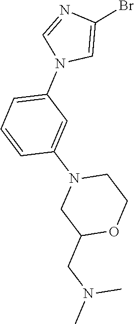 [SMILES: CN(C)CC1CN(c2cccc(-n3cnc(Br)c3)c2)CCO1] | 1-(4-(3-(4-Bromo-1H- imidazol-1- yl)phenyl)morpholin-2-yl)- N,N-dimethylmethanamine | Intermediate 33, CAS: 122894-56-8; Yellow solid; Yield 115 mg, 31.9%; LCMS (ES⁺): 366.1 [MH]⁺ |
| 109 | 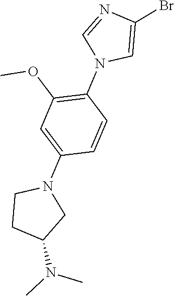 [SMILES: COc1cc(N2CC[C@@H](N(C)C)C2)ccc1-n1cnc(Br)c1] | (R)-1-(4-(4-Bromo-1H- imidazol-1-yl)-3- methoxyphenyl)-N,N- dimethylpyrrolidin-3-amine | Intermediate 21, CAS: 132958-72-6; Yellow solid; Yield 220 mg, 66.7%; LCMS (ES⁺): 366.9 [MH]⁺ |
| 110 | 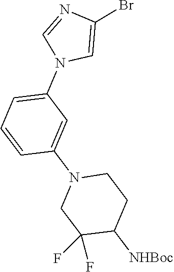 [SMILES: CC(C)(C)OC(=O)NC1CCN(c2cccc(-n3cnc(Br)c3)c2)CC1(F)F] | tert-butyl (1-(3-(4-Bromo- 1H-imidazol-1-yl)phenyl)- 3,3-difluoropiperidin-4- yl)carbamate | Intermediate 33, CAS: 1263180-22-8; Yellow solid; Yield 315 mg, 41.6%; LCMS (ES⁺): 457.3 [MH]⁺ |
| 111 | 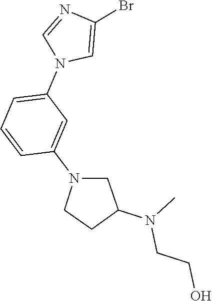 [SMILES: CN(CCO)C1CCN(c2cccc(-n3cnc(Br)c3)c2)C1] | 2-((1-(3-(4-Bromo-1H- imidazol-1- yl)phenyl)pyrrolidin-3- yl)(methyl)amino)ethan-1-ol | Intermediate 33, CAS: 1181265-05-3; Yellow solid; Yield 100 mg, 16.5%; LCMS (ES⁺): 365.1 [MH]⁺ |
| 112 | 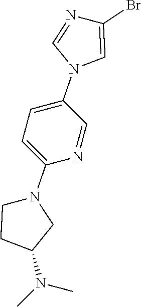 [SMILES: CN(C)[C@@H]1CCN(c2ccc(-n3cnc(Br)c3)cn2)C1] | (R)-1-(6-(4-Bromo-1H- imidazol-1-yl)pyridin-3-yl)- N,N-dimethylpyrrolidin-3- amine | Intermediate 49, CAS: 132958-72-6; Yellow solid; Yield 206 mg, 18.6%; LCMS (ES⁺): 336.1 [MH]⁺ |
| 113 | 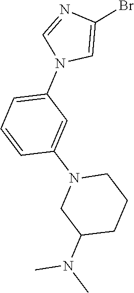 [SMILES: CN(C)C1CCCN(c2cccc(-n3cnc(Br)c3)c2)C1] | 1-(3-(4-Bromo-1H-imidazol- 1-yl)phenyl)-N,N- dimethylpiperidin-3-amine | Intermediate 33, CAS: 50534-49-1; Yellow solid; Yield 320 mg, 46.1%; LCMS (ES⁺): 351.0 [MH]⁺ |
| 114 | 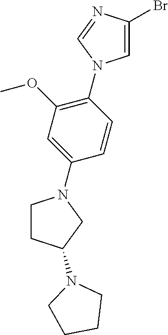 [SMILES: COc1cc(N2CC[C@@H](N3CCCC3)C2)ccc1-n1cnc(Br)c1] | (R)-1′-(4-(4-Bromo-1H- imidazol-1-yl)-3- methoxyphenyl)-1,3′- bipyrrolidine | Intermediate 21, CAS: 900164-95-6; Yellow solid; Yield 120 mg, 33.9%; LCMS (ES⁺): 391.1 [MH]⁺ |
| 115 | 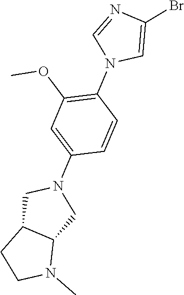 [SMILES: COc1cc(N2C[C@H]3CCN(C)[C@H]3C2)ccc1-n1cnc(Br)c1] | (3aR,6aR)-5-(4-(4-Bromo- 1H-imidazol-1-yl)-3- methoxyphenyl)-1- methyloctahydropyrrolo [3,4-b]pyrrole | Intermediate 21, CAS: 877212-98-1; Yellow solid; Yield 25.0 mg, 5.5%; LCMS (ES⁺): 377.3 [MH]⁺ |
| 116 | 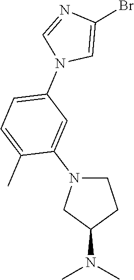 [SMILES: Cc1ccc(-n2cnc(Br)c2)cc1N1CC[C@@H](N(C)C)C1] | (R)-1-(5-(4-Bromo-1H- imidazol-1-yl)-2- methylphenyl)-N,N- dimethylpyrrolidin-3-amine | Intermediate 36, CAS: 132958-72-6; Brown solid; Yield 200 mg, 45.2%; LCMS (ES⁺): 349.1 [MH]⁺ |
| 117 | 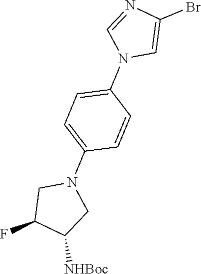 [SMILES: CC(C)(C)OC(=O)N[C@H]1CN(c2ccc(-n3cnc(Br)c3)cc2)C[C@@H]1F] | tert-Butyl ((3S,4S)-1-(4-(4- bromo-1H-imidazol-1- yl)phenyl)-4-fluoropyrrolidin- 3-yl)carbamate | Intermediate 45, CAS: 186201-09-2; Dark yellow solid; Yield 120 mg, 51.0%; LCMS (ES⁺): 425.1 [MH]⁺ |
| 118 | 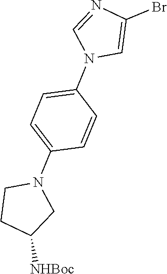 [SMILES: CC(C)(C)OC(=O)N[C@@H]1CCN(c2ccc(-n3cnc(Br)c3)cc2)C1] | tert-Butyl (R)-1-(4-(4- bromo-1H-imidazol-1- yl)phenyl)pyrrolidin-3- yl)carbamate | Intermediate 45, CAS: 122536-77-0; Yellow solid; Yield 185 mg, 45.7%; LCMS (ES⁺): 409.3 [MH]⁺ |
| 119 | 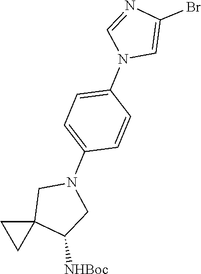 [SMILES: CC(C)(C)OC(=O)N[C@H]1CN(c2ccc(-n3cnc(Br)c3)cc2)CC12CC2] | tert-Butyl (R)-(5-(4-(4- bromo-1H-imidazol-1- yl)phenyl)-5- azaspiro[2.4]heptan-7- yl)carbamate | Intermediate 45, CAS: 127199-44-4; Brown yellow solid; Yield 87.0 mg 30.3%; LCMS (ES⁺): 433.1 [MH]⁺ |
| 120 | 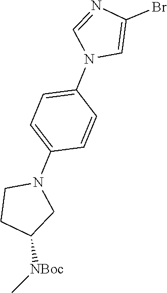 [SMILES: CN(C(=O)OC(C)(C)C)[C@@H]1CCN(c2ccc(-n3cnc(Br)c3)cc2)C1] | tert-Butyl (R)-1-(4-(4- bromo-1H-imidazol-1- yl)phenyl)pyrrolidin-3- yl)(methyl)carbamate | Intermediate 45, CAS: 392338-15-7; Yellow solid; Yield 720 mg, 34.4%; LCMS (ES⁺): 422.8 [MH]⁺ |
| 121 | 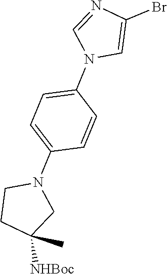 [SMILES: CC(C)(C)OC(=O)N[C@]1(C)CCN(c2ccc(-n3cnc(Br)c3)cc2)C1] | tert-Butyl (R)-(1-(4-(4- bromo-1H-imidazol-1- yl)phenyl)-3- methylpyrrolidin-3- yl)carbamate | Intermediate 45, CAS: 167888-15-5; Yellow solid; Yield 170 mg, 26.9%; LCMS (ES⁺): 421.3 [MH]⁺ |
| 122 | 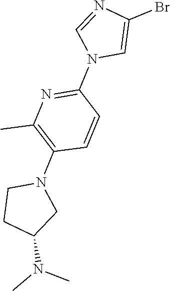 [SMILES: Cc1nc(-n2cnc(Br)c2)ccc1N1CC[C@@H](N(C)C)C1] | (R)-1-(6-(4-Bromo-1H- imidazol-1-yl)-2- methylpyridin-3-yl)-N,N- dimethylpyrrolidin-3-amine | Intermediate 53, CAS: 132958-72-6; White solid; Yield 27.0 mg, 24.4%; LCMS (ES⁺): 350.4 [MH]⁺ |
| 123 | 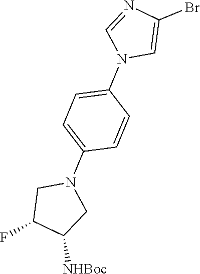 [SMILES: CC(C)(C)OC(=O)N[C@H]1CN(c2ccc(-n3cnc(Br)c3)cc2)C[C@H]1F] | tert-Butyl ((3S,4R)-1-(4-(4- bromo-1H-imidazol-1- yl)phenyl)-4-fluoropyrrolidin- 3-yl)carbamate | Intermediate 45, CAS: 169750-42-9; White solid; Yield 320 mg, 56.9%; LCMS (ES⁺): 426.8 [MH]⁺ |
| 124 | 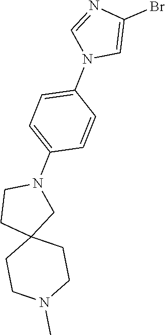 [SMILES: CN1CCC2(CC1)CCN(c1ccc(-n3cnc(Br)c3)cc1)C2] | 2-(4-(4-Bromo-1H-imidazol- 1-yl)phenyl)-8-methyl-2,8- diazaspiro[4.5]decane | Intermediate 45, CAS: 1158750-98-1; Yellow solid; Yield 95.0 mg, 38.2%; LCMS (ES⁺): 375.3 [MH]⁺ |
| 125 | 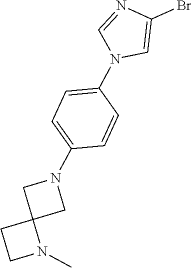 [SMILES: CN1CCC12CN(c1ccc(-n3cnc(Br)c3)cc1)C2] | 6-(4-(4-Bromo-1H-imidazol- 1-yl)phenyl)-1-methyl-1,6- diazaspiro[3.3]heptane | Intermediate 45, CAS: 1454909-03-5; Brown yellow solid; Yield 170 mg, 38.5%; LCMS (ES⁺): 333.1 [MH]⁺ |
| 126 | 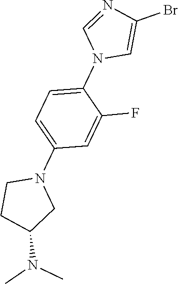 | (R)-1-(4-(4-Bromo-1H- imidazol-1-yl)-3- fluorophenyl)-N,N- dimethylpyrrolidin-3-amine | Intermediate 27, CAS: 132958-72-6; Yellow solid; Yield 170 mg, 34.2%; LCMS (ES⁺): 425.1 [MH]⁺ |
| 127 | 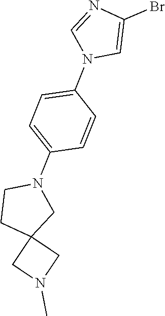 [SMILES: CN1CC2(CCN(c3ccc(-n4cnc(Br)c4)cc3)C2)C1] | 6-(4-(4-Bromo-1H-imidazol- 1-yl)phenyl)-2-methyl-2,6- diazaspiro[3.4]octane | Intermediate 45, CAS: 135380-30-2; Brown yellow oil; Yield 170 mg, 37.0%; LCMS (ES⁺): 347.1 [MH]⁺ |
| 128 | 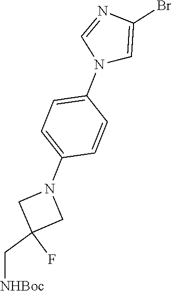 [SMILES: CC(C)(C)OC(=O)NCC1(F)CN(c2ccc(-n3cnc(Br)c3)cc2)C1] | tert-Butyl ((1-(4-(4-bromo- 1H-imidazol-1-yl)phenyl)-3- fluoroazetidin-3- yl)methyl)carbamate | Intermediate 45, CAS: 1374657-58-5; Yellow solid; Yield 130 mg, 43.3%; LCMS (ES⁺): 425.1 [MH]⁺ |
| 129 | 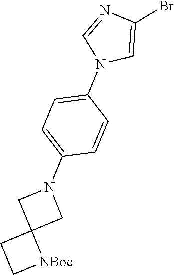 [SMILES: CC(C)(C)OC(=O)N1CCC12CN(c1ccc(-n3cnc(Br)c3)cc1)C2] | tert-Butyl 6-(4-(4-bromo-1H- imidazol-1-yl)phenyl)-1,6- diazaspiro[3.3]heptane-1- carboxylate | Intermediate 45, CAS: 1330763-95-5; Brown solid; Yield 105 mg, 18.9%; LCMS (ES⁺): 419.1 [MH]⁺ |
| 130 | 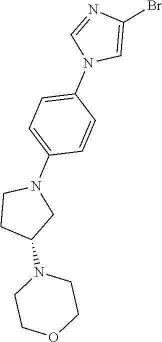 [SMILES: Brc1cn(-c2ccc(N3CC[C@@H](N4CCOCC4)C3)cc2)cn1] | (R)-4-(1-(4-(4-Bromo-1H- imidazol-1- yl)phenyl)pyrrolidin-3- yl)morpholine | Intermediate 45, CAS: 511295-94-6; White solid; Yield 150 mg, 40.0%; LCMS (ES⁺): 377.2 [MH]⁺ |
| 131 | 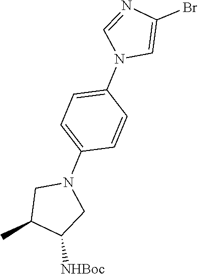 [SMILES: C[C@H]1CN(c2ccc(-n3cnc(Br)c3)cc2)C[C@@H]1NC(=O)OC(C)(C)C] | tert-Butyl ((3R,4S)-1-(4-(4- bromo-1H-imidazol-1- yl)phenyl)-4- methylpyrrolidin-3- yl)carbamate | Intermediate 45, CAS: 1334481-84-3; Yellow solid; Yield 120 mg, 28.7%; LCMS (ES⁺): 423.2 [MH]⁺ |
| 132 | 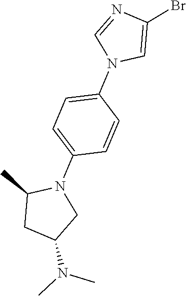 [SMILES: C[C@@H]1C[C@@H](N(C)C)CN1c1ccc(-n2cnc(Br)c2)cc1] | (3R,5R)-1-(4-(4-Bromo-1H- imidazol-1-yl)phenyl)-N,N,5- trimethylpyrrolidin-3-amine | Intermediate 45, Intermediate 9; Brown solid; Yield 92.4 mg, 14.9%; LCMS (ES⁺): 349.2 [MH]⁺ |
| 133 | 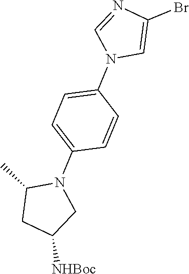 [SMILES: C[C@H]1C[C@@H](NC(=O)OC(C)(C)C)CN1c1ccc(-n2cnc(Br)c2)cc1] | tert-Butyl ((3R,5S)-1-(4-(4- bromo-1H-imidazol-1- yl)phenyl)-5- methylpyrrolidin-3- yl)carbamate | Intermediate 45, CAS: 1626343-40-5; Brown yellow solid; Yield 320 mg, 32.8%; LCMS (ES⁺): 421.1 [MH]⁺ |
| 134 | 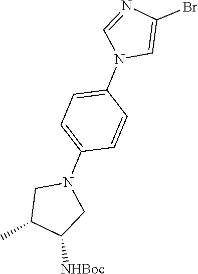 [SMILES: C[C@@H]1CN(c2ccc(-n3cnc(Br)c3)cc2)C[C@@H]1NC(=O)OC(C)(C)C] | tert-Butyl ((3R,4R)-1-(4-(4- bromo-1H-imidazol-1- yl)phenyl)-4- methylpyrrolidin-3- yl)carbamate | Intermediate 45, CAS: 127199-55-7; Yellow solid; Yield 120 mg, 17.2%; LCMS (ES⁺): 422.9 [MH]⁺ |
| 135 | 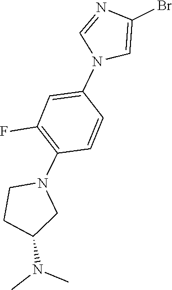 | (R)-1-(4-(4-Bromo-1H- imidazol-1-yl)-2- fluorophenyl)-N,N- dimethylpyrrolidin-3-amine | Intermediate 38, CAS: 132958-72-6; Red oil; Yield 150 mg, 15.1%; LCMS (ES⁺): 339.9 [MH]⁺ |
| 136 | 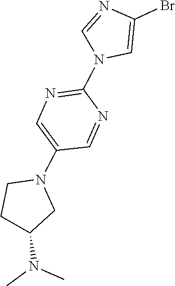 [SMILES: CN(C)[C@@H]1CCN(c2cnc(-n3cnc(Br)c3)nc2)C1] | (R)-1-(2-(4-Bromo-1H- imidazol-1-yl)pyrimidin-5-yl)- N,N-dimethylpyrrolidin-3- amine | Intermediate 54, CAS: 132958-72-6; Yellow solid; Yield 170 mg, 36.5%; LCMS (ES⁺): 337.1 [MH]⁺ |
| 137 | 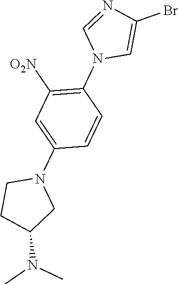 [SMILES: CN(C)[C@@H]1CCN(c2ccc(-n3cnc(Br)c3)c([N+](=O)[O-])c2)C1] | (R)-1-(4-(4-Bromo-1H- imidazol-1-yl)-3- nitrophenyl)-N,N- dimethylpyrrolidin-3-amine | Intermediate 55, CAS: 132958-72-6; Brown solid; Yield 3.34 g, 48.4%; LCMS (ES⁺): 380.2 [MH]⁺ |
| 138 | 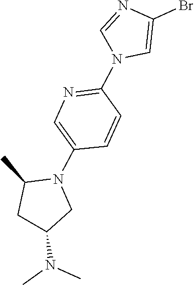 [SMILES: C[C@@H]1C[C@@H](N(C)C)CN1c1ccc(-n2cnc(Br)c2)nc1] | (3R,5R)-1-(6-(4-Bromo-1H- imidazol-1-yl)pyridin-3-yl)- N,N,5-trimethylpyrrolidin-3- amine | Intermediate 49, Intermediate 9; Pale brown solid; Yield 45.6 mg, 12.5%; LCMS (ES⁺): 350.2 [MH]⁺ |
| 139 | 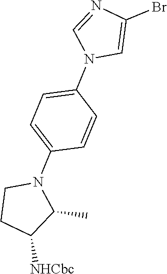 | Benzyl ((2R,3R)-1-(4-(4- bromo-1H-imidazol-1- yl)phenyl)-2- methylpyrrolidin-3- yl)carbamate | Intermediate 45, CAS: 1932285-90-9; Yellow solid; Yield 197 mg, 26.7%; LCMS (ES⁺): 455.1 [MH]⁺ |
| 140 | 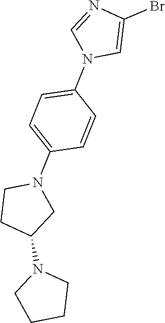 [SMILES: Brc1cn(-c2ccc(N3CC[C@@H](N4CCCC4)C3)cc2)cn1] | (R)-1′-(4-(4-Bromo-1H- imidazol-1-yl)phenyl)-1,3′- bipyrrolidine | Intermediate 45, CAS: 900164-95-6; Yellow solid; Yield 240 mg, 50.0%; LCMS (ES⁺): 360.8 [MH]⁺ |
| 141 | 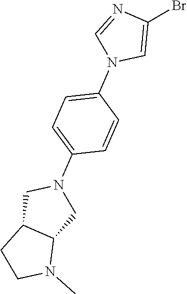 [SMILES: CN1CC[C@@H]2CN(c3ccc(-n4cnc(Br)c4)cc3)C[C@@H]21] | (3aR,6aR)-5-(4-(4-Bromo- 1H-imidazol-1-yl)phenyl)-1- methyloctahydropyrrolo[3,4- b]pyrrole | Intermediate 45, CAS: 877212-98-1; Yellow solid; Yield 160 mg, 34.8%; LCMS (ES⁺): 347.1 [MH]⁺ |
| 142 | 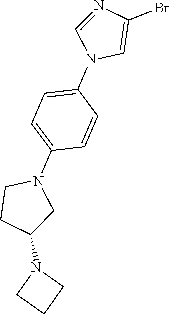 [SMILES: Brc1cn(-c2ccc(N3CC[C@@H](N4CCC4)C3)cc2)cn1] | 1-(4-(3-(Azetidin-1- yl)pyrrolidin-1-yl)phenyl)-4- bromo-1H-imidazole | Intermediate 45, CAS: 1018443-34-9; Yellow solid; Yield 370 mg, 33.9%; LCMS (ES⁺): 347.1 [MH]⁺ |
| 143 | 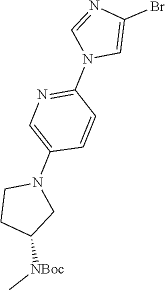 [SMILES: CN(C(=O)OC(C)(C)C)[C@@H]1CCN(c2ccc(-n3cnc(Br)c3)nc2)C1] | tert-Butyl (R)-1-(6-(4- bromo-1H-imidazol-1- yl)pyridin-3-yl)pyrrolidin-3- yl)(methyl)carbamate | Intermediate 49, CAS: 392338-15-7; Yellow oil; Yield 110 mg, 12.5%; LCMS (ES⁺): 422.1 [MH]⁺ |
| 144 | 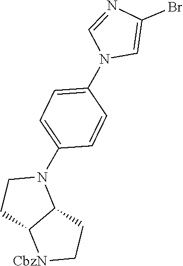 [SMILES: O=C(OCc1ccccc1)N1CC[C@@H]2[C@H]1CCN2c1ccc(-n2cnc(Br)c2)cc1] | Benzyl (3aR,6aR)-4-(4-(4- bromo-1H-imidazol-1- yl)phenyl)hexahydropyrrolo [3,2-b]pyrrole-1(2H)- carboxylate | Intermediate 45, CAS: 1295578-04-9; Yellow solid; Yield 320 mg, 41.5%; LCMS (ES⁺): 467.3 [MH]⁺ |
| 145 | 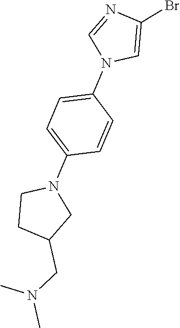 [SMILES: CN(C)CC1CCN(c2ccc(-n3cnc(Br)c3)cc2)C1] | 1-(1-(4-(4-Bromo-1H- imidazol-1- yl)phenyl)pyrrolidin-3-yl)- N,N-dimethylmethanamine | Intermediate 45, CAS: 99724-17-1; Yellow solid; Yield 353 mg, 30.6%; LCMS (ES⁺): 349.0 [MH]⁺ |
| 146 | 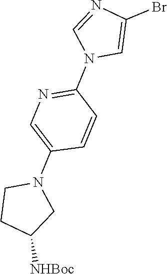 [SMILES: CC(C)(C)OC(=O)N[C@@H]1CCN(c2ccc(-n3cnc(Br)c3)nc2)C1] | tert-Butyl (R)-(1-(6-(4- bromo-1H-imidazol-1- yl)pyridin-3-yl)pyrrolidin-3- yl)carbamate | Intermediate 49, CAS: 122536-77-0; Yellow solid; Yield 250 mg, 18.6%; LCMS (ES⁺): 408.0 [MH]⁺ |
| 147 | 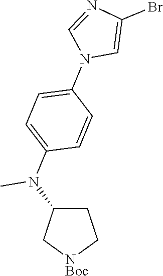 [SMILES: CN(c1ccc(-n2cnc(Br)c2)cc1)[C@@H]1CCN(C(=O)OC(C)(C)C)C1] | tert-Butyl (R)-3-((4-(4- bromo-1H-imidazol-1- yl)phenyl)(methyl)amino)pyrrolidine- 1-carboxylate | Intermediate 45, CAS: 199336-83-9; Yellow solid; Yield 400 mg, 28.7%; LCMS (ES⁺): 420.8 [MH]⁺ |
| 148 | 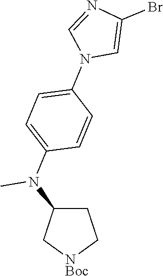 [SMILES: CN(c1ccc(-n2cnc(Br)c2)cc1)[C@H]1CCN(C(=O)OC(C)(C)C)C1] | tert-Butyl (S)-3-((4-(4- bromo-1H-imidazol-1-yl) phenyl)(methyl)amino)pyrrolidine- 1-carboxylate | Intermediate 45, CAS: 147081-59-2; Yellow solid; Yield 400 mg, 28.7%; LCMS (ES⁺): 421.0 [MH]⁺ |
| 149 | 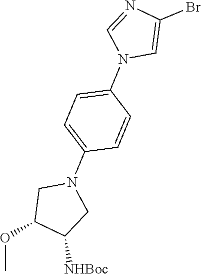 [SMILES: CO[C@@H]1CN(c2ccc(-n3cnc(Br)c3)cc2)C[C@@H]1NC(=O)OC(C)(C)C] | tert-Butyl ((3S,4R)-1-(4-(4- bromo-1H-imidazol-1- yl)phenyl)-4- methoxypyrrolidin-3- yl)carbamate | Intermediate 45, CAS: 128739-89-9; Yellow solid; Yield 156 mg, 34.7%; LCMS (ES⁺): 437.0 [MH]⁺ |
| 150 | 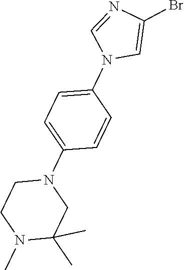 [SMILES: CN1CCN(c2ccc(-n3cnc(Br)c3)cc2)CC1(C)C] | 4-(4-(4-Bromo-1H-imidazol- 1-yl)phenyl)-1,2,2- trimethylpiperazine | Intermediate 45, CAS: 396133-32-7; Brown oil; Yield 144 mg, 21.7%; LCMS (ES⁺): 349.0 [MH]⁺ |
| 151 | 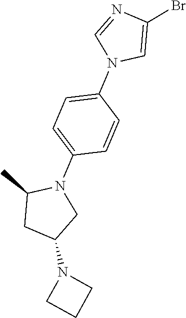 [SMILES: C[C@@H]1C[C@@H](N2CCC2)CN1c1ccc(-n2cnc(Br)c2)cc1] | 1-(4-((2R,4R)-4-(Azetidin-1- yl)-2-methylpyrrolidin-1- yl)phenyl)-4-bromo-1H- imidazole | Intermediate 45, Intermediate 10; Pale brown gum; Yield 314 mg, 18.5%; LCMS (ES⁺): 361.0 [MH]⁺ |
| 152 | 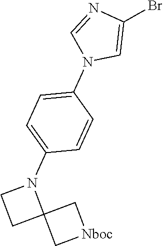 | tert-Butyl 1-(4-(4-bromo-1H- imidazol-1-yl)phenyl)-1,6- diazaspiro[3.3]heptane-6- carboxylate | Intermediate 45, CAS: 1272412-72-2; Off-white solid; Yield 850 mg, 40.8%; LCMS (ES⁺): 419.1 [MH]⁺ |
| 153 | 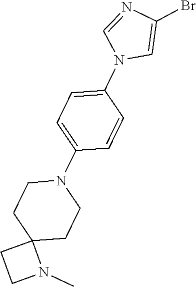 [SMILES: CN1CCC12CCN(c1ccc(-n3cnc(Br)c3)cc1)CC2] | 7-(4-(4-Bromo-1H-imidazol- 1-yl)phenyl)-1-methyl-1,7- diazaspiro[3.5]nonane | Intermediate 45, CAS: 1258073-94-7; Yellow oil; Yield 280 mg, 39.0%; LCMS (ES⁺): 361.1 [MH]⁺ |
| 154 | 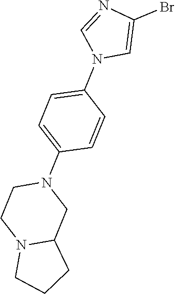 [SMILES: Brc1cn(-c2ccc(N3CCN4CCCC4C3)cc2)cn1] | 2-(4-(4-Bromo-1H-imidazol- 1- yl)phenyl)octahydropyrrolo [1,2-a]pyrazine | Intermediate 45, CAS: 5654-83-1; White solid; Yield 165 mg, 17.9%; LCMS (ES⁺): 347.1 [MH]⁺ |
| 155 | 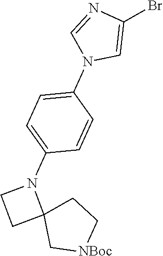 [SMILES: CC(C)(C)OC(=O)N1CCC2(CCN2c2ccc(-n3cnc(Br)c3)cc2)C1] | tert-Butyl 1-(4-(4-bromo-1H- imidazol-1-yl)phenyl)-1,6- diazaspiro[3.4]octane-6- carboxylate | Intermediate 45, CAS: 1158749-79-1; White solid; Yield 200 mg, 15.5%; LCMS (ES⁺): 433.2 [MH]⁺ |
| 156 | 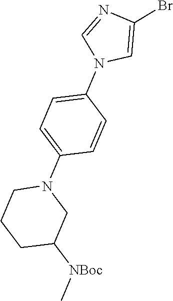 [SMILES: CN(C(=O)OC(C)(C)C)C1CCCN(c2ccc(-n3cnc(Br)c3)cc2)C1] | tert-Butyl (1-(4-(4-bromo- 1H-imidazol-1- yl)phenyl)piperidin-3- yl)(methyl)carbamate | Intermediate 45; Yellow oil; Yield 800 mg, 39.6%; LCMS (ES⁺): 435.1 [MH]⁺ |
| 157 | 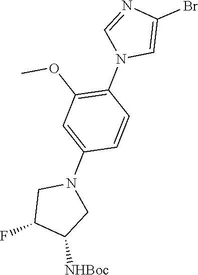 [SMILES: COc1cc(N2C[C@@H](F)[C@@H](NC(=O)OC(C)(C)C)C2)ccc1-n1cnc(Br)c1] | tert-Butyl ((3S,4R)-1-(4-(4- bromo-1H-imidazol-1-yl)-3- methoxyphenyl)-4- fluoropyrrolidin-3- yl)carbamate | Intermediate 21, CAS: 169750-42-9; Brown solid; Yield 240 mg, 40.2%; LCMS (ES⁺): 455.2 [MH]⁺ |
| 158 | 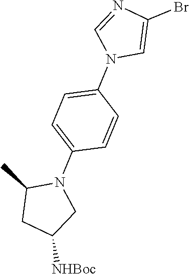 [SMILES: C[C@@H]1C[C@@H](NC(=O)OC(C)(C)C)CN1c1ccc(-n2cnc(Br)c2)cc1] | tert-Butyl ((3R,5R)-1-(4-(4- bromo-1H-imidazol-1- yl)phenyl)-5- methylpyrrolidin-3- yl)carbamate | Intermediate 45, CAS: 1932651-04-1; White solid; Yield 545 mg, 25.3%; LCMS (ES⁺): 421.0 [MH]⁺ |
| 159 | 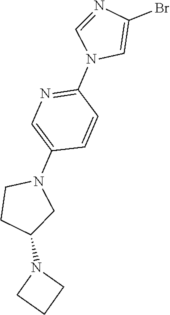 [SMILES: Brc1cn(-c2ccc(N3CC[C@@H](N4CCC4)C3)cn2)cn1] | (R)-5-(3-(Azetidin-1- yl)pyrrolidin-1-yl)-2-(4- bromo-1H-imidazol-1- yl)pyridine | Intermediate 49, Intermediate 11; Tan oil; Yield 153 mg, 26.6%; LCMS (ES⁺): 448.0 [MH]⁺ |
| 160 | 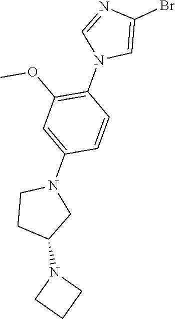 [SMILES: COc1cc(N2CC[C@@H](N3CCC3)C2)ccc1-n1cnc(Br)c1] | (R)-1-(4-(3-(Azetidin-1- yl)pyrrolidin-1-yl)-2- methoxyphenyl)-4-bromo- 1H-imidazole | Intermediate 21, Intermediate 11; Tan oil; Yield 223 mg, 32.7%; LCMS (ES⁺): 377.1 [MH]⁺ |
| 161 | 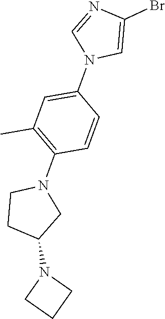 [SMILES: Cc1cc(-n2cnc(Br)c2)ccc1N1CC[C@@H](N2CCC2)C1] | (R)-1-(4-(3-(Azetidin-1- yl)pyrrolidin-1-yl)-3- methylphenyl)-4-bromo-1H- imidazole | Intermediate 31, Intermediate 11; Tan oil; Yield 130 mg, 22.7%; LCMS (ES⁺): 361.1 [MH]⁺ |

### Intermediates 162 and 163

### tert-Butyl 6-(4-(4-bromo-1H-imidazol-1-yl)phenyl)-1,6-diazaspiro[3.4]octane-1-carboxylate

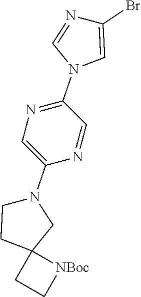 [SMILES: CC(C)(C)OC(=O)N1CCC12CCN(c1cnc(-n3cnc(Br)c3)cn1)C2]

Intermediates 162 and 163 were prepared similarly to Intermediate 75 via XPhos Pd G3-catalysed Buchwald-Hartwig coupling of Intermediate 45 (1.76 g, 5.83 mmol) with tert-butyl 1,6-diazaspiro[3.4]octane-1-carboxylate hemioxalate (2.10 g, 4.08 mmol) then purification via NP column chromatography to afford the title compound (900 mg, 35.6%) as a yellow solid.

The racemic product was separated via prep-SFC Method 1 to afford each enantiomer of the title compound as a single enantiomer of unknown absolute stereochemistry.

Intermediate 162 (Enantiomer 1): SFC Rt 2.28 min to afford the title compound (401 mg, 15.9%) as a yellow solid. LCMS (ES⁺): 433.1 [MH]⁺.

Intermediate 163 (Enantiomer 2): SFC Rt 4.02 min to afford the title compound (430 mg, 17.0%) as a yellow solid. LCMS (ES⁺): 433.1 [MH]⁺.

### Intermediate 164

### 1-(4-(4-Bromo-1H-imidazol-1-yl)-3-(trifluoromethoxy)phenyl)-4-methylpiperazine

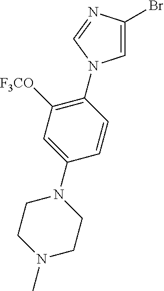 [SMILES: CN1CCN(c2ccc(-n3cnc(Br)c3)c(OC(F)(F)F)c2)CC1]

To a stirred mixture of Intermediate 32 (500 mg, 1.30 mmol) and 1-methyl-piperazine (194 mg, 1.94 mmol) in DXN (10 mL) were added tBuXPhos Pd G3 (103 mg, 0.13 mmol), Me₄tBuXPhos (60.0 mg, 0.13 mmol) and Cs₂CO₃ (848 mg, 2.60 mmol) at RT under N₂. The mixture was heated to 100° C. and stirred for 16 h, filtered, and the filter cake was washed with MeCOH. The filtrate was concentrated in vacuo and purified via RP column chromatography to afford the title compound (190 mg, 36.2%) as a red solid. LCMS (ES⁺): 407.0 [MH]⁺.

### Intermediate 165

### (R)-1-(4-(4-Bromo-1H-imidazol-1-yl)phenyl)-N,N-dimethylpyrrolidin-3-amine

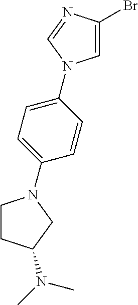 [SMILES: CN(C)[C@@H]1CCN(c2ccc(-n3cnc(Br)c3)cc2)C1]

Intermediate 45 (2.00 g, 6.56 mmol, 99.1% purity), (R)-(+)-3-(dimethylamino)pyrrolidine (1.00 mL, 7.88 mmol), BINAP (307 mg, 0.49 mmol) and NaOtBu (883 mg, 9.19 mmol) in PhMe (40 mL) was de-gassed using N₂ for 10 min, then Pd₂dba₃ (150 mg, 0.16 mmol) was added, the mixture de-gassed for a further 5 min then heated to 110° C. for 18 h under N₂. The reaction mixture was diluted with DCM (100 mL), filtered through Celite®, concentrated in vacuo and purified via NP column chromatography to afford the title compound (1.07 g, 46.9%) as a beige solid. LCMS (ES⁺): 335.1 [MH]⁺.

### Intermediates 166 to 182

Intermediates 166 to 182 were prepared similarly to Intermediate 165 via Pd₂dba₃-catalysed Buchwald-Hartwig coupling of the specified aryl bromide Intermediate with the appropriate amine; see Table 7 below.

TABLE 7
Pd₂dba₃-catalysed Buchwald-Hartwig coupling of the appropriate
(hetero)aryl bromide Intermediate with the appropriate amine

| | | | Reactant(s) used; |
|---|---|---|---|
| Int. | Structure | Name | Form; Yield; LCMS |
| 166 | 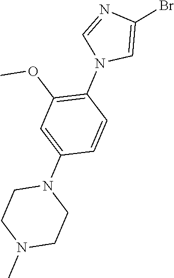 [SMILES: COc1cc(N2CCN(C)CC2)ccc1-n1cnc(Br)c1] | 1-(4-(4-Bromo-1H- imidazol-1-yl)-3- methoxyphenyl)-4- methylpiperazine | Intermediate 21, CAS: 109-01-3; Yellow solid; Yield 317 mg, 59.9%; LCMS (ES⁺): 350.7 [MH]⁺ |
| 167 | 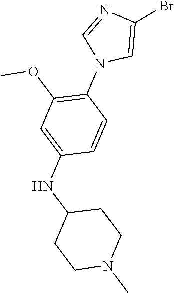 [SMILES: COc1cc(NC2CCN(C)CC2)ccc1-n1cnc(Br)c1] | N-(4-(4-Bromo-1H- imidazol-1-yl)-3- methoxyphenyl)-1- methylpiperidin-4-amine | Intermediate 21, CAS: 41838-46-4; Yellow gum; Yield 55.0 mg, 31.9%; LCMS (ES⁺): 365.2 [MH]⁺ |
| 168 | 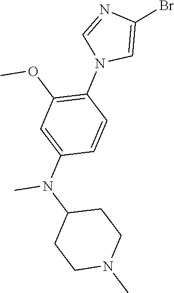 [SMILES: COc1cc(N(C)C2CCN(C)CC2)ccc1-n1cnc(Br)c1] | N-(4-(4-Bromo-1H- imidazol-1-yl)-3- methoxyphenyl)-N,1- dimethylpiperidin-4- amine | Intermediate 21, CAS: 73579-08-5; Yellow gum; Yield 25.0 mg, 9.37%; LCMS (ES⁺): 379.1 [MH]⁺ |
| 169 | 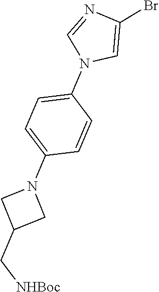 [SMILES: CC(C)(C)OC(=O)NCC1CN(c2ccc(-n3cnc(Br)c3)cc2)C1] | tert-Butyl ((1-(4-(4- bromo-1H-imidazol-1- yl)phenyl)azetidin-3- yl)methyl)carbamate | Intermediate 45, CAS: 91188-15-7; Off-white solid; Yield 250 mg, 41.8%; LCMS (ES⁺): 406.9 [MH]⁺ |
| 170 | 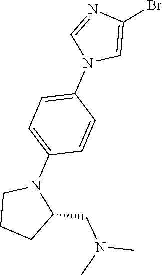 [SMILES: CN(C)C[C@@H]1CCCN1c1ccc(-n2cnc(Br)c2)cc1] | (S)-1-(1-(4-(4-Bromo- 1H-imidazol-1- yl)phenyl)pyrrolidin-2- yl)-N,N- dimethylmethanamine | Intermediate 45, CAS: 29618-57-3; Off-white solid; Yield 41.7 mg, 16.7%; LCMS (ES⁺): 348.9 [MH]⁺ |
| 171 | 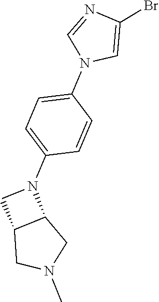 [SMILES: CN1C[C@@H]2CN(c3ccc(-n4cnc(Br)c4)cc3)[C@@H]2C1] | (1R,5S)-6-(4-(4-Bromo- 1H-imidazol-1- yl)phenyl)-3-methyl-3,6- diazabicyclo[3.2.0] heptane | Intermediate 45, CAS: 1419075-95-8; Off-white solid; Yield 49.2 mg, 20.2%; LCMS (ES⁺): 332.8 [MH]⁺ |
| 172 | 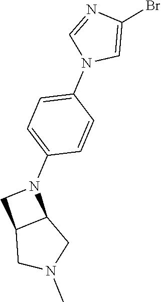 [SMILES: CN1C[C@H]2CN(c3ccc(-n4cnc(Br)c4)cc3)[C@H]2C1] | (1S,5R)-6-(4-(4-Bromo- 1H-imidazol-1- yl)phenyl)-3-methyl-3,6- diazabicyclo[3.2.0] heptane | Intermediate 45, CAS: 1419075-96-9; Off-white solid; Yield 62.7 mg, 27.7%; LCMS (ES⁺): 332.8 [MH]⁺ |
| 173 | 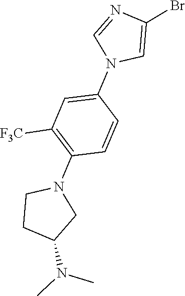 [SMILES: CN(C)[C@@H]1CCN(c2ccc(-n3cnc(Br)c3)cc2C(F)(F)F)C1] | (R)-1-(4-(4-Bromo-1H- imidazol-1-yl)-2- (trifluoromethyl)phenyl)- N,N-dimethylpyrrolidin- 3-amine | Intermediate 46, CAS: 132958-72-6; Orange liquid; Yield 523 mg, 93.6%; LCMS (ES⁺): 402.8 [MH]⁺ |
| 174 | 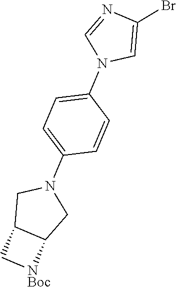 | tert-Butyl (1S,5R)-3-(4- (4-bromo-1H-imidazol- 1-yl)phenyl)-3,6- diazabicyclo[3.2.0] heptane-6-carboxylate | Intermediate 45, CAS: 370882-66-9; Off-white solid; Yield 232 mg, 41.3%; LCMS (ES⁺): 420.9 [MH]⁺ |
| 175 | 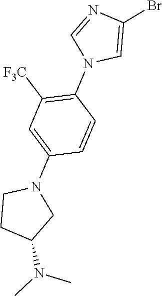 [SMILES: CN(C)[C@@H]1CCN(c2ccc(-n3cnc(Br)c3)c(C(F)(F)F)c2)C1] | (R)-1-(4-(4-Bromo-1H- imidazol-1-yl)-3- (trifluoromethyl)phenyl)- N,N-dimethylpyrrolidin- 3-amine | Intermediate 47, CAS: 132958-72-6; Beige solid; Yield 183 mg, 63.1%; LCMS (ES⁺): 402.8 [MH]⁺ |
| 176 | 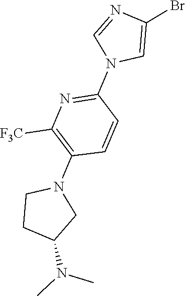 [SMILES: CN(C)[C@@H]1CCN(c2ccc(-n3cnc(Br)c3)nc2C(F)(F)F)C1] | (R)-1-(6-(4-Bromo-1H- imidazol-1-yl)-2- (trifluoromethyl)pyridin- 3-yl)-N,N- dimethylpyrrolidin-3- amine | Intermediate 48, CAS: 132958-72-6; Beige solid; Yield 175 mg, 56.2%; LCMS (ES⁺): 404.2 [MH]⁺ |
| 177 | 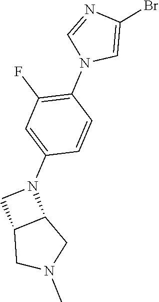 [SMILES: CN1C[C@@H]2CN(c3ccc(-n4cnc(Br)c4)c(F)c3)[C@@H]2C1] | (1R,5S)-6-(4-(4-Bromo- 1H-imidazol-1-yl)-3- fluorophenyl)-3-methyl- 3,6- diazabicyclo[3.2.0] heptane | Intermediate 27, CAS: 1419075-95-8; Off-white solid; Yield 35.5 mg, 12.5%; LCMS (ES⁺): 351.2 [MH]⁺ |
| 178 | 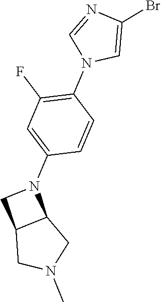 [SMILES: CN1C[C@H]2CN(c3ccc(-n4cnc(Br)c4)c(F)c3)[C@H]2C1] | (1S,5R)-6-(4-(4-Bromo- 1H-imidazol-1-yl)-3- fluorophenyl)-3-methyl- 3,6- diazabicyclo[3.2.0] heptane | Intermediate 27, CAS: 1419075-96-9; Off-white solid; Yield 37.0 mg, 16.2%; LCMS (ES⁺): 351.2 [MH]⁺ |
| 179 | 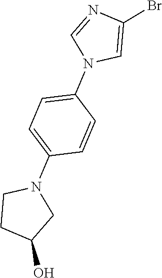 [SMILES: O[C@H]1CCN(c2ccc(-n3cnc(Br)c3)cc2)C1] | (S)-1-(4-(4-Bromo-1H- imidazol-1- yl)phenyl)pyrrolidin-3-ol | Intermediate 45, CAS: 100243-39-8; Off-white solid; Yield 243 mg, 42.0%; LCMS (ES⁺): 308.1 [MH]⁺ |
| 180 | 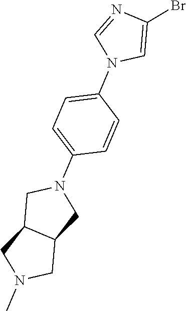 [SMILES: CN1C[C@@H]2CN(c3ccc(-n4cnc(Br)c4)cc3)C[C@@H]2C1] | (3aR,6aS)-2-(4-(4- Bromo-1H-imidazol-1- yl)phenyl)-5- methyloctahydropyrrolo [3,4-c]pyrrole | Intermediate 45, CAS: 172739-03-6; Off-white solid; Yield 198 mg, 40.5%; LCMS (ES⁺): 346.9 [MH]⁺ |
| 181 | 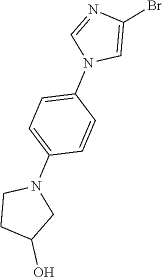 [SMILES: OC1CCN(c2ccc(-n3cnc(Br)c3)cc2)C1] | 1-(4-(4-Bromo-1H- imidazol-1- yl)phenyl)pyrrolidin-3-ol | Intermediate 45, CAS: 40499-83-0; Yellow solid; Yield 10.0 g, 65.3%; LCMS (ES⁺): 308.1 [MH]⁺ |

### Intermediate 182

### (R)-1-(4-(4-Bromo-1H-imidazol-1-yl)-3-(dimethylamino)phenyl)-N,N-dimethylpyrrolidin-3-amine

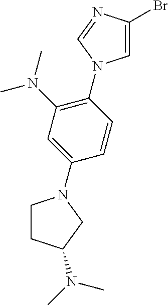 [SMILES: CN(C)c1cc(N2CC[C@@H](N(C)C)C2)ccc1-n1cnc(Br)c1]

Step 1: To a stirred mixture of Intermediate 137 (1.70 g, 4.47 mmol) and iron powder (2.00 g, 35.8 mmol) in EtOH (50 mL) were added NH₄Cl (1.91 g, 35.8 mmol) and water (403 μL, 22.4 mmol) at RT. The resulting mixture was stirred for 2 h at 80° C. then filtered and the filter cake washed with EtOH (3×20 mL). The filtrate was concentrated in vacuo, then diluted with water (100 mL) and extracted with EtOAc (5×100 mL). The combined organic layers were washed with brine (5×20 mL), dried (Na₂SO₄), then the residue purified via NP column chromatography to afford (R)-1-(3-amino-4-(4-bromo-1H-imidazol-1-yl)phenyl)-N,N-dimethylpyrrolidin-3-amine (936 mg, 59.8%) as a brown solid. LCMS (ES⁺): 350.3 [MH]⁺.

Step 2: A mixture of (R)-1-(3-amino-4-(4-bromo-1H-imidazol-1-yl)phenyl)-N,N-dimethylpyrrolidin-3-amine (936 mg, 2.67 mmol) in MeCOH (20 mL) was treated with formaldehyde (2.20 mL, 22.1 mmol) for 10 min followed by the addition of NaBH₃CN (336 mg, 5.34 mmol) at RT. The resulting mixture was stirred for 16 h at 60° C., concentrated in vacuo and purified via RP column chromatography to afford the title compound (289 mg, 28.6%) as a yellow solid. LCMS (ES⁺): 378.3 [MH]⁺.

### Intermediate 183

### (R)-1-(4-(4-Bromo-1H-imidazol-1-yl)phenyl)-N,N-bis(methyl-d3)pyrrolidin-3-amine

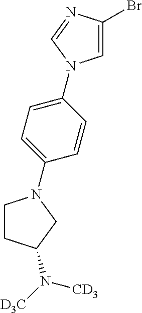 [SMILES: [2H]C([2H])([2H])N([C@@H]1CCN(c2ccc(-n3cnc(Br)c3)cc2)C1)C([2H])([2H])[2H]]

Step 1: To a solution of Intermediate 179 (243 mg, 86.0% purity, 0.68 mmol) and pyridine (220 μL, 2.73 mmol) in DCM (5.0 mL) was added p-toluenesulfonyl chloride (388 mg, 2.04 mmol). The reaction mixture was stirred at RT for 64 h then further pyridine (220 μL, 2.73 mmol) and p-toluenesulfonyl chloride (388 mg, 2.04 mmol) were added and the mixture stirred for a further 24 h. The reaction mixture was concentrated in vacuo and purified via NP column chromatography to afford [(3S)-1-[4-(4-bromoimidazol-1-yl)phenyl]pyrrolidin-3-yl]4-methylbenzenesulfonate (51.4 mg, 12.8%) as a pale brown gum. LCMS (ES⁺): 462.1 [MH]⁺.

Step 2: [(3S)-1-[4-(4-Bromoimidazol-1-yl)phenyl]pyrrolidin-3-yl]4-methylbenzenesulfonate (51.4 mg, 78.2% purity, 86.9 μmol), K₂CO₃ (60.0 mg, 0.43 mmol) and dimethyl-d₆-amine-HCl (23.0 mg, 0.26 mmol) in MeCN (2.0 mL) were stirred at 80° C. in a sealed tube for 18 h. Further K₂CO₃ (60.0 mg, 0.43 mmol) and dimethyl-d₆-amine-HCl (23.0 mg, 0.26 mmol) were added and the mixture stirred for a further 6 h at 80° C. then concentrated in vacuo and purified via NP column chromatography to afford the title compound (13.8 mg, 46.6%) as a pale-yellow solid. LCMS (ES⁺): 343.2 [MH]⁺.

### Intermediate 184

### (3S,4R)-1-(4-(4-Bromo-1H-imidazol-1-yl)phenyl)-4-fluoro-N-methylpyrrolidin-3-amine

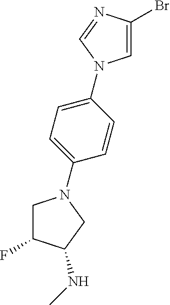 [SMILES: CN[C@H]1CN(c2ccc(-n3cnc(Br)c3)cc2)C[C@H]1F]

Step 1: A mixture of Intermediate 123 (520 mg, 1.22 mmol) in DMF (5.0 mL) was treated with NaH (40% by weight, 73.0 mg, 1.83 mmol) for 30 min at 0° C. under N₂ followed by the addition of Mel (113 μL, 1.83 mmol) dropwise at 0° C. The mixture was warmed to RT, stirred for 16 h, quenched with water (20 mL) then extracted with EtOAc (6×50 mL). The combined organic layers were washed with brine (6×20 mL), dried (Na₂SO₄), concentrated in vacuo and purified via prep-TLC (PE/EtOAc 2:1) to afford tert-butyl ((3S,4R)-1-(4-(4-bromo-1H-imidazol-1-yl)phenyl)-4-fluoropyrrolidin-3-yl)(methyl)carbamate (380 mg, 70.7%) as a yellow solid. LCMS (ES⁺): 439.0 [MH]⁺.

Step 2: A mixture of tert-butyl N-[(3S,4R)-1-[4-(4-bromoimidazol-1-yl)phenyl]-4-fluoropyrrolidin-3-yl]-N-methylcarbamate (380 mg, 0.87 mmol) and TFA (1.0 mL) in DCM (10 mL) was stirred for 2 h at RT. The mixture was concentrated in vacuo and purified via prep-TLC (DCM/MeCOH 20:1) to afford the title compound (220 mg, 74.6%) as a yellow solid. LCMS (ES⁺): 338.9 [MH]⁺.

### Intermediate 185

### tert-Butyl ((3S,4R)-1-(4-(4-bromo-1H-imidazol-1-yl)phenyl)-4-methoxypyrrolidin-3-yl)(methyl)carbamate

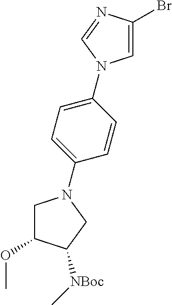 [SMILES: CO[C@@H]1CN(c2ccc(-n3cnc(Br)c3)cc2)C[C@@H]1N(C)C(=O)OC(C)(C)C]

A solution of Intermediate 149 (156 mg, 0.36 mmol) in DMF (3.0 mL) was treated with NaH (22.0 mg, 0.55 mmol) for 40 min at 0° C. under N₂ followed by the addition of Mel (33.0 μL, 0.53 mmol) dropwise. The resulting mixture was stirred for 1 h at RT then concentrated in vacuo and purified via RP column chromatography to afford the title compound (152 mg, 93.2%) as a yellow solid. LCMS (ES⁺): 451.0 [MH]⁺.

### Intermediate 186

### tert-Butyl ((3S,4R)-1-(4-(4-bromo-1H-imidazol-1-yl)-3-methoxyphenyl)-4-fluoropyrrolidin-3-yl)(methyl)carbamate

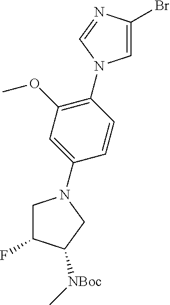 [SMILES: COc1cc(N2C[C@@H](F)[C@@H](N(C)C(=O)OC(C)(C)C)C2)ccc1-n1cnc(Br)c1]

Intermediate 186 was prepared similarly to Intermediate 185 via alkylation of Intermediate 157 (220 mg, 0.48 mmol) with Mel (45.0 μL, 0.72 mmol) then purification via prep-TLC (PE/EtOAc 1:1) to afford the title compound (200 mg, 88.2%) as a brown solid. LCMS (ES⁺): 469.1 [MH]⁺.

### Intermediate 187

### tert-Butyl (R)-(1-(4-(4-bromo-1H-imidazol-1-yl)phenyl)pyrrolidin-3-yl)(ethyl)carbamate

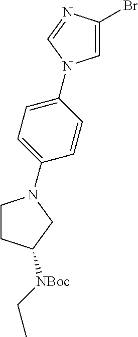 [SMILES: CCN(C(=O)OC(C)(C)C)[C@@H]1CCN(c2ccc(-n3cnc(Br)c3)cc2)C1]

Intermediate 187 was prepared similarly to Intermediate 185 via alkylation of Intermediate 118 (200 mg, 0.49 mmol) with EtI (60.0 μL, 0.74 mmol) to afford the title compound (200 mg, 93.6%) as a white solid. LCMS (ES⁺): 435.1 [MH]⁺.

### Intermediate 188

### 1-(4-(4-Bromo-1H-imidazol-1-yl)phenyl)pyrrolidin-3-one

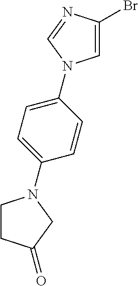 [SMILES: O=C1CCN(c2ccc(-n3cnc(Br)c3)cc2)C1]

A solution of DMSO (6.50 mL, 91.5 mmol) in DCM (200 mL) was treated with oxalyl chloride (3.90 mL, 46.1 mmol) for 5 min at −78° C. under N₂ then Intermediate 181 (10.0 g, 32.5 mmol) in DCM (50 mL) was added dropwise at −78° C. The resulting mixture was warmed to RT, stirred for 1 h, then Et₃N (28.0 mL, 202 mmol) was added and the mixture stirred for 16 h. DCM (200 mL) was added then the reaction was quenched through addition of sat aq NaHCO₃ (200 mL), washed with water (6×100 mL), dried (Na₂SO₄) and concentrated in vacuo. Purification via NP column chromatography afforded the title compound (6.00 g, 60.4%) as a yellow solid. LCMS (ES⁺): 306.0 [MH]⁺.

### Intermediate 189

### (2R,3R)-1-(4-(4-Bromo-1H-imidazol-1-yl)phenyl)-N,N,2-trimethylpyrrolidin-3-amine

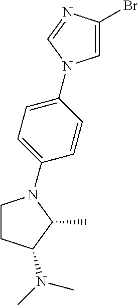 [SMILES: C[C@@H]1[C@H](N(C)C)CCN1c1ccc(-n2cnc(Br)c2)cc1]

Step 1: Intermediate 139 (185 mg, 0.41 mmol) in AcOH (4.0 mL) was treated with HBr (2.0 mL, 40% in AcOH) for 1 h at RT then concentrated in vacuo. Purification via RP column chromatography afforded (2R,3R)-1-[4-(4-bromoimidazol-1-yl)phenyl]-2-methylpyrrolidin-3-amine (130 mg, 98.8%) as a yellow solid. LCMS (ES⁺): 321.0 [MH]⁺.

Step 2: A solution of (2R,3R)-1-[4-(4-bromoimidazol-1-yl)phenyl]-2-methylpyrrolidin-3-amine (130 mg, 0.40 mmol) in MeCOH (5.0 mL) was treated with formaldehyde (122 μL, 1.23 mmol) for 10 min followed by the addition of NaBH₃CN (76.0 mg, 1.22 mmol) at RT. The resulting mixture was stirred for 2 h at RT, quenched with water (10 mL), extracted with EtOAc (3×20 mL) and the combined organic layers were washed with brine (3×5.0 mL), dried (Na₂SO₄) then concentrated in vacuo. Purification via prep-TLC (DCM/MeOH 10:1) afforded the title compound (90.0 mg, 64.7%) as a yellow solid. LCMS (ES⁺) 348.9 [MH]⁺.

### Intermediate 190

### (3aR,6aR)-1-(4-(4-Bromo-1H-imidazol-1-yl)phenyl)-4-methyloctahydropyrrolo[3,2-b]pyrrole

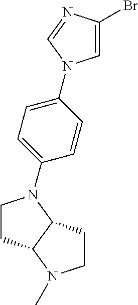 [SMILES: CN1CC[C@@H]2[C@H]1CCN2c1ccc(-n2cnc(Br)c2)cc1]

Intermediate 190 was prepared similarly to Intermediate 189 via Cbz deprotection of Intermediate 144 (300 mg, 0.64 mmol) then reductive amination with formaldehyde using NaBH₃CN to afford the title compound (167 mg, 75.2% over 2 steps) as a yellow solid. LCMS (ES⁺): 347.1 [MH]⁺.

### Intermediate 191

### 4-Bromo-1-(4-((((3S,4S)-4-fluoro-1-methylpyrrolidin-3-yl)oxy)methyl)phenyl)-1H-imidazole

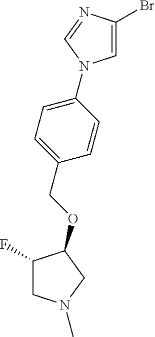 [SMILES: CN1C[C@H](OCc2ccc(-n3cnc(Br)c3)cc2)[C@@H](F)C1]

Step 1: A solution of Intermediate 56 (510 mg, 1.16 mmol) and TFA (2.0 mL) in DCM (10 mL) was stirred for 1 h at RT. The resulting mixture was concentrated in vacuo to afford 4-bromo-1-(4-((((3S,4S)-4-fluoropyrrolidin-3-yl)oxy)methyl)phenyl)-1H-imidazole (390 mg) as a red oil, which was used directly in the next step without further purification. LCMS (ES⁺): 339.9 [MH]⁺.

Step 2: To a stirred solution of 4-bromo-1-(4-((((3S,4S)-4-fluoropyrrolidin-3-yl)oxy)methyl)phenyl)-1H-imidazole (390 mg, 1.15 mmol) and formaldehyde (108 μL, 1.09 mmol) in THF (5.0 mL) was added NaBH₃CN (80.0 mg, 1.27 mmol) at RT. The solution was stirred for 1 h at RT then concentrated in vacuo and purified via RP column chromatography to afford the title compound (210 mg, 51.2% over 2 steps) as a yellow oil. LCMS (ES⁺): 353.9 [MH]⁺.

### Intermediates 192 to 210

Intermediates 192 to 210 were prepared similarly to Intermediate 191 via Boc deprotection of the specified amine Intermediate using TFA, then reductive amination with the appropriate aldehyde or ketone using NaBH₃CN, NaBH₄ or STAB as the reducing agent; see Table 8 below.

TABLE 8
Boc removal then reductive amination of the specified amine
Intermediates

| | | | Reactant(s) used; |
|---|---|---|---|
| | | | Reducing agent; |
| Int. | Structure | Name | Form; Yield; LCMS |
| 192 | 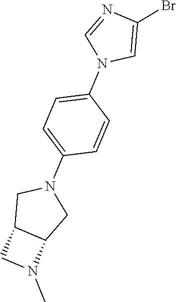 [SMILES: CN1C[C@@H]2CN(c3ccc(-n4cnc(Br)c4)cc3)C[C@@H]21] | (1R,5R)-3-(4-(4-Bromo- 1H-imidazol-1-yl)phenyl)- 6-methyl-3,6- diazabicyclo[3.2.0]heptane | Intermediate 174, CAS: 50-00-0; STAB; White solid; Yield 181 mg, 57.2% over 2 steps; LCMS (ES⁺): 332.8 [MH]⁺ |
| 193 | 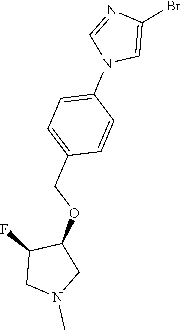 [SMILES: CN1C[C@@H](F)[C@@H](OCc2ccc(-n3cnc(Br)c3)cc2)C1] | 4-Bromo-1-(4-((((3S,4R)- 4-fluoro-1- methylpyrrolidin-3- yl)oxy)methyl)phenyl)-1H- imidazole | Intermediate 57, CAS: 50-00-0; NaBH₄; Yellow solid; Yield 400 mg, 97.5% over 2 steps; LCMS (ES⁺): 354.0 [MH]⁺ |
| 194 | 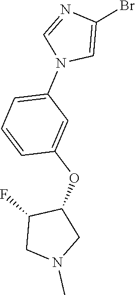 [SMILES: CN1C[C@H](F)[C@H](Oc2cccc(-n3cnc(Br)c3)c2)C1] | 4-bromo-1-(3-(((3R,4S)-4- fluoro-1-methylpyrrolidin- 3-yl)oxy)phenyl)-1H- imidazole | Intermediate 74, CAS: 50-00-0; NaBH₃CN; White solid; Yield 150 mg, 59.4% over 2 steps; LCMS (ES⁺): 340.1 [MH]⁺ |
| 195 | 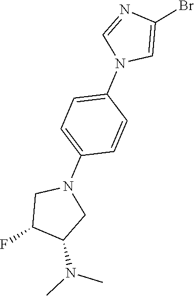 [SMILES: CN(C)[C@H]1CN(c2ccc(-n3cnc(Br)c3)cc2)C[C@H]1F] | (3S,4R)-1-(4-(4-Bromo- 1H-imidazol-1-yl)phenyl)- 4-fluoro-N,N- dimethylpyrrolidin-3-amine | Intermediate 123, CAS: 50-00-0; NaBH₃CN; White solid; Yield 334 mg, 73.1% over 2 steps; LCMS (ES⁺): 353.0 [MH]⁺ |
| 196 | 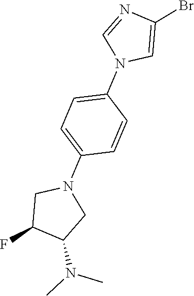 [SMILES: CN(C)[C@H]1CN(c2ccc(-n3cnc(Br)c3)cc2)C[C@@H]1F] | (3S,4S)-1-(4-(4-Bromo- 1H-imidazol-1-yl)phenyl)- 4-fluoro-N,N- dimethylpyrrolidin-3-amine | Intermediate 117, CAS: 50-00-0; NaBH₃CN; Yellow solid; Yield 130 mg, 56.0% over 2 steps; LCMS (ES⁺): 353.1 [MH]⁺ |
| 197 | 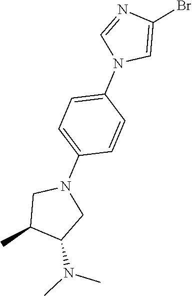 [SMILES: C[C@H]1CN(c2ccc(-n3cnc(Br)c3)cc2)C[C@@H]1N(C)C] | (3R,4S)-1-(4-(4-Bromo- 1H-imidazol-1-yl)phenyl)- N,N,4-trimethylpyrrolidin- 3-amine | Intermediate 131, CAS: 50-00-0; NaBH₃CN; Yellow solid; Yield 80.0 mg, 70.1% over 2 steps; LCMS (ES⁺): 351.2 [MH]⁺ |
| 198 | 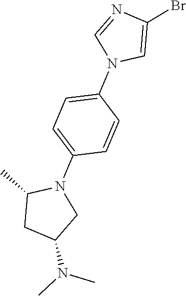 [SMILES: C[C@H]1C[C@@H](N(C)C)CN1c1ccc(-n2cnc(Br)c2)cc1] | (3R,5S)-1-(4-(4-Bromo- 1H-imidazol-1-yl)phenyl)- N,N,5-trimethylpyrrolidin- 3-amine | Intermediate 133, CAS: 50-00-0; NaBH₃CN; Off-white solid; Yield 130 mg, 47.0% over 2 steps; LCMS (ES⁺): 349.1 [MH]⁺ |
| 199 | 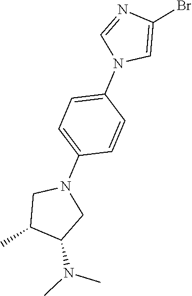 [SMILES: C[C@@H]1CN(c2ccc(-n3cnc(Br)c3)cc2)C[C@@H]1N(C)C] | (3R,4R)-1-(4-(4-Bromo- 1H-imidazol-1-yl)phenyl)- N,N,4-trimethylpyrrolidin- 3-amine | Intermediate 134, CAS: 50-00-0; NaBH₃CN; Yellow solid; Yield 90.0 mg, 82.8% over 2 steps; LCMS (ES⁺): 348.9 [MH]⁺ |
| 200 | 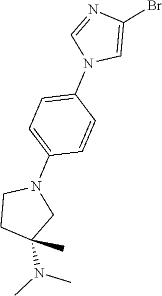 [SMILES: CN(C)[C@]1(C)CCN(c2ccc(-n3cnc(Br)c3)cc2)C1] | (R)-1-(4-(4-Bromo-1H- imidazol-1-yl)phenyl)- N,N,3-trimethylpyrrolidin- 3-amine | Intermediate 121, CAS: 50-00-0; NaBH₃CN; Yellow solid; Yield 150 mg, 83.3% over 3 steps; LCMS (ES⁺): 349.2 [MH]⁺ |
| 201 | 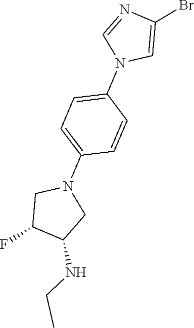 [SMILES: CCN[C@H]1CN(c2ccc(-n3cnc(Br)c3)cc2)C[C@H]1F] | (3S,4R)-1-(4-(4-Bromo- 1H-imidazol-1-yl)phenyl)- N-ethyl-4-fluoropyrrolidin- 3-amine | Intermediate 123, CAS: 75-07-0; NaBH₃CN; Yellow solid; Yield 123 mg, 28.4% over 2 steps; LCMS (ES⁺): 353.1 [MH]⁺ |
| 202 | 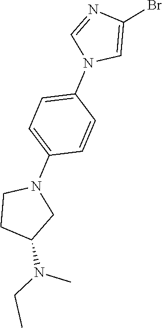 [SMILES: CCN(C)[C@@H]1CCN(c2ccc(-n3cnc(Br)c3)cc2)C1] | (R)-1-(4-(4-Bromo-1H- imidazol-1-yl)phenyl)-N- ethyl-N-methylpyrrolidin-3- amine | Intermediate 120, CAS: 75-07-0; NaBH₃CN; White solid; Yield 76.0 mg, 51.0% over 2 steps; LCMS (ES⁺): 349.1 [MH]⁺ |
| 203 | 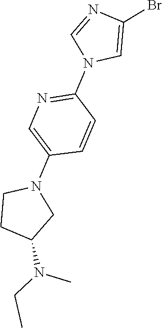 [SMILES: CCN(C)[C@@H]1CCN(c2ccc(-n3cnc(Br)c3)nc2)C1] | (R)-1-(6-(4-Bromo-1H- imidazol-1-yl)pyridin-3-yl)- N-ethyl-N- methylpyrrolidin-3-amine | Intermediate 143, CAS: 75-07-0; NaBH₃CN; White solid; Yield 60 mg, 65.9% over two steps; LCMS (ES⁺): 350.1 [MH]⁺ |
| 204 | 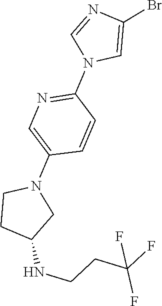 [SMILES: FC(F)(F)CCN[C@@H]1CCN(c2ccc(-n3cnc(Br)c3)nc2)C1] | (R)-1-(6-(4-Bromo-1H- imidazol-1-yl)pyridin-3-yl)- N-(3,3,3- trifluoropropyl)pyrrolidin-3- amine | Intermediate 146, CAS: 460-40-2; NaBH₃CN; Yellow solid; Yield 170 mg, 41.2% over 2 steps; LCMS (ES⁺): 404.0 [MH]⁺ |
| 205 | 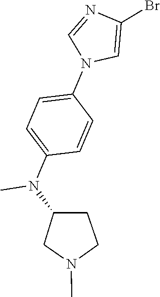 [SMILES: CN1CC[C@@H](N(C)c2ccc(-n3cnc(Br)c3)cc2)C1] | (R)-N-(4-(4-Bromo-1H- imidazol-1-yl)phenyl)-N,1- dimethylpyrrolidin-3-amine | Intermediate 147, CAS: 50-00-0; NaBH₃CN; Brown oil; Yield 303 mg, 95.2% over 2 steps; LCMS (ES⁺): 335.1 [MH]⁺ |
| 206 | 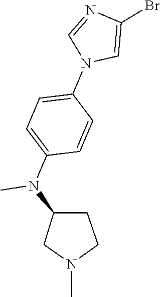 [SMILES: CN1CC[C@H](N(C)c2ccc(-n3cnc(Br)c3)cc2)C1] | (S)-N-(4-(4-Bromo-1H- imidazol-1-yl)phenyl)-N,1- dimethylpyrrolidin-3-amine | Intermediate 148, CAS: 50-00-0; NaBH₃CN; Colourless oil; Yield 290 mg, 91.1% over two steps; LCMS (ES⁺): 335.2 [MH]⁺ |
| 207 | 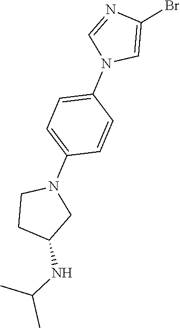 [SMILES: CC(C)N[C@@H]1CCN(c2ccc(-n3cnc(Br)c3)cc2)C1] | (R)-1-(4-(4-Bromo-1H- imidazol-1-yl)phenyl)-N- isopropylpyrrolidin-3- amine | Intermediate 118, CAS: 67-64-1; NaBH₃CN; White solid; Yield 160 mg, 82.2% over two steps; LCMS (ES⁺): 349.2 [MH]⁺ |
| 208 | 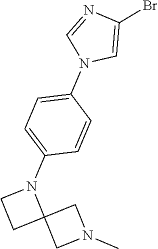 [SMILES: CN1CC2(CCN2c2ccc(-n3cnc(Br)c3)cc2)C1] | 1-(4-(4-Bromo-1H- imidazol-1-yl)phenyl)-6- methyl-1,6- diazaspiro[3.3]heptane | Intermediate 152, CAS: 50-00-0; NaBH₃CN; Yellow solid; Yield 310 mg, 47.2% over 2 steps; LCMS (ES⁺): 333.2 [MH]⁺ |
| 209 | 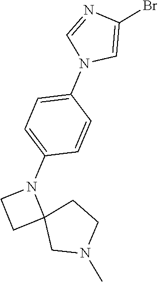 [SMILES: CN1CCC2(CCN2c2ccc(-n3cnc(Br)c3)cc2)C1] | 1-(4-(4-Bromo-1H- imidazol-1-yl)phenyl)-6- methyl-1,6- diazaspiro[3.4]octane | Intermediate 155, CAS: 50-00-0; NaBH₃CN; Yellow solid; Yield 70.0 mg, 43.6% over two steps; LCMS (ES⁺): 347.1 [MH]⁺ |
| 210 | 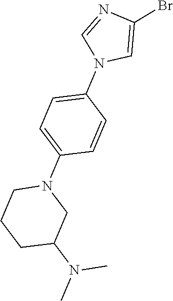 [SMILES: CN(C)C1CCCN(c2ccc(-n3cnc(Br)c3)cc2)C1] | 1-(4-(4-Bromo-1H- imidazol-1-yl)phenyl)-N,N- dimethylpiperidin-3-amine | Intermediate 156, CAS: 50-00-0; NaBH₃CN; Brown solid; Yield 385 mg, 60.0% over two steps; LCMS (ES⁺): 349.1 [MH]⁺ |

### Intermediates 211 and 212

### 6-(4-(4-Bromo-1H-imidazol-1-yl)phenyl)-1-methyl-1,6-diazaspiro[3.4]octane

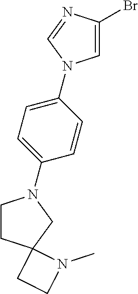 [SMILES: CN1CCC12CCN(c1ccc(-n3cnc(Br)c3)cc1)C2]

Intermediates 211 and 212 were prepared from Intermediates 162 and 163, respectively, similarly to Intermediate 191 via Boc deprotection of Intermediates 162 and 163 using TFA, then reductive amination with the formaldehyde using NaBH₃CN as the reducing agent. Intermediates 162 and 163 were isolated as single enantiomers of unknown absolute stereochemistry, therefore Intermediates 211 and 212 are also single enantiomers of absolute unknown stereochemistry.

Intermediate 211 (Enantiomer 1): Isolated 80.0 mg, 24.9% over 2 steps as a yellow oil. LCMS (ES⁺): 347.0 [MH]⁺.

Intermediate 212 (Enantiomer 2): Isolated 150 mg, 43.5% over 2 steps as a yellow oil. LCMS (ES⁺): 347.1 [MH]⁺.

### Intermediate 213

### 4-Bromo-1-(4-(3-(3,3-dimethylazetidin-1-yl)pyrrolidin-1-yl)phenyl)-1H-imidazole

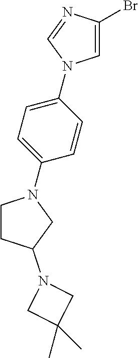 [SMILES: CC1(C)CN(C2CCN(c3ccc(-n4cnc(Br)c4)cc3)C2)C1]

A mixture of Intermediate 188 (400 mg, 1.31 mmol), 3,3-dimethylazetidine-HCl (175 mg, 1.44 mmol) and STAB (831 mg, 3.92 mmol) in DCM (10 mL) was stirred for 1 h at RT. The resulting mixture was concentrated in vacuo and purified via prep-TLC (DCM/MeOH=50:1) to afford crude product (420 mg) as a yellow solid, which was further purified via prep-HPLC to afford the title compound (120 mg 24.5%) as a yellow solid. LCMS (ES⁺): 375.1 [MH]⁺.

### Intermediate 214

### 4-Bromo-1-(4-(3-(3-methylazetidin-1-yl)pyrrolidin-1-yl)phenyl)-1H-imidazole

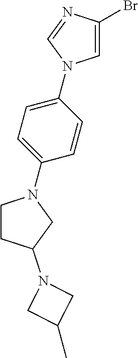 [SMILES: CC1CN(C2CCN(c3ccc(-n4cnc(Br)c4)cc3)C2)C1]

Intermediate 214 was prepared similarly to Intermediate 213, via reductive amination of Intermediate 188 (400 mg, 1.31 mmol) with 3-methylazetidine-HCl (155 mg, 1.44 mmol) then purification via prep-TLC (EtOAc) to afford the title compound (200 mg, 42.4%) as a yellow solid. LCMS (ES⁺): 361.1 [MH]⁺.

### Intermediates 215 to 226

Intermediates 215 to 226 were prepared similarly to Intermediate 213 via reductive amination of Intermediate 188 with the appropriate amine, but with a subsequent isomer separation step using either prep-SFC or chiral prep-HPLC to afford two Intermediates as either single enantiomers or single diastereomers with unknown absolute stereochemistry; see Table 9 below.

TABLE 9
Reductive amination using Intermediate 188, then isomer
separation

| | | | Isomer | Isomer; Reactant |
|---|---|---|---|---|
| | | | separation | used; Form; Yield; |
| Int. | Structure | Name | method | LCMS |
| 215 | 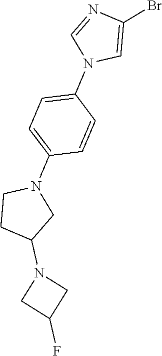 [SMILES: FC1CN(C2CCN(c3ccc(-n4cnc(Br)c4)cc3)C2)C1] | 4-Bromo-1-(4-(3- (3-fluoroazetidin- 1-yl)pyrrolidin-1- yl)phenyl)-1H- imidazole | Prep-SFC Method 2 Rt: 7.50 min | Enantiomer 1; CAS: 690257-76-2; Yellow oil; Yield 214 mg, 35.9%; LCMS (ES⁺): 365.1 [MH]⁺ |
| 216 | | | Prep-SFC Method 2 Rt: 8.75 min | Enantiomer 2; CAS: 690257-76-2; Yellow oil; Yield 111 mg, 18.6%; LCMS (ES⁺): 365.1 [MH]⁺ |
| 217 | | 2-(1-(4-(4-Bromo- 1H-imidazol-1- yl)phenyl)pyrrolidin- 3-yl)-2- azaspiro[3.3]heptane | Chiral Prep- HPLC Method 1 Rt: 7.25 min | Enantiomer 1; CAS: 665-04-3; White solid; Yield 100 mg, 41.7%; LCMS (ES⁺): 386.1 [MH]⁺ |
| 218 | 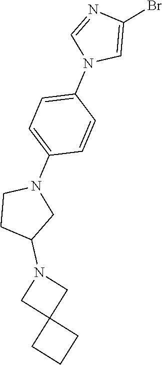 [SMILES: Brc1cn(-c2ccc(N3CCC(N4CC5(CCC5)C4)C3)cc2)cn1] | | Chiral Prep- HPLC Method 1 Rt: 8.78 min | Enantiomer 2; CAS: 665-04-3; White solid; Yield 100 mg, 41.7%; LCMS (ES⁺): 386.1 [MH]⁺ |
| 219 | 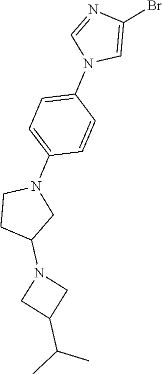 [SMILES: CC(C)C1CN(C2CCN(c3ccc(-n4cnc(Br)c4)cc3)C2)C1] | 4-Bromo-1-(4-(3- (3- isopropylazetidin- 1-yl)pyrrolidin-1- yl)phenyl)-1H- imidazole | Chiral Prep- HPLC Method 1 Rt: 6.60 min | Enantiomer 1; White solid; CAS: 1423116-93-1; Yield 100 mg, 45.4%; LCMS (ES⁺): 389.1 [MH]⁺ |
| 220 | | | Chiral Prep- HPLC Method 1 Rt: 8.90 min | Enantiomer 2; White solid; CAS: 1423116-93-1; Yield 100 mg, 45.4%; LCMS (ES⁺): 389.1 [MH]⁺ |
| 221 | 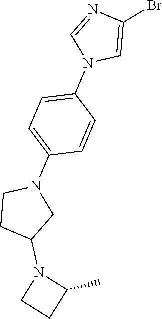 [SMILES: C[C@@H]1CCN1C1CCN(c2ccc(-n3cnc(Br)c3)cc2)C1] | 4-Bromo-1-(4-(3- ((R)-2- methylazetidin-1- yl)pyrrolidin-1- yl)phenyl)-1H- imidazole | Chiral Prep- HPLC Method 2 Rt: 11.91 min | Diastereomer 1; CAS: 791614-71-6; Yellow solid; Yield 65.0 mg, 13.8%; LCMS (ES⁺): 361.1 [MH]⁺ |
| 222 | | | Chiral Prep- HPLC Method 2 Rt: 15.37 min | Diastereomer 2; CAS: 791614-71-6; Yellow solid; Yield 79.0 mg, 16.7%; LCMS (ES⁺): 361.1 [MH]⁺ |
| 223 | 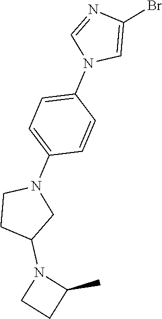 [SMILES: C[C@H]1CCN1C1CCN(c2ccc(-n3cnc(Br)c3)cc2)C1] | 4-Bromo-1-(4-(3- ((S)-2- methylazetidin-1- yl)pyrrolidin-1- yl)phenyl)-1H- imidazole | Chiral Prep- HPLC Method 3 Rt: 88 min | Diastereomer 1; CAS: 52265-79-9; White solid; Yield 67.5 mg, 27.0%; LCMS (ES⁺): 361.1 [MH]⁺ |
| 224 | | | Chiral Prep- HPLC Method 3 Rt: 120 min | Diastereomer 2; CAS: 52265-79-9; White solid; Yield 57.7 mg, 23.1%; LCMS (ES⁺): 361.1 [MH]⁺ |
| 225 | 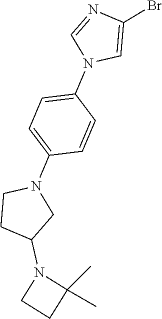 [SMILES: CC1(C)CCN1C1CCN(c2ccc(-n3cnc(Br)c3)cc2)C1] | 4-Bromo-1-(4-(3- (2,2- dimethylazetidin- 1-yl)pyrrolidin-1- yl)phenyl)-1H- imidazole | Chiral Prep- HPLC Method 3 Rt: 21.75 min | Enantiomer 1; CAS: 1086266-55-8; White solid; Yield 46.9 mg, 36.1%; LCMS (ES⁺): 375.1 [MH]⁺ |
| 226 | | | Chiral Prep- HPLC method 3 Rt: 24.25 min | Enantiomer 2; CAS: 1086266-55-8; White solid; Yield 48.0 mg, 36.9%; LCMS (ES⁺): 375.1 [MH]⁺ |

### EXAMPLES

### Example 1

### 5-((1-(4-((1R,5R)-6-Methyl-3,6-diazabicyclo[3.2.0]heptan-3-yl)phenyl)-1H-imidazol-4-yl)amino)pyrazine-2-carbonitrile

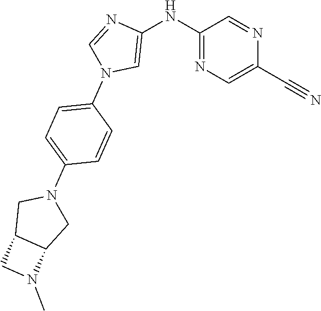 [SMILES: CN1C[C@@H]2CN(c3ccc(-n4cnc(Nc5cnc(C#N)cn5)c4)cc3)C[C@@H]21]

Intermediate 192 (181 mg, 95.1% purity, 0.52 mmol), 5-aminopyrazine-2-carbonitrile (78.0 mg, 0.65 mmol), Cs₂CO₃ (336 mg, 1.03 mmol), and AdBrettPhos (33.00 mg, 51.5 μmol) in DXN (3.0 mL) was sparged with N₂ for 5 min, then AdBrettPhos Pd G3 (52.0 mg, 51.5 μmol) was added and the mixture was sparged with N₂ for a further 5 min then heated to 100° C. in a sealed tube and allowed to stir for 15 h. The reaction mixture was concentrated in vacuo and purified via NP column chromatography. Further purification via NH₃-buffered RP HPLC then drying in a vacuum oven at 60° C. for 16 h afforded the title compound (36.2 mg, 18.6%) as a pale-yellow solid. LCMS (ES⁺): 373.2 [MH]⁺. UPLC: Rt 3.14 min, 99.0% purity.

### Examples 2 to 104

Examples 2 to 104 were prepared similarly to Example 1 via AdBrettPhos Pd G3-catalysed Buchwald-Hartwig coupling of the specified (hetero)aryl bromide Intermediate with 5-aminopyrazine-2-carbonitrile; see Table 10 below.

TABLE 10
AdBrettPhos Pd G3-catalysed Buchwald-Hartwig coupling
reactions of 5-aminopyrazine-2-carbonitrile with specified (hetero)aryl
bromide

| | | | Intermediate used; |
|---|---|---|---|
| | | | Form (salt); Yield; LCMS; |
| Ex. | Structure | Name | HPLC/UPLC |
| 2 | 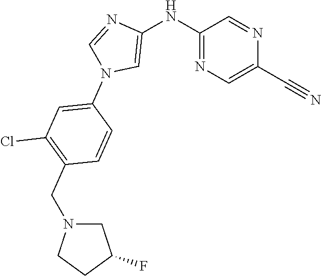 [SMILES: N#Cc1cnc(Nc2cn(-c3ccc(CN4CC[C@@H](F)C4)c(Cl)c3)cn2)cn1] | (R)-5-((1-(3-Chloro-4- ((3-fluoropyrrolidin-1- yl)methyl)phenyl)-1H- imidazol-4- yl)amino)pyrazine-2- carbonitrile | Intermediate 59; Off-white solid; Yield 6.3 mg, 21.7%; LCMS (ES⁺): 398.0 [MH]⁺; UPLC: Rt 3.54 min, 100% purity |
| 3 | 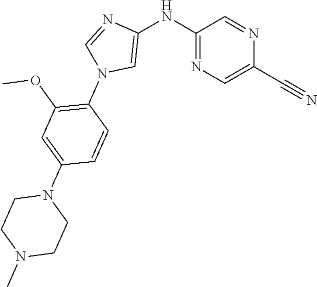 [SMILES: COc1cc(N2CCN(C)CC2)ccc1-n1cnc(Nc2cnc(C#N)cn2)c1] | 5-((1-(2-Methoxy-4-(4- methylpiperazin-1- yl)phenyl)-1H-imidazol- 4-yl)amino)pyrazine-2- carbonitrile | Intermediate 166; Light yellow solid; Yield 5.1 mg, 15.0%; LCMS (ES⁺): 391.1 [MH]⁺; UPLC: Rt 3.14 min, 99.3% purity |
| 4 | 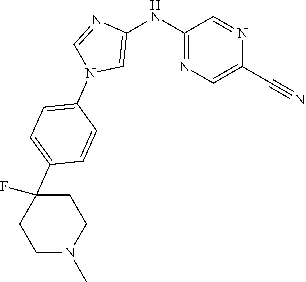 [SMILES: CN1CCC(F)(c2ccc(-n3cnc(Nc4cnc(C#N)cn4)c3)cc2)CC1] | 5-((1-(4-(4-Fluoro-1- methylpiperidin-4- yl)phenyl)-1H-imidazol- 4-yl)amino)pyrazine-2- carbonitrile | Intermediate 22; Yellow solid; Yield 35.4 mg, 63.5%; LCMS (ES⁺): 378.1 [MH]⁺; HPLC: Rt 7.20 min, 96.2% purity |
| 5 | 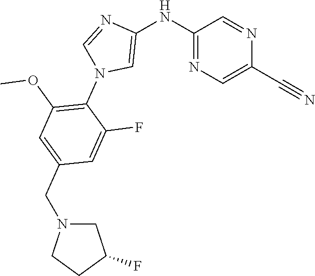 [SMILES: COc1cc(CN2CC[C@@H](F)C2)cc(F)c1-n1cnc(Nc2cnc(C#N)cn2)c1] | (R)-5-((1-(2-Fluoro-4- ((3-fluoropyrrolidin-1- yl)methyl)-6- methoxyphenyl)-1H- imidazol-4- yl)amino)pyrazine-2- carbonitrile | Intermediate 60; Yellow solid; Yield 8.7 mg, 30.4%; LCMS (ES⁺): 412.1 [MH]⁺; UPLC: Rt 3.47 min, 99.7% purity |
| 6 | 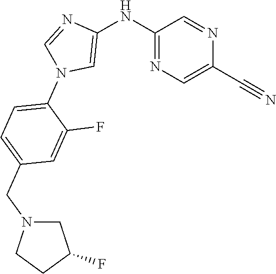 [SMILES: N#Cc1cnc(Nc2cn(-c3ccc(CN4CC[C@@H](F)C4)cc3F)cn2)cn1] | (R)-5-((1-(2-Fluoro-4- ((3-fluoropyrrolidin-1- yl)methyl)phenyl)-1H- imidazol-4- yl)amino)pyrazine-2- carbonitrile | Intermediate 61; Yellow solid; Yield 16.5 mg, 48.4%; LCMS (ES⁺): 382.2 [MH]⁺; UPLC: Rt 3.33 min, 100% purity |
| 7 | 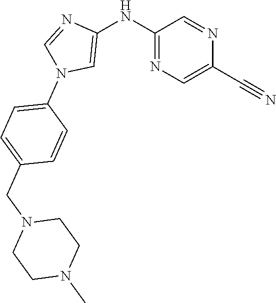 [SMILES: CN1CCN(Cc2ccc(-n3cnc(Nc4cnc(C#N)cn4)c3)cc2)CC1] | 5-((1-(4-((4- Methylpiperazin-1- yl)methyl)phenyl)-1H- imidazol-4- yl)amino)pyrazine-2- carbonitrile | Intermediate 58; Yellow solid; Yield 42.2 mg, 17.2%; LCMS (ES⁺): 375.3 [MH]⁺; HPLC: Rt 2.20 min, 97.2% purity |
| 8 | 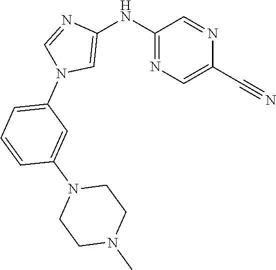 [SMILES: CN1CCN(c2cccc(-n3cnc(Nc4cnc(C#N)cn4)c3)c2)CC1] | 5-((1-(3-(4- Methylpiperazin-1- yl)phenyl)-1H-imidazol- 4-yl)amino)pyrazine-2- carbonitrile | Intermediate 24; Yellow solid; Yield 11.3 mg, 11.1%; LCMS (ES⁺): 361.2 [MH]⁺; HPLC: Rt 1.64 min, 97.2% purity |
| 9 | 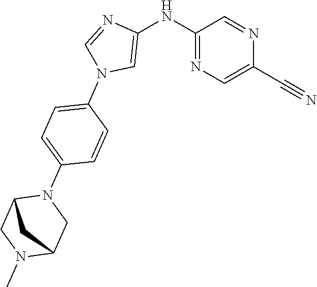 [SMILES: CN1C[C@H]2C[C@@H]1CN2c1ccc(-n2cnc(Nc3cnc(C#N)cn3)c2)cc1] | 5-((1-(4-((1R,4R)-5- Methyl-2,5- diazabicyclo[2.2.1] heptan-2-yl)phenyl)-1H- imidazol-4- yl)amino)pyrazine-2- carbonitrile | Intermediate 75; Yellow solid; Yield 5.3 mg, 4.6%; LCMS (ES⁺): 373.2 [MH]⁺; HPLC: Rt 1.30 min, 97.5% purity |
| 10 | 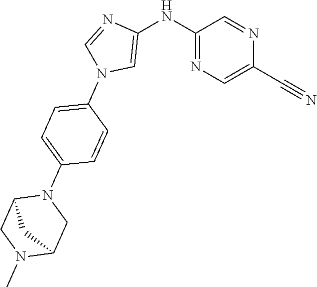 [SMILES: CN1C[C@@H]2C[C@H]1CN2c1ccc(-n2cnc(Nc3cnc(C#N)cn3)c2)cc1] | 5-((1-(4-((1S,4S)-5- Methyl-2,5- diazabicyclo[2.2.1] heptan-2-yl)phenyl)-1H- imidazol-4- yl)amino)pyrazine-2- carbonitrile | Intermediate 76; Yellow solid; Yield 4.4 mg, 3.4%; LCMS (ES⁺): 373.2 [MH]⁺; HPLC: Rt 4.19 min, 95.0% purity |
| 11 | 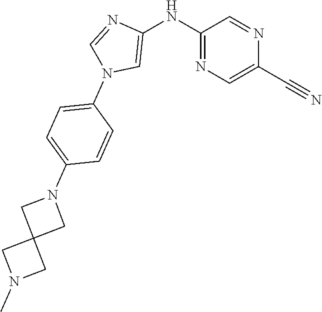 [SMILES: CN1CC2(C1)CN(c1ccc(-n3cnc(Nc4cnc(C#N)cn4)c3)cc1)C2] | 5-((1-(4-(6-Methyl-2,6- diazaspiro[3.3]heptan- 2-yl)phenyl)-1H- imidazol-4- yl)amino)pyrazine-2- carbonitrile | Intermediate 77; Yellow solid (TFA); Yield 17.0 mg, 6.5%; LCMS (ES⁺): 373.4 [MH]⁺; HPLC: Rt 0.88 min, 95.1% purity |
| 12 | 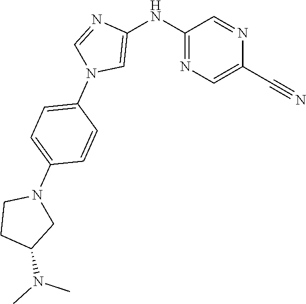 [SMILES: CN(C)[C@@H]1CCN(c2ccc(-n3cnc(Nc4cnc(C#N)cn4)c3)cc2)C1] | (R)-5-((1-(4-(3- (Dimethylamino) pyrrolidin- 1-yl)phenyl)-1H- imidazol-4- yl)amino)pyrazine-2- carbonitrile | Intermediate 165; Yellow solid (TFA); Yield 40.0 mg, 16.2%; LCMS (ES⁺): 375.1 [MH]⁺; UPLC: Rt 3.20 min, 99.4% purity |
| 13 | 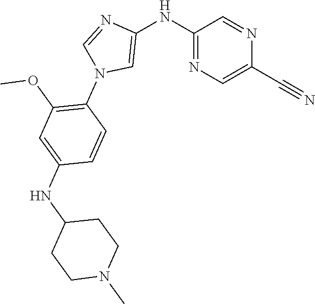 [SMILES: COc1cc(NC2CCN(C)CC2)ccc1-n1cnc(Nc2cnc(C#N)cn2)c1] | 5-((1-(2-Methoxy-4-((1- methylpiperidin-4- yl)amino)phenyl)-1H- imidazol-4- yl)amino)pyrazine-2- carbonitrile | Intermediate 167; Yellow solid; Yield 10.3 mg, 25.6%; LCMS (ES⁺): 405.2 [MH]⁺; UPLC: Rt 3.20 min, 96.6% purity |
| 14 | 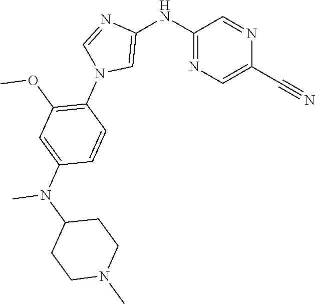 [SMILES: COc1cc(N(C)C2CCN(C)CC2)ccc1-n1cnc(Nc2cnc(C#N)cn2)c1] | 5-((1-(2-Methoxy-4- (methyl(1- methylpiperidin-4- yl)amino)phenyl)-1H- imidazol-4- yl)amino)pyrazine-2- carbonitrile | Intermediate 168; Yellow solid; Yield 2.6 mg, 9.0%; LCMS (ES⁺): 419.2 [MH]⁺; UPLC: Rt 3.38 min, 94.7% purity |
| 15 | 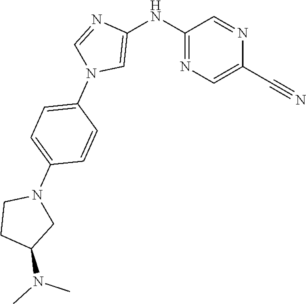 [SMILES: CN(C)[C@H]1CCN(c2ccc(-n3cnc(Nc4cnc(C#N)cn4)c3)cc2)C1] | (S)-5-((1-(4-(3- (Dimethylamino) pyrrolidin- 1-yl)phenyl)-1H- imidazol-4- yl)amino)pyrazine-2- carbonitrile | Intermediate 79; Yellow solid (TFA); Yield 12.7 mg, 2.4%; LCMS (ES⁺): 375.3 [MH]⁺; HPLC: Rt 0.73 min, 97.2% purity |
| 16 | 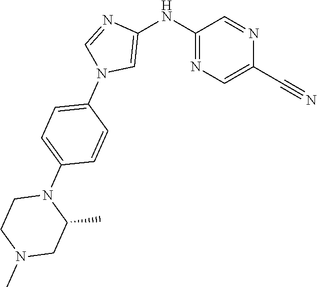 [SMILES: C[C@@H]1CN(C)CCN1c1ccc(-n2cnc(Nc3cnc(C#N)cn3)c2)cc1] | (R)-5-((1-(4-(2,4- Dimethylpiperazin-1- yl)phenyl)-1H-imidazol- 4-yl)amino)pyrazine-2- carbonitrile | Intermediate 80; Yellow solid (TFA); Yield 18.8 mg, 6.1%; LCMS (ES⁺): 375.2 [MH]⁺; HPLC: Rt 1.55 min, 95.8% purity |
| 17 | 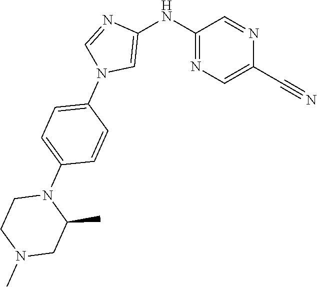 [SMILES: C[C@H]1CN(C)CCN1c1ccc(-n2cnc(Nc3cnc(C#N)cn3)c2)cc1] | (S)-5-((1-(4-(2,4- Dimethylpiperazin-1- yl)phenyl)-1H-imidazol- 4-yl)amino)pyrazine-2- carbonitrile | Intermediate 81; Yellow solid; Yield 12.6 mg, 4.0%; LCMS (ES⁺): 375.2 [MH]⁺; HPLC: Rt 0.64 min, 97.1% purity |
| 18 | 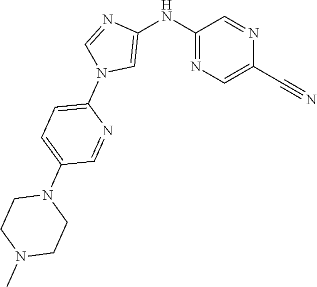 [SMILES: CN1CCN(c2ccc(-n3cnc(Nc4cnc(C#N)cn4)c3)nc2)CC1] | 5-((1-(5-(4- Methylpiperazin-1- yl)pyridin-2-yl)-1H- imidazol-4- yl)amino)pyrazine-2- carbonitrile | Intermediate 82; Yellow solid; Yield 10.0 mg, 8.9%; LCMS (ES⁺): 362.2 [MH]⁺; HPLC: Rt 0.56 min, 99.5% purity |
| 19 | 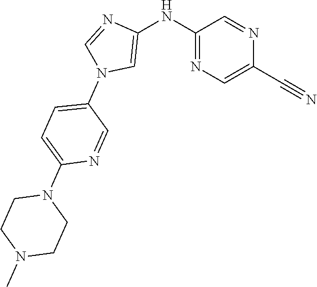 [SMILES: CN1CCN(c2ccc(-n3cnc(Nc4cnc(C#N)cn4)c3)cn2)CC1] | 5-((1-(6-(4- Methylpiperazin-1- yl)pyridin-3-yl)-1H- imidazol-4- yl)amino)pyrazine-2- carbonitrile | Intermediate 83; Yellow solid; Yield 8.6 mg, 7.4%; LCMS (ES⁺): 362.3 [MH]⁺; HPLC: Rt 0.57 min, 95.9% purity |
| 20 | 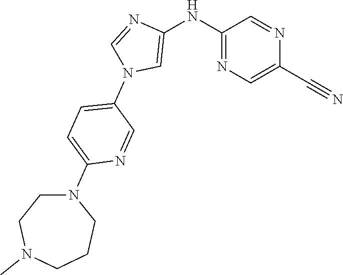 [SMILES: CN1CCCN(c2ccc(-n3cnc(Nc4cnc(C#N)cn4)c3)cn2)CC1] | 5-((1-(6-(4-methyl-1,4- Diazepan-1-yl)pyridin- 3-yl)-1H-imidazol-4- yl)amino)pyrazine-2- carbonitrile | Intermediate 26; Yellow solid; Yield 8.0 mg, 9.0%; LCMS (ES⁺): 376.3 [MH]⁺; HPLC: Rt 0.59 min, 99.7% purity |
| 21 | 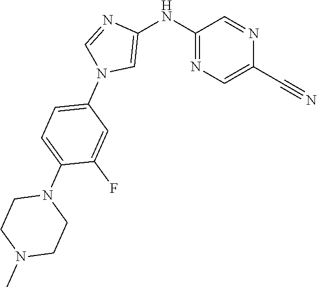 [SMILES: CN1CCN(c2ccc(-n3cnc(Nc4cnc(C#N)cn4)c3)cc2F)CC1] | 5-((1-(3-Fluoro-4-(4- methylpiperazin-1- yl)phenyl)-1H-imidazol- 4-yl)amino)pyrazine-2- carbonitrile | Intermediate 18; Yellow solid; Yield 1.5 mg, 1.3%; LCMS (ES⁺): 379.3 [MH]⁺; HPLC: Rt 0.81 min, 97.0% purity |
| 22 | 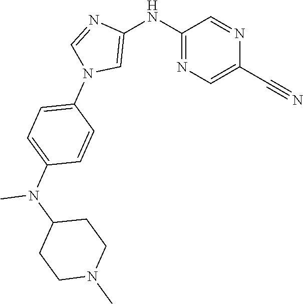 [SMILES: CN1CCC(N(C)c2ccc(-n3cnc(Nc4cnc(C#N)cn4)c3)cc2)CC1] | 5-((1-(4-(Methyl(1- methylpiperidin-4- yl)amino)phenyl)-1H- imidazol-4- yl)amino)pyrazine-2- carbonitrile | Intermediate 84; Yellow solid; Yield 16.0 mg, 12.0%; LCMS (ES⁺): 389.2 [MH]⁺; HPLC: Rt 1.49 min, 95.1% purity |
| 23 | 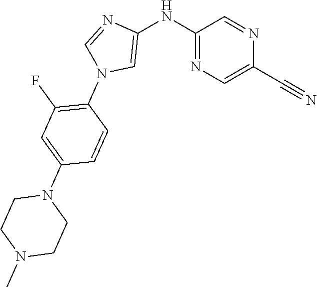 [SMILES: CN1CCN(c2ccc(-n3cnc(Nc4cnc(C#N)cn4)c3)c(F)c2)CC1] | 5-((1-(2-Fluoro-4-(4- methylpiperazin-1- yl)phenyl)-1H-imidazol- 4-yl)amino)pyrazine-2- carbonitrile | Intermediate 85; White solid; Yield 145 mg, 43.2%; LCMS (ES⁺): 379.2 [MH]⁺; HPLC: Rt 1.31 min, 97.1% purity |
| 24 | 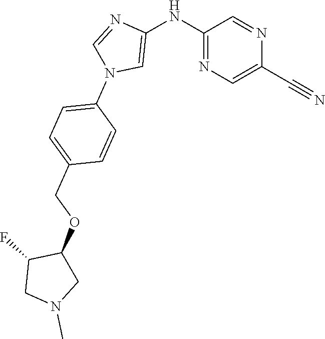 [SMILES: CN1C[C@H](OCc2ccc(-n3cnc(Nc4cnc(C#N)cn4)c3)cc2)[C@@H](F)C1] | 5-((1-(4-((((3S,4S)-4- Fluoro-1- methylpyrrolidin-3- yl)oxy)methyl)phenyl)- 1H-imidazol-4- yl)amino)pyrazine-2- carbonitrile | Intermediate 191; Yellow solid; Yield 3.1 mg, 2.8%; LCMS (ES⁺): 394.3 [MH]⁺; HPLC: Rt 0.70 min, 99.8% purity |
| 25 | 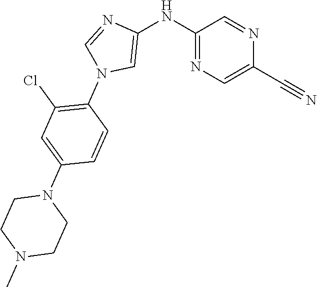 [SMILES: CN1CCN(c2ccc(-n3cnc(Nc4cnc(C#N)cn4)c3)c(Cl)c2)CC1] | 5-((1-(2-Chloro-4-(4- methylpiperazin-1- yl)phenyl)-1H-imidazol- 4-yl)amino)pyrazine-2- carbonitrile | Intermediate 86; Yellow solid; Yield 22.2 mg, 12.5%; LCMS (ES⁺): 395.2 [MH]⁺; HPLC: Rt 3.57 min, 99.4% purity |
| 26 | 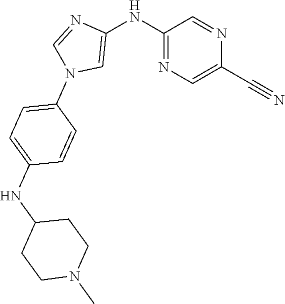 [SMILES: CN1CCC(Nc2ccc(-n3cnc(Nc4cnc(C#N)cn4)c3)cc2)CC1] | 5-((1-(4-((1- Methylpiperidin-4- yl)amino)phenyl)-1H- imidazol-4- yl)amino)pyrazine-2- carbonitrile | Intermediate 87; Red Solid; Yield 100 mg, 17.9%; LCMS (ES⁺): 375.4 [MH]⁺; HPLC: Rt 0.64 min, 99.2% purity |
| 27 | 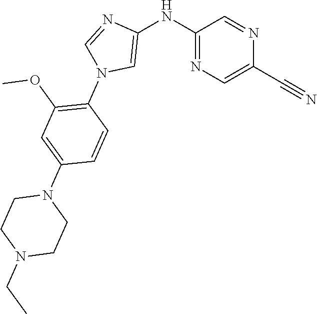 [SMILES: CCN1CCN(c2ccc(-n3cnc(Nc4cnc(C#N)cn4)c3)c(OC)c2)CC1] | 5-((1-(4-(4- Ethylpiperazin-1-yl)-2- methoxyphenyl)-1H- imidazol-4- yl)amino)pyrazine-2- carbonitrile | Intermediate 89; Yellow solid; Yield 16.5 mg, 18.1%; LCMS (ES⁺): 405.2 [MH]⁺; HPLC: Rt 0.81 min, 97.0% purity |
| 28 | 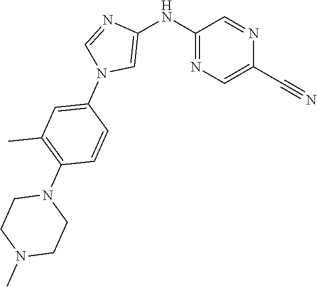 [SMILES: Cc1cc(-n2cnc(Nc3cnc(C#N)cn3)c2)ccc1N1CCN(C)CC1] | 5-((1-(3-Methyl-4-(4- methylpiperazin-1- yl)phenyl)-1H-imidazol- 4-yl)amino)pyrazine-2- carbonitrile | Intermediate 90; Yellow solid; Yield 22.2 mg, 7.5%; LCMS (ES⁺): 375.2 [MH]⁺; HPLC: Rt 0.70 min, 95.6% purity |
| 29 | 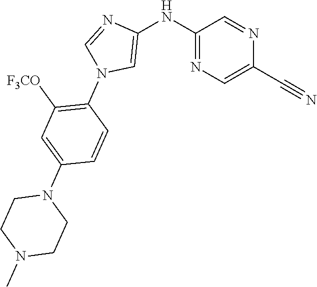 [SMILES: CN1CCN(c2ccc(-n3cnc(Nc4cnc(C#N)cn4)c3)c(OC(F)(F)F)c2)CC1] | 5-((1-(4-(4- Methylpiperazin-1-yl)- 2- (trifluoromethoxy) phenyl)-1H-imidazol-4- yl)amino)pyrazine-2- carbonitrile | Intermediate 164; Yellow solid; Yield 3.0 mg, 1.8%; LCMS (ES⁺): 445.2 [MH]⁺; HPLC: Rt 4.51 min, 97.8% purity |
| 30 | 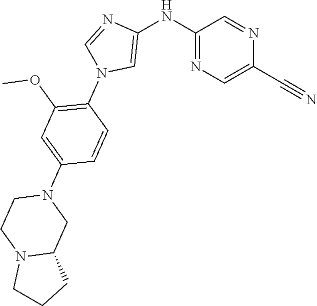 [SMILES: COc1cc(N2CCN3CCC[C@H]3C2)ccc1-n1cnc(Nc2cnc(C#N)cn2)c1] | (S)-5-((1-(4- (Hexahydropyrrolo[1,2- a]pyrazin-2(1H)-yl)-2- methoxyphenyl)-1H- imidazol-4- yl)amino)pyrazine-2- carbonitrile | Intermediate 91; Yellow solid; Yield 12.7 mg, 14.4%; LCMS (ES⁺): 417.2 [MH]⁺; HPLC: Rt 1.71 min, 93.8% purity |
| 31 | 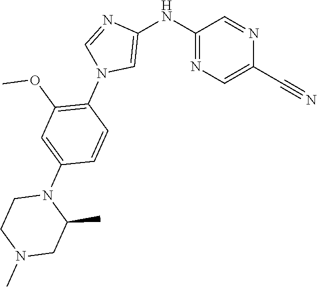 [SMILES: COc1cc(N2CCN(C)C[C@@H]2C)ccc1-n1cnc(Nc2cnc(C#N)cn2)c1] | (S)-5-((1-(4-(2,4- Dimethylpiperazin-1- yl)-2-methoxyphenyl)- 1H-imidazol-4- yl)amino)pyrazine-2- carbonitrile | Intermediate 92; Yellow solid; Yield 3.9 mg, 3.9%; LCMS (ES⁺): 405.2 [MH]⁺; HPLC: Rt 1.64 min, 95.0% purity |
| 32 | 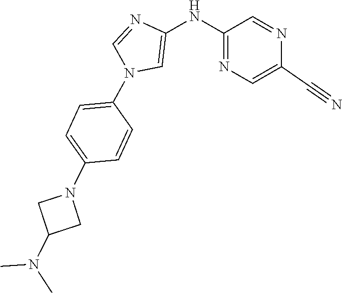 [SMILES: CN(C)C1CN(c2ccc(-n3cnc(Nc4cnc(C#N)cn4)c3)cc2)C1] | 5-((1-(4-(3- (Dimethylamino)azetidin- 1-yl)phenyl)-1H- imidazol-4- yl)amino)pyrazine-2- carbonitrile | Intermediate 93; Yellow solid; Yield 9.6 mg, 10.7%; LCMS (ES⁺): 361.2 [MH]⁺; HPLC: Rt 0.62 min, 97.0% purity |
| 33 | 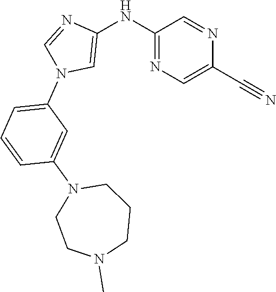 [SMILES: CN1CCCN(c2cccc(-n3cnc(Nc4cnc(C#N)cn4)c3)c2)CC1] | 5-((1-(3-(4-Methyl-1,4- diazepan-1-yl)phenyl)- 1H-imidazol-4- yl)amino)pyrazine-2- carbonitrile | Intermediate 94; Yellow solid (TFA); Yield 10.0 mg, 5.9%; LCMS (ES⁺): 375.2 [MH]⁺; HPLC: Rt 2.72 min, 98.5% purity |
| 34 | 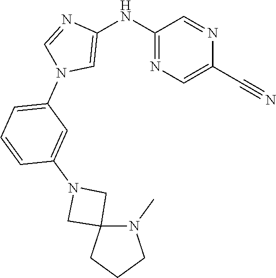 [SMILES: CN1CCCC12CN(c1cccc(-n3cnc(Nc4cnc(C#N)cn4)c3)c1)C2] | 5-((1-(3-(5-Methyl-2,5- diazaspiro[3.4]octan-2- yl)phenyl)-1H-imidazol- 4-yl)amino)pyrazine-2- carbonitrile | Intermediate 95; Yellow solid; Yield 5.7 mg, 4.6%; LCMS (ES⁺): 387.2 [MH]⁺; HPLC: Rt 1.70 min, 96.5% purity |
| 35 | 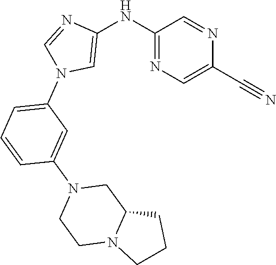 [SMILES: N#Cc1cnc(Nc2cn(-c3cccc(N4CCN5CCC[C@H]5C4)c3)cn2)cn1] | (S)-5-((1-(3- (Hexahydropyrrolo[1,2- a]pyrazin-2(1H)- yl)phenyl)-1H-imidazol- 4-yl)amino)pyrazine-2- carbonitrile | Intermediate 96; Off-white solid; Yield 10.1 mg, 7.9%; LCMS (ES⁺): 387.2 [MH]⁺; HPLC: Rt 0.95 min, 96.6% purity |
| 36 | 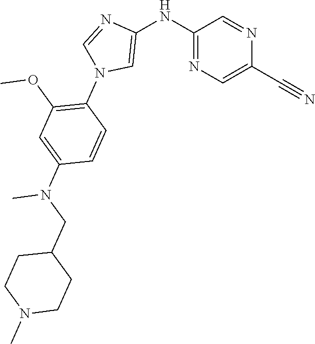 [SMILES: COc1cc(N(C)CC2CCN(C)CC2)ccc1-n1cnc(Nc2cnc(C#N)cn2)c1] | 5-((1-(2-Methoxy-4- (methyl((1- methylpiperidin-4- yl)methyl)amino)phenyl)- 1H-imidazol-4- yl)amino)pyrazine-2- carbonitrile | Intermediate 97; Yellow solid (TFA); Yield 14.1 mg, 9.6%; LCMS (ES⁺): 433.2 [MH]⁺; HPLC: Rt 0.76 min, 99.8% purity |
| 37 | 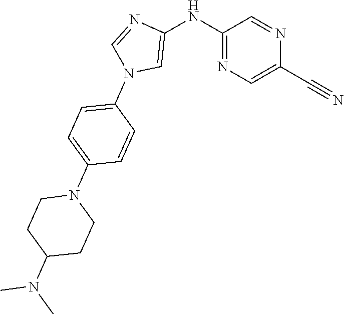 [SMILES: CN(C)C1CCN(c2ccc(-n3cnc(Nc4cnc(C#N)cn4)c3)cc2)CC1] | 5-((1-(4-(4- (Dimethylamino) piperidin- 1-yl)phenyl)-1H- imidazol-4- yl)amino)pyrazine-2- carbonitrile | Intermediate 98; Yellow solid; Yield 31.6 mg, 18.8%; LCMS (ES⁺): 389.1 [MH]⁺; HPLC: Rt 0.66 min, 99.2% purity |
| 38 | 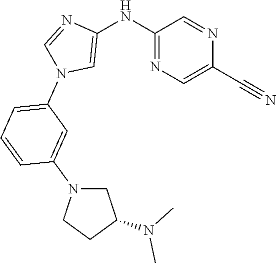 [SMILES: CN(C)[C@@H]1CCN(c2cccc(-n3cnc(Nc4cnc(C#N)cn4)c3)c2)C1] | (R)-5-((1-(3-(3- (Dimethylamino) pyrrolidin- 1-yl)phenyl)-1H- imidazol-4- yl)amino)pyrazine-2- carbonitrile | Intermediate 99; Yellow solid; Yield 7.9 mg, 8.8%; LCMS (ES⁺): 375.2 [MH]⁺; HPLC: Rt 1.71 min, 97.3% purity |
| 39 | 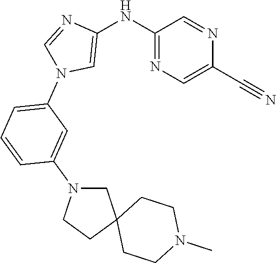 [SMILES: CN1CCC2(CC1)CCN(c1cccc(-n3cnc(Nc4cnc(C#N)cn4)c3)c1)C2] | 5-((1-(3-(8-Methyl-2,8- diazaspiro[4.5]decan- 2-yl)phenyl)-1H- imidazol-4- yl)amino)pyrazine-2- carbonitrile | Intermediate 100; Yellow solid (TFA); Yield 17.1 mg, 12.8%; LCMS (ES⁺): 415.2 [MH]⁺; HPLC: Rt 0.85 min, 98.5% purity |
| 40 | 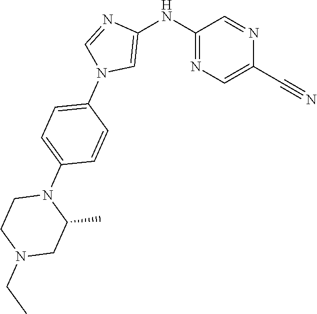 [SMILES: CCN1CCN(c2ccc(-n3cnc(Nc4cnc(C#N)cn4)c3)cc2)[C@H](C)C1] | (R)-5-((1-(4-(4-Ethyl-2- methylpiperazin-1- yl)phenyl)-1H-imidazol- 4-yl)amino)pyrazine-2- carbonitrile | Intermediate 101; Yellow solid; Yield 24.8 mg, 14.8%; LCMS (ES⁺): 389.1 [MH]⁺; HPLC: Rt 0.87 min, 98.5% purity |
| 41 | 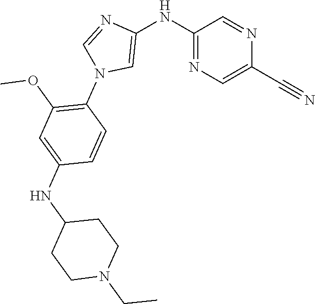 [SMILES: CCN1CCC(Nc2ccc(-n3cnc(Nc4cnc(C#N)cn4)c3)c(OC)c2)CC1] | 5-((1-(4-((1- Ethylpiperidin-4- yl)amino)-2- methoxyphenyl)-1H- imidazol-4- yl)amino)pyrazine-2- carbonitrile | Intermediate 102; Yellow solid; Yield 18.9 mg, 10.4%; LCMS (ES⁺): 419.1 [MH]⁺; HPLC: Rt 0.88 min, 97.2% purity |
| 42 | 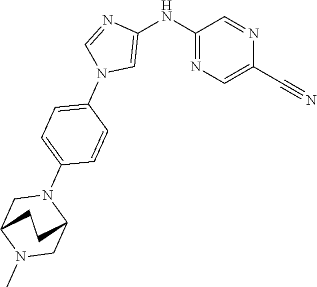 [SMILES: CN1C[C@@H]2CC[C@H]1CN2c1ccc(-n2cnc(Nc3cnc(C#N)cn3)c2)cc1] | 5-((1-(4-((1S,4S)-5- Methyl-2,5- diazabicyclo[2.2.2] octan-2-yl)phenyl)-1H- imidazol-4- yl)amino)pyrazine-2- carbonitrile | Intermediate 103; Yellow solid; Yield 26.7 mg, 16.1%; LCMS (ES⁺): 387.1 [MH]⁺; HPLC: Rt 0.77 min, 95.1% purity |
| 43 | 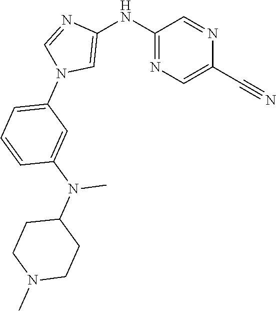 [SMILES: CN1CCC(N(C)c2cccc(-n3cnc(Nc4cnc(C#N)cn4)c3)c2)CC1] | 5-((1-(3-(Methyl(1- methylpiperidin-4- yl)amino)phenyl)-1H- imidazol-4- yl)amino)pyrazine-2- carbonitrile | Intermediate 104; Off-white solid; Yield 13.9 mg, 12.5%; LCMS (ES⁺): 389.1 [MH]⁺; HPLC: Rt 0.76 min, 95.8% purity |
| 44 | 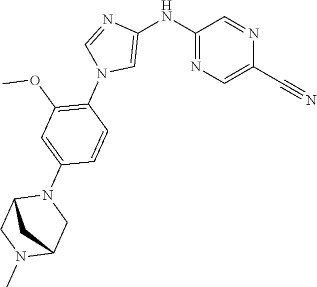 [SMILES: COc1cc(N2C[C@H]3C[C@@H]2CN3C)ccc1-n1cnc(Nc2cnc(C#N)cn2)c1] | 5-((1-(2-Methoxy-4- ((1R,4R)-5-methyl-2,5- diazabicyclo[2.2.1] heptan-2-yl)phenyl)-1H- imidazol-4- yl)amino)pyrazine-2- carbonitrile | Intermediate 105; Yellow solid (TFA); Yield 32.1 mg, 10.9%; LCMS (ES⁺): 403.3 [MH]⁺; HPLC: Rt 1.24 min, 96.9% purity |
| 45 | 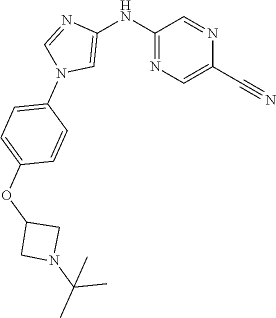 [SMILES: CC(C)(C)N1CC(Oc2ccc(-n3cnc(Nc4cnc(C#N)cn4)c3)cc2)C1] | 5-((1-(4-((1-(tert- Butyl)azetidin-3- yl)oxy)phenyl)-1H- imidazol-4- yl)amino)pyrazine-2- carbonitrile | Intermediate 66; Yellow solid; Yield 32.1 mg, 14.1%; LCMS (ES⁺): 390.2 [MH]⁺; HPLC: Rt 0.91 min, 97.6% purity |
| 46 | 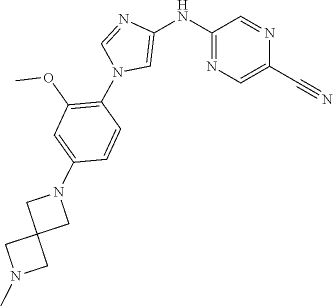 [SMILES: COc1cc(N2CC3(CN(C)C3)C2)ccc1-n1cnc(Nc2cnc(C#N)cn2)c1] | 5-((1-(2-Methoxy-4-(6- methyl-2,6- diazaspiro[3.3]heptan- 2-yl)phenyl)-1H- imidazol-4- yl)amino)pyrazine-2- carbonitrile | Intermediate 106; Yellow solid (TFA); Yield 62.3 mg, 38.1%; LCMS (ES⁺): 403.1 [MH]⁺; HPLC: Rt 0.65 min, 96.2% purity |
| 47 | 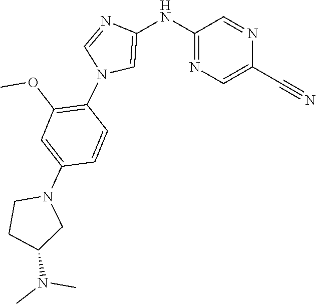 [SMILES: COc1cc(N2CC[C@@H](N(C)C)C2)ccc1-n1cnc(Nc2cnc(C#N)cn2)c1] | (R)-5-((1-(4-(3- (Dimethylamino) pyrrolidin-1-yl)-2- methoxyphenyl)-1H- imidazol-4- yl)amino)pyrazine-2- carbonitrile | Intermediate 109; Yellow solid; Yield 10.4 mg, 9.4%; LCMS (ES⁺): 405.1 [MH]⁺; HPLC: Rt 0.65 min, 97.9% purity |
| 48 | 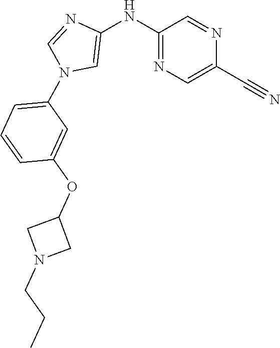 [SMILES: CCCN1CC(Oc2cccc(-n3cnc(Nc4cnc(C#N)cn4)c3)c2)C1] | 5-((1-(3-((1- Propylazetidin-3- yl)oxy)phenyl)-1H- imidazol-4- yl)amino)pyrazine-2- carbonitrile | Intermediate 68; Yellow solid; Yield 14.6 mg, 25.7%; LCMS (ES⁺): 376.1 [MH]⁺; HPLC: Rt 1.00 min, 98.3% purity |
| 49 | 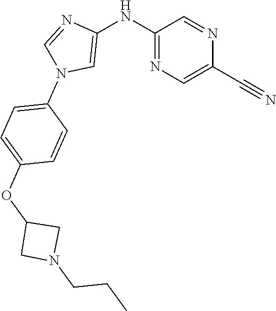 [SMILES: CCCN1CC(Oc2ccc(-n3cnc(Nc4cnc(C#N)cn4)c3)cc2)C1] | 5-((1-(4-((1- Propylazetidin-3- yl)oxy)phenyl)-1H- imidazol-4- yl)amino)pyrazine-2- carbonitrile | Intermediate 70; Yellow solid; Yield 3.2 mg, 7.9%; LCMS (ES⁺): 376.2 [MH]⁺; HPLC: Rt 1.23 min, 96.9% purity |
| 50 | 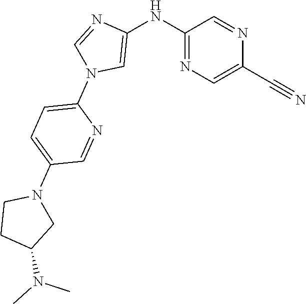 [SMILES: CN(C)[C@@H]1CCN(c2ccc(-n3cnc(Nc4cnc(C#N)cn4)c3)nc2)C1] | (R)-5-((1-(5-(3- (Dimethylamino) pyrrolidin- 1-yl)pyridin-2-yl)- 1H-imidazol-4- yl)amino)pyrazine-2- carbonitrile | Intermediate 112; Yellow solid; Yield 361 mg, 11.4%; LCMS (ES⁺): 376.2 [MH]⁺; UPLC: Rt 3.12 min, 98.8% purity |
| 51 | 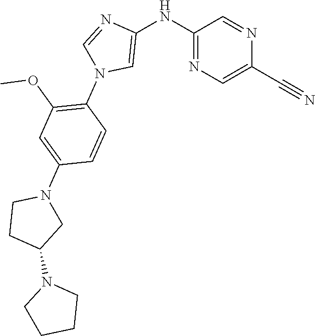 [SMILES: COc1cc(N2CC[C@@H](N3CCCC3)C2)ccc1-n1cnc(Nc2cnc(C#N)cn2)c1] | (R)-5-((1-(4-([1,3′- Bipyrrolidin]-1′-yl)-2- methoxyphenyl)-1H- imidazol-4- yl)amino)pyrazine-2- carbonitrile | Intermediate 114; Yellow solid; Yield 12.2 mg, 8.8%; LCMS (ES⁺): 431.1 [MH]⁺; HPLC: Rt 0.95 min, 95.3% purity |
| 52 | 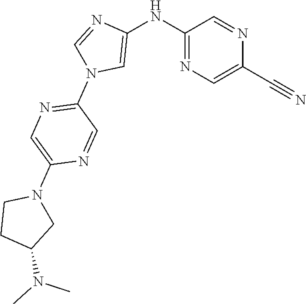 [SMILES: CN(C)[C@@H]1CCN(c2cnc(-n3cnc(Nc4cnc(C#N)cn4)c3)cn2)C1] | (R)-5-((1-(5-(3- (Dimethylamino) pyrrolidin- 1-yl)pyrazin-2-yl)- 1H-imidazol-4- yl)amino)pyrazine-2- carbonitrile | Intermediate 64; Yellow solid; Yield 3.5 mg, 3.1%; LCMS (ES⁺): 377.1 [MH]⁺; HPLC: Rt 0.64 min, 97.3% purity |
| 53 | 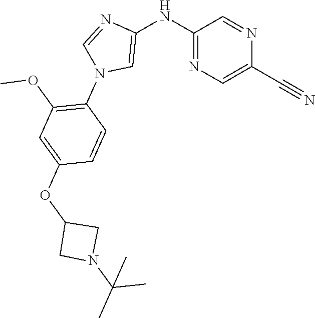 [SMILES: COc1cc(OC2CN(C(C)(C)C)C2)ccc1-n1cnc(Nc2cnc(C#N)cn2)c1] | 5-((1-(4-((1-(tert- Butyl)azetidin-3- yl)oxy)-2- methoxyphenyl)-1H- imidazol-4- yl)amino)pyrazine-2- carbonitrile | Intermediate 73; Yellow solid (FA); Yield 15.0 mg, 19.1%; LCMS (ES⁺): 420.1 [MH]⁺; HPLC: Rt 0.77 min, 99.1% purity |
| 54 | 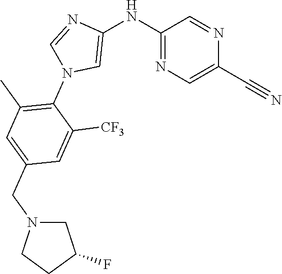 [SMILES: Cc1cc(CN2CC[C@@H](F)C2)cc(C(F)(F)F)c1-n1cnc(Nc2cnc(C#N)cn2)c1] | (R)-5-((1-(4-((3- Fluoropyrrolidin-1- yl)methyl)-2-methyl-6- (trifluoromethyl)phenyl)- 1H-imidazol-4- yl)amino)pyrazine-2- carbonitrile | Intermediate 62; Yellow solid; Yield 19.8 mg, 3.3%; LCMS (ES⁺): 446.0 [MH]⁺; HPLC: Rt 1.23 min, 99.1% purity |
| 55 | 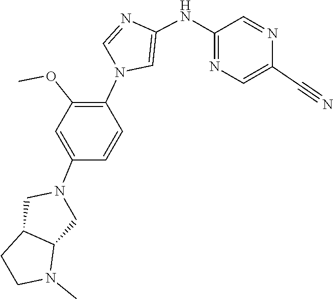 [SMILES: COc1cc(N2C[C@H]3CCN(C)[C@H]3C2)ccc1-n1cnc(Nc2cnc(C#N)cn2)c1] | 5-((1-(2-Methoxy-4- ((3aR,6aR)-1- methylhexahydropyrrolo [3,4-b]pyrrol-5(1H)- yl)phenyl)-1H-imidazol- 4-yl)amino)pyrazine-2- carbonitrile | Intermediate 115; Yellow solid; Yield 1.0 mg, 3.6%; LCMS (ES⁺): 417.1 [MH]⁺; HPLC: Rt 0.80 min, 90.8% purity |
| 56 | 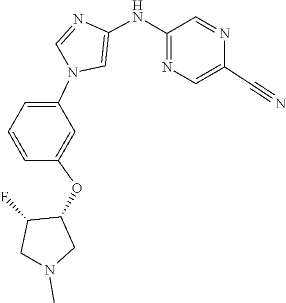 [SMILES: CN1C[C@H](F)[C@H](Oc2cccc(-n3cnc(Nc4cnc(C#N)cn4)c3)c2)C1] | 5-((1-(3-(((3R,4S)-4- fluoro-1- methylpyrrolidin-3- yl)oxy)phenyl)-1H- imidazol-4- yl)amino)pyrazine-2- carbonitrile | Intermediate 194; Yellow solid; Yield 1.1 mg, 3.0%; LCMS (ES⁺): 380.4 [MH]⁺; HPLC: Rt 0.70 min, 92.7% purity |
| 57 | 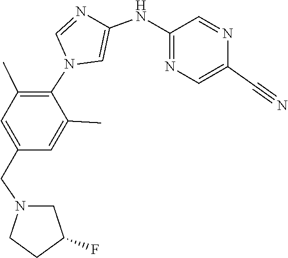 [SMILES: Cc1cc(CN2CC[C@@H](F)C2)cc(C)c1-n1cnc(Nc2cnc(C#N)cn2)c1] | (R)-5-((1-(4-((3- Fluoropyrrolidin-1- yl)methyl)-2,6- dimethylphenyl)-1H- imidazol-4- yl)amino)pyrazine-2- carbonitrile | Intermediate 63; Yellow solid; Yield 15.5 mg, 13.8%; LCMS (ES⁺): 392.2 [MH]⁺; HPLC: Rt 1.19 min, 95.9% purity |
| 58 | 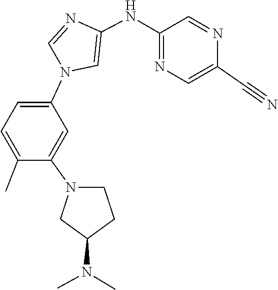 [SMILES: Cc1ccc(-n2cnc(Nc3cnc(C#N)cn3)c2)cc1N1CC[C@@H](N(C)C)C1] | (R)-5-((1-(3-(3- (Dimethylamino) pyrrolidin-1-yl)-4- methylphenyl)-1H- imidazol-4- yl)amino)pyrazine-2- carbonitrile | Intermediate 116; Yellow solid; Yield 2.1 mg, 0.8%; LCMS (ES⁺): 389.3 [MH]⁺; HPLC: Rt 1.54 min, 98.1% purity |
| 59 | 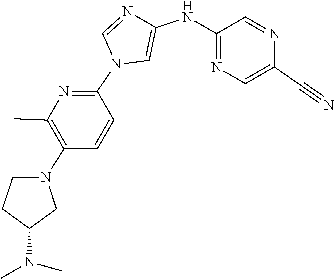 [SMILES: Cc1nc(-n2cnc(Nc3cnc(C#N)cn3)c2)ccc1N1CC[C@@H](N(C)C)C1] | (R)-5-((1-(5-(3- (Dimethylamino) pyrrolidin-1-yl)-6- methylpyridin-2-yl)-1H- imidazol-4- yl)amino)pyrazine-2- carbonitrile | Intermediate 122; Yellow solid (FA); Yield 17.4 mg, 42.5%; LCMS (ES⁺): 390.0 [MH]⁺; HPLC: Rt 0.66 min, 96.3% purity |
| 60 | 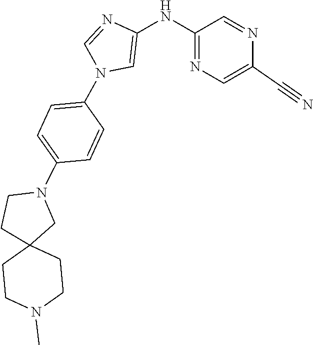 [SMILES: CN1CCC2(CC1)CCN(c1ccc(-n3cnc(Nc4cnc(C#N)cn4)c3)cc1)C2] | 5-((1-(4-(8-Methyl-2,8- diazaspiro[4.5]decan- 2-yl)phenyl)-1H- imidazol-4- yl)amino)pyrazine-2- carbonitrile | Intermediate 124; Yellow solid (TFA); Yield 12.8 mg, 4.5%; LCMS (ES⁺): 415.5 [MH]⁺; HPLC: Rt 0.74 min, 97.1% purity |
| 61 | 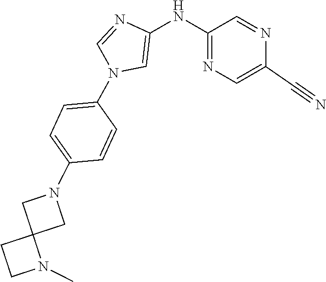 [SMILES: CN1CCC12CN(c1ccc(-n3cnc(Nc4cnc(C#N)cn4)c3)cc1)C2] | 5-((1-(4-(1-Methyl-1,6- diazaspiro[3.3]heptan- 6-yl)phenyl)-1H- imidazol-4- yl)amino)pyrazine-2- carbonitrile | Intermediate 125; Yellow solid (FA); Yield 26.5 mg, 12.4%; LCMS (ES⁺): 373.0 [MH]⁺; HPLC: Rt 0.77 min, 99.2% purity |
| 62 | 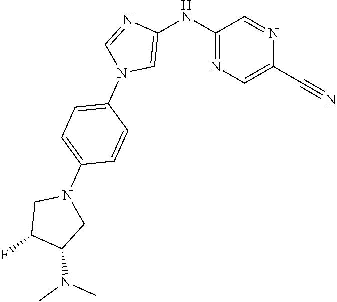 [SMILES: CN(C)[C@H]1CN(c2ccc(-n3cnc(Nc4cnc(C#N)cn4)c3)cc2)C[C@H]1F] | 5-((1-(4-((3S,4R)-3- (Dimethylamino)-4- fluoropyrrolidin-1- yl)phenyl)-1H-imidazol- 4-yl)amino)pyrazine-2- carbonitrile | Intermediate 195; Yellow solid (TFA); Yield 82.0 mg, 38.1%; LCMS (ES⁺): 393.1 [MH]⁺; HPLC: Rt 0.83 min, 96.7% purity |
| 63 | 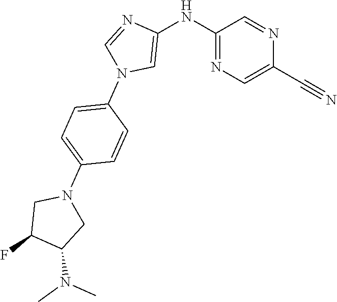 [SMILES: CN(C)[C@H]1CN(c2ccc(-n3cnc(Nc4cnc(C#N)cn4)c3)cc2)C[C@@H]1F] | 5-((1-(4-((3S,4S)-3- (Dimethylamino)-4- fluoropyrrolidin-1- yl)phenyl)-1H-imidazol- 4-yl)amino)pyrazine-2- carbonitrile | Intermediate 196; Yellow solid (FA); Yield 24.1 mg, 16.4%; LCMS (ES⁺): 393.1 [MH]⁺; HPLC: Rt 0.68 min, 95.1% purity |
| 64 | 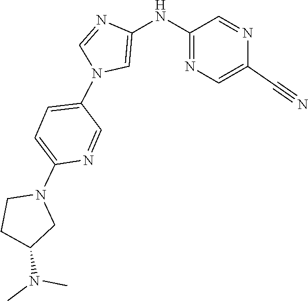 [SMILES: CN(C)[C@@H]1CCN(c2ccc(-n3cnc(Nc4cnc(C#N)cn4)c3)cn2)C1] | (R)-5-((1-(6-(3- (Dimethylamino) pyrrolidin- 1-yl)pyridin-3-yl)- 1H-imidazol-4- yl)amino)pyrazine-2- carbonitrile | Intermediate 65; Yellow solid (TFA); Yield 30.0 mg, 10.3%; LCMS (ES⁺): 376.1 [MH]⁺; HPLC: Rt 0.79 min, 98.2% purity |
| 65 | 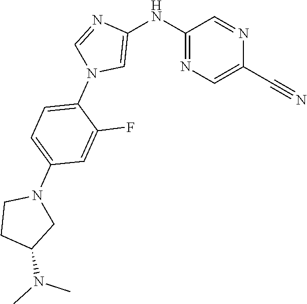 [SMILES: CN(C)[C@@H]1CCN(c2ccc(-n3cnc(Nc4cnc(C#N)cn4)c3)c(F)c2)C1] | (R)-5-((1-(4-(3- (Dimethylamino) pyrrolidin-1-yl)-2- fluorophenyl)-1H- imidazol-4- yl)amino)pyrazine-2- carbonitrile | Intermediate 126; Yellow solid (FA); Yield 25.3 mg, 12.4%; LCMS (ES⁺): 393.2 [MH]⁺; HPLC: Rt 1.00 min, 98.5% purity |
| 66 | 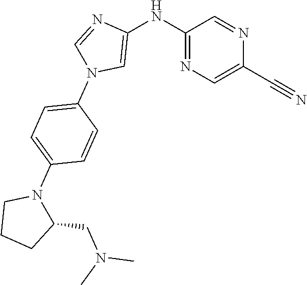 [SMILES: CN(C)C[C@@H]1CCCN1c1ccc(-n2cnc(Nc3cnc(C#N)cn3)c2)cc1] | (S)-5-((1-(4-(2- ((Dimethylamino) methyl)pyrrolidin-1- yl)phenyl)-1H-imidazol- 4-yl)amino)pyrazine-2- carbonitrile | Intermediate 170; Pale yellow solid; Yield 3.7 mg, 8.7%; LCMS (ES⁺): 389.3 [MH]⁺; UPLC: Rt 3.51 min, 99.6% purity |
| 67 | 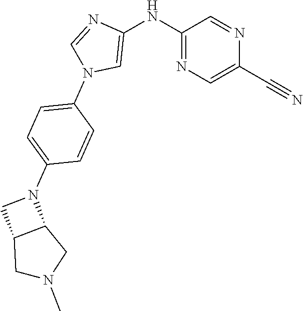 [SMILES: CN1C[C@@H]2CN(c3ccc(-n4cnc(Nc5cnc(C#N)cn5)c4)cc3)[C@@H]2C1] | 5-((1-(4-((1R,5S)-3- Methyl-3,6- diazabicyclo[3.2.0] heptan-6-yl)phenyl)-1H- imidazol-4- yl)amino)pyrazine-2- carbonitrile | Intermediate 171; Off-white solid; Yield 20.6 mg, 41.8%; LCMS (ES⁺): 373.2 [MH]⁺; UPLC: Rt 3.14 min, 99.2% purity |
| 68 | 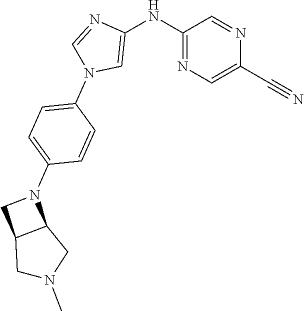 [SMILES: CN1C[C@H]2CN(c3ccc(-n4cnc(Nc5cnc(C#N)cn5)c4)cc3)[C@H]2C1] | 5-((1-(4-((1S,5R)-3- Methyl-3,6- diazabicyclo[3.2.0] heptan-6-yl)phenyl)-1H- imidazol-4- yl)amino)pyrazine-2- carbonitrile | Intermediate 172; Off-white solid; Yield 23.9 mg, 35.4%; LCMS (ES⁺): 373.2 [MH]⁺; UPLC: Rt 3.11 min, 99.6% purity |
| 69 | 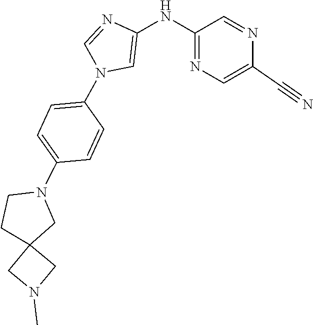 [SMILES: CN1CC2(CCN(c3ccc(-n4cnc(Nc5cnc(C#N)cn5)c4)cc3)C2)C1] | 5-((1-(4-(2-Methyl-2,6- diazaspiro[3.4]octan-6- yl)phenyl)-1H-imidazol- 4-yl)amino)pyrazine-2- carbonitrile | Intermediate 127; Yellow solid; Yield 31.5 mg, 13.7%; LCMS (ES⁺): 387.1 [MH]⁺; HPLC: Rt 0.84 min, 96.9% purity |
| 70 | 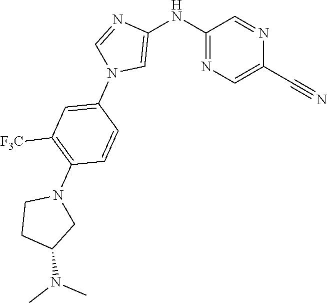 [SMILES: CN(C)[C@@H]1CCN(c2ccc(-n3cnc(Nc4cnc(C#N)cn4)c3)cc2C(F)(F)F)C1] | (R)-5-((1-(4-(3- (Dimethylamino) pyrrolidin-1-yl)-3- (trifluoromethyl)phenyl)- 1H-imidazol-4- yl)amino)pyrazine-2- carbonitrile | Intermediate 173; Light yellow solid; Yield 31.4 mg, 11.9%; LCMS (ES⁺): 443.1 [MH]⁺; UPLC: Rt 3.94 min, 99.5% purity |
| 71 | 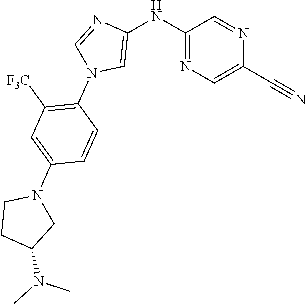 [SMILES: CN(C)[C@@H]1CCN(c2ccc(-n3cnc(Nc4cnc(C#N)cn4)c3)c(C(F)(F)F)c2)C1] | (R)-5-((1-(4-(3- (Dimethylamino) pyrrolidin-1-yl)-2- (trifluoromethyl)phenyl)- 1H-imidazol-4- yl)amino)pyrazine-2- carbonitrile | Intermediate 175; Light yellow solid; Yield 31.5 mg, 33.9%; LCMS (ES⁺): 443.1 [MH]⁺; UPLC: Rt 3.77 min, 99.8% purity |
| 72 | 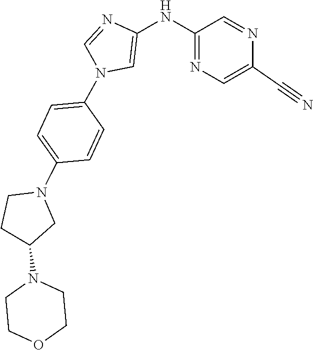 [SMILES: N#Cc1cnc(Nc2cn(-c3ccc(N4CC[C@@H](N5CCOCC5)C4)cc3)cn2)cn1] | (R)-5-((1-(4-(3- Morpholinopyrrolidin-1- yl)phenyl)-1H-imidazol- 4-yl)amino)pyrazine-2- carbonitrile | Intermediate 130; Yellow solid; Yield 78.5 mg, 94.8%; LCMS (ES⁺): 417.1 [MH]⁺; HPLC: Rt 0.74 min, 97.5% purity |
| 73 | 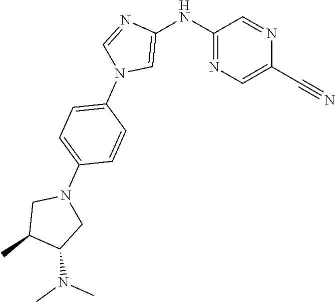 [SMILES: C[C@H]1CN(c2ccc(-n3cnc(Nc4cnc(C#N)cn4)c3)cc2)C[C@@H]1N(C)C] | 5-((1-(4-((3R,4S)-3- (Dimethylamino)-4- methylpyrrolidin-1- yl)phenyl)-1H-imidazol- 4-yl)amino)pyrazine-2- carbonitrile | Intermediate 197; Yellow solid; Yield 30.1 mg, 33.8%; LCMS (ES⁺): 389.1 [MH]⁺; HPLC: Rt 0.92 min, 99.3% purity |
| 74 | 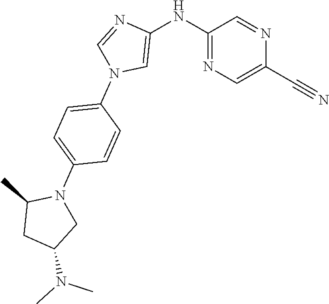 [SMILES: C[C@@H]1C[C@@H](N(C)C)CN1c1ccc(-n2cnc(Nc3cnc(C#N)cn3)c2)cc1] | 5-((1-(4-((2R,4R)-4- (Dimethylamino)-2- methylpyrrolidin-1- yl)phenyl)-1H-imidazol- 4-yl)amino)pyrazine-2- carbonitrile | Intermediate 132; Pale yellow solid; Yield 17.1 mg, 4.8%; LCMS (ES⁺): 389.2 [MH]⁺; UPLC: Rt 3.40 min, 100% purity |
| 75 | 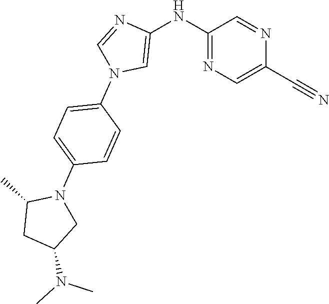 [SMILES: C[C@H]1C[C@@H](N(C)C)CN1c1ccc(-n2cnc(Nc3cnc(C#N)cn3)c2)cc1] | 5-((1-(4-((2S,4R)-4- (Dimethylamino)-2- methylpyrrolidin-1- yl)phenyl)-1H-imidazol- 4-yl)amino)pyrazine-2- carbonitrile | Intermediate 198; Yellow solid; Yield 17.8 mg, 10.6%; LCMS (ES⁺): 389.0 [MH]⁺; HPLC: Rt 0.87 min, 97.1% purity |
| 76 | 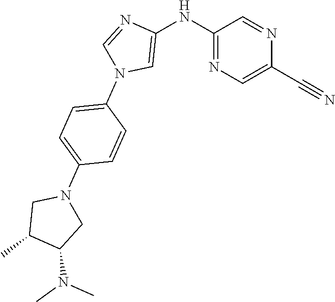 [SMILES: C[C@@H]1CN(c2ccc(-n3cnc(Nc4cnc(C#N)cn4)c3)cc2)C[C@@H]1N(C)C] | 5-((1-(4-((3R,4R)-3- (Dimethylamino)-4- methylpyrrolidin-1- yl)phenyl)-1H-imidazol- 4-yl)amino)pyrazine-2- carbonitrile | Intermediate 199; Yellow solid (TFA); Yield 16.4 mg, 12.7%; LCMS (ES⁺): 389.3 [MH]⁺; HPLC: Rt 1.80 min, 96.0% purity |
| 77 | 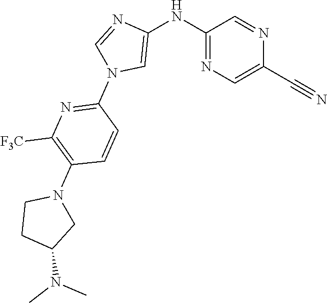 [SMILES: CN(C)[C@@H]1CCN(c2ccc(-n3cnc(Nc4cnc(C#N)cn4)c3)nc2C(F)(F)F)C1] | (R)-5-((1-(5-(3- (Dimethylamino) pyrrolidin-1-yl)-6- (trifluoromethyl)pyridin- 2-yl)-1H-imidazol-4- yl)amino)pyrazine-2- carbonitrile | Intermediate 176; Yellow solid (FA); Yield 4.6 mg, 4.8%; LCMS (ES⁺): 444.1 [MH]⁺; UPLC: Rt 4.03 min, 97.0% purity |
| 78 | 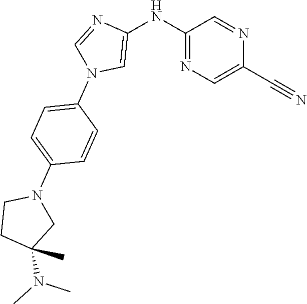 [SMILES: CN(C)[C@]1(C)CCN(c2ccc(-n3cnc(Nc4cnc(C#N)cn4)c3)cc2)C1] | (R)-5-((1-(4-(3- (Dimethylamino)-3- methylpyrrolidin-1- yl)phenyl)-1H-imidazol- 4-yl)amino)pyrazine-2- carbonitrile | Intermediate 200; Yellow solid; Yield 22.7 mg, 15.1%; LCMS (ES⁺): 389.1 [MH]⁺; HPLC: Rt 0.86 min, 96.5% purity |
| 79 | 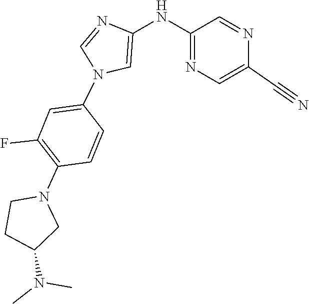 [SMILES: CN(C)[C@@H]1CCN(c2ccc(-n3cnc(Nc4cnc(C#N)cn4)c3)cc2F)C1] | (R)-5-((1-(4-(3- (Dimethylamino) pyrrolidin-1-yl)-3- fluorophenyl)-1H- imidazol-4- yl)amino)pyrazine-2- carbonitrile | Intermediate 135; Yellow solid (TFA); Yield 64.8 mg, 30.1%; LCMS (ES⁺): 393.0 [MH]⁺; HPLC: Rt 0.89 min, 97.0% purity |
| 80 | 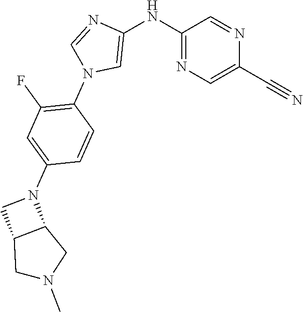 [SMILES: CN1C[C@@H]2CN(c3ccc(-n4cnc(Nc5cnc(C#N)cn5)c4)c(F)c3)[C@@H]2C1] | 5-((1-(2-Fluoro-4- ((1R,5S)-3-methyl-3,6- diazabicyclo[3.2.0] heptan-6-yl)phenyl)-1H- imidazol-4- yl)amino)pyrazine-2- carbonitrile | Intermediate 177; Pale yellow solid; Yield 2.9 mg, 9.5%; LCMS (ES⁺): 391.2 [MH]⁺; UPLC: Rt 3.28 min, 99.5% purity |
| 81 | 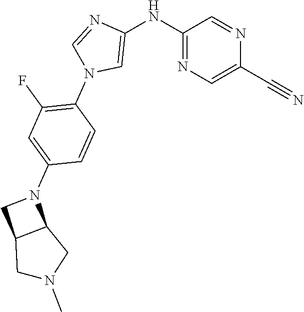 [SMILES: CN1C[C@H]2CN(c3ccc(-n4cnc(Nc5cnc(C#N)cn5)c4)c(F)c3)[C@H]2C1] | 5-((1-(2-Fluoro-4- ((1S,5R)-3-methyl-3,6- diazabicyclo[3.2.0] heptan-6-yl)phenyl)-1H- imidazol-4- yl)amino)pyrazine-2- carbonitrile | Intermediate 178; Pale yellow solid; Yield 9.0 mg, 22.8%; LCMS (ES⁺): 391.2 [MH]⁺; UPLC: Rt 3.29 min, 100% purity |
| 82 | 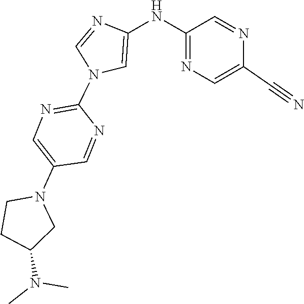 [SMILES: CN(C)[C@@H]1CCN(c2cnc(-n3cnc(Nc4cnc(C#N)cn4)c3)nc2)C1] | (R)-5-((1-(5-(3- (Dimethylamino) pyrrolidin- 1-yl)pyrimidin-2-yl)- 1H-imidazol-4- yl)amino)pyrazine-2- carbonitrile | Intermediate 136; Yellow green solid; Yield 4.2 mg, 3.7%; LCMS (ES⁺): 377.4 [MH]⁺; HPLC: Rt 0.81 min, 99.2% purity |
| 83 | 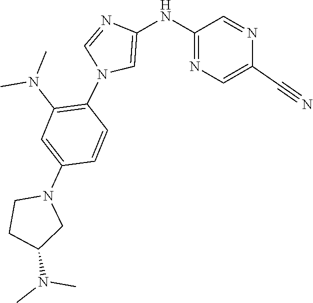 [SMILES: CN(C)c1cc(N2CC[C@@H](N(C)C)C2)ccc1-n1cnc(Nc2cnc(C#N)cn2)c1] | (R)-5-((1-(2- (Dimethylamino)-4-(3- (dimethylamino) pyrrolidin- 1-yl)phenyl)-1H- imidazol-4- yl)amino)pyrazine-2- carbonitrile | Intermediate 182; White solid (TFA); Yield 110 mg, 29.3%; LCMS (ES⁺): 417.5 [MH]⁺; HPLC: Rt 0.81 min, 99.5% purity |
| 84 | 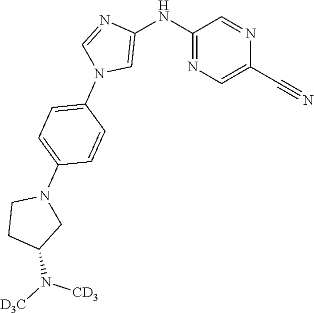 [SMILES: [2H]C([2H])([2H])N([C@@H]1CCN(c2ccc(-n3cnc(Nc4cnc(C#N)cn4)c3)cc2)C1)C([2H])([2H])[2H]] | (R)-5-((1-(4-(3- (Bis(methyl- d3)amino)pyrrolidin-1- yl)phenyl)-1H-imidazol- 4-yl)amino)pyrazine-2- carbonitrile | Intermediate 183; Pale yellow solid; Yield 9.5 mg, 33.6%; LCMS (ES⁺): 381.3 [MH]⁺; UPLC: Rt 3.19 min, 99.1% purity |
| 85 | 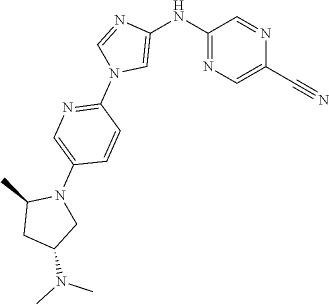 [SMILES: C[C@@H]1C[C@@H](N(C)C)CN1c1ccc(-n2cnc(Nc3cnc(C#N)cn3)c2)nc1] | 5-((1-(5-((2R,4R)-4- (Dimethylamino)-2- methylpyrrolidin-1- yl)pyridin-2-yl)-1H- imidazol-4- yl)amino)pyrazine-2- carbonitrile | Intermediate 138; Pale yellow solid; Yield 14.0 mg, 29.8%; LCMS (ES⁺): 390.0 [MH]⁺; UPLC: Rt 3.35 min, 100% purity |
| 86 | 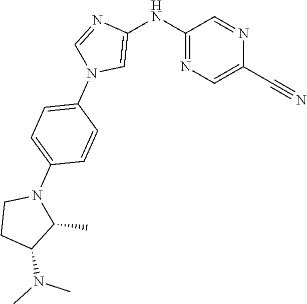 [SMILES: C[C@@H]1[C@H](N(C)C)CCN1c1ccc(-n2cnc(Nc3cnc(C#N)cn3)c2)cc1] | 5-((1-(4-((2R,3R)-3- (Dimethylamino)-2- methylpyrrolidin-1- yl)phenyl)-1H-imidazol- 4-yl)amino)pyrazine-2- carbonitrile | Intermediate 189; Yellow solid (FA); Yield 18.0 mg, 27.2%; LCMS (ES⁺): 389.1 [MH]⁺; HPLC: Rt 1.46 min, 97.0% purity |
| 87 | 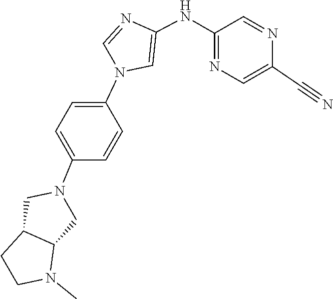 [SMILES: CN1CC[C@@H]2CN(c3ccc(-n4cnc(Nc5cnc(C#N)cn5)c4)cc3)C[C@@H]21] | 5-((1-(4-((3aR,6aR)-1- Methylhexahydropyrrolo [3,4-b]pyrrol-5(1H)- yl)phenyl)-1H-imidazol- 4-yl)amino)pyrazine-2- carbonitrile | Intermediate 141; White solid; Yield 2.8 mg, 2.4%; LCMS (ES⁺): 387.0 [MH]⁺; HPLC: Rt 1.50 min, 95.1% purity |
| 88 | 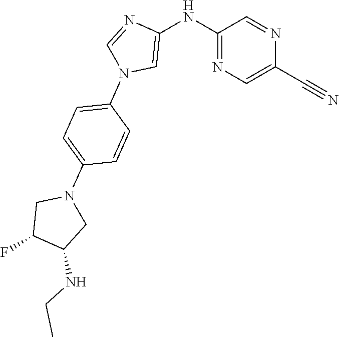 [SMILES: CCN[C@H]1CN(c2ccc(-n3cnc(Nc4cnc(C#N)cn4)c3)cc2)C[C@H]1F] | 5-((1-(4-((3S,4R)-3- (Ethylamino)-4- fluoropyrrolidin-1- yl)phenyl)-1H-imidazol- 4-yl)amino)pyrazine-2- carbonitrile | Intermediate 201; Yellow solid (TFA); Yield 18.9 mg, 12.8%; LCMS (ES⁺): 393.0 [MH]⁺; HPLC: Rt 1.52 min, 97.8% purity |
| 89 | 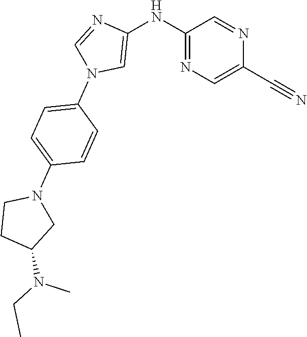 [SMILES: CCN(C)[C@@H]1CCN(c2ccc(-n3cnc(Nc4cnc(C#N)cn4)c3)cc2)C1] | (R)-5-((1-(4-(3- (Ethyl(methyl)amino) pyrrolidin-1-yl)phenyl)- 1H-imidazol-4- yl)amino)pyrazine-2- carbonitrile | Intermediate 202; Yellow solid; Yield 8.8 mg, 10.4%; LCMS (ES⁺): 389.1 [MH]⁺; HPLC: Rt 1.84 min, 99.6% purity |
| 90 | 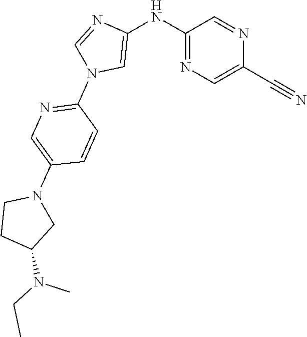 [SMILES: CCN(C)[C@@H]1CCN(c2ccc(-n3cnc(Nc4cnc(C#N)cn4)c3)nc2)C1] | (R)-5-((1-(5-(3- (Ethyl(methyl)amino) pyrrolidin-1-yl)pyridin-2- yl)-1H-imidazol-4- yl)amino)pyrazine-2- carbonitrile | Intermediate 203; Yellow solid (TFA); Yield 4.0 mg, 4.7%; LCMS (ES⁺): 390.1 [MH]⁺; HPLC: Rt 1.48 min, 96.0% |
| 91 | 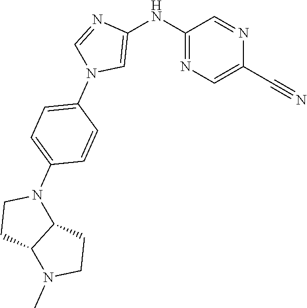 [SMILES: CN1CC[C@@H]2[C@H]1CCN2c1ccc(-n2cnc(Nc3cnc(C#N)cn3)c2)cc1] | 5-((1-(4-((3aR,6aR)-4- Methylhexahydropyrrolo [3,2-b]pyrrol-1(2H)- yl)phenyl)-1H-imidazol- 4-yl)amino)pyrazine-2- carbonitrile | Intermediate 190; Yellow solid (TFA); Yield 63.5 mg, 30.2%; LCMS (ES⁺): 387.1 [MH]⁺; HPLC: Rt 0.73 min, 96.1% purity |
| 92 | 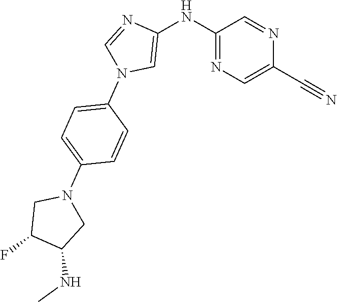 [SMILES: CN[C@H]1CN(c2ccc(-n3cnc(Nc4cnc(C#N)cn4)c3)cc2)C[C@H]1F] | 5-((1-(4-((3R,4S)-3- Fluoro-4- (methylamino)pyrrolidin- 1-yl)phenyl)-1H- imidazol-4- yl)amino)pyrazine-2- carbonitrile | Intermediate 184; Yellow solid; Yield: 65.0 mg, 26.4%; LCMS (ES⁺): 379.0 [MH]⁺; HPLC: Rt 0.67 min, 98.2% purity |
| 93 | 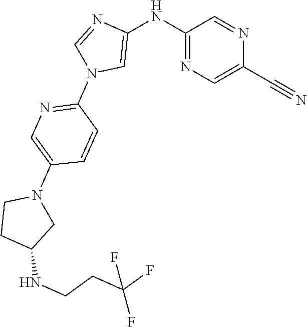 [SMILES: N#Cc1cnc(Nc2cn(-c3ccc(N4CC[C@@H](NCCC(F)(F)F)C4)cn3)cn2)cn1] | (R)-5-((1-(5-(3-((3,3,3- Trifluoropropyl)amino) pyrrolidin-1-yl)pyridin-2- yl)-1H-imidazol-4- yl)amino)pyrazine-2- carbonitrile | Intermediate 204; Yellow solid; Yield 50.5 mg, 27.1%; LCMS (ES⁺): 444.0 [MH]⁺; HPLC: Rt 1.62 min, 98.5% purity |
| 94 | 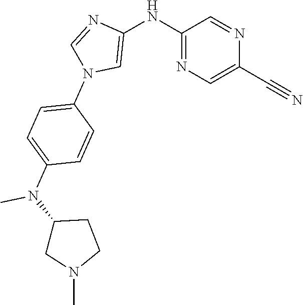 [SMILES: CN1CC[C@@H](N(C)c2ccc(-n3cnc(Nc4cnc(C#N)cn4)c3)cc2)C1] | (R)-5-((1-(4-(Methyl(1- methylpyrrolidin-3- yl)amino)phenyl)-1H- imidazol-4- yl)amino)pyrazine-2- carbonitrile | Intermediate 205; Yellow solid (TFA); Yield 10.1 mg, 3.5%; LCMS (ES⁺): 375.0 [MH]⁺; HPLC: Rt 0.89 min, 99.3% purity |
| 95 | 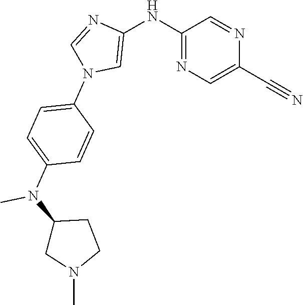 [SMILES: CN1CC[C@H](N(C)c2ccc(-n3cnc(Nc4cnc(C#N)cn4)c3)cc2)C1] | (S)-5-((1-(4-(Methyl(1- methylpyrrolidin-3- yl)amino)phenyl)-1H- imidazol-4- yl)amino)pyrazine-2- carbonitrile | Intermediate 206; Yellow solid (TFA); Yield 13.2 mg, 6.4%; LCMS (ES⁺): 375.0 [MH]⁺; HPLC: Rt 1.56 min, 97.7% purity |
| 96 | 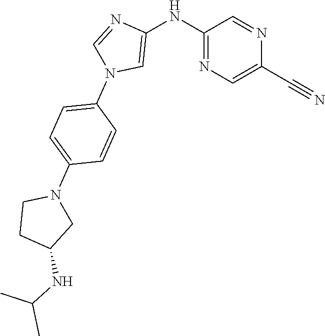 [SMILES: CC(C)N[C@@H]1CCN(c2ccc(-n3cnc(Nc4cnc(C#N)cn4)c3)cc2)C1] | (R)-5-((1-(4-(3- (Isopropylamino) pyrrolidin- 1-yl)phenyl)-1H- imidazol-4- yl)amino)pyrazine-2- carbonitrile | Intermediate 207; Yellow solid (TFA); Yield 4.8 mg, 3.3%; LCMS (ES⁺): 389.1 [MH]⁺; HPLC: Rt 0.83 min, 97.7% purity |
| 97 | 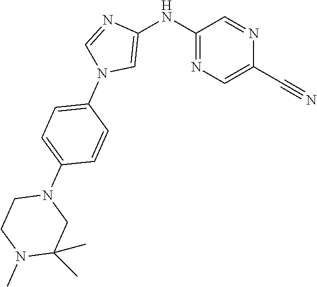 [SMILES: CN1CCN(c2ccc(-n3cnc(Nc4cnc(C#N)cn4)c3)cc2)CC1(C)C] | 5-((1-(4-(3,3,4- Trimethylpiperazin-1- yl)phenyl)-1H-imidazol- 4-yl)amino)pyrazine-2- carbonitrile | Intermediate 150; Yellow solid (TFA); Yield 22.0 mg, 12.1%; LCMS (ES⁺): 389.4 [MH]⁺; HPLC: Rt 0.79 min, 98.8% purity |
| 98 | 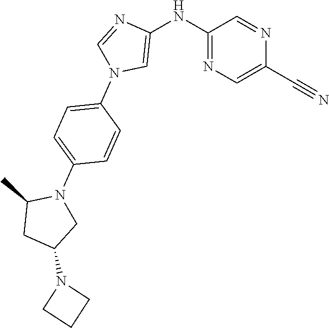 [SMILES: C[C@@H]1C[C@@H](N2CCC2)CN1c1ccc(-n2cnc(Nc3cnc(C#N)cn3)c2)cc1] | 5-((1-(4-((2R,4R)-4- (Azetidin-1-yl)-2- methylpyrrolidin-1- yl)phenyl)-1H-imidazol- 4-yl)amino)pyrazine-2- carbonitrile | Intermediate 151; Off-white solid; Yield 70.3 mg, 17.4%; LCMS (ES⁺): 401.2 [MH]⁺; HPLC: Rt 3.45 min, 100% purity |
| 99 | 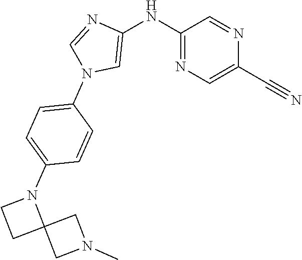 [SMILES: CN1CC2(CCN2c2ccc(-n3cnc(Nc4cnc(C#N)cn4)c3)cc2)C1] | 5-((1-(4-(6-Methyl-1,6- diazaspiro[3.3]heptan- 1-yl)phenyl)-1H- imidazol-4- yl)amino)pyrazine-2- carbonitrile | Intermediate 208; Yellow solid; Yield 22.6 mg, 6.5%; LCMS (ES⁺): 373.4 [MH]⁺; HPLC: Rt 0.73 min, 97.2% purity |
| 100 | 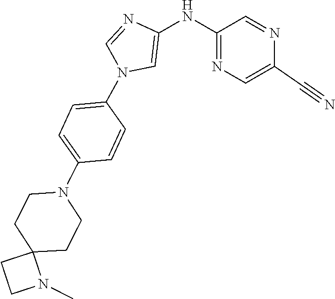 [SMILES: CN1CCC12CCN(c1ccc(-n3cnc(Nc4cnc(C#N)cn4)c3)cc1)CC2] | 5-((1-(4-(1-Methyl-1,7- diazaspiro[3.5]nonan- 7-yl)phenyl)-1H- imidazol-4- yl)amino)pyrazine-2- carbonitrile | Intermediate 153; Yellow solid (TFA); Yield 27.4 mg, 19.0%; LCMS (ES⁺): 401.4 [MH]⁺; HPLC: Rt 0.77 min, 98.8% purity |
| 101 | 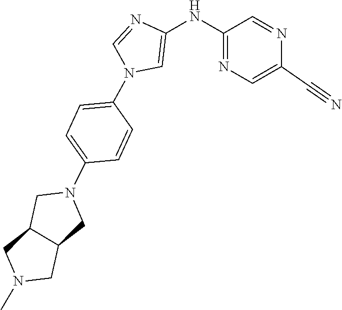 [SMILES: CN1C[C@@H]2CN(c3ccc(-n4cnc(Nc5cnc(C#N)cn5)c4)cc3)C[C@@H]2C1] | 5-((1-(4-((3aR,6aS)-5- Methylhexahydropyrrolo [3,4-c]pyrrol-2(1H)- yl)phenyl)-1H-imidazol- 4-yl)amino)pyrazine-2- carbonitrile | Intermediate 180; Off-white solid; Yield 76.4 mg, 38.3%; LCMS (ES⁺): 387.2 [MH]⁺; HPLC: Rt 3.30 min, 100% purity |
| 102 | 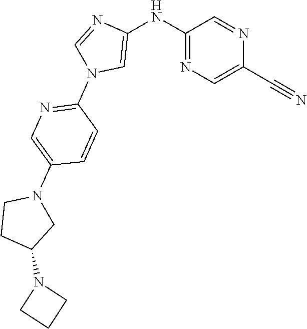 [SMILES: N#Cc1cnc(Nc2cn(-c3ccc(N4CC[C@@H](N5CCC5)C4)cn3)cn2)cn1] | (R)-5-((1-(5-(3- (Azetidin-1- yl)pyrrolidin-1- yl)pyridin-2-yl)-1H- imidazol-4- yl)amino)pyrazine-2- carbonitrile | Intermediate 159; Yellow solid; Yield 16.2 mg, 11.0%; LCMS (ES⁺): 388.2 [MH]⁺; HPLC: Rt 0.59 min, 95.6% purity |
| 103 | 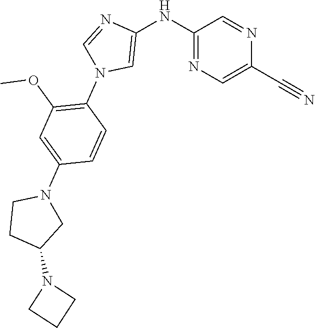 [SMILES: COc1cc(N2CC[C@@H](N3CCC3)C2)ccc1-n1cnc(Nc2cnc(C#N)cn2)c1] | (R)-5-((1-(4-(3- (Azetidin-1- yl)pyrrolidin-1-yl)-2- methoxyphenyl)-1H- imidazol-4- yl)amino)pyrazine-2- carbonitrile | Intermediate 160; Yellow solid; Yield 27.5 mg, 12.5%; LCMS (ES⁺): 417.2 [MH]⁺; HPLC: Rt 1.68 min, 98.3% purity |
| 104 | 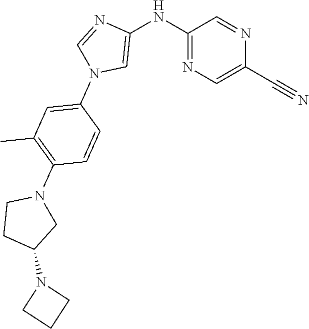 [SMILES: Cc1cc(-n2cnc(Nc3cnc(C#N)cn3)c2)ccc1N1CC[C@@H](N2CCC2)C1] | (R)-5-((1-(4-(3- (Azetidin-1- yl)pyrrolidin-1-yl)-3- methylphenyl)-1H- imidazol-4- yl)amino)pyrazine-2- carbonitrile | Intermediate 161; Yellow solid (FA); Yield 16.0 mg, 13.0%; LCMS (ES⁺): 401.2 [MH]⁺; HPLC: Rt 1.40 min, 98.3% purity |

### Examples 105 to 126

Examples 105 to 126 were prepared similarly to Example 1, via AdBrettPhos Pd G3-catalysed Buchwald-Hartwig coupling of the specified aryl bromide Intermediate with 5-aminopyrazine-2-carbonitrile, but with a subsequent enantiomer separation step using either prep-SFC or chiral prep-HPLC to isolate single enantiomers of unknown absolute configuration; see Table 11 below.

TABLE 11
AdBrettPhos Pd G3-catalysed Buchwald-Hartwig coupling
reactions then enantiomer separation

| | | | Enantiomer | Intermediate(s) used; |
|---|---|---|---|---|
| | | | separation | Form (salt); Yield; LCMS; |
| Ex. | Structure | Name | method | HPLC |
| 105 | 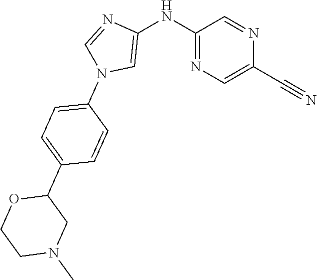 [SMILES: CN1CCOC(c2ccc(-n3cnc(Nc4cnc(C#N)cn4)c3)cc2)C1] | 5-((1-(4-(4- Methyl- morpholin-2- yl)phenyl)- 1H-imidazol- 4-yl)amino) pyrazine-2- carbonitrile | Enantiomer 1 Prep-SFC Method 3 CRt: 10.58 min | Intermediate 23; Yellow solid; Yield 2.2 mg, 1.5%; LCMS (ES⁺): 362.3 [MH]⁺; HPLC: Rt 0.69 min, 99.0% purity |
| 106 | | | Enantiomer 2 | Intermediate 23; |
| | | | Prep-SFC | Yellow solid; |
| | | | Method 3 | Yield 3.5 mg, 1.0%; |
| | | | Rt: 23.08 min | LCMS (ES⁺): |
| | | | | 362.3 [MH]⁺; |
| | | | | HPLC: Rt 0.69 |
| | | | | min, 99.0% |
| | | | | purity |
| 107 | 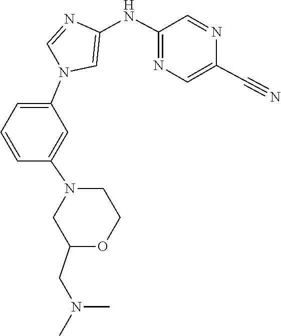 [SMILES: CN(C)CC1CN(c2cccc(-n3cnc(Nc4cnc(C#N)cn4)c3)c2)CCO1] | 5-((1-(3-(2- ((Dimethyl- amino)methyl) morpholino) phenyl)-1H- imidazol-4- yl)amino) pyrazine-2- carbonitrile | Enantiomer 1 Prep-SFC Method 4 Rt: 11.86 min | Intermediate 108; Yellow solid; Yield 20.9 mg, 4.6%; LCMS (ES⁺): 405.1 [MH]⁺; HPLC: Rt 0.75 min, 98.6% purity |
| 108 | | | Enantiomer 2 | Intermediate 108; |
| | | | Prep-SFC | Yellow solid; |
| | | | Method 4 | Yield 18.7 mg, 8.8%; |
| | | | Rt: 20.69 min | LCMS (ES⁺): 405.1 |
| | | | | [MH]⁺; HPLC: Rt |
| | | | | 0.74 min, 99.1% |
| | | | | purity |
| 109 | 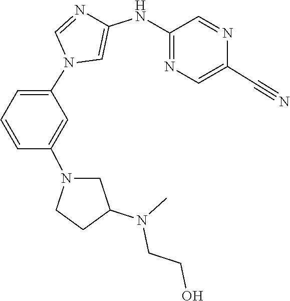 [SMILES: CN(CCO)C1CCN(c2cccc(-n3cnc(Nc4cnc(C#N)cn4)c3)c2)C1] | 5-((1-(3-(3- ((2-Hydroxyethyl) (methyl)amino) pyrrolidin-1-yl) phenyl)-1H- imidazol-4- yl)amino) pyrazine-2- carbonitrile | HPLC Method 4 Enantiomer 1 Chiral Prep- Rt: 6.43 min | Intermediate 111; Yellow solid; Yield 2.2 mg, 2.5%; LCMS (ES⁺): 405.1 [MH]⁺; HPLC: Rt 0.74 min, 99.6% purity |
| 110 | | | Enantiomer 2 | Intermediate 111; |
| | | | Chiral Prep- | Yellow solid; |
| | | | HPLC Method 4 | Yield 1.7 mg, 1.9%; |
| | | | Rt: 8.24min | LCMS (ES⁺): |
| | | | | 405.1 [MH]⁺; |
| | | | | HPLC: Rt 1.11 |
| | | | | min, 98.8% |
| | | | | purity |
| 111 | 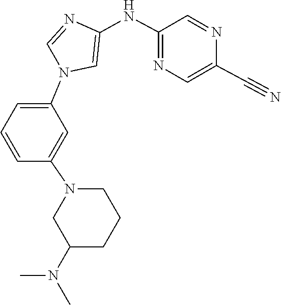 [SMILES: CN(C)C1CCCN(c2cccc(-n3cnc(Nc4cnc(C#N)cn4)c3)c2)C1] | 5-((1-(3-(3- (Dimethylamino) piperidin- 1-yl)phenyl)- 1H-imidazol- 4-yl)amino) pyrazine-2- carbonitrile | Enantiomer 1 Chiral Prep- HPLC Method 5 Rt: 1.56 min | Intermediate 113; Yellow solid; Yield 22.8 mg, 6.2%; LCMS (ES⁺): 389.1 [MH]⁺; HPLC: Rt 0.80 min, 99.1% purity |
| 112 | | | Enantiomer 2 | Intermediate 113; |
| | | | Chiral Prep- | Yellow solid; |
| | | | HPLC Method 5 | Yield 21.7 mg, 5.9%; |
| | | | Rt: 2.29 min | LCMS (ES+): 389.1 |
| | | | | [MH]⁺; HPLC: Rt |
| | | | | 0.80 min, 99.0% |
| | | | | purity |
| 113 | 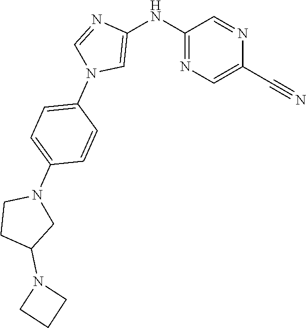 [SMILES: N#Cc1cnc(Nc2cn(-c3ccc(N4CCC(N5CCC5)C4)cc3)cn2)cn1] | 5-((1-(4-(3- (Azetidin-1- yl)pyrrolidin- 1-yl)phenyl)- 1H-imidazol- 4-yl)amino) pyrazine-2- carbonitrile | Enantiomer 1 Chiral Prep- HPLC Method 6 Rt: 18.32 min | Intermediate 142; Yellow solid; Yield 2.30 mg, 0.6%; LCMS (ES⁺): 387.0 [MH]⁺; HPLC: Rt 1.72 min, 98.4% purity |
| 114 | | | Enantiomer 2 | Intermediate 142; |
| | | | Chiral Prep- | Yellow solid; |
| | | | HPLC Method 6 | Yield 2.8 mg, 0.7%; |
| | | | Rt: 28.16 min | LCMS (ES⁺): |
| | | | | 387.1 [MH]⁺; |
| | | | | HPLC: Rt 0.80 |
| | | | | min, 97.3% |
| | | | | purity |
| 115 | 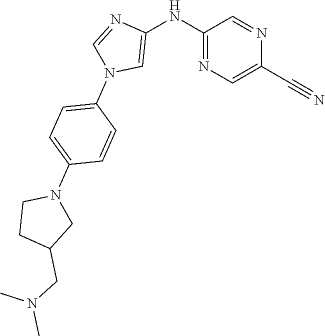 [SMILES: CN(C)CC1CCN(c2ccc(-n3cnc(Nc4cnc(C#N)cn4)c3)cc2)C1] | 5-((1-(4-(3- ((Dimethylamino) methyl) pyrrolidin-1- yl)phenyl)- 1H-imidazol- 4-yl)amino) pyrazine-2- carbonitrile | Enantiomer 1 Prep-SFC Method 5 Rt: 27.05 min | Intermediate 145; Yellow solid; Yield: 1.1 mg, 0.7%; LCMS (ES⁺): 389.0 [MH]⁺; HPLC: Rt 0.86 min, 95.3% purity |
| 116 | | | Enantiomer 2 | Intermediate 145; |
| | | | Prep-SFC | Yellow solid; |
| | | | Method 5 | Yield: 0.4 mg, 0.3%; |
| | | | Rt: 37.53 min | LCMS (ES⁺): |
| | | | | 389.0 [MH]⁺; |
| | | | | HPLC: Rt 0.87 |
| | | | | min, 98.2% |
| | | | | purity |
| 117 | 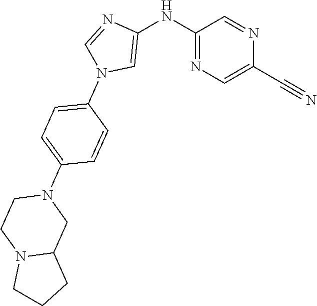 [SMILES: N#Cc1cnc(Nc2cn(-c3ccc(N4CCN5CCCC5C4)cc3)cn2)cn1] | 5-((1-(4- (Hexahydropyrrolo [1,2-a]pyrazin- 2(1H)- yl)phenyl)- 1H-imidazol- 4-yl)amino) pyrazine-2- carbonitrile | Enantiomer 1 Prep-SFC Method 6 Rt: 21.15 min | Intermediate 154; Yellow solid; Yield 1.3 mg, 1.2%; LCMS (ES⁺): 387.4 [MH]⁺; HPLC: Rt 0.76 min, 98.3% purity |
| 118 | | | Enantiomer 2 | Intermediate 154; |
| | | | Prep-SFC | Yellow solid; |
| | | | Method 6 | Yield 4.2 mg, 3.7%; |
| | | | Rt: 35.75 min | LCMS (ES⁺): |
| | | | | 386.5 [MH]⁺; |
| | | | | HPLC: Rt 0.75 |
| | | | | min, 98.6% |
| | | | | purity |
| 119 | 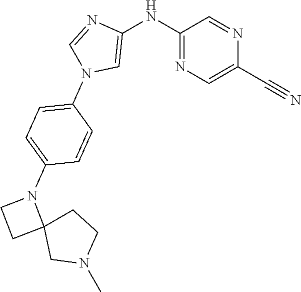 [SMILES: CN1CCC2(CCN2c2ccc(-n3cnc(Nc4cnc(C#N)cn4)c3)cc2)C1] | 5-((1-(4-(6- Methyl-1,6- diazaspiro[3.4] octan-1- yl)phenyl)- 1H-imidazol- 4-yl)amino) pyrazine-2- carbonitrile | Enantiomer 1 Chiral Prep- HPLC Method 7 Rt: 6.8 min | Intermediate 209; Yellow solid; Yield 1.4 mg, 1.8%; LCMS (ES⁺): 387.4 [MH]⁺; HPLC: Rt 0.81 min, 96.4% purity |
| 120 | | | Enantiomer 2 | Intermediate 209; |
| | | | Chiral Prep- | Yellow solid; |
| | | | HPLC Method 7 | Yield 1.0 mg, 1.3%; |
| | | | Rt: 18.7 min | LCMS (ES⁺): |
| | | | | 387.4 [MH]⁺; |
| | | | | HPLC: Rt 0.82 |
| | | | | min, 97.8% |
| | | | | purity |
| 121 | 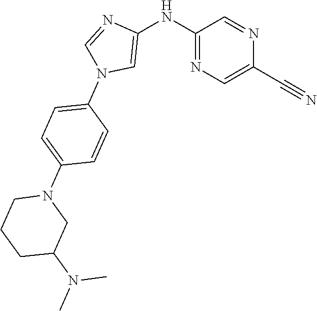 [SMILES: CN(C)C1CCCN(c2ccc(-n3cnc(Nc4cnc(C#N)cn4)c3)cc2)C1] | 5-((1-(4-(3- (Dimethylamino) piperidin-1- yl)phenyl)- 1H-imidazol- 4-yl)amino) pyrazine-2- carbonitrile | HPLC Method 7 Enantiomer 1 Chiral Prep- Rt: 12.0 min | Intermediate 210; Green solid; Yield 24.6 mg, 8.5%; LCMS (ES⁺): 389.4 [MH]⁺; HPLC: Rt 0.75 min, 98.5% purity |
| 122 | | | Enantiomer 2 | Intermediate 210; |
| | | | Chiral Prep- | Green solid; |
| | | | HPLC Method 7 | Yield 23.6 mg, 8.2%; |
| | | | Rt: 18.0 min | LCMS (ES⁺): |
| | | | | 389.4 [MH]⁺; |
| | | | | HPLC: Rt 0.75 |
| | | | | min, 98.7% |
| | | | | purity |
| 123 | 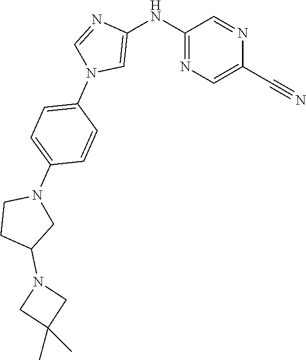 [SMILES: CC1(C)CN(C2CCN(c3ccc(-n4cnc(Nc5cnc(C#N)cn5)c4)cc3)C2)C1] | 5-((1-(4-(3-(3,3- Dimethylazetidin- 1-yl)pyrrolidin- 1-yl)phenyl)- 1H-imidazol- 4-yl)amino) pyrazine-2- carbonitrile | HPLC Method 8 Enantiomer 1 Chiral Prep- Rt: 18.4 min | Intermediate 213; Yellow solid; Yield 11.9 mg, 39.7%; LCMS (ES⁺): 415.3 [MH]⁺; HPLC: Rt 0.86 min, 95.4% purity |
| 124 | | | Enantiomer 2 | Intermediate 213; |
| | | | Chiral Prep- | Yellow solid; |
| | | | HPLC Method 8 | Yield 10.7 mg, 35.7%; |
| | | | Rt: 27.77 min | LCMS (ES⁺): |
| | | | | 415.3 [MH]⁺; |
| | | | | HPLC: Rt 0.87 |
| | | | | min, 96.8% |
| | | | | purity |
| 125 | 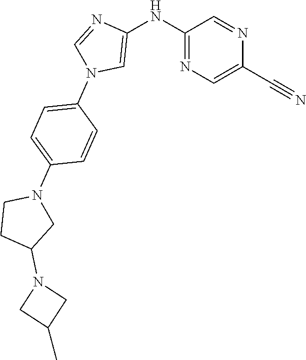 [SMILES: CC1CN(C2CCN(c3ccc(-n4cnc(Nc5cnc(C#N)cn5)c4)cc3)C2)C1] | 5-((1-(4-(3-(3- Methylazetidin- 1-yl)pyrrolidin- 1-yl)phenyl)- 1H-imidazol- 4-yl)amino) pyrazine-2- carbonitrile | Enantiomer 1 Chiral Prep- HPLC Method 9 Rt: 17.2 min | Intermediate 214; Yellow solid; Yield 25.6 mg, 14.5%; LCMS (ES⁺): 401.2 [MH]⁺; HPLC: Rt 1.64 min, 96.7% purity |
| 126 | | | Enantiomer 2 | Intermediate 214; |
| | | | Chiral Prep- | Yellow solid; |
| | | | HPLC Method 9 | Yield 21.8 mg, 12.3%; |
| | | | Rt: 22.0 min | LCMS (ES⁺): |
| | | | | 401.2 [MH]⁺; |
| | | | | HPLC: Rt 1.64 |
| | | | | min, 95.3% |
| | | | | purity |

### Examples 127 to 140

Examples 127 to 140 were prepared similarly to Example 1 via AdBrettPhos Pd G3-catalysed Buchwald-Hartwig coupling of the specified aryl bromide Intermediate with 5-aminopyrazine-2-carbonitrile. Intermediates 211-212, & 215-226 were single enantiomers or diastereomers of unknown absolute configuration due to a prior isomer separation step using either chiral prep-HPLC or SFC, and therefore Examples 127-140 are also single enantiomers or diastereomers of unknown absolute configuration; see Table 12 below.

TABLE 12
AdBrettPhos Pd G3-catalysed Buchwald-Hartwig coupling
reactions using (hetero)aryl bromide intermediates of unknown absolute
configuration

| | | | Isomer; |
|---|---|---|---|
| | | | Intermediate(s) used; |
| | | | Form (salt); Yield; LCMS; |
| Ex. | Structure | Name | HPLC |
| 127 | 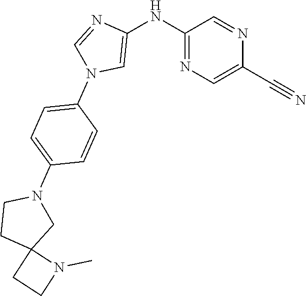 [SMILES: CN1CCC12CCN(c1ccc(-n3cnc(Nc4cnc(C#N)cn4)c3)cc1)C2] | 5-((1-(4-(1-Methyl-1,6- diazaspiro[3.4]octan-6- yl)phenyl)-1H-imidazol- 4-yl)amino)pyrazine-2- carbonitrile | Enantiomer 1 Intermediate 211; Yellow solid; Yield 9.6 mg, 10.6%; LCMS (ES⁺): 387.3 [MH]⁺; HPLC: Rt 0.83 min, 98.5% purity |
| 128 | | | Enantiomer 2 |
| | | | Intermediate 212; |
| | | | Yellow solid; |
| | | | Yield 39.2 mg, 23.5%; |
| | | | LCMS (ES⁺): 387.2 [MH]⁺; |
| | | | HPLC: Rt 1.34 min, 99.4% |
| | | | purity |
| 129 | 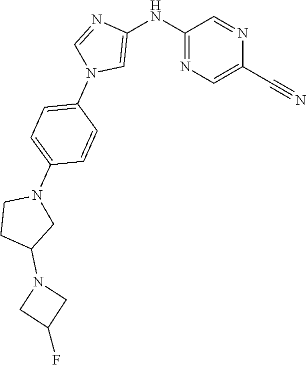 [SMILES: N#Cc1cnc(Nc2cn(-c3ccc(N4CCC(N5CC(F)C5)C4)cc3)cn2)cn1] | 5-((1-(4-(3-(3- Fluoroazetidin-1- yl)pyrrolidin-1- yl)phenyl)-1H-imidazol- 4-yl)amino)pyrazine-2- carbonitrile | Enantiomer 1 Intermediate 215; Yellow solid; Yield 24.6 mg, 10.4%; LCMS (ES⁺): 405.3 [MH]⁺; HPLC: Rt 0.81 min, 98.9% purity |
| 130 | | | Enantiomer 2 |
| | | | Intermediate 216; |
| | | | Yellow solid; |
| | | | Yield 47.7 mg, 38.8%; |
| | | | LCMS (ES⁺): 405.3 [MH]⁺; |
| | | | HPLC: Rt 0.82 min, 97.4% |
| | | | purity |
| 131 | 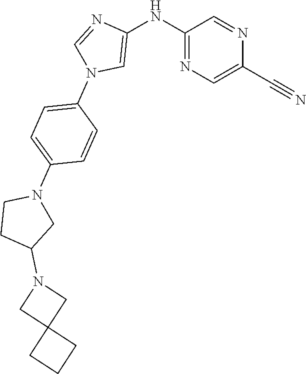 [SMILES: N#Cc1cnc(Nc2cn(-c3ccc(N4CCC(N5CC6(CCC6)C5)C4)cc3)cn2)cn1] | 5-((1-(4-(3-(2- Azaspiro[3.3]heptan-2- yl)pyrrolidin-1- yl)phenyl)-1H-imidazol- 4-yl)amino)pyrazine-2- carbonitrile | Enantiomer 1 Intermediate 217; Yellow solid (FA); Yield 28.1 mg, 25.6%; LCMS (ES⁺): 427.3 [MH]⁺; HPLC: Rt 2.16 min, 95.4% purity |
| 132 | | | Enantiomer 2 |
| | | | Intermediate 218; |
| | | | Yellow solid (FA); |
| | | | Yield 49.4 mg, 40.5%; |
| | | | LCMS (ES⁺): 427.2 [MH]⁺; |
| | | | HPLC: Rt 1.85 min, 97.7% |
| | | | purity |
| 133 | 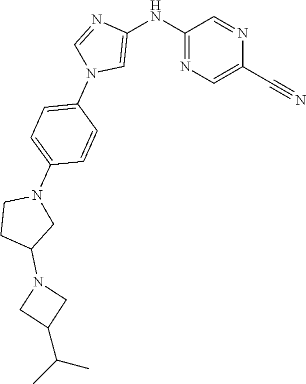 [SMILES: CC(C)C1CN(C2CCN(c3ccc(-n4cnc(Nc5cnc(C#N)cn5)c4)cc3)C2)C1] | 5-((1-(4-(3-(3- Isopropylazetidin-1- yl)pyrrolidin-1- yl)phenyl)-1H-imidazol- 4-yl)amino)pyrazine-2- carbonitrile | Enantiomer 1 Intermediate 219; Yellow solid (FA); Yield 8.2 mg, 7.5%; LCMS (ES⁺): 429.0 [MH]⁺; HPLC: Rt 1.22 min, 99.4% purity |
| 134 | | | Enantiomer 2 |
| | | | Intermediate 220; |
| | | | Yellow solid (FA); |
| | | | Yield 14.5 mg, 11.9%; |
| | | | LCMS (ES⁺): 429.4 [MH]⁺; |
| | | | HPLC: Rt 1.05 min, 96.9% |
| | | | purity |
| 135 | 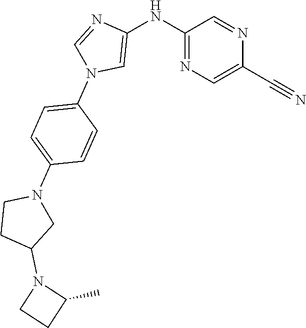 [SMILES: C[C@@H]1CCN1C1CCN(c2ccc(-n3cnc(Nc4cnc(C#N)cn4)c3)cc2)C1] | 5-((1-(4-(3-((R)-2- Methylazetidin-1- yl)pyrrolidin-1- yl)phenyl)-1H-imidazol- 4-yl)amino)pyrazine-2- carbonitrile | Diastereomer 1 Intermediate 221; Yellow solid; Yield 26.5 mg, 36.8%; LCMS (ES⁺): 401.2 [MH]⁺; HPLC: Rt 1.56 min, 97.3% purity |
| 136 | | | Diastereomer 2 |
| | | | Intermediate 222; |
| | | | Yellow solid; |
| | | | Yield 32.4 mg, 36.9%; |
| | | | LCMS (ES⁺): 401.2 [MH]⁺; |
| | | | HPLC: Rt 1.55 min, 98.8% |
| | | | purity |
| 137 | 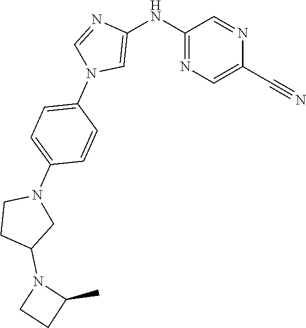 [SMILES: C[C@H]1CCN1C1CCN(c2ccc(-n3cnc(Nc4cnc(C#N)cn4)c3)cc2)C1] | 5-((1-(4-(3-((S)-2- Methylazetidin-1- yl)pyrrolidin-1- yl)phenyl)-1H-imidazol- 4-yl)amino)pyrazine-2- carbonitrile | Diastereomer 1 Intermediate 223; Yellow solid; Yield 11.5 mg, 15.4%; LCMS (ES⁺): 401.3 [MH]⁺; HPLC: Rt 0.83 min, 97.8% purity |
| 138 | | | Diastereomer 2 |
| | | | Intermediate 224; |
| | | | Yellow solid; |
| | | | Yield 9.6 mg, 15.0%; |
| | | | LCMS (ES+): 401.3 [MH]⁺; |
| | | | HPLC: Rt 0.84min, 98.4% |
| | | | purity |
| 139 | 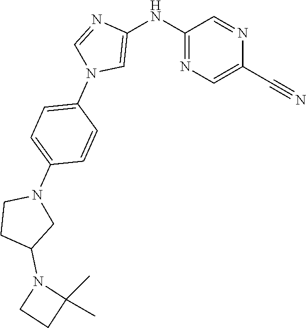 [SMILES: CC1(C)CCN1C1CCN(c2ccc(-n3cnc(Nc4cnc(C#N)cn4)c3)cc2)C1] | 5-((1-(4-(3-(2,2- Dimethylazetidin-1- yl)pyrrolidin-1- yl)phenyl)-1H-imidazol- 4-yl)amino)pyrazine-2- carbonitrile | Enantiomer 1 Intermediate 225; Yellow solid; Yield 17.9 mg, 34.5%; LCMS (ES⁺): 415.4 [MH]⁺; HPLC: Rt 0.90 min, 98.9% purity |
| 140 | | | Enantiomer 2 |
| | | | Intermediate 226; |
| | | | Yellow solid; |
| | | | Yield 16.3 mg, 30.7%; |
| | | | LCMS (ES⁺): 415.4 [MH]⁺; |
| | | | HPLC: Rt 0.90 min, 98.2% |
| | | | purity |

### Example 141

### (R)-1-(4-(4-((5-Cyanopyrazin-2-yl)amino)-1H-imidazol-1-yl)phenyl)-N,N-dimethylpyrrolidin-3-amine oxide

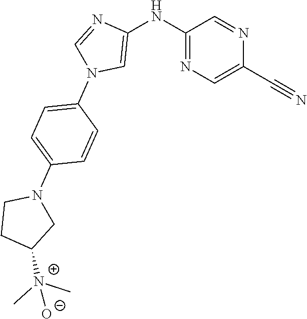 [SMILES: C[N+](C)([O-])[C@@H]1CCN(c2ccc(-n3cnc(Nc4cnc(C#N)cn4)c3)cc2)C1]

To Example 12 (100 mg, 0.26 mmol) in DCM (8.0 mL) was added mCPBA, (75% in water, 90.0 mg, 0.39 mmol). The mixture was stirred at RT for 30 min then purified via a catch and elute cartridge. Further purification via NP column chromatography then RP HPLC afforded the title compound (15.3 mg, 15.0%) as a green solid. LCMS (ES⁺): 391.2 [MH]⁺. UPLC: Rt 3.30 min, 99.6% purity.

### Example 142

### 5-((1-(4-(Morpholinomethyl)phenyl)-1H-imidazol-4-yl)amino)pyrazine-2-carbonitrile

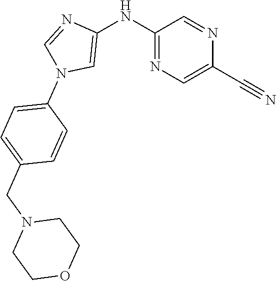 [SMILES: N#Cc1cnc(Nc2cn(-c3ccc(CN4CCOCC4)cc3)cn2)cn1]

Intermediate 15 (150 mg, 0.47 mmol) was reacted with 5-aminopyrazine-2-carbonitrile (83.9 mg, 0.70 mmol) similarly to Example 1, but using EPhos Pd G4 and Ephos instead of AdBrettPhos Pd G3 and AdBrettPhos, respectively. Purification via RP HPLC afforded the title compound (5.4 mg, 3.2%) as a yellow solid. LCMS (ES⁺): 362.3 [MH]⁺. HPLC: Rt 0.91 min, 98.8% purity.

### Examples 143 to 147

Examples 143 to 147 were prepared similarly to Example 142, via EPhos Pd G4-catalysed Buchwald-Hartwig coupling of the specified aryl bromide Intermediates with 5-aminopyrazine-2-carbonitrile; see Table 13 below.

TABLE 13
Ephos Pd G4-catalysed Buchwald-Hartwig coupling reactions

| | | | Intermediate used; |
|---|---|---|---|
| | | | Form (salt); Yield; LCMS; |
| Ex. | Structure | Name | HPLC/UPLC |
| 143 | 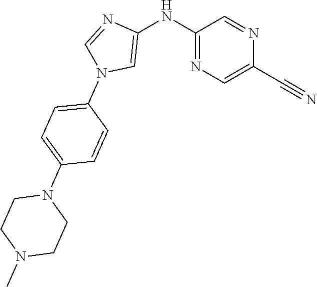 [SMILES: CN1CCN(c2ccc(-n3cnc(Nc4cnc(C#N)cn4)c3)cc2)CC1] | 5-((1-(4-(4- Methylpiperazin-1- yl)phenyl)-1H- imidazol-4- yl)amino)pyrazine-2- carbonitrile | Intermediate 12; Yellow solid; Yield 16.7 mg, 6.7%; LCMS (ES⁺): 361.2 [MH]⁺; HPLC: Rt 0.56 min; 99.4% purity |
| 144 | 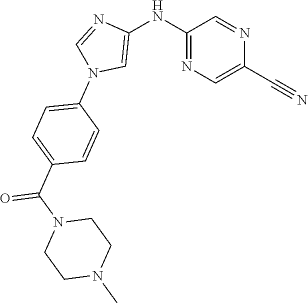 [SMILES: CN1CCN(C(=O)c2ccc(-n3cnc(Nc4cnc(C#N)cn4)c3)cc2)CC1] | 5-((1-(4-(4- Methylpiperazine-1- carbonyl)phenyl)-1H- imidazol-4- yl)amino)pyrazine-2- carbonitrile | Intermediate 14; Pale yellow solid; Yield 2.5 mg, 1.9%; LCMS (ES⁺): 389.1 [MH]⁺; UPLC: Rt 2.97 min, 97.2% purity |
| 145 | 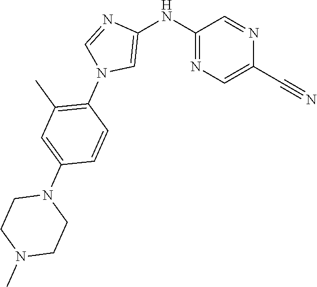 [SMILES: Cc1cc(N2CCN(C)CC2)ccc1-n1cnc(Nc2cnc(C#N)cn2)c1] | 5-((1-(2-methyl-4-(4- Methylpiperazin-1- yl)phenyl)-1H- imidazol-4- yl)amino)pyrazine-2- carbonitrile | Intermediate 88; White solid (TFA); Yield 4.9 mg, 2.6%; LCMS (ES⁺): 375.2 [MH]⁺; HPLC: Rt 0.62 min, 98.0% purity |
| 146 | 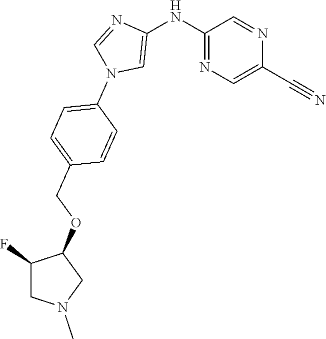 [SMILES: CN1C[C@@H](F)[C@@H](OCc2ccc(-n3cnc(Nc4cnc(C#N)cn4)c3)cc2)C1] | 5-((1-(4-((((3S,4R)- 4-fluoro-1- Methylpyrrolidin-3- yl)oxy)methyl) phenyl)-1H- imidazol-4- yl)amino)pyrazine- 2-carbonitrile | Intermediate 193; Yellow solid; Yield 1.0 mg, 1.8%; LCMS (ES⁺): 394.2 [MH]⁺; HPLC: Rt 2.68 min, 98.4% purity |
| 147 | 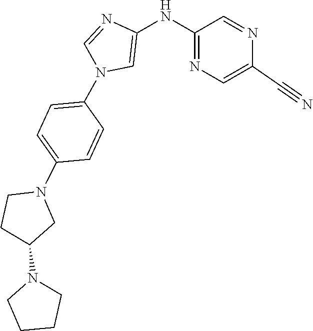 [SMILES: N#Cc1cnc(Nc2cn(-c3ccc(N4CC[C@@H](N5CCCC5)C4)cc3)cn2)cn1] | (R)-5-((1-(4-([1,3′- Bipyrrolidin]-1′- yl)phenyl)-1H- imidazol-4- yl)amino)pyrazine- 2-carbonitrile | Intermediate 140; Yellow solid (FA); Yield 1.1 mg, 0.9%; LCMS (ES⁺): 401.1 [MH]⁺; HPLC: Rt 1.51 min, 96.1% purity |

### Example 148

### 5-((1-(3-(3-Aminopropoxy)phenyl)-1H-imidazol-4-yl)amino)pyrazine-2-carbonitrile

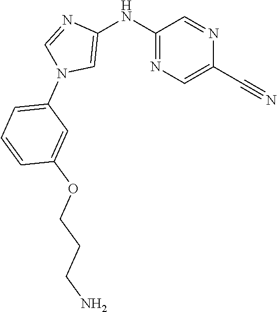 [SMILES: N#Cc1cnc(Nc2cn(-c3cccc(OCCCN)c3)cn2)cn1]

Step 1: Intermediate 69 (200 mg, 0.51 mmol) was reacted with 5-aminopyrazine-2-carbonitrile (90.9 mg, 0.76 mmol) similarly to Example 1 using AdBrettPhos Pd G3. Purification via RP column chromatography afforded tert-butyl N-[3-(3-{4-[(5-cyanopyrazin-2-yl)amino]imidazol-1-yl}phenoxy)propyl]carbamate (94.8 mg, 43.1%) as a light brown solid. LCMS (ES⁺): 436.2 [MH]⁺.

Step 2: A mixture of tert-butyl N-[3-(3-{4-[(5-cyanopyrazin-2-yl)amino]imidazol-1-yl}phenoxy)propyl]carbamate (90.0 mg, 0.21 mmol) and TFA (1.0 mL) in DCM (5.0 mL) was stirred for 30 min at RT. The resulting mixture was concentrated in vacuo and purified via RP HPLC to afford the title compound (21.0 mg, 28.9%) as a yellow solid. LCMS (ES⁺): 336.1 [MH]⁺. HPLC: Rt 1.02 min, 95.4% purity.

### Examples 149 to 165

Examples 149 to 165 were prepared similarly to Example 148, via AdBrettPhos Pd G3-catalysed Buchwald-Hartwig coupling of the specified aryl bromide Intermediate with 5-aminopyrazine-2-carbonitrile, then Boc deprotection using TFA; see Table 14 below.

TABLE 14
AdBrettPhos Pd G3-catalysed Buchwald-Hartwig coupling then
Boc-deprotection reactions

| | | | Intermediate used; |
|---|---|---|---|
| | | | Form (salt); Yield; LCMS; |
| Ex. | Structure | Name | HPLC/UPLC |
| 149 | 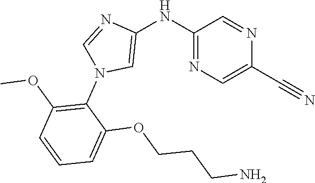 [SMILES: COc1cccc(OCCCN)c1-n1cnc(Nc2cnc(C#N)cn2)c1] | 5-((1-(2-(3- Aminopropoxy)-6- methoxyphenyl)-1H- imidazol-4- yl)amino)pyrazine-2- carbonitrile | Intermediate 29; Yellow solid; Yield 16.9 mg, 24.3% over 2 steps; LCMS (ES⁺): 366.1 [MH]⁺; HPLC: Rt 0.93 min, 98.5% purity |
| 150 | 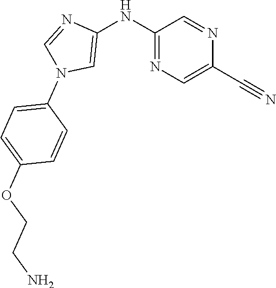 [SMILES: N#Cc1cnc(Nc2cn(-c3ccc(OCCN)cc3)cn2)cn1] | 5-((1-(4-(2- Aminoethoxy)phenyl)- 1H-imidazol-4- yl)amino)pyrazine-2- carbonitrile | Intermediate 67; Yellow solid (FA); Yield 11.5 mg, 8.0% over 2 steps; LCMS (ES⁺): 322.2 [MH]⁺; HPLC: Rt 2.14 min, 98.9% purity |
| 151 | 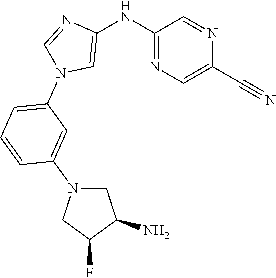 [SMILES: N#Cc1cnc(Nc2cn(-c3cccc(N4C[C@@H](N)[C@@H](F)C4)c3)cn2)cn1] | 5-((1-(3-((3R,4S)-3- Amino-4- fluoropyrrolidin-1- yl)phenyl)-1H- imidazol-4- yl)amino)pyrazine-2- carbonitrile | Intermediate 107; Yellow solid (TFA); Yield 76.2 mg, 41.1% over 2 steps; LCMS (ES⁺): 365.0 [MH]; HPLC: Rt 0.72 min, 99.4% purity |
| 152 | 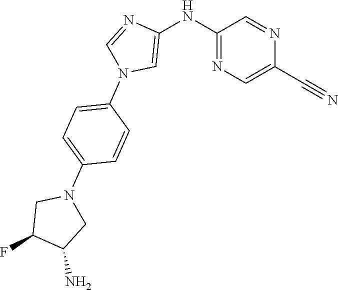 [SMILES: N#Cc1cnc(Nc2cn(-c3ccc(N4C[C@H](N)[C@@H](F)C4)cc3)cn2)cn1] | 5-((1-(4-((3S,4S)-3- Amino-4- fluoropyrrolidin-1- yl)phenyl)-1H- imidazol-4- yl)amino)pyrazine-2- carbonitrile | Intermediate 117; Yellow solid (FA); Yield 11.1 mg, 9.6% over 2 steps; LCMS (ES⁺): 365.2 [MH]⁺; HPLC: Rt 0.83 min, 95.0% purity |
| 153 | 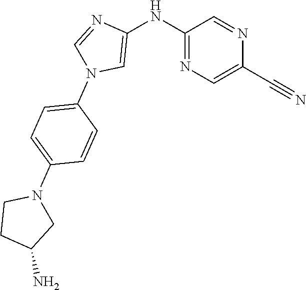 [SMILES: N#Cc1cnc(Nc2cn(-c3ccc(N4CC[C@@H](N)C4)cc3)cn2)cn1] | (R)-5-((1-(4-(3- Aminopyrrolidin-1- yl)phenyl)-1H- imidazol-4- yl)amino)pyrazine-2- carbonitrile | Intermediate 118; Yellow solid (TFA); Yield 4.2 mg, 8.3% over 2 steps; LCMS (ES⁺): 347.0 [MH]⁺; HPLC: Rt 0.74 min, 98.7% purity |
| 154 | 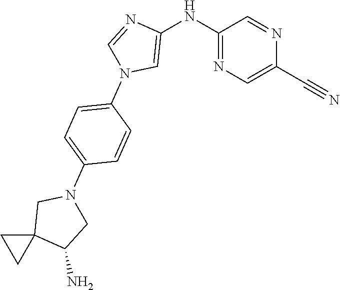 [SMILES: N#Cc1cnc(Nc2cn(-c3ccc(N4C[C@H](N)C5(CC5)C4)cc3)cn2)cn1] | (R)-5-((1-(4-(7- Amino-5- azaspiro[2.4]heptan- 5-yl)phenyl)-1H- imidazol-4- yl)amino)pyrazine-2- carbonitrile | Intermediate 119; Yellow solid; Yield 11.1 mg, 15.4% over 2 steps; LCMS (ES⁺): 373.2 [MH]; HPLC: Rt 1.62 min, 98.8% purity |
| 155 | 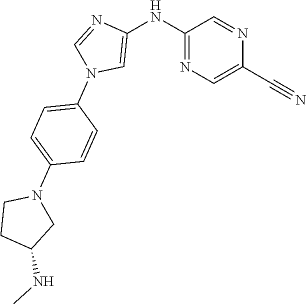 [SMILES: CN[C@@H]1CCN(c2ccc(-n3cnc(Nc4cnc(C#N)cn4)c3)cc2)C1] | (R)-5-((1-(4-(3- (Methylamino)pyrrolidin- 1-yl)phenyl)-1H- imidazol-4- yl)amino) pyrazine-2-carbonitrile | Yield 2.2 mg, 3.1% over 2 steps; Intermediate 120; Yellow solid; LCMS (ES⁺): 361.2 [MH]⁺; HPLC: Rt 1.46 min, 97.0% purity |
| 156 | 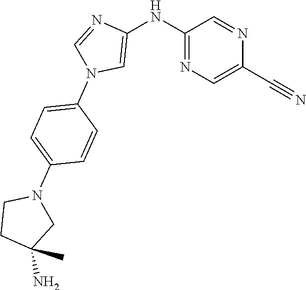 [SMILES: C[C@@]1(N)CCN(c2ccc(-n3cnc(Nc4cnc(C#N)cn4)c3)cc2)C1] | (R)-5-((1-(4-(3-Amino- 3-methylpyrrolidin-1- yl)phenyl)-1H- imidazol-4- yl)amino)pyrazine-2- carbonitrile | Intermediate 121; Yellow solid (TFA); Yield 17.9 mg, 22% over 2 steps; LCMS (ES⁺): 361.0 [MH]⁺; HPLC: Rt 0.83 min, 98.9% purity |
| 157 | 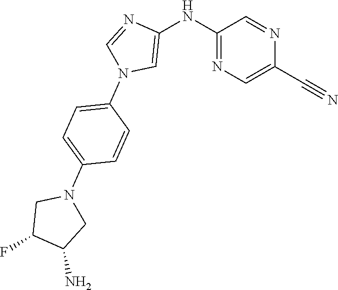 [SMILES: N#Cc1cnc(Nc2cn(-c3ccc(N4C[C@@H](F)[C@@H](N)C4)cc3)cn2)cn1] | 5-((1-(4-((3S,4R)-3- Amino-4- fluoropyrrolidin-1- yl)phenyl)-1H- imidazol-4- yl)amino)pyrazine-2- carbonitrile | Intermediate 123; Yellow solid (FA); Yield 2.3 mg, 6.2% over 2 steps; LCMS (ES⁺): 365.0 [MH]⁺; HPLC: Rt 0.81 min, 97.2% purity |
| 158 | 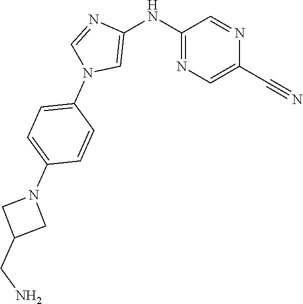 [SMILES: N#Cc1cnc(Nc2cn(-c3ccc(N4CC(CN)C4)cc3)cn2)cn1] | 5-((1-(4-(3- (Aminomethyl)azetidin- 1-yl)phenyl)-1H- imidazol-4- yl)amino)pyrazine-2- carbonitrile | Intermediate 169; Pale yellow solid Yield 3.8 mg, 2.6% over 2 steps LCMS (ES⁺): 347.1 [MH]⁺; UPLC: Rt 3.04 min, 97.7% purity |
| 159 | 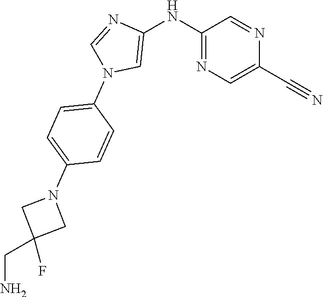 [SMILES: N#Cc1cnc(Nc2cn(-c3ccc(N4CC(F)(CN)C4)cc3)cn2)cn1] | 5-((1-(4-(3- (Aminomethyl)-3- fluoroazetidin-1- yl)phenyl)-1H- imidazol-4- yl)amino)pyrazine-2- carbonitrile | Intermediate 128; Yellow solid Yield 10.0 mg, 9.8% over 2 steps; LCMS (ES⁺): 365.0 [MH]⁺ HPLC: Rt 0.77 min, 97.0% purity |
| 160 | 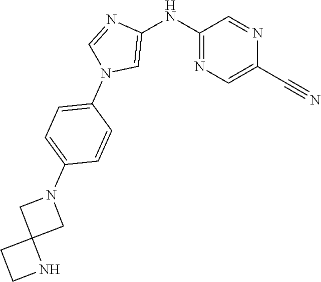 [SMILES: N#Cc1cnc(Nc2cn(-c3ccc(N4CC5(CCN5)C4)cc3)cn2)cn1] | 5-((1-(4-(1,6- Diazaspiro[3.3]heptan- 6-yl)phenyl)-1H- imidazol-4- yl)amino)pyrazine-2- carbonitrile | Intermediate 129; Yellow solid Yield 20.1 mg, 22.4% over 2 steps LCMS (ES⁺): 359.0 [MH]⁺ HPLC: Rt 0.77 min, 98.7% purity |
| 161 | 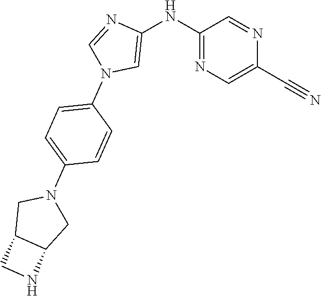 [SMILES: N#Cc1cnc(Nc2cn(-c3ccc(N4C[C@H]5CN[C@H]5C4)cc3)cn2)cn1] | 5-((1-(4-((1R,5R)-3,6- Diazabicyclo[3.2.0] heptan-3-yl)phenyl)-1H- imidazol-4- yl)amino)pyrazine-2- carbonitrile | Intermediate 174; Pale yellow solid Yield 23.6 mg 28.1% over 2 steps LCMS (ES⁺): 359.2 [MH]⁺ UPLC: Rt 3.15 min, 98.8% purity |
| 162 | 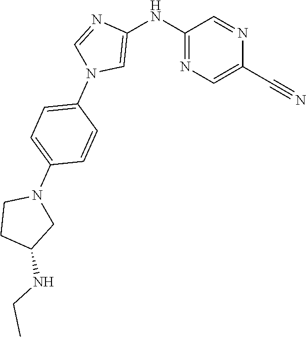 [SMILES: CCN[C@@H]1CCN(c2ccc(-n3cnc(Nc4cnc(C#N)cn4)c3)cc2)C1] | (R)-5-((1-(4-(3- (Ethylamino)pyrrolidin- 1-yl)phenyl)-1H- imidazol-4- yl)amino)pyrazine-2- carbonitrile | Intermediate 187; Yellow solid Yield 20.5 mg, 12.3% over 2 steps LCMS (ES⁺): 375.0 [MH]⁺ HPLC: Rt 1.87 min, 98.5% purity |
| 163 | 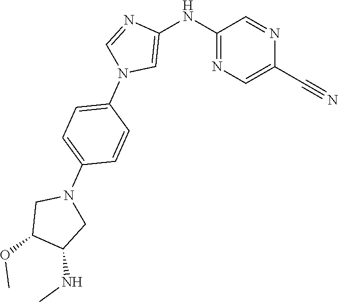 [SMILES: CN[C@H]1CN(c2ccc(-n3cnc(Nc4cnc(C#N)cn4)c3)cc2)C[C@H]1OC] | 5-((1-(4-((3R,4S)-3- Methoxy-4- (methylamino)pyrrolid in-1-yl)phenyl)-1H- imidazol-4- yl)amino)pyrazine-2- carbonitrile | Intermediate 185; Yellow solid Yield 1.8 mg, 1.4% over 2 steps LCMS (ES⁺): 391.0 [MH]⁺ HPLC: Rt 0.69 min, 96.9% purity |
| 164 | 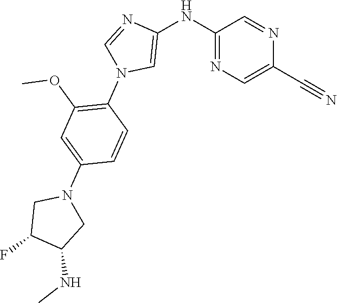 [SMILES: CN[C@H]1CN(c2ccc(-n3cnc(Nc4cnc(C#N)cn4)c3)c(OC)c2)C[C@H]1F] | 5-((1-(4-((3R,4S)-3- Fluoro-4- (methylamino) pyrrolidin-1-yl)-2- methoxyphenyl)-1H- imidazol-4- yl)amino)pyrazine-2- carbonitrile | Intermediate 186; Yellow solid Yield 6.4 mg, 5.2% over 2 steps LCMS (ES⁺): 409.2 [MH]⁺ HPLC: Rt 0.75 min, 96.4% purity |
| 165 | 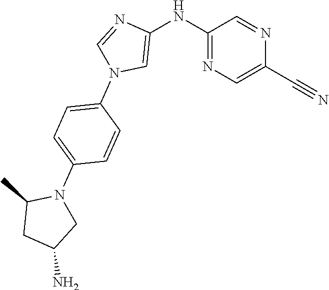 [SMILES: C[C@@H]1C[C@@H](N)CN1c1ccc(-n2cnc(Nc3cnc(C#N)cn3)c2)cc1] | 5-((1-(4-((2R,4R)-4- Amino-2- methylpyrrolidin-1- yl)phenyl)-1H- imidazol-4- yl)amino)pyrazine-2- carbonitrile | Intermediate 158; Pale yellow solid Yield 133 mg, 29.3% over 2 steps LCMS (ES⁺): 361.2 [MH]⁺ HPLC: Rt 3.35 min, 100% purity |

### Examples 166 and 167

### 5-((1-(3-(4-Amino-3,3-difluoropiperidin-1-yl)phenyl)-1H-imidazol-4-yl)amino)pyrazine-2-carbonitrile

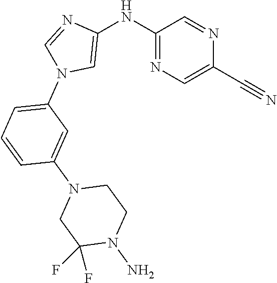 [SMILES: N#Cc1cnc(Nc2cn(-c3cccc(N4CCN(N)C(F)(F)C4)c3)cn2)cn1]

Examples 166 and 167 were prepared similarly to Example 148, via AdBrettPhos Pd G3-catalysed Buchwald-Hartwig coupling of Intermediate 110 with 5-aminopyrazine-2-carbonitrile then Boc deprotection using TFA. The enantiomers were separated using chiral prep-HPLC method 10 to afford each enantiomer of the title compound as a single enantiomer of unknown absolute stereochemistry. Example 166 (Enantiomer 1): chiral prep-HPLC Rt 3.19 min to afford the title compound (13.0 mg, 4.7% over 2 steps) as a yellow solid. LCMS (ES⁺): 397.4 [MH]⁺. HPLC: Rt 0.74 min, 99.5% purity.

Example 167 (Enantiomer 2): chiral prep-HPLC Rt 4.49 min to afford the title compound (11.0 mg, 4.0% over 2 steps) as a yellow solid. LCMS (ES⁺): 397.3 [MH]⁺. HPLC: Rt 0.72 min, 99.9% purity.

### Example 168

### 5-((1-(2-(3-Aminopropoxy)phenyl)-1H-imidazol-4-yl)amino)pyrazine-2-carbonitrile

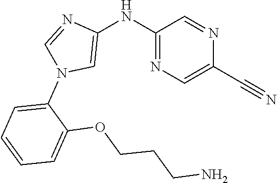 [SMILES: N#Cc1cnc(Nc2cn(-c3ccccc3OCCCN)cn2)cn1]

Example 168 was prepared from Intermediate 13 similarly to Example 148, but EPhos Pd G4 and Ephos were used instead of AdBrettPhos Pd G3 and AdBrettPhos, respectively, in Step 1. Purification via RP HPLC afforded the title compound (5.8 mg, 19.1% over 2 steps) as a light-yellow solid. LCMS (ES⁺): 336.2 [MH]⁺. HPLC: Rt 6.26 min, 97.9% purity.

### Example 169

### (R)-5-((1-(4-((3-Fluoropyrrolidin-1-yl)methyl)-2-methoxyphenyl)-1H-imidazol-4-yl)amino)pyrazine-2-carbonitrile)

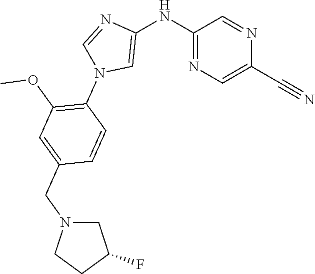 [SMILES: COc1cc(CN2CC[C@@H](F)C2)ccc1-n1cnc(Nc2cnc(C#N)cn2)c1]

Step 1: Intermediate 41 (800 mg, 2.85 mmol) was reacted with 5-aminopyrazine-2-carbonitrile (684 mg, 5.69 mmol) using AdBrettPhos Pd G3 similarly to Example 1. Purification via NP column chromatography afforded 5-[[1-(4-formyl-2-methoxy-phenyl)imidazol-4-yl]amino]pyrazine-2-carbonitrile (95.0 mg, 9.6%) as a yellow solid.

Step 2: 5-[[1-(4-Formyl-2-methoxy-phenyl)imidazol-4-yl]amino]pyrazine-2-carbonitrile (20.0 mg, 57.6 μmol, 92.2% purity), (R)-(−)-3-fluoropyrrolidine-HCl (7.23 mg, 57.6 μmol) and MP-trimethylammonium cyanoborohydride resin (3.82 mmol/g, 37.7 mg, 0.14 mmol) in IPA (0.5 mL) and AcOH (50 μL) was heated using a microwave reactor (100° C.) for 10 min. The reaction mixture was purified via a catch and elute cartridge. Further purification via RP HPLC afforded the title compound (13.9 mg, 61.2%) as a yellow solid. LCMS (ES⁺): 394.1 [MH]⁺. UPLC: Rt 3.21 min, 99.7% purity.

### Examples 170 to 174

Examples 170 to 174 were prepared similarly to Example 169 via AdBrettPhos Pd G3-catalysed Buchwald-Hartwig coupling of Intermediates 41 and 42 with 5-aminopyrazine-2-carbonitrile, then reductive amination with the appropriate amine; see Table 15 below.

TABLE 15
Buchwald-Hartwig coupling then reductive amination reactions

| | | | Reactant(s) used; |
|---|---|---|---|
| | | | Form (salt); Yield; LCMS; |
| Ex. | Structure | Name | HPLC/UPLC |
| 170 | 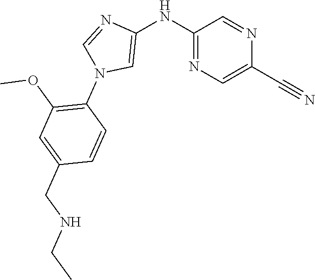 [SMILES: CCNCc1ccc(-n2cnc(Nc3cnc(C#N)cn3)c2)c(OC)c1] | 5-((1-(4- ((Ethylamino) methyl)-2- methoxyphenyl)- 1H-imidazol-4- yl)amino)pyrazine-2- carbonitrile | Intermediate 41, CAS: 75-04-7; Off-white solid; Yield 5.9 mg, 9.0% over 2 steps; LCMS (ES⁺): 350.1 [MH]⁺; UPLC: Rt 3.11 min, 94.6% purity |
| 171 | 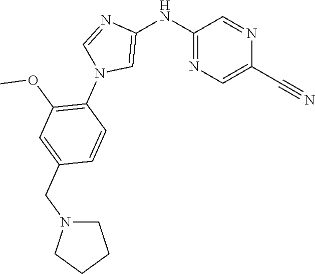 [SMILES: COc1cc(CN2CCCC2)ccc1-n1cnc(Nc2cnc(C#N)cn2)c1] | 5-((1-(2-Methoxy-4- (pyrrolidin-1- ylmethyl)phenyl)- 1H-imidazol-4- yl)amino)pyrazine- 2-carbonitrile | Intermediate 41, CAS: 123-75-1; Light yellow solid; Yield 11.2 mg, 5.0% over 2 steps; LCMS (ES⁺): 376.1 [MH]⁺; UPLC: Rt 3.24 min, 100% purity |
| 172 | 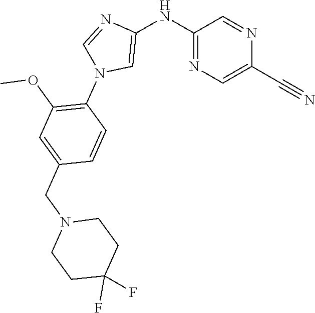 [SMILES: COc1cc(CN2CCC(F)(F)CC2)ccc1-n1cnc(Nc2cnc(C#N)cn2)c1] | 5-((1-(4-((4,4- Difluoropiperidin-1- yl)methyl)-2- methoxyphenyl)-1H- imidazol-4- yl)amino)pyrazine-2- carbonitrile | Intermediate 41, CAS: 21987-29-1; Light yellow solid; Yield 16.4mg, 6.1% over 2 steps; LCMS (ES⁺): 426.1 [MH]⁺; UPLC: Rt 3.50 min, 95.2% purity |
| 173 | 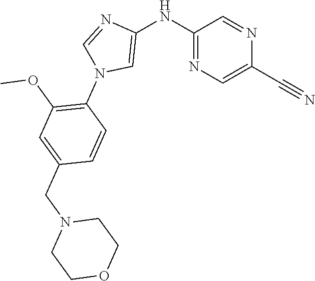 [SMILES: COc1cc(CN2CCOCC2)ccc1-n1cnc(Nc2cnc(C#N)cn2)c1] | 5-((1-(2-Methoxy-4- (morpholinomethyl) phenyl)-1H-imidazol- 4-yl)amino)pyrazine- 2-carbonitrile | Intermediate 41, CAS: 110-91-8; Light yellow solid; Yield: 9.7 mg, 4.1% over 2 steps; LCMS (ES⁺): 392.1 [MH]⁺; UPLC: Rt 3.11 min, 100% purity |
| 174 | 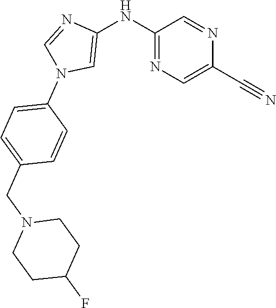 [SMILES: N#Cc1cnc(Nc2cn(-c3ccc(CN4CCC(F)CC4)cc3)cn2)cn1] | 5-((1-(4-((4- Fluoropiperidin-1- yl)methyl)phenyl)- 1H-imidazol-4- yl)amino)pyrazine-2- carbonitrile | Intermediate 42, CAS: 57395-89-8; Yellow solid; Yield 7.9 mg, 2.3% over 2 steps; LCMS (ES⁺): 378.1 [MH]⁺; HPLC: Rt 0.70 min, 95.6% purity |

### Example 175

### 5-((1-(3-((2-Methyl-2-azaspiro[3.3]heptan-6-yl)oxy)phenyl)-1H-imidazol-4-yl)amino)pyrazine-2-carbonitrile

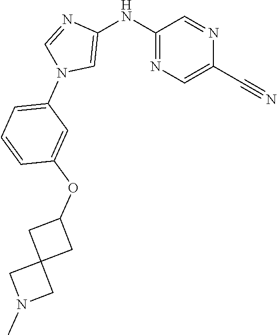 [SMILES: CN1CC2(CC(Oc3cccc(-n4cnc(Nc5cnc(C#N)cn5)c4)c3)C2)C1]

Step 1: Intermediate 72 (60.0 mg, 0.14 mmol) was reacted with 5-aminopyrazine-2-carbonitrile (24.9 mg, 0.21 mmol) similarly to Example 1. Purification via NP column chromatography afforded tert-butyl 6-(3-{4-[(5-cyanopyrazin-2-yl)amino]imidazol-1-yl}phenoxy)-2-azaspiro[3.3]heptane-2-carboxylate (41.2 mg, 63.0%) as a light yellow solid. LCMS (ES⁺): 474.3 [MH]⁺.

Step 2: A solution of tert-butyl 6-(3-{4-[(5-cyanopyrazin-2-yl)amino]imidazol-1-yl}phenoxy)-2-azaspiro[3.3]heptane-2-carboxylate (40.0 mg, 0.08 mmol) and TFA (1.0 mL) in DCM (1.0 mL) was stirred for 30 min at RT. The resulting mixture was concentrated in vacuo to afford crude 5-{[1-(3-{2-azaspiro[3.3]heptan-6-yloxy}phenyl)imidazol-4-yl]amino}pyrazine-2-carbonitrile (20.0 mg), which was used directly in the next step without further purification.

Step 3: A solution of 5-{[1-(3-{2-azaspiro[3.3]heptan-6-yloxy}phenyl)imidazol-4-yl]amino}pyrazine-2-carbonitrile (20.0 mg, 0.05 mmol) and formaldehyde (5.00 μL, 0.05 mmol) in MeCOH (1.0 mL) was stirred for 30 min at RT. NaBH₃CN (6.7 mg, 0.11 mmol) was added at 0° C. and the resulting mixture was stirred at RT for 4 h. The reaction was quenched with water, concentrated in vacuo and purified via RP HPLC (TFA-modifier) to afford the title compound as the TFA salt (2.9 mg, 10.7% over 2 steps) as a yellow solid. LCMS (ES⁺): 388.1 [MH]⁺. HPLC: Rt 1.23 min, 98.9% purity.

### Examples 176 to 180

Examples 176 to 180 were prepared similarly to Example 175, via AdBrettPhos Pd G3-catalysed Buchwald-Hartwig coupling of the specified aryl bromide Intermediate with 5-aminopyrazine-2-carbonitrile, followed by TFA Boc removal, then reductive amination with the appropriate aldehyde; see Table 16 below.

TABLE 16
AdBrettPhos Pd G3-catalysed Buchwald-Hartwig coupling then
Boc removal and subsequent reductive amination reactions

| | | | Reactant(s) used; |
|---|---|---|---|
| Ex. | Structure | Name | Form; Yield; LCMS; HPLC |
| 176 | 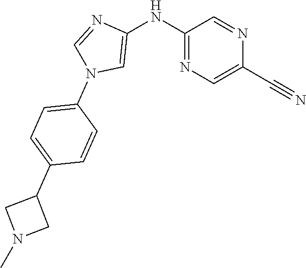 [SMILES: CN1CC(c2ccc(-n3cnc(Nc4cnc(C#N)cn4)c3)cc2)C1] | 5-((1-(4-(1- Methylazetidin-3- yl)phenyl)-1H- imidazol-4- yl)amino)pyrazine- 2-carbonitrile | Intermediate 17, CAS: 50-00-0; Yellow solid; Yield: 2.1 mg, 3.0% over 3 steps; LCMS (ES⁺): 332.2 [MH]⁺; HPLC: Rt 0.58 min, 96.3% purity |
| 177 | 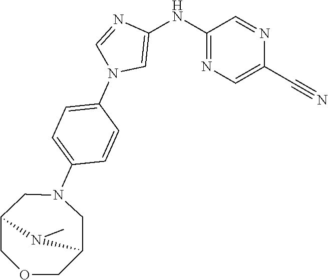 [SMILES: CN1[C@@H]2COC[C@H]1CN(c1ccc(-n3cnc(Nc4cnc(C#N)cn4)c3)cc1)C2] | 5-((1-(4-((1R,5S)-9- Methyl-3-oxa-7,9- diazabicyclo[3.3.1] nonan-7-yl)phenyl)- 1H-imidazol-4- yl)amino)pyrazine-2- carbonitrile | Intermediate 78, CAS: 50-00-0; Yellow solid; Yield: 9.0 mg, 4.0% over 3 steps; LCMS (ES⁺): 403.3 [MH]⁺; HPLC: Rt 3.13 min, 97.3% purity |
| 178 | 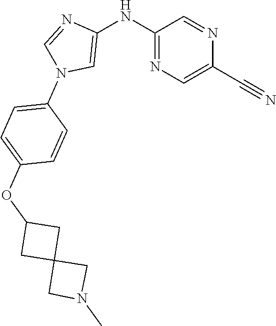 [SMILES: CN1CC2(CC(Oc3ccc(-n4cnc(Nc5cnc(C#N)cn5)c4)cc3)C2)C1] | 5-((1-(4-((2-Methyl-2- azaspiro[3.3]heptan- 6-yl)oxy)phenyl)-1H- imidazol-4- yl)amino)pyrazine- 2-carbonitrile | Intermediate 71, CAS: 50-00-0; Yellow solid; Yield: 6.8 mg, 1.6% over 3 steps; LCMS (ES⁺): 388.1 [MH]⁺ HPLC: Rt 1.19 min, 99.1% purity |
| 179 | 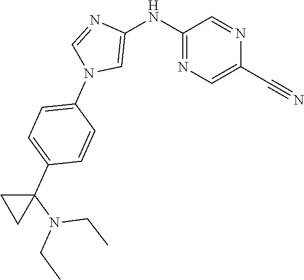 [SMILES: CCN(CC)C1(c2ccc(-n3cnc(Nc4cnc(C#N)cn4)c3)cc2)CC1] | 5-((1-(4-(1- (Diethylamino) cyclopropyl) phenyl)-1H- imidazol-4- yl)amino)pyrazine- 2-carbonitrile | Intermediate 34, CAS: 75-07-0; Yellow solid; Yield: 6.9 mg, 1.0% over 3 steps; LCMS (ES⁺): 374.1 [MH]⁺; HPLC: Rt 1.11 min, 98.1% purity |
| 180 | 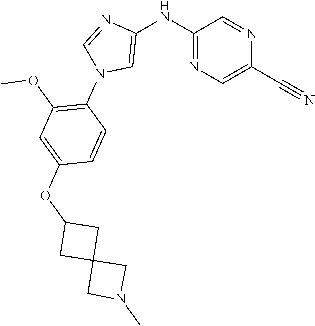 [SMILES: COc1cc(OC2CC3(C2)CN(C)C3)ccc1-n1cnc(Nc2cnc(C#N)cn2)c1] | 5-((1-(2-Methoxy-4- ((2-methyl-2- azaspiro[3.3]heptan- 6-yl)oxy)phenyl)- 1H-imidazol-4- yl)amino)pyrazine- 2-carbonitrile | Intermediate 39, CAS: 50-00-0; Yellow solid Yield: 43.0 mg, 2.8% over 3 steps; LCMS (ES⁺): 418.2 [MH]⁺ HPLC: Rt 1.02 min, 96.8% purity |

### Example 181

### 5-((1-(4-(4-(3,3-Difluorocyclobutyl)piperazin-1-yl)phenyl)-1H-imidazol-4-yl)amino)pyrazine-2-carbonitrile

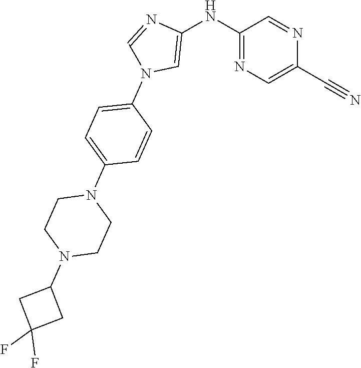 [SMILES: N#Cc1cnc(Nc2cn(-c3ccc(N4CCN(C5CC(F)(F)C5)CC4)cc3)cn2)cn1]

Example 181 was prepared from Intermediate 20 similarly to Example 175, but EPhos Pd G4 and Ephos were used instead of AdBrettPhos Pd G3 and AdBrettPhos, respectively, in Step 1, and 3,3-difluorocyclobutan-1-one was used instead of formaldehyde in Step 3. Purification via RP HPLC afforded the title compound (4.4 mg, 3.3% over 3 steps) as a light-yellow solid. LCMS (ES⁺): 437.0 [MH]⁺. HPLC: Rt 0.81 min, 95.1% purity.

### Example 182

### 5-((1-(4-(4-(Oxetan-3-yl)piperazin-1-yl)phenyl)-1H-imidazol-4-yl)amino)pyrazine-2-carbonitrile

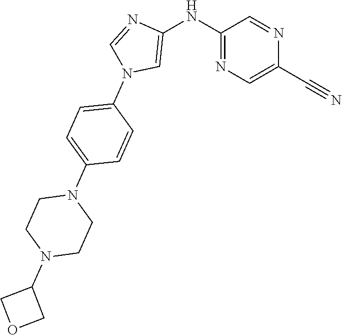 [SMILES: N#Cc1cnc(Nc2cn(-c3ccc(N4CCN(C5COC5)CC4)cc3)cn2)cn1]

Example 182 was prepared from Intermediate 20 similarly to Example 181, but 3-oxetanone was used instead of 3,3-difluorocyclobutan-1-one in Step 3. Purification via RP HPLC afforded the title compound (2.2 mg, 1.6% over 3 steps) as a yellow solid. LCMS (ES⁺): 403.2 [MH]⁺. HPLC: Rt 6.67 min, 95.4% purity.

### Example 183

### 5-((1-(4-(4-(2-Fluoroethyl)piperazin-1-yl)phenyl)-1H-imidazol-4-yl)amino)pyrazine-2-carbonitrile

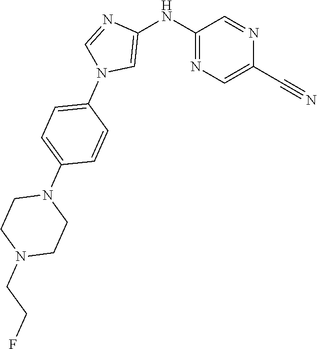 [SMILES: N#Cc1cnc(Nc2cn(-c3ccc(N4CCN(CCF)CC4)cc3)cn2)cn1]

Step 1: Intermediate 20 (800 mg, 2.85 mmol) was reacted with 5-aminopyrazine-2-carbonitrile (684 mg, 5.69 mmol) using EPhos Pd G4 similarly to Example 142. Purification via RP HPLC afforded tert-butyl 4-(4-{4-[(5-cyanopyrazin-2-yl)amino]imidazol-1-yl}phenyl)piperazine-1-carboxylate (80.0 mg, 73.0%) as a brown semi-solid. LCMS (ES)+: 447.2 [MH]⁺.

Step 2: A solution of tert-butyl 4-(4-{4-[(5-cyanopyrazin-2-yl)amino]imidazol-1-yl}phenyl)piperazine-1-carboxylate (75.0 mg, 0.17 mmol) and TFA (0.5 mL) in DCM (2.5 mL) was stirred for 30 min at RT. The resulting mixture was concentrated in vacuo to afford 5-({1-[4-(piperazin-1-yl)phenyl]imidazol-4-yl}amino)pyrazine-2-carbonitrile (40.0 mg, 68.74%) as a brown semi-solid. The resulting mixture was used in the next step directly without further purification. LCMS (ES⁺): 347.1 [MH]⁺.

Step 3: To a stirred solution of 5-({1-[4-(piperazin-1-yl)phenyl]imidazol-4-yl}amino)pyrazine-2-carbonitrile (40.0 mg, 0.115 mmol) and 1-bromo-2-fluoroethane (29.3 mg, 0.23 mmol) in DXN (2.0 mL) were added NaI (34.6 mg, 0.23 mmol) and DIEA (29.8 mg, 0.23 mmol) at RT. The resulting mixture was stirred for 12 h at 100° C., cooled to RT, filtered and the filtrate concentrated in vacuo. Purification via RP HPLC afforded the title compound (4.8 mg, 10.5%) as a yellow solid. LCMS (ES⁺): 393.3 [MH]⁺. HPLC: Rt 0.69 min, 99.2% purity.

### Examples 184 to 186

Examples 184 to 186 were prepared similarly to Example 183, via EPhos Pd G4-catalysed Buchwald-Hartwig coupling of Intermediate 20 with 5-aminopyrazine-2-carbonitrile, then Boc removal using TFA, then alkylation with the appropriate alkyl halide; see Table 17 below.

TABLE 17
EPhos Pd G4-catlaysed Buchwald-Hartwig coupling then Boc
removal then alkylation reactions

| | | | Reactant used; Form (salt); |
|---|---|---|---|
| Ex. | Structure | Name | Yield; LCMS; HPLC |
| 184 | 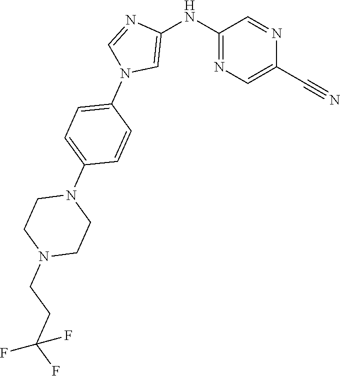 [SMILES: N#Cc1cnc(Nc2cn(-c3ccc(N4CCN(CCC(F)(F)F)CC4)cc3)cn2)cn1] | 5-((1-(4-(4-(3,3,3- Trifluoropropyl) piperazin-1-yl) phenyl)-1H- imidazol-4- yl)amino)pyrazine- 2-carbonitrile | CAS: 460-37-7; Yellow solid; Yield: 3.1 mg, 1.6% over 3 steps; LCMS (ES⁺): 443.3 [MH]⁺; HPLC: Rt 0.79 min, 98.0% purity |
| 185 | 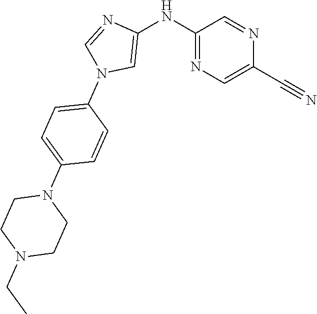 [SMILES: CCN1CCN(c2ccc(-n3cnc(Nc4cnc(C#N)cn4)c3)cc2)CC1] | 5-((1-(4-(4- Ethylpiperazin-1- yl)phenyl)-1H- imidazol-4- yl)amino)pyrazine- 2-carbonitrile | CAS: 75-03-6; Light yellow solid; Yield: 2.6 mg, 3.8% over 3 steps; LCMS (ES⁺): 375.2 [MH]⁺; HPLC: Rt 7.52 min, 95.0% purity |
| 186 | 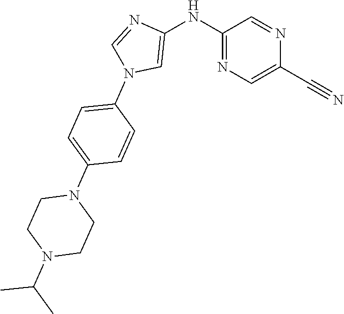 [SMILES: CC(C)N1CCN(c2ccc(-n3cnc(Nc4cnc(C#N)cn4)c3)cc2)CC1] | 5-((1-(4-(4- Isopropylpiperazin- 1-yl)phenyl)-1H- imidazol-4- yl)amino)pyrazine- 2-carbonitrile | CAS: 75-30-9;3 Yellow solid; Yield: 1.2 mg, 0.2% over 3 steps; LCMS (ES⁺): 389.2 [MH]⁺; HPLC: Rt 1.45 min, 99.4% purity |

### Comparator Examples 1 to 4

Four pyrazole-NH-pyrazine-containing Chk1 inhibitors have been highlighted for comparison; see Table 18 below.

TABLE 18
Pyrazole-NH-pyrazine-containing Comparator Examples 1 to 4

| Comparator | | | |
|---|---|---|---|
| Ex. | Structure | Name | Reference |
| 1 | 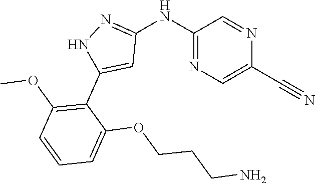 [SMILES: COc1cccc(OCCCN)c1-c1cc(Nc2cnc(C#N)cn2)n[nH]1] | 5-((5-(2-(3- Aminopropoxy)-6- methoxyphenyl)-1H- pyrazol-3- yl)amino)pyrazine- 2-carbonitrile | Angius, G. et al Cancer Chemother. Pharmacol. 2020, 85, 9 |
| 2 | 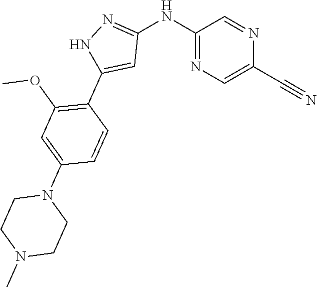 [SMILES: COc1cc(N2CCN(C)CC2)ccc1-c1cc(Nc2cnc(C#N)cn2)n[nH]1] | 5-((5-(2-Methoxy-4-(4- methylpiperazin-1- yl)phenyl)-1H-pyrazol- 3-yl)amino)pyrazine-2- carbonitrile | WO 2015120390 |
| 3 | 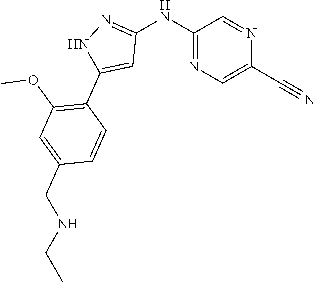 [SMILES: CCNCc1ccc(-c2cc(Nc3cnc(C#N)cn3)n[nH]2)c(OC)c1] | 5-((5-(4- ((Ethylamino)methyl)- 2-methoxyphenyl)-1H- pyrazol-3- yl)amino)pyrazine-2- carbonitrile | WO 2015120390 |
| 4 | 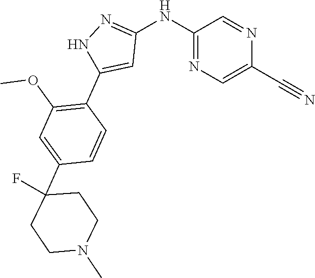 [SMILES: COc1cc(C2(F)CCN(C)CC2)ccc1-c1cc(Nc2cnc(C#N)cn2)n[nH]1] | 5-((5-(4-(4-Fluoro-1- methylpiperidin-4-yl)- 2-methoxyphenyl)-1H- pyrazol-3- yl)amino)pyrazine-2- carbonitrile | WO 2021119236, WO 2015120390 |

### High Resolution Mass Spectrometry (HRMS)

### HRMS Method

This protocol is a non-GLP UPLC-HRMS analytical method for accurate mass analysis. Samples were prepared at a concentration of 250 ng/mL in water:acetonitrile (1:1) and analyzed by UPLC-HRMS using a BEH C18 50×2.1 mm, 1.7 μm column and 0.1% formic acid in water+0.1% acetonitrile as the mobile phase.

Samples were analyzed by a Waters Synapt XS (Quadruple Time of Flight instrument) and Acquity UPLC system and corrected using LockSpray Leucine Enkephalin lockmass. The data was processed using OpenLynx application software within MassLynx. The calculated and observed mass values generated by OpenLynx are for the neutral [MH] or [M−H] or [M+H] species and the accurate mass error is reported in ppm (parts per million) and determined by the following formula:

$$Mass Error ( ppm ) = ( ( Measured Mass - Calculated Mass ) / Calculated Mass ) \times {10}^{6}$$

TABLE 19
HRMS Data

| | Measured | Calculated | | Mass Error | Chemical |
|---|---|---|---|---|---|
| Ex. | Mass | Mass | Δ mDa | (ppm) | Formula |
| 2 | 398.1290 | 398.1296 | −0.6 | −1.5 | C19H18N7FCl |
| 3 | 391.1989 | 391.1995 | −0.6 | −1.5 | C20H23N8O |
| 4 | 378.1839 | 378.1842 | −0.3 | −0.8 | C20H21N7F |
| 5 | 412.1693 | 412.1697 | −0.4 | −1.0 | C20H20N7OF2 |
| 6 | 382.1586 | 382.1592 | −0.6 | −1.6 | C19H18N7F2 |
| 7 | 375.2043 | 375.2046 | −0.3 | −0.8 | C20H23N8 |
| 8 | 361.1887 | 361.1889 | −0.2 | −0.6 | C19H21N8 |
| 9 | 373.1882 | 373.1889 | −0.7 | −1.9 | C20H21N8 |
| 10 | 373.1885 | 373.1889 | −0.4 | −1.1 | C20H21N8 |
| 11 | 373.1882 | 373.1889 | −0.7 | −1.9 | C20H21N8 |
| 12 | 375.2043 | 375.2046 | −0.3 | −0.8 | C20H23N8 |
| 13 | 405.2148 | 405.2151 | −0.3 | −0.7 | C21H25N8O |
| 14 | 419.2303 | 419.2308 | −0.5 | −1.2 | C22H27N8O |
| 15 | 375.2052 | 375.2046 | 0.6 | 1.6 | C20H23N8 |
| 16 | 375.2042 | 375.2046 | −0.4 | −1.1 | C20H23N8 |
| 17 | 375.2046 | 375.2046 | 0.0 | 0.0 | C20H23N8 |
| 18 | 362.1839 | 362.1842 | −0.3 | −0.8 | C18H20N9 |
| 19 | 362.1835 | 362.1842 | −0.7 | −1.9 | C18H20N9 |
| 20 | 376.1990 | 376.1998 | −0.8 | −2.1 | C19H22N9 |
| 21 | 379.1788 | 379.1795 | −0.7 | −1.8 | C19H20N8F |
| 22 | 389.2195 | 389.2202 | −0.7 | −1.8 | C21H25N8 |
| 23 | 379.1793 | 379.1795 | −0.2 | −0.5 | C19H20N8F |
| 24 | 394.1793 | 394.1792 | 0.1 | 0.3 | C20H21N7OF |
| 25 | 395.1497 | 395.1499 | −0.2 | −0.5 | C19H20N8Cl |
| 26 | 375.2050 | 375.2046 | 0.4 | 1.1 | C20H23N8 |
| 27 | 405.2151 | 405.2151 | 0.0 | 0.0 | C21H25N8O |
| 28 | 375.2049 | 375.2046 | 0.3 | 0.8 | C20H23N8 |
| 29 | 445.1712 | 445.1712 | 0.0 | 0.0 | C20H20N8OF3 |
| 30 | 417.2150 | 417.2151 | −0.1 | −0.2 | C22H25N8O |
| 31 | 405.2145 | 405.2151 | −0.6 | −1.5 | C21H25N8O |
| 32 | 361.1879 | 361.1889 | −1.0 | −2.8 | C19H21N8 |
| 33 | 375.2039 | 375.2046 | −0.7 | −1.9 | C20H23N8 |
| 34 | 387.2044 | 387.2046 | −0.2 | −0.5 | C21H23N8 |
| 35 | 387.2043 | 387.2046 | −0.3 | −0.8 | C21H23N8 |
| 36 | 433.2459 | 433.2464 | −0.5 | −1.2 | C23H29N8O |
| 37 | 389.2197 | 389.2202 | −0.5 | −1.3 | C21H25N8 |
| 38 | 375.2043 | 375.2046 | −0.3 | −0.8 | C20H23N8 |
| 39 | 415.2344 | 415.2359 | −1.5 | −3.6 | C23H27N8 |
| 40 | 389.2198 | 389.2202 | −0.4 | −1.0 | C21H25N8 |
| 41 | 419.2304 | 419.2308 | −0.4 | −1.0 | C22H27N8O |
| 42 | 387.2042 | 387.2046 | −0.4 | −1.0 | C21H23N8 |
| 43 | 389.2197 | 389.2202 | −0.5 | −1.3 | C21H25N8 |
| 44 | 403.1989 | 403.1995 | −0.6 | −1.5 | C21H23N8O |
| 45 | 390.2039 | 390.2042 | −0.3 | −0.8 | C21H24N7O |
| 46 | 403.1987 | 403.1995 | −0.8 | −2.0 | C21H23N8O |
| 46 | 403.1992 | 403.1995 | −0.3 | −0.7 | C21H23N8O |
| 48 | 376.1884 | 376.1886 | −0.2 | −0.5 | C20H22N7O |
| 49 | 376.1880 | 376.1886 | −0.6 | −1.6 | C20H22N7O |
| 50 | 376.1995 | 376.1998 | −0.3 | −0.8 | C19H22N9 |
| 51 | 431.2302 | 431.2308 | −0.6 | −1.4 | C23H27N8O |
| 53 | 420.2150 | 420.2148 | 0.2 | 0.5 | C22H26N7O2 |
| 54 | 446.1719 | 446.1716 | 0.3 | 0.7 | C21H20N7F4 |
| 55 | 417.2148 | 417.2151 | −0.3 | −0.7 | C22H25N8O |
| 57 | 392.1996 | 392.1999 | −0.3 | −0.8 | C21H23N7F |
| 59 | 390.2148 | 390.2155 | −0.7 | −1.8 | C20H24N9 |
| 60 | 415.2355 | 415.2359 | −0.4 | −1.0 | C23H27N8 |
| 61 | 373.1886 | 373.1889 | −0.3 | −0.8 | C20H21N8 |
| 62 | 393.1948 | 393.1951 | −0.3 | −0.8 | C20H22N8F |
| 63 | 393.1948 | 393.1951 | −0.3 | −0.8 | C20H22N8F |
| 64 | 376.1993 | 376.1998 | −0.5 | −1.3 | C19H22N9 |
| 65 | 393.1948 | 393.1951 | −0.3 | −0.8 | C20H22N8F |
| 66 | 389.2195 | 389.2202 | −0.7 | −1.8 | C21H25N8 |
| 67 | 373.1883 | 373.1889 | −0.6 | −1.6 | C20H21N8 |
| 68 | 373.1884 | 373.1889 | −0.5 | −1.3 | C20H21N8 |
| 69 | 387.2040 | 387.2046 | −0.6 | −1.5 | C21H23N8 |
| 70 | 443.1914 | 443.1920 | −0.6 | −1.4 | C21H22N8F3 |
| 71 | 443.1912 | 443.1920 | −0.8 | −1.8 | C21H22N8F3 |
| 72 | 417.2145 | 417.2151 | −0.6 | −1.4 | C22H25N8O |
| 73 | 389.2196 | 389.2202 | −0.6 | −1.5 | C21H25N8 |
| 74 | 389.2195 | 389.2202 | −0.7 | −1.8 | C21H25N8 |
| 75 | 389.2197 | 389.2202 | −0.5 | −1.3 | C21H25N8 |
| 76 | 389.2196 | 389.2202 | −0.6 | −1.5 | C21H25N8 |
| 77 | 444.1869 | 444.1872 | −0.3 | −0.7 | C20H21N9F3 |
| 78 | 389.2200 | 389.2202 | −0.2 | −0.5 | C21H25N8 |
| 79 | 393.1943 | 393.1951 | −0.8 | −2.0 | C20H22N8F |
| 80 | 391.1790 | 391.1795 | −0.5 | −1.3 | C20H20N8F |
| 81 | 391.1789 | 391.1795 | −0.6 | −1.5 | C20H20N8F |
| 82 | 377.1947 | 377.1951 | −0.4 | −1.1 | C18H21N1O |
| 83 | 418.2458 | 418.2468 | −1.0 | −2.4 | C22H28N9 |
| 85 | 390.2148 | 390.2155 | −0.7 | −1.8 | C20H24N9 |
| 86 | 389.2194 | 389.2202 | −0.8 | −2.1 | C21H25N8 |
| 87 | 387.2041 | 387.2046 | −0.5 | −1.3 | C21H23N8 |
| 88 | 393.1948 | 393.1951 | −0.3 | −0.8 | C20H22N8F |
| 89 | 389.2193 | 389.2202 | −0.9 | −2.3 | C21H25N8 |
| 90 | 390.2151 | 390.2155 | −0.4 | −1.0 | C20H24N9 |
| 91 | 387.2039 | 387.2046 | −0.7 | −1.8 | C21H23N8 |
| 92 | 379.1786 | 379.1795 | −0.9 | −2.4 | C19H20N8F |
| 93 | 444.1865 | 444.1872 | −0.7 | −1.6 | C20H21N9F3 |
| 94 | 375.2047 | 375.2046 | 0.1 | 0.3 | C20H23N8 |
| 95 | 375.2042 | 375.2046 | −0.4 | −1.1 | C20H23N8 |
| 96 | 389.2195 | 389.2202 | −0.7 | −1.8 | C21H25N8 |
| 97 | 389.2198 | 389.2202 | −0.4 | −1.0 | C21H25N8 |
| 98 | 401.2198 | 401.2202 | −0.4 | −1.0 | C22H25N8 |
| 99 | 373.1882 | 373.1889 | −0.7 | −1.9 | C20H21N8 |
| 100 | 401.2200 | 401.2202 | −0.2 | −0.5 | C22H25N8 |
| 100 | 401.2199 | 401.2202 | −0.3 | −0.7 | C22H25N8 |
| 101 | 387.2041 | 387.2046 | −0.5 | −1.3 | C21H23N8 |
| 102 | 388.1994 | 388.1998 | −0.4 | −1.0 | C20H22N9 |
| 103 | 417.2147 | 417.2151 | −0.4 | −1.0 | C22H25N8O |
| 104 | 401.2194 | 401.2202 | −0.8 | −2.0 | C22H25N8 |
| 105 | 362.1724 | 362.1729 | −0.5 | −1.4 | C19H20N7O |
| 106 | 362.1723 | 362.1729 | −0.6 | −1.7 | C19H20N7O |
| 107 | 405.2147 | 405.2151 | −0.4 | −1.0 | C21H25N8O |
| 108 | 405.2146 | 405.2151 | −0.5 | −1.2 | C21H25N8O |
| 109 | 405.2145 | 405.2151 | −0.6 | −1.5 | C21H25N8O |
| 110 | 405.2148 | 405.2151 | −0.3 | −0.7 | C21H25N8O |
| 111 | 389.2198 | 389.2202 | −0.4 | −1.0 | C21H25N8 |
| 112 | 389.2201 | 389.2202 | −0.1 | −0.3 | C21H25N8 |
| 113 | 387.2041 | 387.2046 | −0.5 | −1.3 | C21H23N8 |
| 114 | 387.2035 | 387.2046 | −1.1 | −2.8 | C21H23N8 |
| 117 | 387.2042 | 387.2046 | −0.4 | −1.0 | C21H23N8 |
| 118 | 387.2043 | 387.2046 | −0.3 | −0.8 | C21H23N8 |
| 119 | 387.2040 | 387.2046 | −0.6 | −1.5 | C21H23N8 |
| 121 | 389.2194 | 389.2202 | −0.8 | −2.1 | C21H25N8 |
| 122 | 389.2201 | 389.2202 | −0.1 | −0.3 | C21H25N8 |
| 123 | 415.2358 | 415.2359 | −0.1 | −0.2 | C23H27N8 |
| 124 | 415.2354 | 415.2359 | −0.5 | −1.2 | C23H27N8 |
| 125 | 401.2198 | 401.2202 | −0.4 | −1.0 | C22H25N8 |
| 126 | 401.2200 | 401.2202 | −0.2 | −0.5 | C22H25N8 |
| 127 | 387.2050 | 387.2046 | 0.4 | 1.0 | C21H23N8 |
| 128 | 387.2039 | 387.2046 | −0.7 | −1.8 | C21H23N8 |
| 129 | 405.1948 | 405.1951 | −0.3 | −0.7 | C21H22N8F |
| 130 | 405.1945 | 405.1951 | −0.6 | −1.5 | C21H22N8F |
| 131 | 427.2355 | 427.2359 | −0.4 | −0.9 | C24H27N8 |
| 132 | 427.2355 | 427.2359 | −0.4 | −0.9 | C24H27N8 |
| 133 | 429.2514 | 429.2515 | −0.1 | −0.2 | C24H29N8 |
| 134 | 429.2508 | 429.2515 | −0.7 | −1.6 | C24H29N8 |
| 135 | 401.2197 | 401.2202 | −0.5 | −1.2 | C22H25N8 |
| 136 | 401.2194 | 401.2202 | −0.8 | −2.0 | C22H25N8 |
| 137 | 401.2193 | 401.2202 | −0.9 | −2.2 | C22H25N8 |
| 138 | 401.2196 | 401.2202 | −0.6 | −1.5 | C22H25N8 |
| 139 | 415.2352 | 415.2359 | −0.7 | −1.7 | C23H27N8 |
| 140 | 415.2357 | 415.2359 | −0.2 | −0.5 | C23H27N8 |
| 142 | 362.1722 | 362.1729 | −0.7 | −1.9 | C19H20N7O |
| 142 | 362.1722 | 362.1729 | −0.7 | −1.9 | C19H20N7O |
| 143 | 361.1881 | 361.1889 | −0.8 | −2.2 | C19H21N8 |
| 144 | 389.1839 | 389.1838 | 0.1 | 0.3 | C20H21N8O |
| 145 | 375.2050 | 375.2046 | 0.4 | 1.1 | C20H23N8 |
| 146 | 394.1789 | 394.1792 | −0.3 | −0.8 | C20H21N7OF |
| 149 | 366.1673 | 366.1678 | −0.5 | −1.4 | C18H20N7O2 |
| 150 | 322.1410 | 322.1416 | −0.6 | −1.9 | C16H16N7O |
| 151 | 365.1629 | 365.1638 | −0.9 | −2.5 | C18H18N8F |
| 152 | 365.1632 | 365.1638 | −0.6 | −1.6 | C18H18N8F |
| 153 | 347.1719 | 347.1733 | −1.4 | −4.0 | C18H19N8 |
| 154 | 373.1880 | 373.1889 | −0.9 | −2.4 | C20H21N8 |
| 155 | 361.1878 | 361.1889 | −1.1 | −3.0 | C19H21N8 |
| 156 | 361.1879 | 361.1889 | −1.0 | −2.8 | C19H21N8 |
| 158 | 347.1721 | 347.1733 | −1.2 | −3.5 | C18H19N8 |
| 159 | 365.1631 | 365.1638 | −0.7 | −1.9 | C18H18N8F |
| 160 | 359.1729 | 359.1733 | −0.4 | −1.1 | C19H19N8 |
| 161 | 359.1728 | 359.1733 | −0.5 | −1.4 | C19H19N8 |
| 162 | 375.2045 | 375.2046 | −0.1 | −0.3 | C20H23N8 |
| 164 | 409.1888 | 409.1901 | −1.3 | −3.2 | C20H22N8OF |
| 165 | 361.1881 | 361.1889 | −0.8 | −2.2 | C19H21N8 |
| 166 | 397.1695 | 397.1701 | −0.6 | −1.5 | C19H19N8F2 |
| 167 | 397.1696 | 397.1701 | −0.5 | −1.3 | C19H19N8F2 |
| 169 | 394.1788 | 394.1792 | −0.4 | −1.0 | C20H21N7OF |
| 170 | 350.1724 | 350.1729 | −0.5 | −1.4 | C18H20N7O |
| 171 | 376.1881 | 376.1886 | −0.5 | −1.3 | C20H22N7O |
| 172 | 426.1851 | 426.1854 | −0.3 | −0.7 | C21H22N7OF2 |
| 173 | 392.1836 | 392.1835 | 0.1 | 0.3 | C20H22N7O2 |
| 174 | 378.1841 | 378.1842 | −0.1 | −0.3 | C20H21N7F |
| 175 | 388.1886 | 388.1886 | 0.0 | 0.0 | C21H22N7O |
| 176 | 332.1611 | 332.1624 | −1.3 | −3.9 | C18H18N7 |
| 177 | 403.1988 | 403.1995 | −0.7 | −1.7 | C21H23N8O |
| 178 | 388.1887 | 388.1886 | 0.1 | 0.3 | C21H22N7O |
| 178 | 388.1883 | 388.1886 | −0.3 | −0.8 | C21H22N7O |
| 179 | 374.2086 | 374.2093 | −0.7 | −1.9 | C21H24N7 |
| 180 | 418.1992 | 418.1991 | 0.1 | 0.2 | C22H24N7O2 |
| 181 | 437.2008 | 437.2014 | −0.6 | −1.4 | C22H23N8F2 |
| 182 | 403.1988 | 403.1995 | −0.7 | −1.7 | C21H23N8O |
| 183 | 393.1948 | 393.1951 | −0.3 | −0.8 | C20H22N8F |
| 184 | 443.1919 | 443.1920 | −0.1 | −0.2 | C21H22N8F3 |
| 185 | 375.2040 | 375.2046 | −0.6 | −1.6 | C20H23N8 |

### Biological Data

### Human Chk1 HTRF (Homogeneous Time Resolved Fluorescence) Enzyme Assay

### Method

2 μL enzyme/substrate solution (1 nM Chk-1 (Carna Bioscience #02-177), in solution with 30 μM ATP, 4 μM STK1 S1 (Cisbio #62ST1PEB) in assay buffer (20 mM HEPES pH7.5, 500 mM NaCl₂, 0.1% BGG, 1 mM DTT, 0.005% Tween-20)) and different concentrations of compounds were incubated at room temperature for 30 minutes in 384-well plates (proxiplate-384 plus, Perkin Elmer cat #6008289). MgCl₂ cofactor solution was added (2 μL, 20 mM) and incubation continued at room temperature for 30 minutes. 4 μL kit detection reagent (Cisbio #62ST1PEB) was added and incubation continued for 60 minutes at room temperature. Plates were read on the PHERAstar FS using TR-FRET technology, channel A: 337/665, channel B: 337/620. % effects of a compound at a given concentration was calculated based on inhibition produced in the DMSO control wells and inhibited control wells control wells in each assay plate. Compound IC₅₀s were determined with a four-parameter logistic dose response equation using 11 point dose response curves, analysing within the Dotmatics platform.

The exemplified compounds of the invention were tested for their inhibitory potential of human Chk1 kinase in the HTRF assay and the PIC₅₀ data is shown in Table 20. pIC₅₀ is the negative log of the IC₅₀ value when converted to molar. All of the exemplified compounds of the invention have a pIC₅₀ value >6. This data shows that the compounds of the invention can inhibit Chk1.

TABLE 20
Chk1 pIC₅₀ data (A: 6-6.5, B: 6.5-7.0,
C: 7.0-7.5, D: 7.5-8.0, E: ≥8.0)

| | Chk1 |
|---|---|
| Example | pIC₅₀ |
| 1 | E |
| 2 | A |
| 3 | E |
| 4 | C |
| 5 | A |
| 6 | B |
| 7 | B |
| 8 | C |
| 9 | D |
| 10 | D |
| 11 | D |
| 12 | E |
| 13 | D |
| 14 | D |
| 15 | C |
| 16 | D |
| 17 | C |
| 18 | D |
| 19 | B |
| 20 | B |
| 21 | C |
| 22 | D |
| 23 | C |
| 24 | C |
| 25 | B |
| 26 | D |
| 27 | E |
| 28 | D |
| 29 | C |
| 30 | E |
| 31 | D |
| 32 | E |
| 33 | C |
| 34 | D |
| 35 | C |
| 36 | C |
| 37 | E |
| 38 | D |
| 39 | D |
| 40 | E |
| 41 | D |
| 42 | E |
| 43 | C |
| 44 | D |
| 45 | D |
| 46 | D |
| 47 | E |
| 48 | C |
| 49 | D |
| 50 | E |
| 51 | E |
| 52 | C |
| 53 | D |
| 54 | B |
| 55 | D |
| 56 | B |
| 57 | A |
| 58 | C |
| 59 | E |
| 60 | C |
| 61 | D |
| 62 | D |
| 63 | E |
| 64 | D |
| 65 | E |
| 66 | C |
| 67 | C |
| 68 | C |
| 69 | D |
| 70 | C |
| 71 | B |
| 72 | D |
| 73 | E |
| 74 | E |
| 75 | E |
| 76 | D |
| 77 | D |
| 78 | E |
| 79 | E |
| 80 | C |
| 81 | C |
| 82 | C |
| 83 | A |
| 84 | E |
| 85 | E |
| 86 | C |
| 87 | D |
| 88 | D |
| 89 | E |
| 90 | D |
| 91 | D |
| 92 | E |
| 93 | D |
| 94 | C |
| 95 | C |
| 96 | D |
| 97 | C |
| 98 | E |
| 99 | D |
| 100 | D |
| 101 | E |
| 102 | D |
| 103 | E |
| 104 | D |
| 105 | C |
| 106 | C |
| 107 | C |
| 108 | D |
| 109 | D |
| 110 | D |
| 111 | C |
| 112 | C |
| 113 | D |
| 114 | E |
| 115 | C |
| 116 | C |
| 117 | D |
| 118 | D |
| 119 | D |
| 120 | D |
| 121 | E |
| 122 | D |
| 123 | D |
| 124 | D |
| 125 | D |
| 126 | D |
| 127 | D |
| 128 | D |
| 129 | C |
| 130 | D |
| 131 | D |
| 132 | D |
| 133 | E |
| 134 | D |
| 135 | D |
| 136 | C |
| 137 | D |
| 138 | E |
| 139 | C |
| 140 | E |
| 141 | B |
| 142 | B |
| 143 | D |
| 144 | B |
| 145 | A |
| 146 | C |
| 147 | D |
| 148 | C |
| 149 | A |
| 150 | D |
| 151 | C |
| 152 | D |
| 153 | D |
| 154 | E |
| 155 | E |
| 156 | D |
| 157 | E |
| 158 | D |
| 159 | D |
| 160 | D |
| 161 | D |
| 162 | E |
| 163 | D |
| 164 | E |
| 165 | E |
| 166 | C |
| 167 | C |
| 168 | B |
| 169 | D |
| 170 | D |
| 171 | D |
| 172 | C |
| 173 | C |
| 174 | C |
| 175 | C |
| 176 | C |
| 177 | B |
| 178 | C |
| 179 | B |
| 180 | C |
| 181 | C |
| 182 | C |
| 183 | C |
| 184 | B |
| 185 | D |
| 186 | B |

### Cancer Cell Line Data

### In Vitro Cell Viability Assay

GBM LN18 cells were incubated with increasing concentrations of selected compounds of the invention for 72 hours prior to the measurement of ATP in metabolically viable cells with the Cell Titre® Glo Luminescent reagent. A 10-point concentration response curve up to a maximum concentration of 10 μM was tested.

The percent (%) effect i.e., inhibition at each concentration of compound was calculated based on, and relative to the amount of inhibition produced in the vehicle control and inhibited control wells contained within each plate. The log molar concentrations and % inhibition values were plotted and the concentration of compound required for 50% inhibition (IC₅₀) was determined using the four-parameter logistic dose response equation.

### ELISA

Cell-based on-target kinase activity was measured for selected compounds of the invention using a sandwich ELISA assay kit (pChk1 PathScan ELISA kit, Cell Signalling Technologies, Cat #7870) with pChk1(Ser296) as the detection antibody.

SW620 cells were seeded in 96-well plates and incubated overnight at 37° C., 5% CO₂ prior to being treated with the DNA damage-inducer neocarzinostatin (7.5 μM) and increasing concentrations of test agent. Cells were returned to the incubator for 2 hours and thereafter lysed in 30 μl of the lysis buffer provided in the kit (1:10 dilution of 10× cell lysis buffer #9803 supplemented with protease and phosphatase inhibitors). ELISA assays were performed as per the manufacturer's protocol. However, an alternative detection antibody pChk1(Ser296; Cat #2349, Cell Signalling Technology) was used instead of the pChk1(Ser317) antibody supplied with the kit. Selected compounds of the invention were tested as a 8-point concentration response up to a maximum concentration of either 1 μM, 3 μM or 10 μM. The percent (%) effect i.e. inhibition at each concentration of compound was calculated based on, and relative to, the amount of inhibition produced in the vehicle control and inhibited control wells contained within each plate. The concentrations and % inhibition values for tested compounds were plotted on a dose response curve and the concentration of compound required for 50% inhibition (IC₅₀) was determined with a four-parameter logistic dose response equation.

A selection of the exemplified compounds of the invention were tested for their inhibitory potential in the human-derived glioblastoma (LN18) and colorectal cancer (SW620) cell lines, and the data is shown in Table 21. pIC₅₀ is the negative log of the IC₅₀ value when converted to molar. These data show that the compounds of the invention are effective in cancer cell lines at therapeutically relevant concentrations.

TABLE 21
Cellular pIC₅₀ data (A: 5.0-6.0, B: >6.0)

| | LN18 | SW620 |
|---|---|---|
| Ex. | pIC₅₀ | pIC₅₀ |
| 1 | B | A |
| 12 | B | B |
| 47 | B | B |
| 50 | B | B |
| 52 | A | |
| 59 | B | A |
| 65 | B | A |
| 67 | B | |
| 68 | B | |
| 69 | A | |
| 71 | B | |
| 73 | B | B |
| 74 | B | B |
| 75 | B | |
| 76 | B | |
| 78 | B | B |
| 79 | B | |
| 83 | A | |
| 84 | B | A |
| 85 | B | B |
| 89 | B | |
| 90 | B | |
| 92 | B | B |
| 96 | B | |
| 98 | B | B |
| 113 | B | A |
| 114 | B | B |
| 141 | A | |
| 150 | A | |
| 152 | A | |
| 153 | A | A |
| 154 | B | A |
| 155 | B | B |
| 156 | B | A |
| 157 | A | |
| 158 | A | |
| 165 | B | |
| 168 | A | A |

### Kinome Selectivity

Selected compounds of the invention were tested for wider kinome selectivity against the Eurofins KinaseProfiler™ kinase screen consisting of 373 human wild-type kinases, using a 9-point concentration response curve up to a top concentration of 10 μM. The radiometric kinase activity assays were run at approximately the ATP Kₘ for each kinase. The number of kinases inhibited within 10-fold and 100-fold Chk1 IC₅₀ are shown in Table 22, which indicates that these compounds have excellent selectivity for Chk1 over the wider kinome.

TABLE 22
Selectivity for Chk1 within a panel of 373 human wild-type kinases

Number of 372 kinases inhibited
relative to Chk1 IC₅₀

| | Within | Within |
|---|---|---|
| Example | 10-fold | 100-fold |
| 12 | 1 | 5 |
| 50 | 1 | 5 |
| 98 | 1 | 8 |
| 143 | 3 | 10 |
| 155 | 1 | 6 |

### Human Chk2 HTRF Enzyme Assay

### Method

2 μL enzyme/substrate solution (2 nM Chk-1 (Carna Bioscience #02-162), in solution with 200 μM ATP, 4 μM STK1 S1 (Cisbio #62ST1PEB) in assay buffer (20 mM HEPES pH7.5, 500 mM NaCl₂, 0.1% BGG, 1 mM DTT, 0.005% Tween-20)) and different concentrations of compounds were incubated at room temperature for 30 minutes in 384-well plates (Proxiplate-384 plus, Perkin Elmer cat #6008289). MgCl₂ cofactor solution was added (2 μL, 20 mM) and incubation continued at room temperature for 30 minutes. 4 μL kit detection reagent (Cisbio #62ST1PEB) was added and incubation continued for 60 minutes at room temperature. Plates were read on the PHERAstar FS using TR-FRET technology, channel A: 337/665, channel B: 337/620. % effects of a compound at a given concentration was calculated based on inhibition produced in the DMSO control wells and inhibited control wells control wells in each assay plate. Compound IC₅₀s were determined with a four-parameter logistic dose response equation, using 11 point dose response curves, analysing within the Dotmatics platform.

The exemplified compounds of the invention were tested for their inhibitory potential of human Chk2 kinase in the HTRF assay and the PIC₅₀ data is shown in Table 23. All of the exemplified compounds of the invention have a pIC₅₀ value <6. This data shows that the compounds of the invention are not potent Chk2 inhibitors.

TABLE 23
Chk2 pIC₅₀ data (A: ≤4.0, B: 5.0-4.0, C: 6.0-5.0)

| | Chk2 |
|---|---|
| Example | pIC₅₀ |
| 1 | C |
| 2 | A |
| 3 | C |
| 4 | B |
| 5 | A |
| 6 | A |
| 7 | A |
| 8 | B |
| 9 | B |
| 10 | B |
| 11 | B |
| 12 | C |
| 13 | B |
| 14 | B |
| 15 | B |
| 16 | B |
| 17 | B |
| 18 | C |
| 19 | B |
| 20 | B |
| 21 | B |
| 22 | B |
| 23 | A |
| 24 | A |
| 25 | C |
| 26 | B |
| 27 | C |
| 28 | B |
| 29 | A |
| 30 | B |
| 31 | B |
| 32 | A |
| 33 | B |
| 34 | B |
| 35 | A |
| 36 | B |
| 37 | C |
| 38 | B |
| 39 | B |
| 40 | B |
| 41 | B |
| 42 | B |
| 43 | B |
| 44 | B |
| 45 | B |
| 46 | B |
| 47 | C |
| 48 | A |
| 49 | B |
| 50 | C |
| 51 | C |
| 52 | B |
| 53 | B |
| 54 | A |
| 55 | B |
| 56 | B |
| 57 | A |
| 58 | B |
| 59 | C |
| 60 | B |
| 61 | B |
| 62 | C |
| 63 | C |
| 64 | C |
| 65 | C |
| 66 | B |
| 67 | B |
| 68 | B |
| 69 | B |
| 70 | C |
| 71 | A |
| 72 | B |
| 73 | C |
| 74 | C |
| 75 | C |
| 76 | B |
| 77 | B |
| 78 | C |
| 79 | C |
| 80 | B |
| 81 | C |
| 82 | C |
| 83 | B |
| 84 | n/r |
| 85 | C |
| 86 | B |
| 87 | B |
| 88 | C |
| 89 | C |
| 90 | C |
| 91 | B |
| 92 | C |
| 93 | A |
| 94 | B |
| 95 | B |
| 96 | B |
| 97 | B |
| 98 | C |
| 99 | B |
| 100 | B |
| 101 | B |
| 102 | C |
| 103 | C |
| 104 | B |
| 105 | B |
| 106 | B |
| 107 | B |
| 108 | B |
| 109 | A |
| 110 | B |
| 111 | B |
| 112 | B |
| 113 | B |
| 114 | C |
| 115 | n/r |
| 116 | n/r |
| 117 | A |
| 118 | A |
| 119 | B |
| 120 | n/r |
| 121 | A |
| 122 | B |
| 123 | A |
| 124 | C |
| 125 | C |
| 126 | A |
| 127 | A |
| 128 | A |
| 129 | A |
| 130 | C |
| 131 | C |
| 132 | C |
| 133 | B |
| 134 | A |
| 135 | B |
| 136 | A |
| 137 | A |
| 138 | C |
| 139 | A |
| 140 | C |
| 141 | B |
| 142 | B |
| 143 | B |
| 144 | n/r |
| 145 | B |
| 146 | B |
| 147 | n/r |
| 148 | B |
| 149 | B |
| 150 | B |
| 151 | B |
| 152 | B |
| 153 | C |
| 154 | C |
| 155 | C |
| 156 | B |
| 157 | B |
| 158 | B |
| 159 | C |
| 160 | C |
| 161 | C |
| 162 | C |
| 163 | n/r |
| 164 | C |
| 165 | C |
| 166 | A |
| 167 | A |
| 168 | B |
| 169 | B |
| 170 | B |
| 171 | B |
| 172 | B |
| 173 | B |
| 174 | B |
| 175 | B |
| 176 | B |
| 177 | B |
| 178 | B |
| 179 | A |
| 180 | B |
| 181 | A |
| 182 | B |
| 183 | B |
| 184 | A |
| 185 | B |
| 186 | A |

### RSK1-4 Kinase Assay

Selected compounds of the invention were tested for their inhibitory potential of RSK1, RSK2, RSK3 and RSK4 kinases via the Eurofins KinaseProfiler™ service, using a 9-point concentration response curve up to a top concentration of 10 μM. The radiometric kinase activity assays were run at approximately the ATP Kₘ for each kinase and the IC₅₀ data is shown in Table 24. This data shows that the compounds of the invention are not potent RSK1-4 inhibitors.

TABLE 24
RSK1-4 IC₅₀ data (nM)

| Example | RSK1 IC₅₀ | RSK2 IC₅₀ | RSK3 IC₅₀ | RSK4 IC₅₀ |
|---|---|---|---|---|
| Comparator | 20 | 2 | 4 | 5 |
| Ex. 1 | | | | |
| (Prexasertib) | | | | |
| Comparator | 259 | 80 | 24 | 77 |
| Ex. 2 | | | | |
| Comparator | 570 | 172 | 60 | 163 |
| Ex. 3 | | | | |
| Comparator | 329 | 59 | 35 | 58 |
| Ex. 4 | | | | |
| 4 | >10000 | 2460 | 2210 | 4960 |
| 12 | >10000 | 6620 | 4340 | 9200 |
| 50 | 4470 | 6670 | 1020 | 3140 |
| 61 | 6780 | 2670 | 1920 | 4070 |
| 98 | 3550 | 786 | 651 | 2470 |
| 143 | 9450 | 3040 | 2070 | >10000 |
| 155 | 3420 | 1120 | 822 | 1960 |
| 168 | >10000 | 7070 | 5830 | >10000 |

### MDCK-MDR1 and MDCK-BCRP Efflux Assay

Assay protocol: Wild-type (WT) MDCK, MDR1-MDCK and BCRP-MDCK cells were seeded into 24 well Transwell plates and cultured for 3 days to form monolayers. Test compounds were prepared at 1 μM in Hanks' Balanced Salt Solution containing 25 mM HEPES and loaded into the donor compartments of Transwell plates bearing the cell monolayers (pH 7.4 for both donor and receiver compartments). Lucifer Yellow was added to the apical buffer in all wells to assess integrity of the cell monolayer. Duplicate wells were prepared and incubated at 37° C. in a CO₂ incubator. Samples were removed at time 0 and 60 min and test compound analysed by LC-MS/MS. Concentrations of Lucifer Yellow in the samples were measured using a fluorescence plate reader. The apparent permeability (Pₐₚₚ) values of test compound were determined for both the apical to basal (A>B) and basal to apical (B>A) permeation and the efflux ratio (B>A:A>B) determined in each cell line.

Compounds that can freely permeate the blood-brain barrier (BBB) typically have ≤1 hydrogen bond donors, tPSA ≤90 Å² (Hitchcock, J Med Chem, 2012, 4877-4895), high passive permeability with apparent permeability (Pₐₚₚ) values >15×10e⁻⁶ cm/s, and low efflux by P-glycoprotein (P-gp), encoded by the MDR1 gene, and BCRP with efflux ratios ≤2.5 (Doan et al., J Pharmacol Exp Ther, 2002, 1029-1037). The data in Table 25 suggests that many of the compounds of the invention may be able to efficiently cross the blood-brain barrier to treat cancers of the brain and central nervous system.

TABLE 25
CNS-relevant properties of selected Examples

| | | | | MDCK- | MDCK- |
|---|---|---|---|---|---|
| | | | MDCK WT | MDR1 | BCRP |
| | | tPSA | Pₐₚₚ A-B | efflux | efflux |
| Example | HBD | (Å²) | (10e⁻⁶ cm/s) | ratio | ratio |
| Comparator | 4 | 135 | 5 | 20 | >79 |
| Ex. 1 | | | | | |
| Prexasertib | | | | | |
| Comparator | 2 | 103 | 19 | 16 | 12 |
| ex 4 | | | | | |
| 12 | 1 | 86 | 58 | 1.5 | 1.5 |
| 32 | 1 | 86 | 109 | 1.5 | 1.8 |
| 40 | 1 | 86 | 86 | 1.5 | 2.3 |
| 62 | 1 | 86 | 119 | 1.3 | 1.3 |
| 74 | 1 | 86 | 73 | 2.4 | 1.1 |
| 75 | 1 | 86 | 63 | 1.2 | 0.9 |
| 76 | 1 | 86 | 50 | 0.3 | 0.4 |
| 84 | 1 | 86 | 32 | 1.2 | 1.7 |
| 86 | 1 | 86 | 36 | 0.6 | 0.6 |
| 89 | 1 | 86 | 28 | 2.3 | 2.2 |
| 91 | 1 | 86 | 151 | 0.4 | 0.4 |
| 114 | 1 | 86 | 41 | 2.0 | 1.5 |
| 147 | 1 | 86 | 17 | 2.3 | 2.0 |
| 172 | 1 | 92 | 47 | 1.6 | 0.8 |
| 179 | 1 | 83 | 54 | 0.7 | 1.3 |
| 181 | 1 | 86 | 39 | 1.9 | 1.5 |
| 183 | 1 | 86 | 84 | 0.7 | 1.4 |
| 184 | 1 | 86 | 43 | 0.9 | 1.1 |
| 185 | 1 | 86 | 52 | 1.5 | 2.3 |
| 186 | 1 | 86 | 70 | 2.1 | 1.0 |

### Determination of CNS Penetration In Vivo

Male Sprague Dawley Rats 300-350 g (Charles River, UK) were group housed, n=2, under a 12 h light/dark cycle with food and water available ad libitum. Two days prior to dosing, animals were anaesthetised with inhaled isoflurane, and the right jugular vein was exposed and surgically cannulated. Animals were then housed singly for recovery, and throughout the remaining procedure. On the day of dosing animals were weighed, tail marked and dosed intravenously via the indwelling cannula with compound at 0.25 mg/kg in a volume of 3 mL/kg. Animals were culled at 15 min post dose via intravenous administration of pentobarbital. Post-mortem blood was withdrawn via cardiac puncture, and briefly stored in K2 EDTA blood tubes on ice before being spun at 14,000 g for 4 min at 4° C. Plasma was withdrawn into a 96 well plate, placed on dry ice and stored at −80° C. Brains were quickly dissected and placed on dry ice before storage at −80° C.

Following dosing of test compound (intravenous) to Male Sprague-Dawley Rats, animals are sacrificed at 15 min timepoint. Plasma is isolated from whole blood following cardiac exsanguination by centrifugal blood fractionation and whole brains isolated. Samples are stored on-ice and transferred to the Bioanalytical lab storage at −80° C. Bioanalysis of plasma and brain samples is performed as detailed below.

### Plasma Bioanalysis

Typically, a 1.00 mg/mL DMSO stock was used to prepare calibration standards of test compound in the range 1.00 to 6,000 ng/mL. Calibration lines were prepared by printing known masses of analyte into a 96-well plate in the range 25 to 150,000 pg. A volume of 25 μL of control male Sprague-Dawley Rat plasma was added to each well to prepare calibration standards at the appropriate concentration across the calibration range. Experimental samples were thawed to room temperature and 25 μL aliquots were added to the 96-well precipitation plate alongside the calibration lines. Samples were extracted using protein precipitation (agitation for at least 5 min at RT with 300 μL of MeCN containing 25 ng/mL tolbutamide as an internal standard). Protein precipitates were separated from the extracted test compound by centrifugation at 4000 rpm for 5 min, 4° C. The resulting supernatants were diluted in a ratio of 1:2 with diluent, 1:1 MeOH:H₂O.

Samples were analysed by UPLC-MS/MS on either an AB Sciex API6500 QTrap or Waters TQ-S mass spectrometer using previously optimised analytical MRM (multiple reaction monitoring) methods, specific to the test compound.

The concentration of test compound in isolated samples was determined following analysis of the samples against the two replicates of the calibration line, injected before and after the sample set with an appropriate regression and weighting used. Only calibrators within ±15% of the expected test concentration value were included in the calibration line (±20% at the LLoQ) and any samples that fell outside of the limits of the calibration line were deemed to be less than or above the limit of quantification (LLoQ/ALoQ).

### Brain Bioanalysis

Typically, a 1.00 mg/mL DMSO stock was used to prepare calibration standards of test compound in the range 3.00 to 18,000 ng/mL. Calibration lines were prepared by printing known masses of analyte into a 96-well plate in the range 25 to 150,000 pg. A volume of 25 μL of control male Sprague-Dawley Rat brain homogenate (containing 8.33 mg of brain tissue) was added to each well to prepare calibration standards at the appropriate concentration across the calibration range.

To prepare control and experimental brain homogenates, brains were thawed at room temperature, weighed and a volume of diluent added (50:50 MeCN/H₂O) in the ratio of 2 mL per gram of brain. Homogenisation of brains was performed by bead-beater homogenisation using Precellys Evolution and CKMix50 7 mL mixed ceramic bead homogenisation tubes.

Aliquots of 25 μL experimental sample were extracted alongside the calibration lines using protein precipitation (agitation for at least 5 min at room temperature with 300 μL of MeCN containing 25 ng/mL tolbutamide as an internal standard). Protein precipitates were separated from the extracted test compound by centrifugation at 4000 rpm for 5 min, 4° C. The resulting supernatants were diluted in a ratio of 1:2 with diluent, 1:1 MeOH:H₂O.

Samples were analysed by UPLC-MS/MS on either an AB Sciex AP16500 QTrap or Waters TQ-S mass spectrometer using previously optimised analytical MRM (multiple reaction monitoring) methods, specific to the test compound.

The concentration of test compound in isolated samples was determined following analysis of the samples against the two replicates of the calibration line, injected before and after the sample set with an appropriate regression and weighting used. Only calibrators within ±15% of the expected test concentration value (±20% at the LLoQ) were included in the calibration line and any samples that fell outside of the limits of the calibration line were deemed to be less than or above the limit of quantification (LLoQ/ALoQ).

### Determination of Unbound Brain to Plasma Ratio

The free drug hypothesis states that only unbound compound is able to interact with and elicit a pharmacological effect. Therefore, it is desirable for compounds to have a high free brain concentration. To calculate the free concentrations in each matrix, the determined concentrations are multiplied by the % free value as determined by plasma protein binding and brain tissue binding studies using rapid equilibrium dialysis.

The Kₚᵤᵤ is calculated as the ratio of free drug fraction unbound in brain to free drug unbound in plasma. The results (Table 26) show that the compounds of the invention have a high free brain concentration (Kₚᵤᵤ). That is, they have a higher ratio of free drug fraction unbound in brain to free drug unbound in plasma. This makes them better able to elicit a pharmacological effect in the brain.

TABLE 26
Unbound Brain to plasma partitioning
(Kₚᵤᵤ) for selected compounds

| | | | Dose | |
|---|---|---|---|---|
| Ex. | Formulation | Route | (mg/kg) | Kₚᵤᵤ |
| 12 | 10% NMP/90% HP-β-CD | IV | 1 | 1.2 |
| | (30% w/v in saline) | | | |
| 50 | 10% NMP/90% HP-β-CD | IV | 1 | 0.5 |
| | (30% w/v in saline) | | | |
| 98 | 10% NMP/90% HP-β-CD | IV | 1 | 0.8 |
| | (30% w/v in saline) | | | |
| 143 | 10% NMP/90% HP-β-CD | IV | 1 | 0.4 |
| | (30% w/v in saline) | | | |

1. A compound of Formula (I): 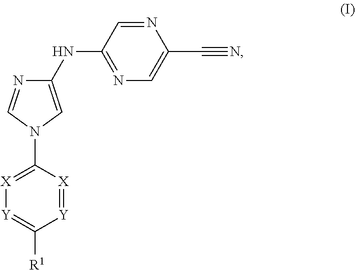
or a pharmaceutically acceptable salt, solvate, hydrate, tautomer, enantiomer, diastereoisomer, isotopic form, N-oxide, and/or prodrug thereof, wherein
each X is independently N or CR²;
each Y is independently N or CR³;
0, 1, or 2 of X and Y are N;
R¹, R² and R³ are independently selected from
(a) H, halo, (C₁-C₆)alkyl, (C₁-C₆)haloalkyl, (C₃-C₉)cycloalkyl, —OR⁴, —NR⁵R⁶, —C(O)R⁷, wherein the (C₁-C₆)alkyl, (C₁-C₆)haloalkyl, and (C₃-C₉)cycloalkyl are optionally substituted with one or more R⁸;
(b) 4- to 7-membered non-aromatic heterocycle optionally substituted with one or more R⁹; and
(c) 7- to 10-membered spiro, bridged or fused heterocyclic system, each of which is optionally substituted with one or more of (C₁-C₆)alkyl, (C₁-C₆)haloalkyl, and —NR₂;
R⁴ is selected from (C₁-C₆)alkyl, (C₁-C₆)haloalkyl, (C₁-C₆)alkylene-NR₂, 4- to 6-membered non-aromatic heterocycle, and 6- to 9-membered heterocyclic spiro system, wherein the 4- to 6-membered non-aromatic heterocycle and 6- to 9-membered heterocyclic spiro system are optionally substituted with one or more of halo and (C₁-C₆)alkyl;
R⁵ is selected from H, (C₁-C₆)alkyl, and (C₁-C₆)haloalkyl;
R⁶ is selected from H, (C₁-C₆)alkyl, (C₁-C₆)haloalkyl, and 4- to 7-membered non-aromatic heterocycle, wherein the heterocycle is optionally substituted with one or more of halo and (C₁-C₆)alkyl;
R⁷ is selected from H, (C₁-C₆)alkyl, (C₁-C₆)haloalkyl, —NR₂, and 4- to 7-membered non-aromatic heterocycle, wherein the heterocycle is optionally substituted with one or more of halo and (C₁-C₆)alkyl;
R⁸ is selected from —NR₂, and —(O)ₙ-(4- to 7-membered non-aromatic heterocycle), wherein the 4- to 7-membered non-aromatic heterocycle is optionally substituted with one or more of halo and (C₁-C₆)alkyl;
R⁹ is selected from halo, (C₁-C₆)alkyl, (C₁-C₆)haloalkyl, (C₃-C₆)cycloalkyl, (C₁-C₆)alkoxy, —NR₂, —N(R)(C₁-C₆)hydroxyalkyl, (C₁-C₆)alkylene-NR₂, 4- to 6-membered non-aromatic heterocycle, and 5- to 8-membered heterocyclic spiro system, wherein the (C₃-C₆)cycloalkyl, 4- to 6-membered non-aromatic heterocycle, and 5- to 8-membered heterocyclic spiro system are optionally substituted with one or more of halo and (C₁-C₆)alkyl; and
each R is independently selected from H, (C₁-C₆)alkyl and (C₁-C₆)haloalkyl.

2. The compound of claim 1, wherein R¹ is selected from 4- to 7-membered non-aromatic heterocycle optionally substituted with one or more R⁹; and 7- to 10-membered spiro, bridged or fused heterocyclic system optionally substituted with one or more of (C₁-C₆)alkyl, (C₁-C₆)haloalkyl, and —NR₂;
wherein R⁹ is selected from halo, (C₁-C₃)alkyl, (C₁-C₃)haloalkyl, (C₃-C₆)cycloalkyl, (C₁-C₃)alkoxy, —NR₂, (C₁-C₃)alkylene-NR₂, 4- to 6-membered non-aromatic heterocycle, and 5- to 8-membered heterocyclic spiro system, wherein the (C₃-C₆)cycloalkyl, 4- to 6-membered non-aromatic heterocycle, and 5- to 8-membered heterocyclic spiro system are optionally substituted with one or more of halo and (C₁-C₆)alkyl; and
each R is independently selected from H, (C₁-C₆)alkyl and (C₁-C₆)haloalkyl.

3. The compound of claim 2, wherein R⁹ is selected from —NR₂, (C₁-C₃)alkylene-NR₂, 4- to 6-membered non-aromatic heterocycle, and 5- to 8-membered heterocyclic spiro system, wherein the 4- to 6-membered non-aromatic heterocycle and 5- to 8-membered heterocyclic spiro system are optionally substituted with one or more of halo and (C₁-C₆)alkyl.

4. The compound of claim 2, wherein R¹ is 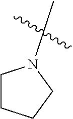 [SMILES: *N1CCCC1]
optionally substituted with one or more of halo, (C₁-C₃)alkyl, (C₁-C₃)alkoxy, NH₂, —N(H)(C₁-C₃)alkyl, —N((C₁-C₃)alkyl)₂, —N(H)(C₁-C₃)haloalkyl, —(C₁-C₃)alkylene-N((C₁-C₃)alkyl)₂, 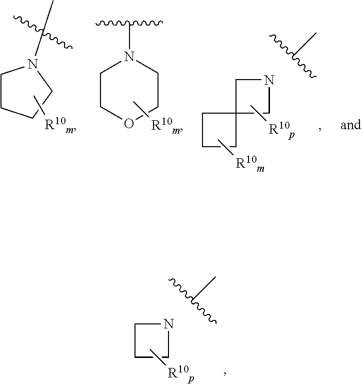
m is 0, 1, 2 or 3;
p is 0, 1, or 2; and
each R¹⁰ is independently selected from halo and (C₁-C₃)alkyl.

5. The compound of claim 1, wherein R¹ is (C₁-C₃)alkyl, optionally substituted with one or more R³;
R⁸ is selected from —NR₂, and —(O)ₙ-(4- to 7-membered non-aromatic heterocycle), wherein the 4- to 7-membered non-aromatic heterocycle is optionally substituted with one or more of halo and (C₁-C₃)alkyl;
n is 0 or 1; and
each R is independently selected from H, (C₁-C₆)alkyl and (C₁-C₆)haloalkyl.

6. The compound of claim 1, wherein R¹ is —NR⁵R⁶;
R⁵ is selected from H, (C₁-C₆)alkyl, and (C₁-C₆)haloalkyl, preferably H and (C₁-C₆)alkyl, more preferably H and Me; and
R⁶ is selected from H, (C₁-C₆)alkyl, (C₁-C₆)haloalkyl, and 4- to 7-membered non-aromatic heterocycle, wherein the 4- to 7-membered non-aromatic heterocycle is optionally substituted with one or more of halo and (C₁-C₆)alkyl,
preferably R⁶ is a 5- or 6-membered non-aromatic heterocycle optionally substituted with one or more (C₁-C₃)alkyl.

7. The compound of claim 1, wherein R¹ is —OR⁴;
R⁴ is selected from (C₁-C₆)alkyl, (C₁-C₆)haloalkyl, (C₁-C₆)alkylene-NR₂, 4- to 6-membered non-aromatic heterocycle, and 6- to 9-membered heterocyclic spiro system, wherein the 4- to 6-membered non-aromatic heterocycle and 6- to 9-membered heterocyclic spiro system are optionally substituted with one or more of halo and (C₁-C₆)alkyl,
preferably R⁴ is selected from (C₁-C₃)alkylene-NR₂, 4- or 5-membered non-aromatic heterocycle, and 7- to 9-membered heterocyclic spiro system, wherein the 4- or 5-membered non-aromatic heterocycle and 7- to 9-membered heterocyclic spiro system are optionally substituted with one or more of halo and (C₁-C₆)alkyl; and
each R is independently selected from H, (C₁-C₆)alkyl and (C₁-C₆)haloalkyl, preferably each R is H.

8. The compound of claim 1, wherein R¹ is —C(O)R⁷;
R⁷ is selected from H, (C₁-C₆)alkyl, (C₁-C₆)haloalkyl, —NR₂, and 4- to 7-membered non-aromatic heterocycle, wherein the 4- to 7-membered non-aromatic heterocycle is optionally substituted with one or more of halo and (C₁-C₆)alkyl,
preferably wherein R⁷ is a 4- to 7-membered non-aromatic heterocycle, optionally substituted with one or more (C₁-C₃)alkyl; and
each R is independently selected from H, (C₁-C₆)alkyl and (C₁-C₆)haloalkyl.

9. The compound of claim 1, wherein R² is selected from H, halo, (C₁-C₃)alkyl, (C₁-C₃)haloalkyl, (C₁-C₃)alkoxy, (C₁-C₃)haloalkoxy, —NR₂ and —O(C₁-C₆)alkylene-NR₂, preferably H, halo, (C₁-C₃)alkyl, (C₁-C₃)haloalkyl, and —NR₂, more preferably H and halo, most preferably H and F; and
each R is independently selected from H, (C₁-C₆)alkyl and (C₁-C₆)haloalkyl.

10. The compound of claim 1, wherein R³ is selected from
(i) H, halo, (C₁-C₆)alkyl, (C₁-C₆)haloalkyl, —OR⁴
(ii) 5- to 7-membered non-aromatic heterocycle optionally substituted with one or more R⁹; and
(iii) 8- to 10-membered spiro or fused heterocyclic system optionally substituted with one or more of halo and (C₁-C₃)alkyl;
R⁴ is selected from (C₁-C₆)alkylene-NR₂, 4- to 6-membered non-aromatic heterocycle, and 6- to 8-membered heterocyclic spiro system, wherein each of the 4- to 6-membered non-aromatic heterocycle or 6- to 8-membered heterocyclic spiro system is optionally substituted with one or more of (C₁-C₃)alkyl or halo;
R⁹ is selected from halo, (C₁-C₃)alkyl, —NR₂, —NR((C₁-C₃)hydroxyalkyl), and (C₁-C₃)alkylene-NR₂; and
each R is independently selected from H, (C₁-C₆)alkyl and (C₁-C₆)haloalkyl.

11. The compound of claim 1, wherein one X is N or CH.

12. The compound of claim 1, wherein the compound is of any of Formulae (II) to (VIII): 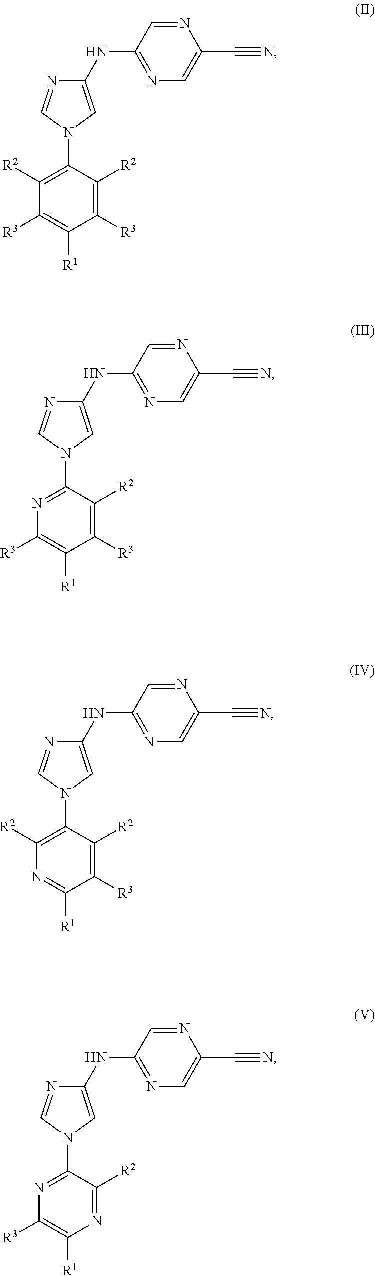 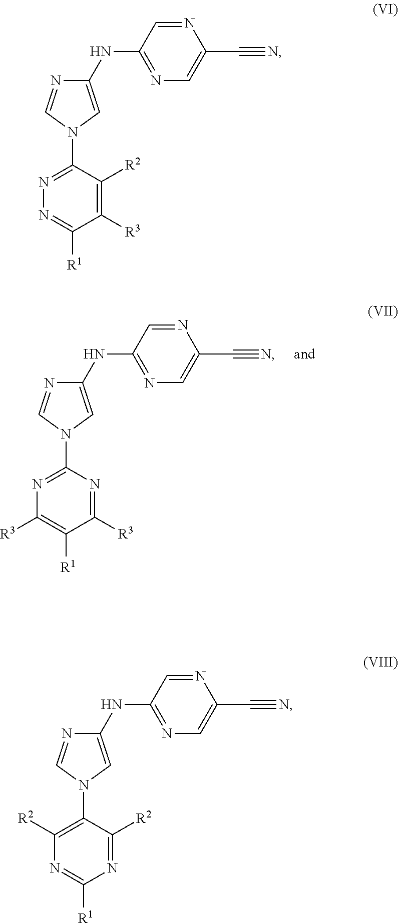
or a pharmaceutically acceptable salt, solvate, hydrate, tautomer, enantiomer, diastereoisomer, isotopic form, N-oxide, or prodrug thereof.

13. The compound of claim 1, wherein the compound is;
5-((1-(4-(6-Methyl-3,6-diazabicyclo[3.2.0]heptan-3-yl)phenyl)-1H-imidazol-4-yl)amino)pyrazine-2-carbonitrile
5-((1-(3-Chloro-4-((3-fluoropyrrolidin-1-yl)methyl)phenyl)-1H-imidazol-4-yl)amino)pyrazine-2-carbonitrile
5-((1-(2-Methoxy-4-(4-methylpiperazin-1-yl)phenyl)-1H-imidazol-4-yl)amino)pyrazine-2-carbonitrile
5-((1-(4-(4-Fluoro-1-methylpiperidin-4-yl)phenyl)-1H-imidazol-4-yl)amino)pyrazine-2-carbonitrile
5-((1-(2-Fluoro-4-((3-fluoropyrrolidin-1-yl)methyl)-6-methoxyphenyl)-1H-imidazol-4-yl)amino)pyrazine-2-carbonitrile
5-((1-(2-Fluoro-4-((3-fluoropyrrolidin-1-yl)methyl)phenyl)-1H-imidazol-4-yl)amino)pyrazine-2-carbonitrile
5-((1-(4-((4-Methylpiperazin-1-yl)methyl)phenyl)-1H-imidazol-4-yl)amino)pyrazine-2-carbonitrile
5-((1-(3-(4-Methylpiperazin-1-yl)phenyl)-1H-imidazol-4-yl)amino)pyrazine-2-carbonitrile
5-((1-(4-(5-Methyl-2,5-diazabicyclo[2.2.1]heptan-2-yl)phenyl)-1H-imidazol-4-yl)amino)pyrazine-2-carbonitrile
5-((1-(4-(6-Methyl-2,6-diazaspiro[3.3]heptan-2-yl)phenyl)-1H-imidazol-4-yl)amino)pyrazine-2-carbonitrile
5-((1-(4-(3-(Dimethylamino)pyrrolidin-1-yl)phenyl)-1H-imidazol-4-yl)amino)pyrazine-2-carbonitrile
5-((1-(2-Methoxy-4-((1-methylpiperidin-4-yl)amino)phenyl)-1H-imidazol-4-yl)amino)pyrazine-2-carbonitrile
5-((1-(2-Methoxy-4-(methyl(1-methylpiperidin-4-yl)amino)phenyl)-1H-imidazol-4-yl)amino)pyrazine-2-carbonitrile
5-((1-(4-(3-(Dimethylamino)pyrrolidin-1-yl)phenyl)-1H-imidazol-4-yl)amino)pyrazine-2-carbonitrile
5-((1-(4-(2,4-Dimethylpiperazin-1-yl)phenyl)-1H-imidazol-4-yl)amino)pyrazine-2-carbonitrile
5-((1-(5-(4-Methylpiperazin-1-yl)pyridin-2-yl)-1H-imidazol-4-yl)amino)pyrazine-2-carbonitrile
5-((1-(6-(4-Methylpiperazin-1-yl)pyridin-3-yl)-1H-imidazol-4-yl)amino)pyrazine-2-carbonitrile
5-((1-(6-(4-methyl-1,4-Diazepan-1-yl)pyridin-3-yl)-1H-imidazol-4-yl)amino)pyrazine-2-carbonitrile
5-((1-(3-Fluoro-4-(4-methylpiperazin-1-yl)phenyl)-1H-imidazol-4-yl)amino)pyrazine-2-carbonitrile
5-((1-(4-(Methyl(1-methylpiperidin-4-yl)amino)phenyl)-1H-imidazol-4-yl)amino)pyrazine-2-carbonitrile
5-((1-(2-Fluoro-4-(4-methylpiperazin-1-yl)phenyl)-1H-imidazol-4-yl)amino)pyrazine-2-carbonitrile
5-((1-(4-(((4-Fluoro-1-methylpyrrolidin-3-yl)oxy)methyl)phenyl)-1H-imidazol-4-yl)amino)pyrazine-2-carbonitrile
5-((1-(2-Chloro-4-(4-methylpiperazin-1-yl)phenyl)-1H-imidazol-4-yl)amino)pyrazine-2-carbonitrile
5-((1-(4-((1-Methylpiperidin-4-yl)amino)phenyl)-1H-imidazol-4-yl)amino)pyrazine-2-carbonitrile
5-((1-(4-(4-Ethylpiperazin-1-yl)-2-methoxyphenyl)-1H-imidazol-4-yl)amino)pyrazine-2-carbonitrile
5-((1-(3-Methyl-4-(4-methylpiperazin-1-yl)phenyl)-1H-imidazol-4-yl)amino)pyrazine-2-carbonitrile
5-((1-(4-(4-Methylpiperazin-1-yl)-2-(trifluoromethoxy)phenyl)-1H-imidazol-4-yl)amino)pyrazine-2-carbonitrile
5-((1-(4-(Hexahydropyrrolo[1,2-a]pyrazin-2(1H)-yl)-2-methoxyphenyl)-1H-imidazol-4-yl)amino)pyrazine-2-carbonitrile
5-((1-(4-(2,4-Dimethylpiperazin-1-yl)-2-methoxyphenyl)-1H-imidazol-4-yl)amino)pyrazine-2-carbonitrile
5-((1-(4-(3-(Dimethylamino)azetidin-1-yl)phenyl)-1H-imidazol-4-yl)amino)pyrazine-2-carbonitrile
5-((1-(3-(4-Methyl-1,4-diazepan-1-yl)phenyl)-1H-imidazol-4-yl)amino)pyrazine-2-carbonitrile
5-((1-(3-(5-Methyl-2,5-diazaspiro[3.4]octan-2-yl)phenyl)-1H-imidazol-4-yl)amino)pyrazine-2-carbonitrile
5-((1-(3-(Hexahydropyrrolo[1,2-a]pyrazin-2(1H)-yl)phenyl)-1H-imidazol-4-yl)amino)pyrazine-2-carbonitrile
5-((1-(2-Methoxy-4-(methyl((1-methylpiperidin-4-yl)methyl)amino)phenyl)-1H-imidazol-4-yl)amino)pyrazine-2-carbonitrile
5-((1-(4-(4-(Dimethylamino)piperidin-1-yl)phenyl)-1H-imidazol-4-yl)amino)pyrazine-2-carbonitrile
5-((1-(3-(3-(Dimethylamino)pyrrolidin-1-yl)phenyl)-1H-imidazol-4-yl)amino)pyrazine-2-carbonitrile
5-((1-(3-(8-Methyl-2,8-diazaspiro[4.5]decan-2-yl)phenyl)-1H-imidazol-4-yl)amino)pyrazine-2-carbonitrile
5-((1-(4-(4-Ethyl-2-methylpiperazin-1-yl)phenyl)-1H-imidazol-4-yl)amino)pyrazine-2-carbonitrile
5-((1-(4-((1-Ethylpiperidin-4-yl)amino)-2-methoxyphenyl)-1H-imidazol-4-yl)amino)pyrazine-2-carbonitrile
5-((1-(4-(5-Methyl-2,5-diazabicyclo[2.2.2]octan-2-yl)phenyl)-1H-imidazol-4-yl)amino)pyrazine-2-carbonitrile
5-((1-(3-(Methyl(1-methylpiperidin-4-yl)amino)phenyl)-1H-imidazol-4-yl)amino)pyrazine-2-carbonitrile
5-((1-(2-Methoxy-4-(-5-methyl-2,5-diazabicyclo[2.2.1]heptan-2-yl)phenyl)-1H-imidazol-4-yl)amino)pyrazine-2-carbonitrile
5-((1-(4-((1-(tert-Butyl)azetidin-3-yl)oxy)phenyl)-1H-imidazol-4-yl)amino)pyrazine-2-carbonitrile
5-((1-(2-Methoxy-4-(6-methyl-2,6-diazaspiro[3.3]heptan-2-yl)phenyl)-1H-imidazol-4-yl)amino)pyrazine-2-carbonitrile
5-((1-(4-(3-(Dimethylamino)pyrrolidin-1-yl)-2-methoxyphenyl)-1H-imidazol-4-yl)amino)pyrazine-2-carbonitrile
5-((1-(3-((1-Propylazetidin-3-yl)oxy)phenyl)-1H-imidazol-4-yl)amino)pyrazine-2-carbonitrile
5-((1-(4-((1-Propylazetidin-3-yl)oxy)phenyl)-1H-imidazol-4-yl)amino)pyrazine-2-carbonitrile
5-((1-(5-(3-(Dimethylamino)pyrrolidin-1-yl)pyridin-2-yl)-1H-imidazol-4-yl)amino)pyrazine-2-carbonitrile
5-((1-(4-([1,3′-Bipyrrolidin]-1′-yl)-2-methoxyphenyl)-1H-imidazol-4-yl)amino)pyrazine-2-carbonitrile
5-((1-(5-(3-(Dimethylamino)pyrrolidin-1-yl)pyrazin-2-yl)-1H-imidazol-4-yl)amino)pyrazine-2-carbonitrile
5-((1-(4-((1-(tert-Butyl)azetidin-3-yl)oxy)-2-methoxyphenyl)-1H-imidazol-4-yl)amino)pyrazine-2-carbonitrile
5-((1-(4-((3-Fluoropyrrolidin-1-yl)methyl)-2-methyl-6-(trifluoromethyl)phenyl)-1H-imidazol-4-yl)amino)pyrazine-2-carbonitrile
5-((1-(2-Methoxy-4-(1-methylhexahydropyrrolo[3,4-b]pyrrol-5(1H)-yl)phenyl)-1H-imidazol-4-yl)amino)pyrazine-2-carbonitrile
5-((1-(3-((-4-fluoro-1-methylpyrrolidin-3-yl)oxy)phenyl)-1H-imidazol-4-yl)amino)pyrazine-2-carbonitrile
5-((1-(4-((3-Fluoropyrrolidin-1-yl)methyl)-2,6-dimethylphenyl)-1H-imidazol-4-yl)amino)pyrazine-2-carbonitrile
5-((1-(3-(3-(Dimethylamino)pyrrolidin-1-yl)-4-methylphenyl)-1H-imidazol-4-yl)amino)pyrazine-2-carbonitrile
5-((1-(5-(3-(Dimethylamino)pyrrolidin-1-yl)-6-methylpyridin-2-yl)-1H-imidazol-4-yl)amino)pyrazine-2-carbonitrile
5-((1-(4-(8-Methyl-2,8-diazaspiro[4.5]decan-2-yl)phenyl)-1H-imidazol-4-yl)amino)pyrazine-2-carbonitrile
5-((1-(4-(1-Methyl-1,6-diazaspiro[3.3]heptan-6-yl)phenyl)-1H-imidazol-4-yl)amino)pyrazine-2-carbonitrile
5-((1-(4-(-3-(Dimethylamino)-4-fluoropyrrolidin-1-yl)phenyl)-1H-imidazol-4-yl)amino)pyrazine-2-carbonitrile
5-((1-(6-(3-(Dimethylamino)pyrrolidin-1-yl)pyridin-3-yl)-1H-imidazol-4-yl)amino)pyrazine-2-carbonitrile
5-((1-(4-(3-(Dimethylamino)pyrrolidin-1-yl)-2-fluorophenyl)-1H-imidazol-4-yl)amino)pyrazine-2-carbonitrile
5-((1-(4-(2-((Dimethylamino)methyl)pyrrolidin-1-yl)phenyl)-1H-imidazol-4-yl)amino)pyrazine-2-carbonitrile
5-((1-(4-(-3-Methyl-3,6-diazabicyclo[3.2.0]heptan-6-yl)phenyl)-1H-imidazol-4-yl)amino)pyrazine-2-carbonitrile
5-((1-(4-(2-Methyl-2,6-diazaspiro[3.4]octan-6-yl)phenyl)-1H-imidazol-4-yl)amino)pyrazine-2-carbonitrile
5-((1-(4-(3-(Dimethylamino)pyrrolidin-1-yl)-3-(trifluoromethyl)phenyl)-1H-imidazol-4-yl)amino)pyrazine-2-carbonitrile
5-((1-(4-(3-(Dimethylamino)pyrrolidin-1-yl)-2-(trifluoromethyl)phenyl)-1H-imidazol-4-yl)amino)pyrazine-2-carbonitrile
5-((1-(4-(3-Morpholinopyrrolidin-1-yl)phenyl)-1H-imidazol-4-yl)amino)pyrazine-2-carbonitrile
5-((1-(4-(-3-(Dimethylamino)-4-methylpyrrolidin-1-yl)phenyl)-1H-imidazol-4-yl)amino)pyrazine-2-carbonitrile
5-((1-(4-(-4-(Dimethylamino)-2-methylpyrrolidin-1-yl)phenyl)-1H-imidazol-4-yl)amino)pyrazine-2-carbonitrile
5-((1-(4-(3-(Dimethylamino)-4-methylpyrrolidin-1-yl)phenyl)-1H-imidazol-4-yl)amino)pyrazine-2-carbonitrile
5-((1-(5-(3-(Dimethylamino)pyrrolidin-1-yl)-6-(trifluoromethyl)pyridin-2-yl)-1H-imidazol-4-yl)amino)pyrazine-2-carbonitrile
5-((1-(4-(3-(Dimethylamino)-3-methylpyrrolidin-1-yl)phenyl)-1H-imidazol-4-yl)amino)pyrazine-2-carbonitrile
5-((1-(4-(3-(Dimethylamino)pyrrolidin-1-yl)-3-fluorophenyl)-1H-imidazol-4-yl)amino)pyrazine-2-carbonitrile
5-((1-(2-Fluoro-4-(3-methyl-3,6-diazabicyclo[3.2.0]heptan-6-yl)phenyl)-1H-imidazol-4-yl)amino)pyrazine-2-carbonitrile
5-((1-(5-(3-(Dimethylamino)pyrrolidin-1-yl)pyrimidin-2-yl)-1H-imidazol-4-yl)amino)pyrazine-2-carbonitrile
5-((1-(2-(Dimethylamino)-4-(3-(dimethylamino)pyrrolidin-1-yl)phenyl)-1H-imidazol-4-yl)amino)pyrazine-2-carbonitrile
5-((1-(4-(3-(Bis(methyl-d3)amino)pyrrolidin-1-yl)phenyl)-1H-imidazol-4-yl)amino)pyrazine-2-carbonitrile
5-((1-(5-(-4-(Dimethylamino)-2-methylpyrrolidin-1-yl)pyridin-2-yl)-1H-imidazol-4-yl)amino)pyrazine-2-carbonitrile
5-((1-(4-(-3-(Dimethylamino)-2-methylpyrrolidin-1-yl)phenyl)-1H-imidazol-4-yl)amino)pyrazine-2-carbonitrile
5-((1-(4-(1-Methylhexahydropyrrolo[3,4-b]pyrrol-5(1H)-yl)phenyl)-1H-imidazol-4-yl)amino)pyrazine-2-carbonitrile
5-((1-(4-(3-(Ethylamino)-4-fluoropyrrolidin-1-yl)phenyl)-1H-imidazol-4-yl)amino)pyrazine-2-carbonitrile
5-((1-(4-(3-(Ethyl(methyl)amino)pyrrolidin-1-yl)phenyl)-1H-imidazol-4-yl)amino)pyrazine-2-carbonitrile
5-((1-(5-(3-(Ethyl(methyl)amino)pyrrolidin-1-yl)pyridin-2-yl)-1H-imidazol-4-yl)amino)pyrazine-2-carbonitrile
5-((1-(4-(4-Methylhexahydropyrrolo[3,2-b]pyrrol-1(2H)-yl)phenyl)-1H-imidazol-4-yl)amino)pyrazine-2-carbonitrile
5-((1-(4-(3-Fluoro-4-(methylamino)pyrrolidin-1-yl)phenyl)-1H-imidazol-4-yl)amino)pyrazine-2-carbonitrile
5-((1-(5-(3-((3,3,3-Trifluoropropyl)amino)pyrrolidin-1-yl)pyridin-2-yl)-1H-imidazol-4-yl)amino)pyrazine-2-carbonitrile
5-((1-(4-(Methyl(1-methylpyrrolidin-3-yl)amino)phenyl)-1H-imidazol-4-yl)amino)pyrazine-2-carbonitrile
5-((1-(4-(3-(Isopropylamino)pyrrolidin-1-yl)phenyl)-1H-imidazol-4-yl)amino)pyrazine-2-carbonitrile
5-((1-(4-(3,3,4-Trimethylpiperazin-1-yl)phenyl)-1H-imidazol-4-yl)amino)pyrazine-2-carbonitrile
5-((1-(4-(4-(Azetidin-1-yl)-2-methylpyrrolidin-1-yl)phenyl)-1H-imidazol-4-yl)amino)pyrazine-2-carbonitrile
5-((1-(4-(6-Methyl-1,6-diazaspiro[3.3]heptan-1-yl)phenyl)-1H-imidazol-4-yl)amino)pyrazine-2-carbonitrile
5-((1-(4-(1-Methyl-1,7-diazaspiro[3.5]nonan-7-yl)phenyl)-1H-imidazol-4-yl)amino)pyrazine-2-carbonitrile
5-((1-(4-(5-Methylhexahydropyrrolo[3,4-c]pyrrol-2(1H)-yl)phenyl)-1H-imidazol-4-yl)amino)pyrazine-2-carbonitrile
5-((1-(5-(3-(Azetidin-1-yl)pyrrolidin-1-yl)pyridin-2-yl)-1H-imidazol-4-yl)amino)pyrazine-2-carbonitrile
5-((1-(4-(3-(Azetidin-1-yl)pyrrolidin-1-yl)-2-methoxyphenyl)-1H-imidazol-4-yl)amino)pyrazine-2-carbonitrile
5-((1-(4-(3-(Azetidin-1-yl)pyrrolidin-1-yl)-3-methylphenyl)-1H-imidazol-4-yl)amino)pyrazine-2-carbonitrile
5-((1-(4-(4-Methylmorpholin-2-yl)phenyl)-1H-imidazol-4-yl)amino)pyrazine-2-carbonitrile
5-((1-(3-(2-((Dimethylamino)methyl)morpholino)phenyl)-1H-imidazol-4-yl)amino)pyrazine-2-carbonitrile
5-((1-(3-(3-((2-Hydroxyethyl)(methyl)amino)pyrrolidin-1-yl)phenyl)-1H-imidazol-4-yl)amino)pyrazine-2-carbonitrile
5-((1-(3-(3-(Dimethylamino)piperidin-1-yl)phenyl)-1H-imidazol-4-yl)amino)pyrazine-2-carbonitrile
5-((1-(4-(3-(Azetidin-1-yl)pyrrolidin-1-yl)phenyl)-1H-imidazol-4-yl)amino)pyrazine-2-carbonitrile
5-((1-(4-(3-((Dimethylamino)methyl)pyrrolidin-1-yl)phenyl)-1H-imidazol-4-yl)amino)pyrazine-2-carbonitrile
5-((1-(4-(Hexahydropyrrolo[1,2-a]pyrazin-2(1H)-yl)phenyl)-1H-imidazol-4-yl)amino)pyrazine-2-carbonitrile
5-((1-(4-(6-Methyl-1,6-diazaspiro[3.4]octan-1-yl)phenyl)-1H-imidazol-4-yl)amino)pyrazine-2-carbonitrile
5-((1-(4-(3-(Dimethylamino)piperidin-1-yl)phenyl)-1H-imidazol-4-yl)amino)pyrazine-2-carbonitrile
5-((1-(4-(3-(3,3-Dimethylazetidin-1-yl)pyrrolidin-1-yl)phenyl)-1H-imidazol-4-yl)amino)pyrazine-2-carbonitrile
5-((1-(4-(3-(3-Methylazetidin-1-yl)pyrrolidin-1-yl)phenyl)-1H-imidazol-4-yl)amino)pyrazine-2-carbonitrile
5-((1-(4-(1-Methyl-1,6-diazaspiro[3.4]octan-6-yl)phenyl)-1H-imidazol-4-yl)amino)pyrazine-2-carbonitrile
5-((1-(4-(3-(3-Fluoroazetidin-1-yl)pyrrolidin-1-yl)phenyl)-1H-imidazol-4-yl)amino)pyrazine-2-carbonitrile
5-((1-(4-(3-(2-Azaspiro[3.3]heptan-2-yl)pyrrolidin-1-yl)phenyl)-1H-imidazol-4-yl)amino)pyrazine-2-carbonitrile
5-((1-(4-(3-(3-Isopropylazetidin-1-yl)pyrrolidin-1-yl)phenyl)-1H-imidazol-4-yl)amino)pyrazine-2-carbonitrile
5-((1-(4-(3-(2-Methylazetidin-1-yl)pyrrolidin-1-yl)phenyl)-1H-imidazol-4-yl)amino)pyrazine-2-carbonitrile
5-((1-(4-(3-(2,2-Dimethylazetidin-1-yl)pyrrolidin-1-yl)phenyl)-1H-imidazol-4-yl)amino)pyrazine-2-carbonitrile 1-(4-(4-((5-Cyanopyrazin-2-yl)amino)-1H-imidazol-1-yl)phenyl)-N,N-dimethylpyrrolidin-3-amine oxide
5-((1-(4-(Morpholinomethyl)phenyl)-1H-imidazol-4-yl)amino)pyrazine-2-carbonitrile
5-((1-(4-(4-Methylpiperazin-1-yl)phenyl)-1H-imidazol-4-yl)amino)pyrazine-2-carbonitrile
5-((1-(4-(4-Methylpiperazine-1-carbonyl)phenyl)-1H-imidazol-4-yl)amino)pyrazine-2-carbonitrile
5-((1-(2-methyl-4-(4-Methylpiperazin-1-yl)phenyl)-1H-imidazol-4-yl)amino)pyrazine-2-carbonitrile
5-((1-(4-([1,3′-Bipyrrolidin]-1′-yl)phenyl)-1H-imidazol-4-yl)amino)pyrazine-2-carbonitrile
5-((1-(3-(3-Aminopropoxy)phenyl)-1H-imidazol-4-yl)amino)pyrazine-2-carbonitrile
5-((1-(2-(3-Aminopropoxy)-6-methoxyphenyl)-1H-imidazol-4-yl)amino)pyrazine-2-carbonitrile
5-((1-(4-(2-Aminoethoxy)phenyl)-1H-imidazol-4-yl)amino)pyrazine-2-carbonitrile
5-((1-(3-((3-Amino-4-fluoropyrrolidin-1-yl)phenyl)-1H-imidazol-4-yl)amino)pyrazine-2-carbonitrile
5-((1-(4-(3-Amino-4-fluoropyrrolidin-1-yl)phenyl)-1H-imidazol-4-yl)amino)pyrazine-2-carbonitrile
5-((1-(4-(3-Aminopyrrolidin-1-yl)phenyl)-1H-imidazol-4-yl)amino)pyrazine-2-carbonitrile
5-((1-(4-(7-Amino-5-azaspiro[2.4]heptan-5-yl)phenyl)-1H-imidazol-4-yl)amino)pyrazine-2-carbonitrile
5-((1-(4-(3-(Methylamino)pyrrolidin-1-yl)phenyl)-1H-imidazol-4-yl)amino)pyrazine-2-carbonitrile
5-((1-(4-(3-Amino-3-methylpyrrolidin-1-yl)phenyl)-1H-imidazol-4-yl)amino)pyrazine-2-carbonitrile
5-((1-(4-(3-Amino-4-fluoropyrrolidin-1-yl)phenyl)-1H-imidazol-4-yl)amino)pyrazine-2-carbonitrile
5-((1-(4-(3-(Aminomethyl)azetidin-1-yl)phenyl)-1H-imidazol-4-yl)amino)pyrazine-2-carbonitrile
5-((1-(4-(3-(Aminomethyl)-3-fluoroazetidin-1-yl)phenyl)-1H-imidazol-4-yl)amino)pyrazine-2-carbonitrile
5-((1-(4-(1,6-Diazaspiro[3.3]heptan-6-yl)phenyl)-1H-imidazol-4-yl)amino)pyrazine-2-carbonitrile
5-((1-(4-(3,6-Diazabicyclo[3.2.0]heptan-3-yl)phenyl)-1H-imidazol-4-yl)amino)pyrazine-2-carbonitrile
5-((1-(4-(3-(Ethylamino)pyrrolidin-1-yl)phenyl)-1H-imidazol-4-yl)amino)pyrazine-2-carbonitrile
5-((1-(4-(3-Methoxy-4-(methylamino)pyrrolidin-1-yl)phenyl)-1H-imidazol-4-yl)amino)pyrazine-2-carbonitrile
5-((1-(4-(3-Fluoro-4-(methylamino)pyrrolidin-1-yl)-2-methoxyphenyl)-1H-imidazol-4-yl)amino)pyrazine-2-carbonitrile
5-((1-(4-(4-Amino-2-methylpyrrolidin-1-yl)phenyl)-1H-imidazol-4-yl)amino)pyrazine-2-carbonitrile
5-((1-(3-(4-Amino-3,3-difluoropiperidin-1-yl)phenyl)-1H-imidazol-4-yl)amino)pyrazine-2-carbonitrile
5-((1-(2-(3-Aminopropoxy)phenyl)-1H-imidazol-4-yl)amino)pyrazine-2-carbonitrile
5-((1-(4-((3-Fluoropyrrolidin-1-yl)methyl)-2-methoxyphenyl)-1H-imidazol-4-yl)amino)pyrazine-2-carbonitrile)
5-((1-(4-((Ethylamino)methyl)-2-methoxyphenyl)-1H-imidazol-4-yl)amino)pyrazine-2-carbonitrile
5-((1-(2-Methoxy-4-(pyrrolidin-1-ylmethyl)phenyl)-1H-imidazol-4-yl)amino)pyrazine-2-carbonitrile
5-((1-(4-((4,4-Difluoropiperidin-1-yl)methyl)-2-methoxyphenyl)-1H-imidazol-4-yl)amino)pyrazine-2-carbonitrile
5-((1-(2-Methoxy-4-(morpholinomethyl)phenyl)-1H-imidazol-4-yl)amino)pyrazine-2-carbonitrile
5-((1-(4-((4-Fluoropiperidin-1-yl)methyl)phenyl)-1H-imidazol-4-yl)amino)pyrazine-2-carbonitrile
5-((1-(3-((2-Methyl-2-azaspiro[3.3]heptan-6-yl)oxy)phenyl)-1H-imidazol-4-yl)amino)pyrazine-2-carbonitrile
5-((1-(4-(1-Methylazetidin-3-yl)phenyl)-1H-imidazol-4-yl)amino)pyrazine-2-carbonitrile
5-((1-(4-(9-Methyl-3-oxa-7,9-diazabicyclo[3.3.1]nonan-7-yl)phenyl)-1H-imidazol-4-yl)amino)pyrazine-2-carbonitrile
5-((1-(4-((2-Methyl-2-azaspiro[3.3]heptan-6-yl)oxy)phenyl)-1H-imidazol-4-yl)amino)pyrazine-2-carbonitrile
5-((1-(4-(1-(Diethylamino)cyclopropyl)phenyl)-1H-imidazol-4-yl)amino)pyrazine-2-carbonitrile
5-((1-(2-Methoxy-4-((2-methyl-2-azaspiro[3.3]heptan-6-yl)oxy)phenyl)-1H-imidazol-4-yl)amino)pyrazine-2-carbonitrile
5-((1-(4-(4-(3,3-Difluorocyclobutyl)piperazin-1-yl)phenyl)-1H-imidazol-4-yl)amino)pyrazine-2-carbonitrile
5-((1-(4-(4-(Oxetan-3-yl)piperazin-1-yl)phenyl)-1H-imidazol-4-yl)amino)pyrazine-2-carbonitrile
5-((1-(4-(4-(2-Fluoroethyl)piperazin-1-yl)phenyl)-1H-imidazol-4-yl)amino)pyrazine-2-carbonitrile
5-((1-(4-(4-(3,3,3-Trifluoropropyl)piperazin-1-yl)phenyl)-1H-imidazol-4-yl)amino)pyrazine-2-carbonitrile
5-((1-(4-(4-Ethylpiperazin-1-yl)phenyl)-1H-imidazol-4-yl)amino)pyrazine-2-carbonitrile or
5-((1-(4-(4-Isopropylpiperazin-1-yl)phenyl)-1H-imidazol-4-yl)amino)pyrazine-2-carbonitrile,
or a pharmaceutically acceptable salt, solvate, hydrate, tautomer, enantiomer, diastereoisomer, isotopic form, N-oxide, or prodrug thereof.

14. A pharmaceutical composition comprising a compound as defined in claim 1 and an excipient.

15. (canceled)

16. (canceled)

17. (canceled)

18. A method of treating cancer in a subject in need thereof, comprising administering to the subject a therapeutically effective amount of a compound as defined in claim 1.

19. The method of treatment of claim 18, wherein the cancer is CNS cancer, glioblastoma multiforme, glioma, neuroblastoma, medulloblastoma, DIPG, secondary brain tumours, brain metastasis, sarcoma, osterosarcoma, rhabdomyosarcoma, Kaposi's sarcoma, leiomyosarcoma, ovarian cancer, high grade serous ovarian cancer, lung cancer, small cell lung cancer, non-small cell lung cancer, breast cancer, endometrial cancer, prostate cancer, pancreatic cancer, renal cell carcinoma, colorectal cancer, colon cancer, rectal cancer, anal cancer, gastrointestinal cancer, gastric cancer, thyroid cancer, bladder cancer, kidney cancer, melanoma, squamous cancer, squamous cell carcinoma, head and neck cancer, leukaemia, or lymphoma.
